(19) United States
(12) Patent Application Publication
(10) Pub. No.: US 2024/0103367 A1
(43) Pub. Date: Mar. 28, 2024

(54) Onium Salt, Acid Diffusion Inhibitor, Resist Composition, And Patterning Process

(71) Applicant: SHIN-ETSU CHEMICAL CO., LTD., Tokyo (JP)

(72) Inventor: Masahiro FUKUSHIMA, Joetsu-shi (JP)

(73) Assignee: SHIN-ETSU CHEMICAL CO., LTD., Tokyo (JP)

(21) Appl. No.: 18/358,150

(22) Filed: Jul. 25, 2023

(30) Foreign Application Priority Data
Aug. 16, 2022 (JP) 2022-129569

(51) Int. Cl. G03F 7/029; G03F 7/004; G03F 7/038; G03F 7/039; G03F 7/20

(52) U.S. Cl. CPC G03F 7/029; G03F 7/0045; G03F 7/0048; G03F 7/0382; G03F 7/0397; G03F 7/2006

(57) ABSTRACT

The present invention is an onium salt represented by the following general formula (1),

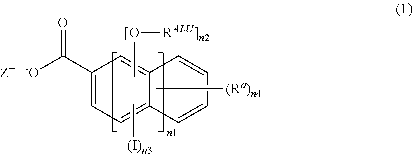

wherein R^{ALU} represents any one of a tertiary ether, tertiary carbonate or acetal formed together with the adjacent oxygen atom; I represents an iodine atom; R^{a} represents a hydrocarbyl group having 1 to 20 carbon atoms and optionally having a heteroatom; and Z⁺ represents an onium cation. This can provide an onium salt used for a resist composition that has high sensitivity, excellent resolution, improved LWR (roughness) and CDU (critical dimension uniformity), and that can inhibit collapse of a resist pattern in lithography.

### TECHNICAL FIELD

The present invention relates to an onium salt, an acid diffusion inhibitor, a resist composition, and a patterning process.

### BACKGROUND ART

As higher integration and higher speed of LSI have been achieved, a pattern rule has been rapidly miniaturized. This is because a high-speed communication with 5G and artificial intelligence (AI) have become widespread, and a high-performance device to process them has been required. As the latest microfabrication technique, 5-nm node devices are industrially manufactured using lithography with extreme ultraviolet ray (EUV) having a wavelength of 13.5 nm. Furthermore, investigation using the EUV lithography is progressed for a 3-nm node device of next generation, and a 2-nm node device of next to the next generation.

The miniaturization in progress causes a problem of blurring an image due to acid diffusion. To achieve resolution with a fine pattern of 45 nm or finer in size, not only improvement of a dissolution contrast, conventionally proposed, but also importance of controlling the acid diffusion is proposed (Non Patent Document 1). However, since a chemically amplified resist composition enhances sensitivity and contrast by utilizing the acid diffusion, inhibiting the acid diffusion to the utmost limit with lowering a temperature or shortening a time of post exposure bake (PEB) considerably deteriorates the sensitivity and the contrast.

Pointed out is a triangle trade-off relationship among sensitivity, resolution, and edge roughness. Although inhibiting the acid diffusion is required in order to improve the resolution, shortening a distance of the acid diffusion deteriorates the sensitivity.

It is effective that an acid generator to generate a bulky acid is added to inhibit the acid diffusion. Accordingly, proposed is containing a repeating unit derived from an onium salt having a polymerizable unsaturated bond into a polymer. In this case, the polymer also functions as an acid generator (polymer-bound acid generator). Patent Document 1 proposes a sulfonium salt and iodonium salt having a polymerizable unsaturated bond to generate a specific sulfonic acid. Patent Document 2 proposes a sulfonium salt in which a sulfonic acid is directly bonded to a main chain.

With an acid-labile group used for a (meth)acrylate polymer for an ArF resist composition, by using a photoacid generator to generate a sulfonic acid having an α-position substituted with a fluorine atom, a deprotection reaction proceeds, however, by using an acid generator to generate a sulfonic acid having an α-position not substituted with a fluorine atom or an acid generator to generate a carboxylic acid, the deprotection reaction does not proceed. By mixing the sulfonium salt or iodonium salt to generate a sulfonic acid having an α-position not substituted with a fluorine atom with the sulfonium salt or iodonium salt to generate the sulfonic acid having an α-position substituted with a fluorine atom, ion exchange proceeds between the sulfonium salt or iodonium salt to generate the sulfonic acid having an α-position not substituted with a fluorine atom and the sulfonic acid having an α-position substituted with a fluorine atom. The photo-generated sulfonic acid having an α-position substituted with a fluorine atom is returned to the sulfonium salt or the iodonium salt with the ion exchange, and thereby the sulfonium salt or iodonium salt of the sulfonic acid having an α-position not substituted with a fluorine atom or a carboxylic acid functions as a quencher. Proposed is a resist composition using a sulfonium salt or iodonium salt to generate a carboxylic acid as a quencher (Patent Document 3).

Various sulfonium salt quenchers to generate a carboxylic acid are proposed. Specifically proposed are sulfonium salts of salicylic acid or a β-hydroxycarboxylic acid (Patent Document 4), a salicylic acid derivative (Patent Documents 5 and 6), fluorosalicylic acid (Patent Document 7), and hydroxynaphthoic acid (Patent Document 8). In particular, salicylic acid has an effect of inhibiting the acid diffusion by virtue of an intramolecular hydrogen bond between the carboxylic acid and the hydroxy group.

Since an iodine atom extremely highly absorbs EUV having a wavelength of 13.5 nm and has an observed effect of generation of a secondary electron from the iodine atom during the exposure, the iodine atom attracts attention in the EUV lithography. Patent Documents 9 and 10 propose a sulfonium salt quencher in which an iodine atom is introduced into benzoic acid or salicylic acid. Although improvement of lithographic performance is observed in a certain degree with this quencher, the iodine atom does not have large solubility in an organic solvent and there is a concern about precipitation in a resist solvent.

With the requirement of further miniaturization, there is a problem of swelling by a developer during, in particular, an alkaline development in a positive-type resist to cause pattern collapse in fine patterning. For such problems in the miniaturization, development of a novel resist composition is important, and desired is development of an onium salt quencher that has good sensitivity, that sufficiently inhibits the acid diffusion, that has excellent solvent solubility, and that effectively inhibits the pattern collapse.

### CITATION LIST

### Patent Literature

Patent Document 1: JP 2006-045311 A
Patent Document 2: JP 2006-178317 A
Patent Document 3: JP 2007-114431 A
Patent Document 4: WO 2018/159560
Patent Document 5: JP 2020-203984 A
Patent Document 6: JP 2020-91404 A
Patent Document 7: JP 2020-91312 A
Patent Document 8: JP 2019-120760 A
Patent Document 9: JP 2022-67056 A
Patent Document 10: JP 6702264 B

### Non Patent Literature

Non Patent Document 1: SPIE Vol. 6520 65203L-1 (2007)

### SUMMARY OF INVENTION

### Technical Problem

The present invention has been made in view of the above circumstances. An object of the present invention is to provide: a novel onium salt used for a resist composition that has high sensitivity and excellent resolution, improved LWR (roughness) and CDU (critical dimension uniformity), and that can inhibit collapse of a resist pattern with both of positive-type and negative-type resists in lithography using far-ultraviolet, EUV, etc.; an acid diffusion inhibitor comprising this onium salt; a resist composition comprising this acid diffusion inhibitor; and a patterning process using this resist composition.

### Solution to Problem

To solve the above problem, the present invention provides an onium salt represented by the following general formula (1),

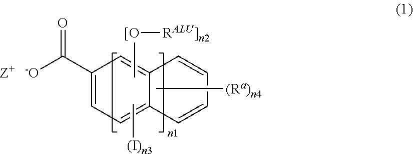

wherein R^{ALU} represents any one of a tertiary ether, tertiary carbonate or acetal formed together with the adjacent oxygen atom, and optionally having a heteroatom; I represents an iodine atom; R^{a} represents a hydrocarbyl group having 1 to 20 carbon atoms and optionally having a heteroatom; n1 represents an integer of 0 or 1; n2 and n3 represent an integer of 1 or 2, when n2 and n3 represent 1, I and —O—R^{ALU} are bonded to carbon atoms adjacent to each other, and when any one or both of n2 and n3 represent 2, one of I and one of —O—R^{ALU} are bonded to carbon atoms adjacent to each other; n4 represents an integer of 0 to 3; when n4≥2, a plurality of R^{a} are optionally bonded to each other to form a cyclic structure together with the carbon atom to which these are bonded; and Z⁺ represents an onium cation.

Such an onium salt is useful as a novel onium salt used for both of positive-type and negative-type resist compositions that has high sensitivity and excellent resolution, that improves LWR and CDU, and that can inhibit collapse of a pattern resist in the lithography. Furthermore, the inventive onium salt can exhibit excellent solvent solubility.

The structure of R^{ALU} in the general formula (1) is preferably represented by the following general formula (ALU-1) or (ALU-2),

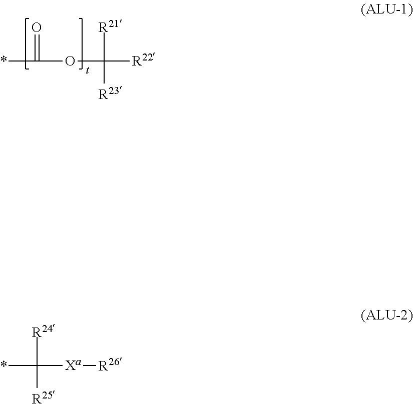

wherein in the formula (ALU-1), R^{21′}, R^{22′}, and R^{23′} each independently represent a hydrocarbyl group having 1 to 12 carbon atoms and optionally having a heteroatom, any two of R^{21′}, R^{22′}, and R^{23′} being optionally bonded to each other to form a ring; “t” represents an integer of 0 or 1; in the formula (ALU-2), R^{24′} and R^{25′} each independently represent a hydrogen atom or a hydrocarbyl group having 1 to 10 carbon atoms; R^{26′} represents a hydrocarbyl group having 1 to 20 carbon atoms, or is optionally bonded to R^{24′} or R^{25′} each other to form a heterocyclic group having 3 to 20 carbon atoms together with X^{a} and the carbon atom to which these are bonded; each of —CH₂— contained in the hydrocarbyl group and the heterocyclic group is optionally substituted with —O— or —S—; X^{a} represents an oxygen atom or a sulfur atom; and “*” represents a bond to the adjacent oxygen atom.

Such an onium salt more favorably works as an acid diffusion inhibitor contained in the resist composition.

Z⁺ in the general formula (1) preferably represents an onium cation represented by any one of the following general formulae (Cation-1) to (Cation-3),

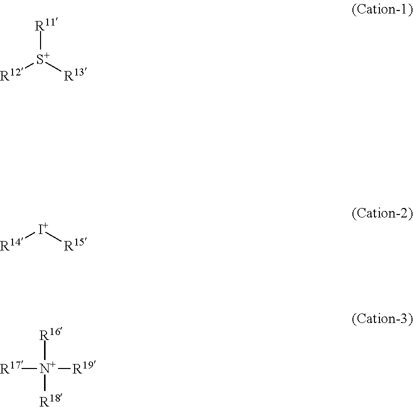

wherein in the formulae (Cation-1) to (Cation-3), R^{11′} to R^{19′} each independently represent a saturated or unsaturated, linear, branched, or cyclic hydrocarbyl group having 1 to 30 carbon atoms and optionally having a heteroatom.

Such an onium salt particularly favorably works as an acid diffusion inhibitor contained in the resist composition.

The present invention also provides an acid diffusion inhibitor comprising the above inventive onium salt.

The inventive onium salt is useful as an acid diffusion inhibitor.

The present invention also provide a resist composition comprising the above inventive acid diffusion inhibitor.

Comprising the above acid diffusion inhibitor yields a good resist composition.

The inventive resist composition preferably further comprises an acid generator to generate an acid.

Such a resist composition allows the above onium salt to function as the acid diffusion inhibitor, and allows the inventive resist composition to function.

The acid generator preferably generates a sulfonic acid, an imide acid, or a methide acid.

Such an acid generator is more suitable for the acid generator.

The inventive resist composition preferably further comprises an organic solvent.

Such a resist composition can dissolve each component, and improves coatability of the composition.

The inventive resist composition preferably further comprises a base polymer.

Such a composition is suitable as the resist composition.

The base polymer preferably comprises a repeating unit represented by the following general formula (a1) and/or a repeating unit represented by the following general formula (a2),

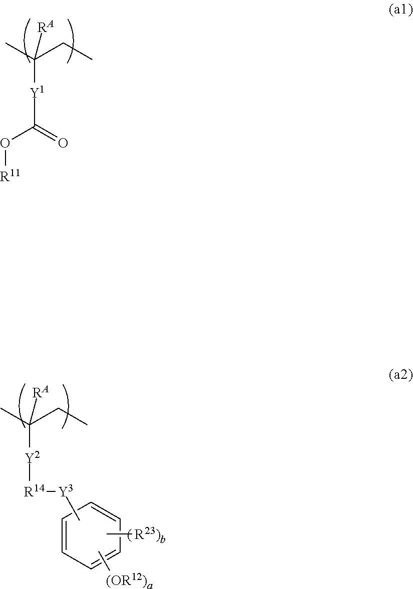

wherein R^{A} each independently represents a hydrogen atom or a methyl group; Y¹ represents a single bond, a phenylene group, a naphthylene group, or a linking group having 1 to 12 carbon atoms and having at least one selected from an ester bond and a lactone ring; Y² represents a single bond or an ester bond; Y³ represents a single bond, an ether bond, or an ester bond; R¹¹ and R¹² each independently represents an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, a part of carbon atoms therein being optionally substituted with an ether bond or an ester bond; “a” represents 1 or 2; “b” represents an integer of 0 to 4, and 1≤a+b≤5.

Such a resist composition, which has the acid-labile group, is suitable as a positive-type resist composition.

The resist composition is preferably a chemically amplified positive-type resist composition.

The inventive resist composition can function as a chemically amplified positive-type resist composition.

Alternatively, the resist composition is preferably a chemically amplified negative-type resist composition.

The inventive resist composition can also function as a chemically amplified negative-type resist composition.

It is also preferable that the base polymer have no acid-labile group.

Such a composition, which has no acid-labile group is suitable as a negative-type resist composition.

The base polymer preferably further comprises at least one selected from repeating units represented by the following general formulae (f1) to (f3),

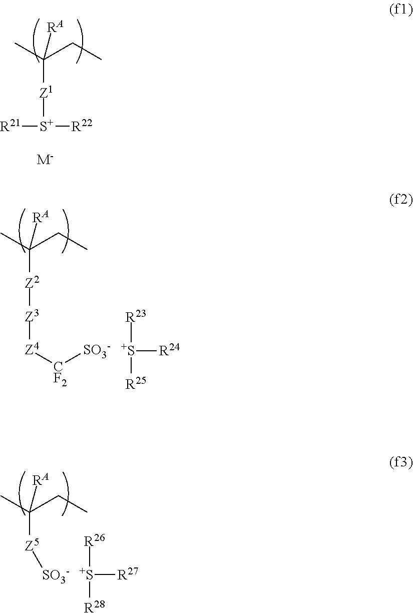

wherein R^{A} each independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)—O—Z¹¹—, or —C(═O)—NH—Z¹¹—; Z¹¹ represents an aliphatic hydrocarbylene group having 1 to 6 carbon atoms, a phenylene group, a naphthylene group, a group having 7 to 18 carbon atoms obtained by combining these groups, Z¹¹ optionally having a carbonyl group, an ester bond, an ether bond, or a hydroxy group; Z² represents a single bond or an ester bond; Z³ represents a single bond, —Z³¹—C(═O)—O—, —Z³¹—O—, or —Z³¹—O—C(═O)—; Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—; Z⁵¹ represents an aliphatic hydrocarbylene group having 1 to 6 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group; R²¹ to R²⁸ each independently represent a halogen atom or a hydrocarbyl group having 1 to 20 carbon atoms and optionally having a heteroatom; R²³ and R²⁴ or R²⁶ and R²⁷ are optionally bonded to each other to form a ring together with the sulfur atom to which these groups are bonded; and M⁻ represents a non-nucleophilic counterion.

Such a repeating unit functions as an acid generator in the base polymer.

The inventive resist composition preferably further comprises a surfactant.

Such a surfactant can improve or regulate coatability of the resist composition.

The present invention also provides a patterning process, comprising steps of: forming a resist film on a substrate by using the above resist composition; exposing the resist film to high-energy ray; and developing the exposed resist film by using a developer.

Such a patterning process can form a good pattern.

KrF excimer laser light, ArF excimer laser light, electron beam, or extreme ultraviolet ray having a wavelength of 3 to 15 nm can be used as the high-energy ray.

Using such high-energy ray can form a better pattern.

### Advantageous Effects of Invention

The inventive novel onium salt favorably functions as the acid diffusion inhibitor (quencher) in a resist composition and has high sensitivity and excellent dissolution contrast, and as a result, the inventive novel onium salt can construct a pattern profile with a small LWR and CDU, excellent rectangularity, and high resolution. Furthermore, a resist composition using the inventive novel onium salt which can inhibit swelling of a resist pattern during alkaline development, can form a pattern hardly collapsed, and is excellent in fine patterning, and a patterning process using this resist composition can be provided.

### DESCRIPTION OF EMBODIMENTS

There have been demands for development of an onium salt quencher that has good sensitivity, sufficiently regulated acid diffusion, and excellent solvent solubility, and that effectively inhibits pattern collapse.

The present inventors have made earnest study to achieve the above object, and consequently found that a resist composition containing an onium salt having a specific structure as an acid diffusion inhibitor has excellent sensitivity and resolution of a resist film, reduced LWR of a line pattern and CDU of a hole pattern and dot pattern, and inhibits swelling during development, which is extremely effective for precise fine patterning. This finding has led to complete the present invention.

Specifically, the present invention is an onium salt represented by the following general formula (1),

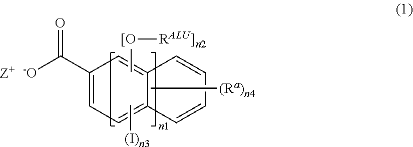

wherein R^{ALU} represents any one of a tertiary ether, tertiary carbonate or acetal formed together with the adjacent oxygen atom, and optionally having a heteroatom; I represents an iodine atom; R^{a} represents a hydrocarbyl group having 1 to 20 carbon atoms and optionally having a heteroatom; n1 represents an integer of 0 or 1; n2 and n3 represent an integer of 1 or 2, when n2 and n3 represent 1, I and —O—R^{ALU} are bonded to carbon atoms adjacent to each other, and when any one or both of n2 and n3 represent 2, one of I and one of —O—R^{ALU} are bonded to carbon atoms adjacent to each other; n4 represents an integer of 0 to 3; when n4≥2, a plurality of R^{a} are optionally bonded to each other to form a cyclic structure together with the carbon atom to which these are bonded; and Z⁺ represents an onium cation.

Hereinafter, the present invention will be described in detail, but the present invention is not limited thereto.

### [Onium Salt]

The inventive onium salt is presented by the following general formula (1),

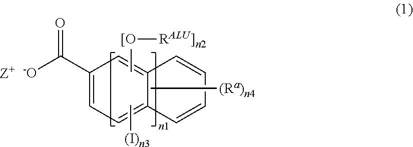

wherein R^{ALU} represents any one of a tertiary ether, tertiary carbonate or acetal formed together with the adjacent oxygen atom, and optionally having a heteroatom; I represents an iodine atom; R^{a} represents a hydrocarbyl group having 1 to 20 carbon atoms and optionally having a heteroatom; n1 represents an integer of 0 or 1; n2 and n3 represent an integer of 1 or 2, when n2 and n3 represent 1, I and —O—R^{ALU} are bonded to carbon atoms adjacent to each other, and when any one or both of n2 and n3 represent 2, one of I and one of —O—R^{ALU} are bonded to carbon atoms adjacent to each other; n4 represents an integer of 0 to 3; when n4≥2, a plurality of R^{a} are optionally bonded to each other to form a cyclic structure together with the carbon atom to which these are bonded; and Z⁺ represents an onium cation.

In the general formula (1), R^{ALU} represents any one of a tertiary ether, tertiary carbonate or acetal formed together with the adjacent oxygen atom, and optionally having a heteroatom. Specifically, R^{ALU} is preferably represented by the following formula (ALU-1) or (ALU-2).

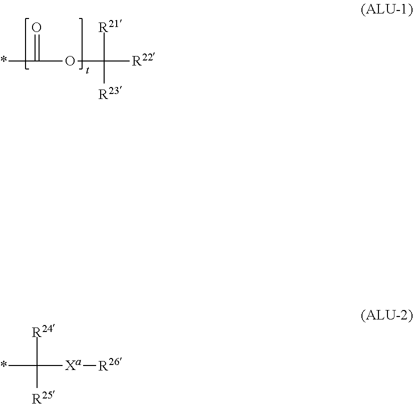

In the general formula (ALU-1), R^{21′}, R^{22′}, and R^{23′} each independently represent a hydrocarbyl group having 1 to 12, preferably 1 to 10, carbon atoms, and any two of R^{21′}, R^{22′}, and R^{23′} are optionally bonded to each other to form a ring. “t” represents an integer of 0 or 1. In the formula (ALU-2), R^{24′} and R^{25′} each independently represent a hydrogen atom or a hydrocarbyl group having 1 to 10 carbon atoms. R^{26′} represents a hydrocarbyl group having 1 to 20 carbon atoms, or is optionally bonded to R^{24′} or R^{25′} each other to form a heterocyclic group having 3 to 20 carbon atoms together with X^{a} and the carbon atom to which these are bonded. Each of —CH₂— contained in the hydrocarbyl group and the heterocyclic group is optionally substituted with —O— or —S—. X^{a} represents an oxygen atom or a sulfur atom. “*” represents a bond to the adjacent oxygen atom.

R^{ALU} in the general formula (1) may have a cyclic structure, or may not have a cyclic structure. When R^{ALU} represented by the general formula (ALU-1) has a cyclic structure and R^{21′}, R^{22′}, and R^{23′} are not bonded to each other to form a ring, at least one of R^{21′}, R^{22′}, and R^{23′} can have a cyclic structure. Similarly, when R^{ALU} represented by the general formula (ALU-2) has a cyclic structure and R^{26′} does not form a heterocyclic group together with R^{24′} or R^{25′}, the carbon atom, and X^{a}, at least one of R^{24′}, R^{25′}, and R^{26′} can have a cyclic structure.

Specific examples of the structure represented by the general formula (ALU-1) include the following structures, but the structure is not limited thereto.

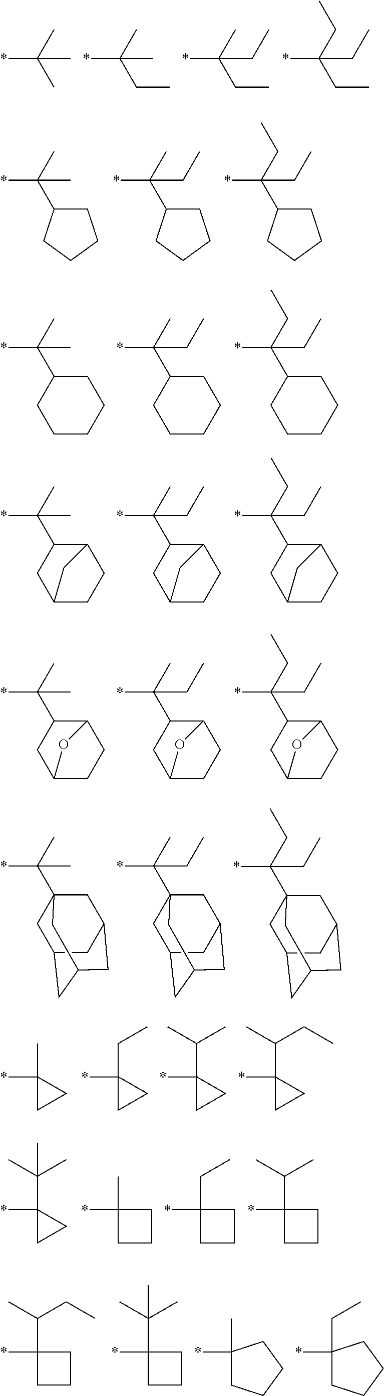

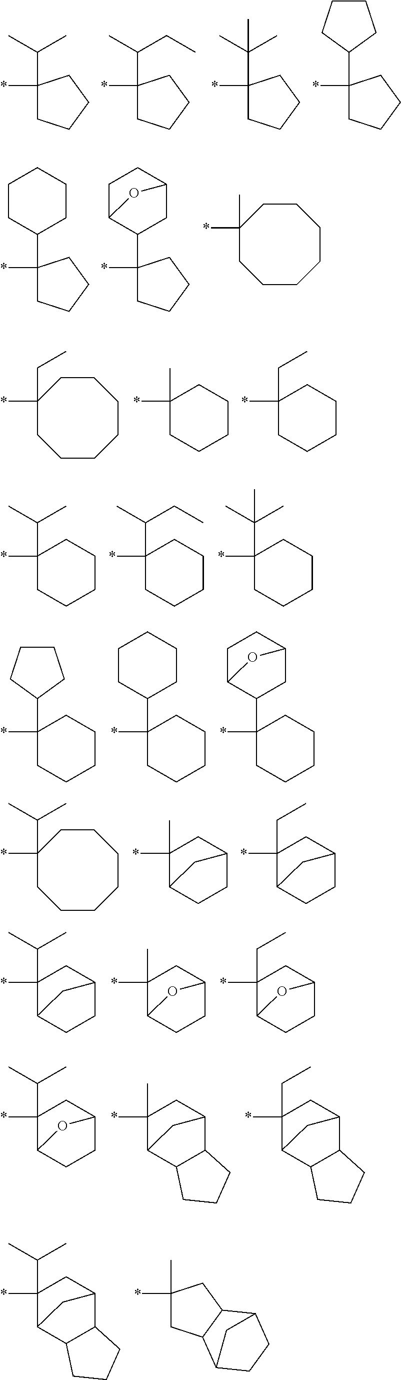

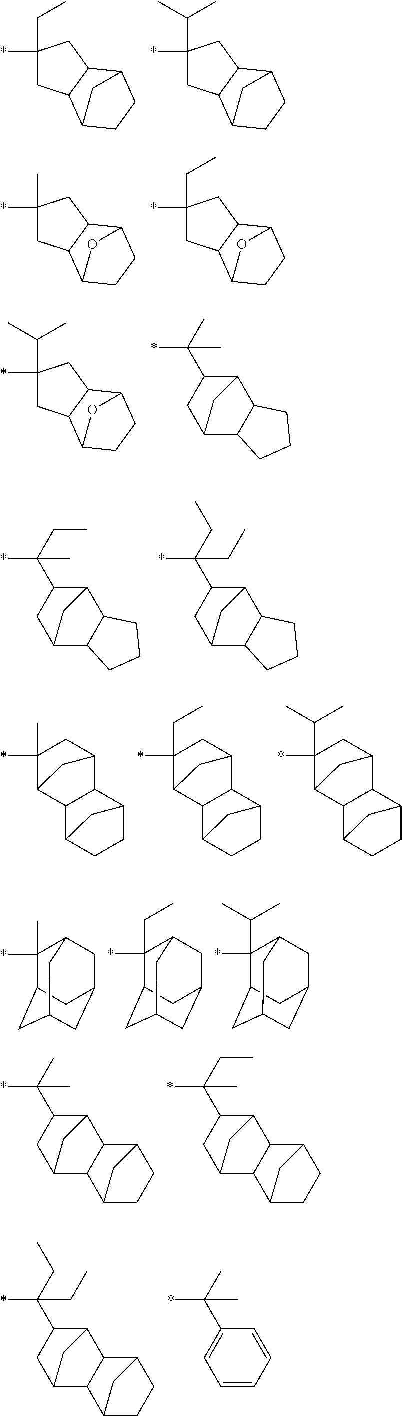

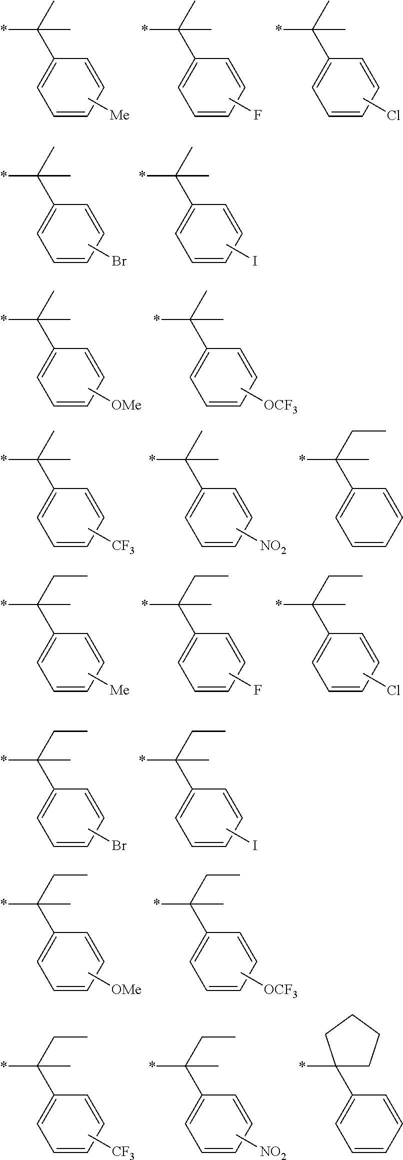

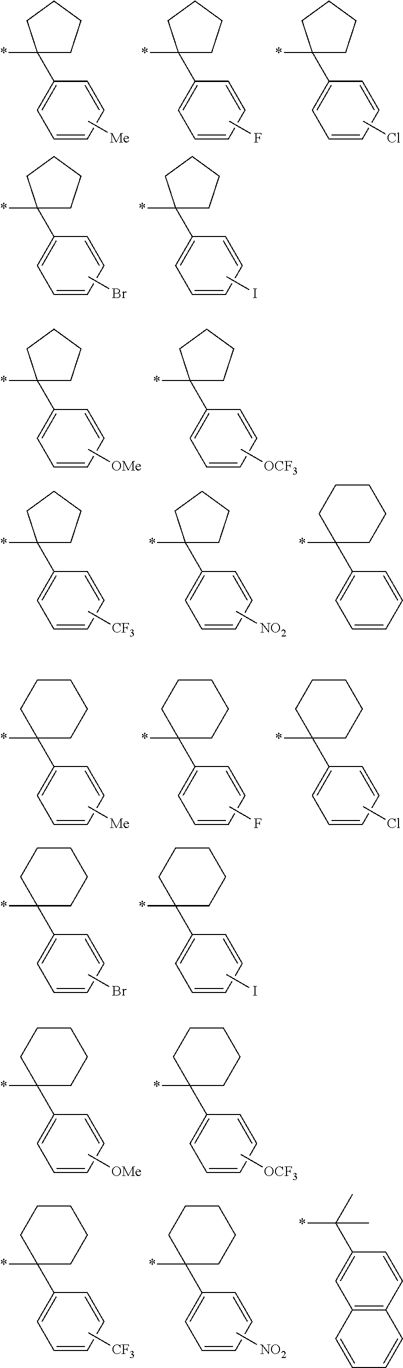

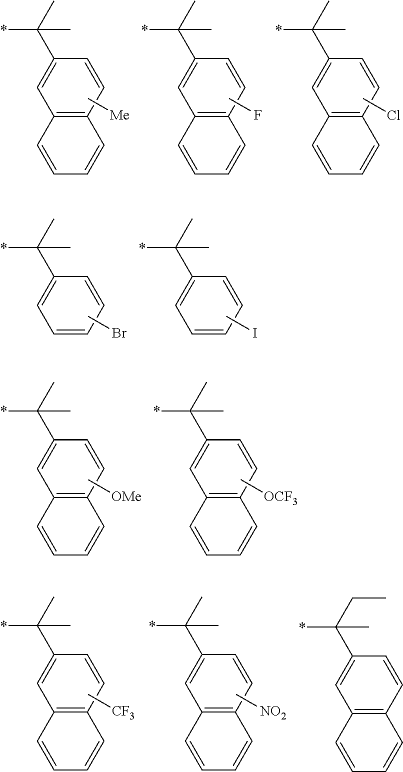

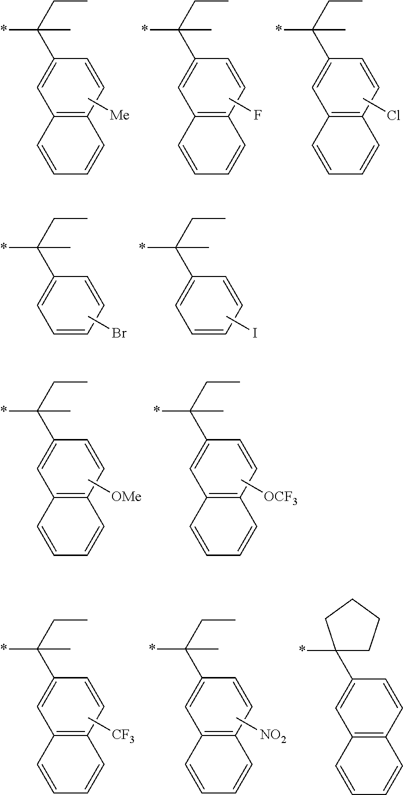

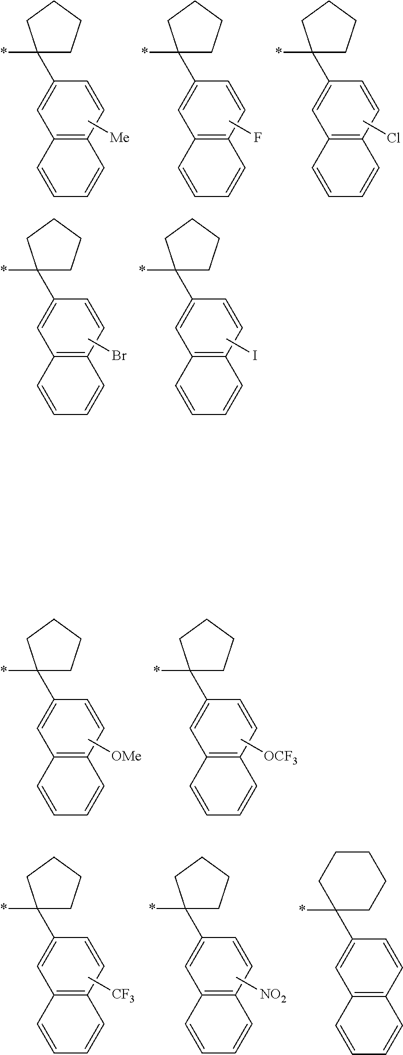

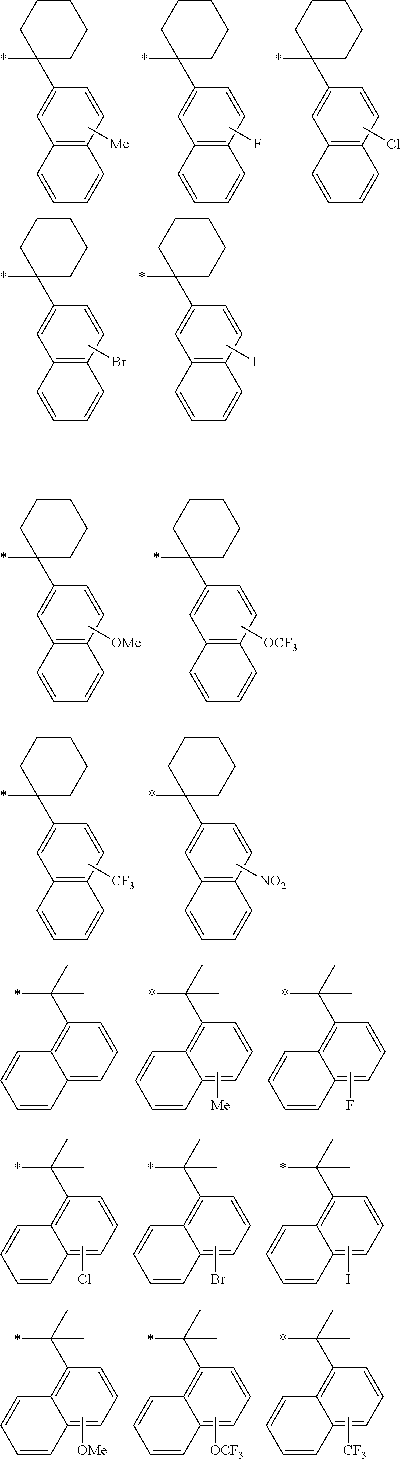

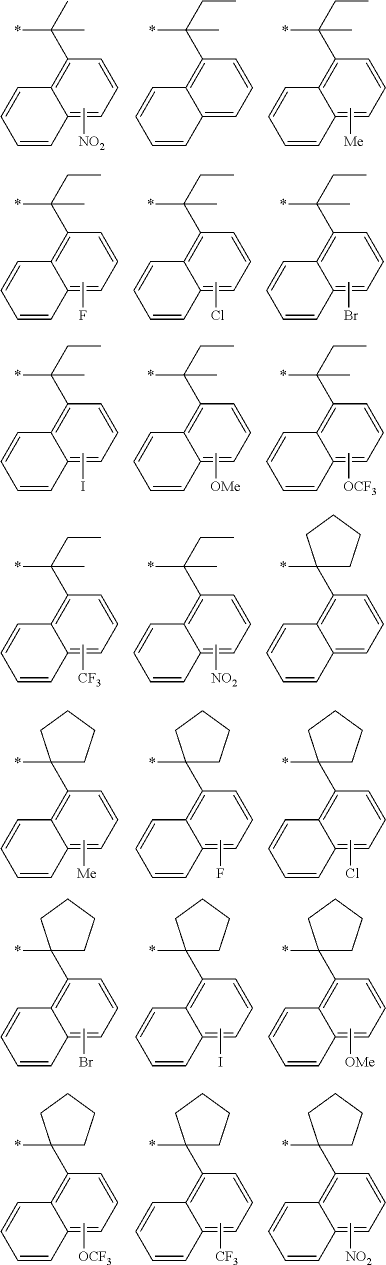

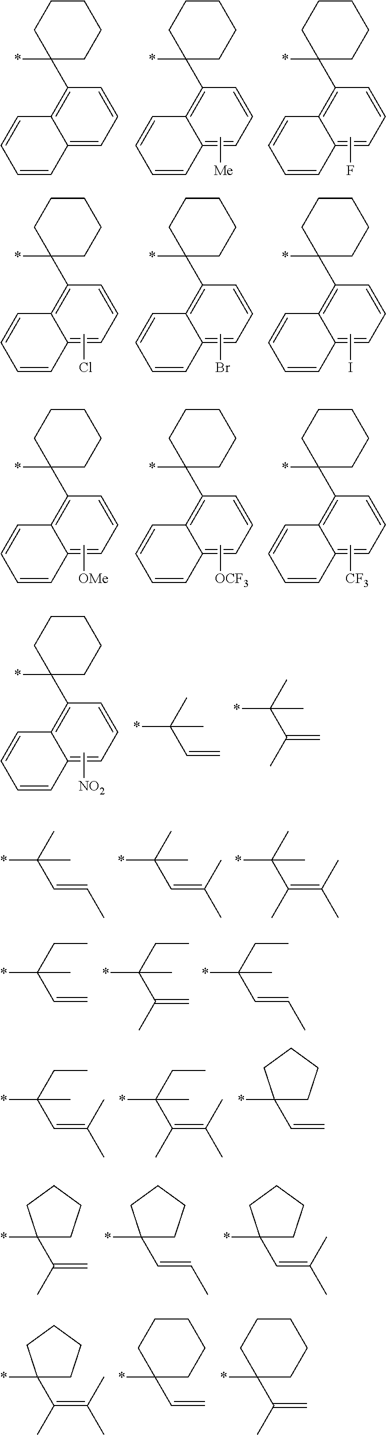

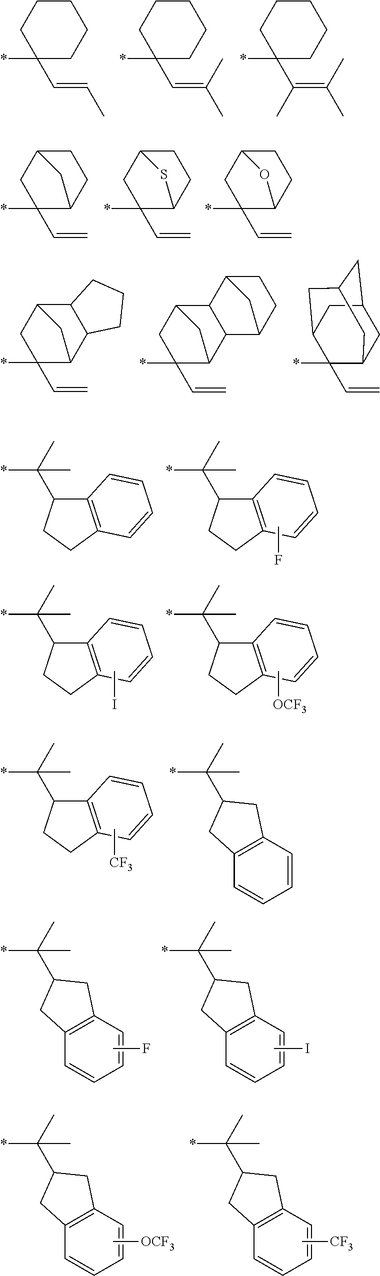

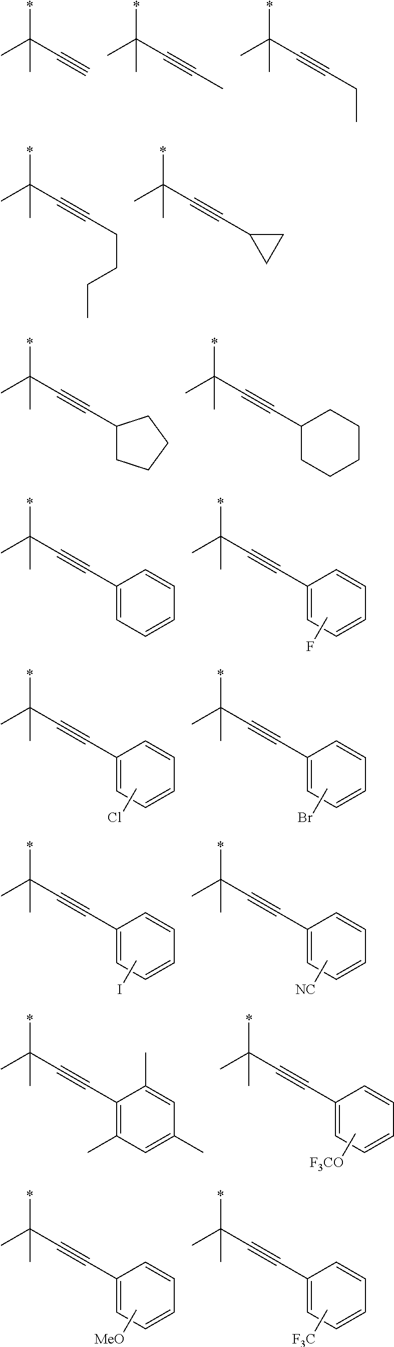

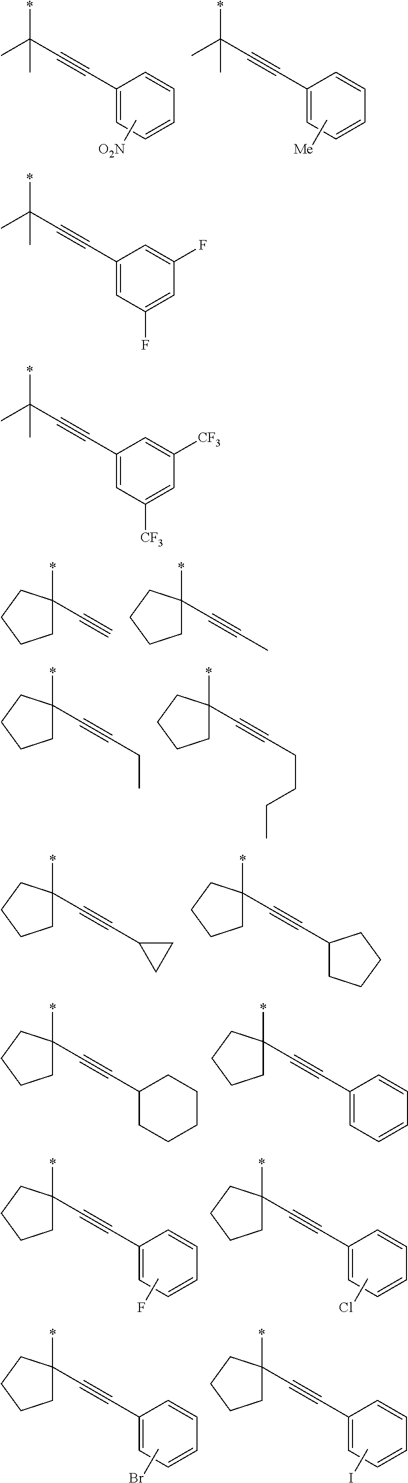

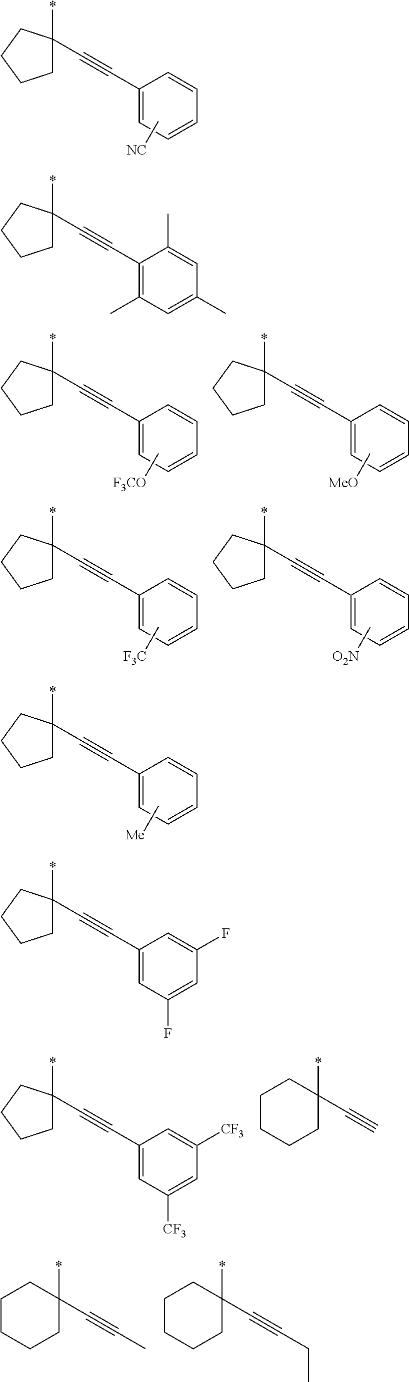

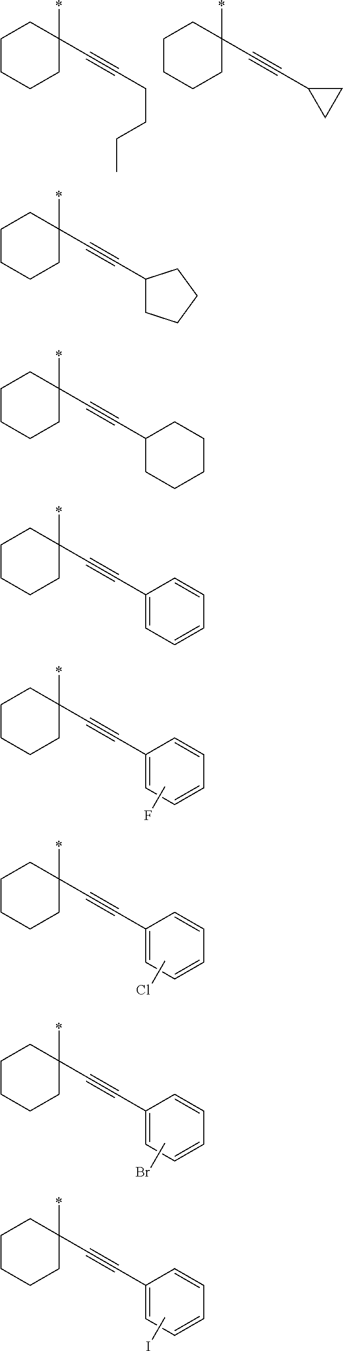

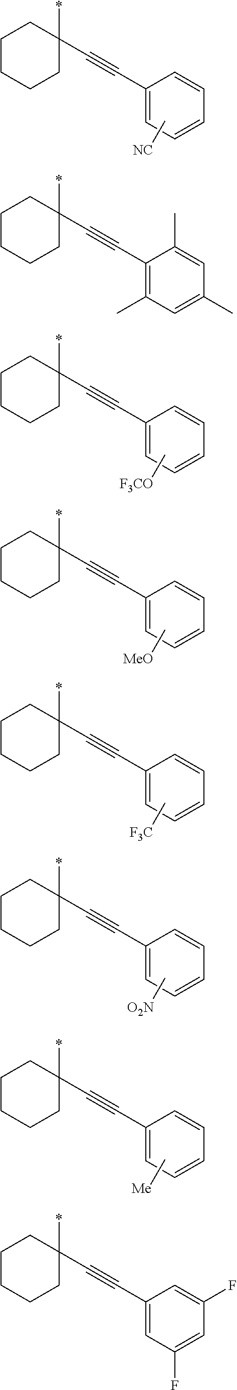

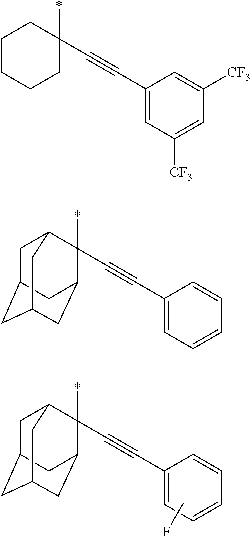

Specific examples of the structure represented by the general formula (ALU-2) include the following structures, but the structure is not limited thereto.

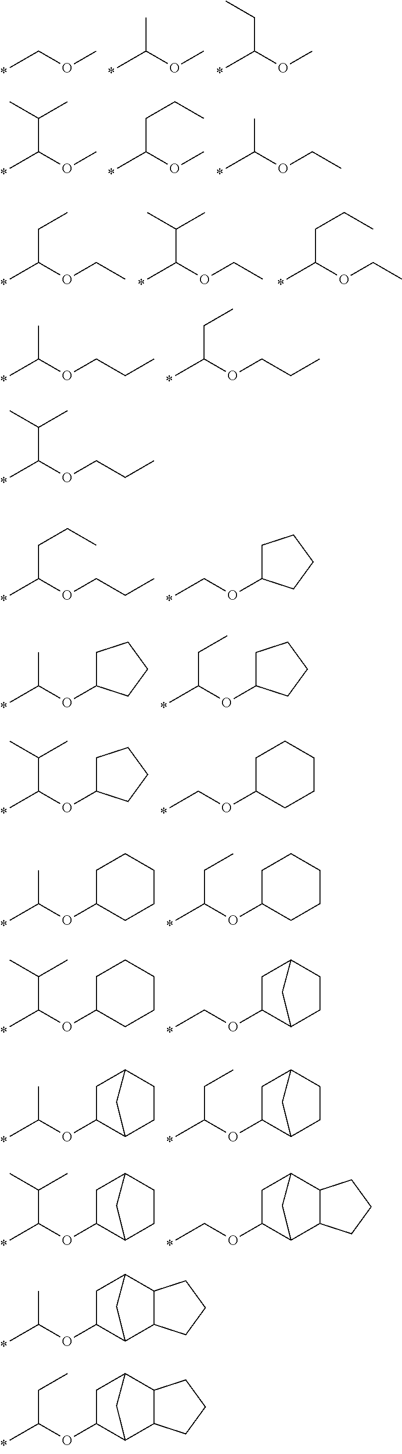

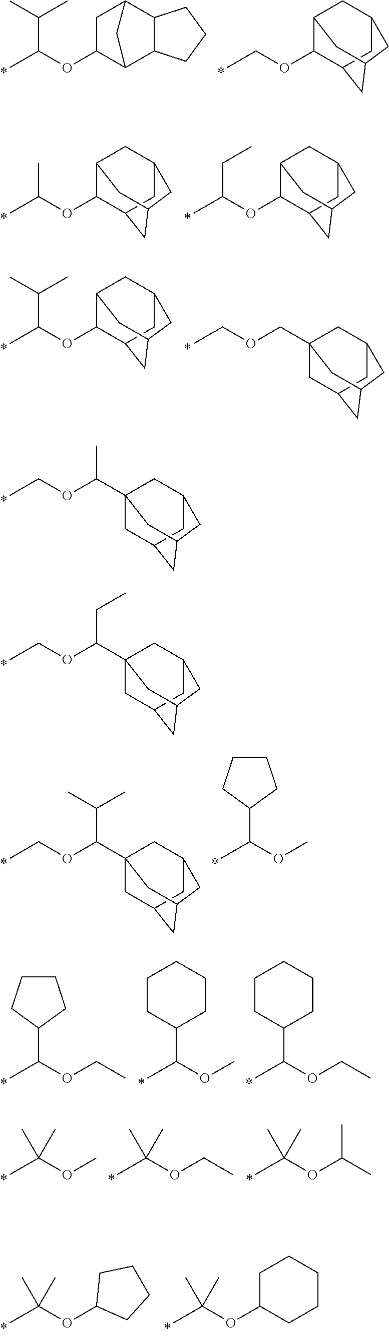

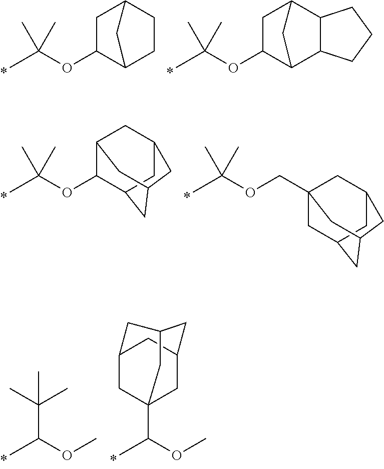

In the general formula (1), I and —O—R^{ALU} should be bonded to carbon atoms adjacent to each other. Specifically, when n2 and n3 represent 1, I and —O—R^{ALU} are bonded to carbon atoms adjacent to each other. When any one or both of n2 and n3 represent 2, one of I and one of —O—R^{ALU} are bonded to carbon atoms adjacent to each other. Since the iodine atom has a large molecular weight, the anion structure having the iodine atom has a feature of small acid diffusion. Furthermore, since an amount of EUV having a wavelength of 13.5 nm absorbed by the iodine atom is extremely high, a secondary electron is generated from the iodine during the exposure to improve the sensitivity. These effects enable to construct the resist with high sensitivity, low LWR, and low CDU. Since the iodine atom also has an electron withdrawing effect, I and —O—R^{ALU} present adjacent to each other increase acidity of an aromatic alcohol generated by eliminating the acid-labile group from —O—R^{ALU} to improve solubility in an alkaline developer.

In the general formula (1), R^{a} represents a hydrocarbyl group having 1 to 20 carbon atoms and optionally having a heteroatom. A part or all of hydrogen atoms in the hydrocarbyl group are optionally substituted with a halogen atom, and —CH₂— constituting the hydrocarbyl group is optionally substituted with —O— or —C(═O)—. The hydrocarbyl group may be a saturated or unsaturated group, and may be any of linear, branched, and cyclic groups. Specific examples thereof include: alkyl groups having 1 to 20 carbon atoms, such as a methyl group, an ethyl group, an n-propyl group, an isopropyl group, an n-butyl group, and a tert-butyl group; cyclic saturated hydrocarbyl groups having 3 to carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group and a naphthyl group; aralkyl groups having 7 to 20 carbon atoms, such as a benzyl group, a 1-phenylethyl group, and a 2-phenylethyl group; and groups obtained by combining these groups. A part or all of hydrogen atoms in the hydrocarbyl group are optionally substituted with a group having a heteroatom such as an oxygen atom, a sulfur atom, a nitrogen atom, and a halogen atom. A part of —CH₂— constituting the hydrocarbyl group is optionally substituted with a group having a heteroatom such as an oxygen atom, a sulfur atom, and a nitrogen atom. As a result, optionally contained is a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride, or a haloalkyl group, etc.

In the general formula (1), n1 represents an integer of 0 or 1. n1=0 represents a benzene ring and n1=1 represents a naphthalene ring. From the viewpoint of the solvent solubility, n1=0 representing a benzene ring is preferable.

In the general formula (1), n2 and n3 represent an integer of 1 or 2. From the viewpoint of availability of a starting material, n2 and n3 each preferably represent an integer of 1.

In the general formula (1), n4 represents an integer of 0 to 3. When n4≥2, a plurality of R^{a} are optionally bonded to each other to form a cyclic structure together with the carbon atom to which these groups are bonded. When the cyclic structure is formed, specific examples of the cyclic structure include five-membered ring and six-membered ring structures.

Examples of the anion of the onium salt represented by the general formula (1) include the following anions, but the anion is not limited thereto. The substitution position of the substituents on the aromatic ring is also not limited thereto as long as the groups with n2 and n3 are present adjacent to each other.

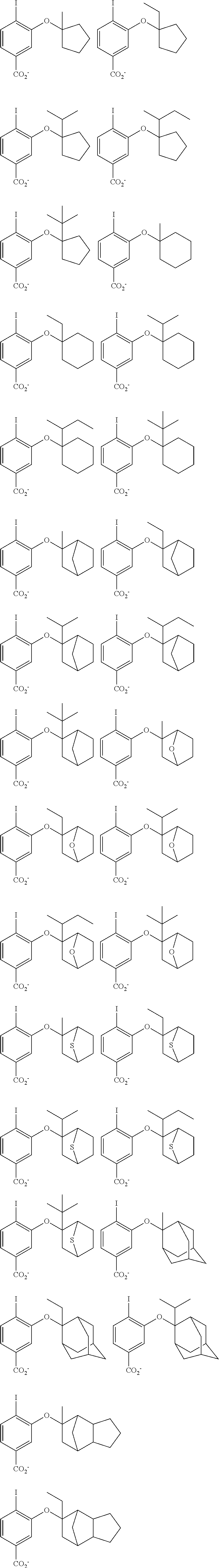

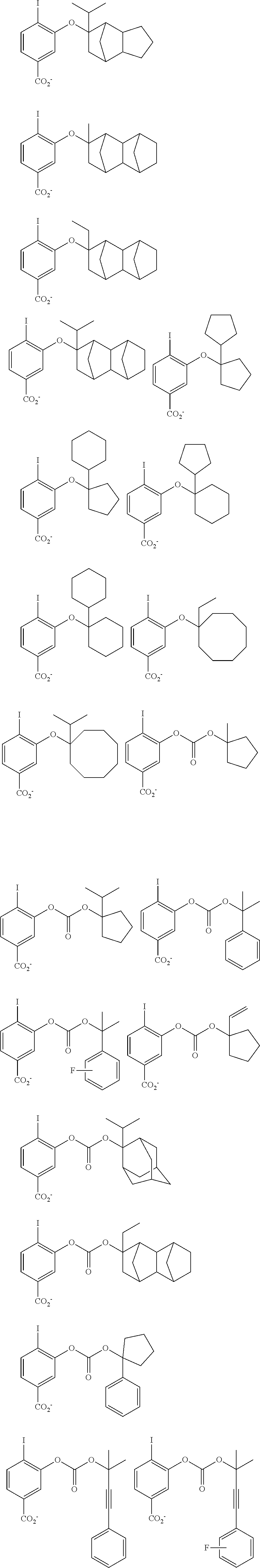

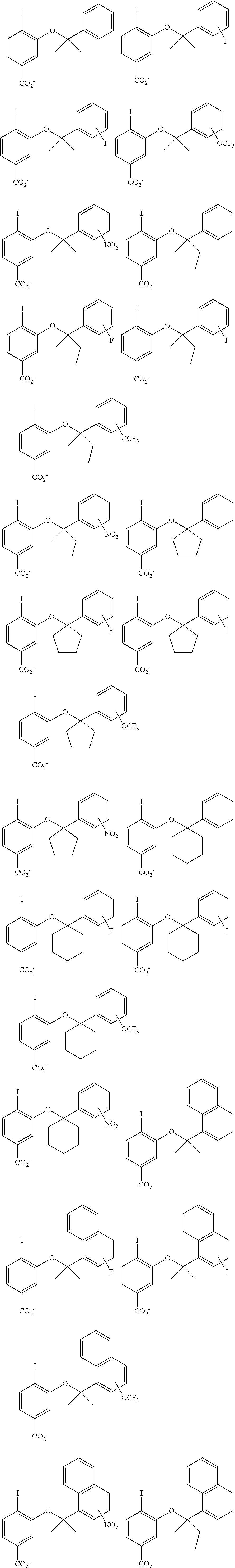

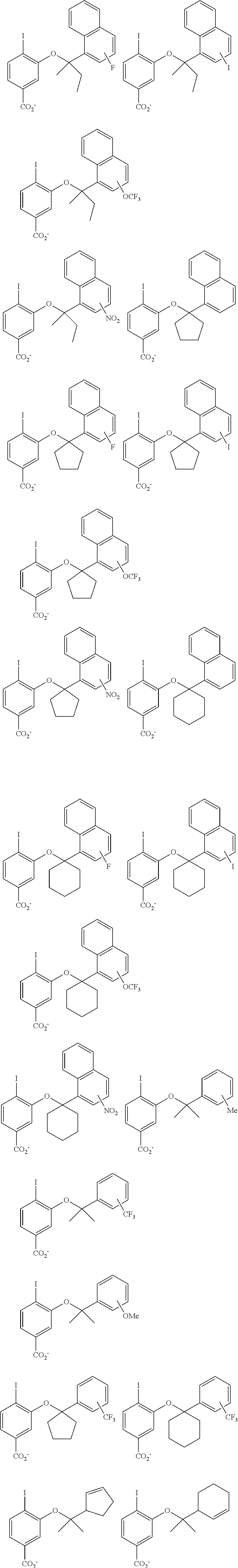

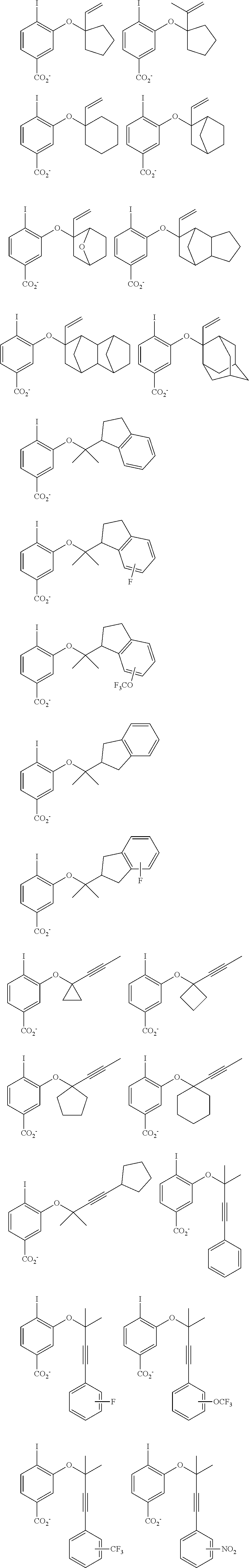

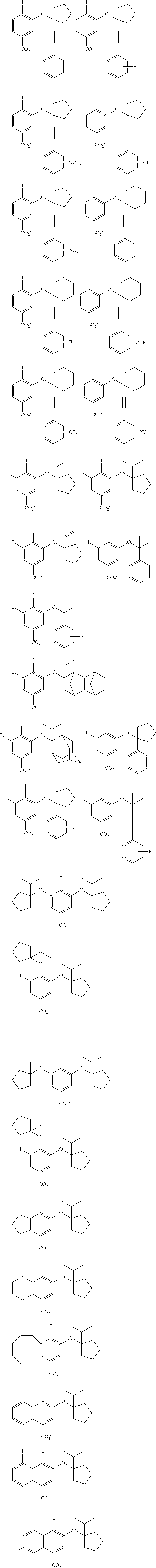

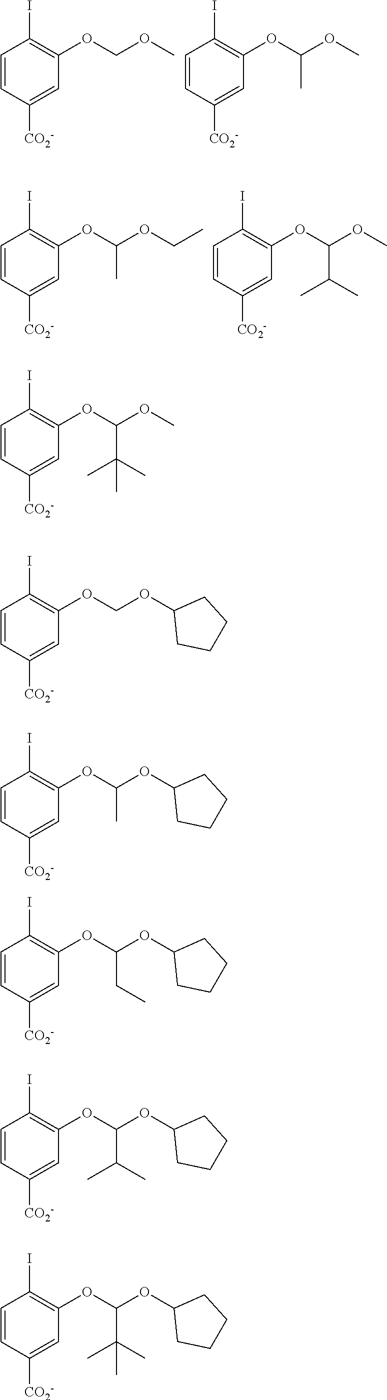

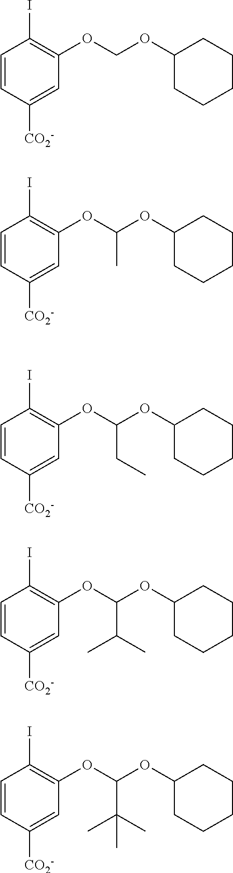

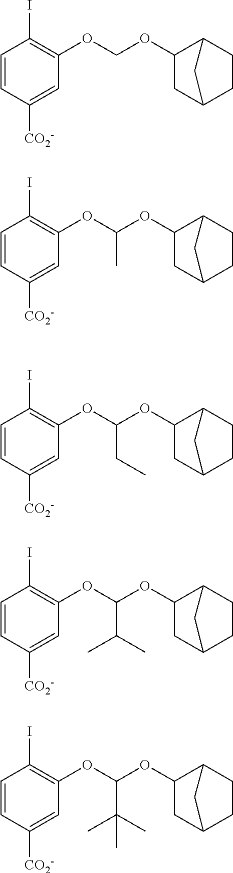

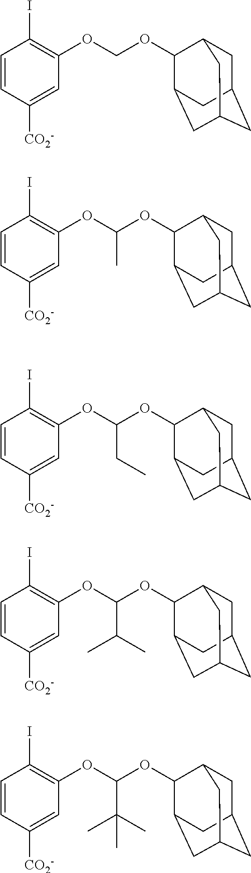

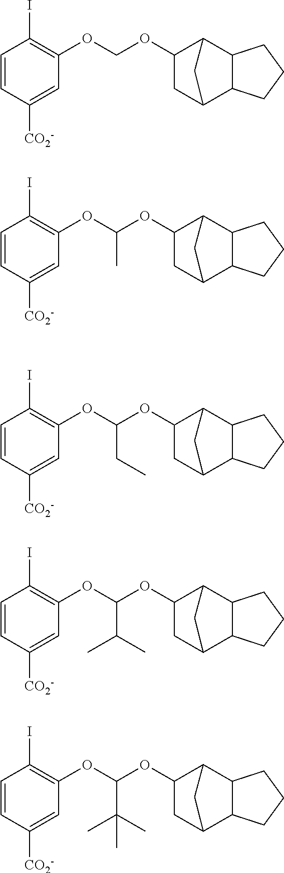

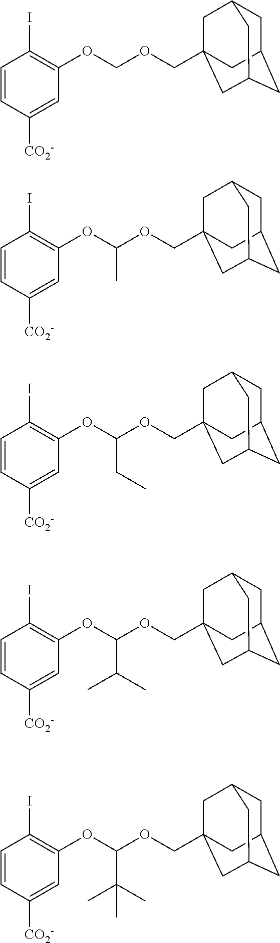

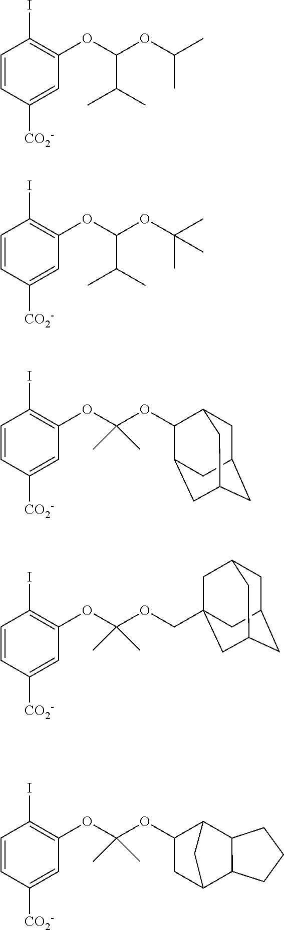

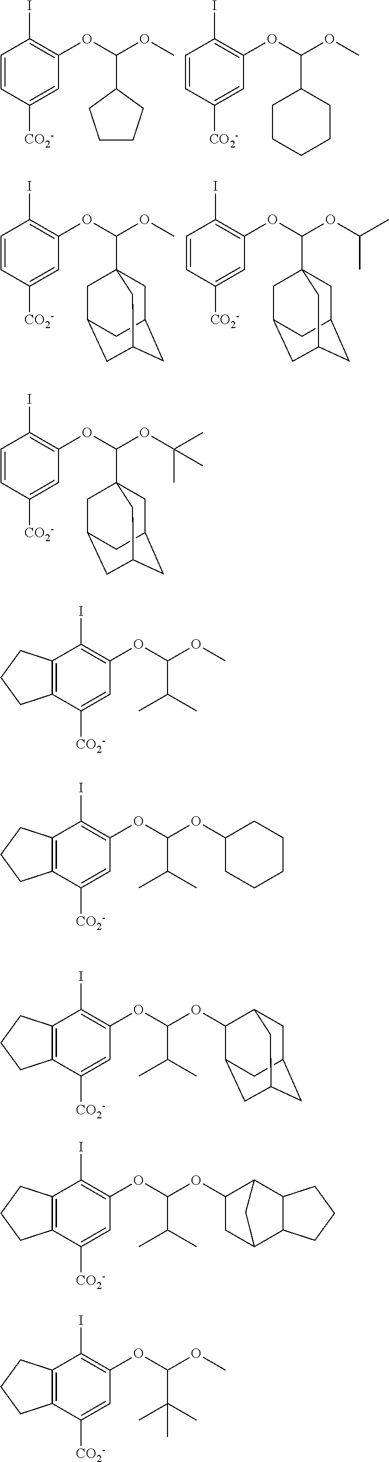

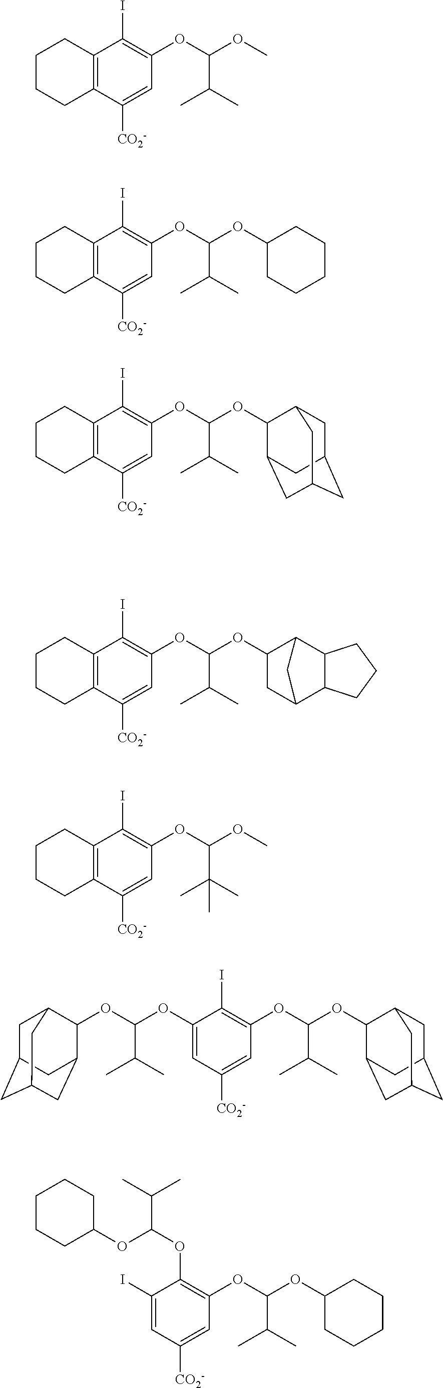

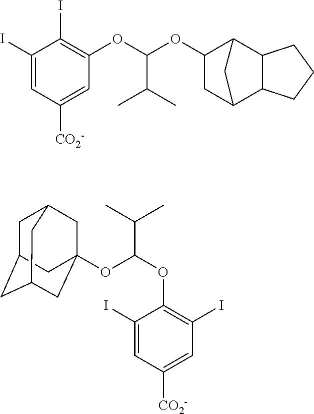

In the general formula (1), Z⁺ represents an onium cation. Specific examples thereof include a sulfonium cation, an iodonium cation, an ammonium cation, and a phosphonium cation. A sulfonium cation, iodonium cation, and ammonium cation represented below are preferable.

In the general formula (1), Z⁺ is preferably represented by any one of the following general formulae (Cation-1) to (Cation-3).

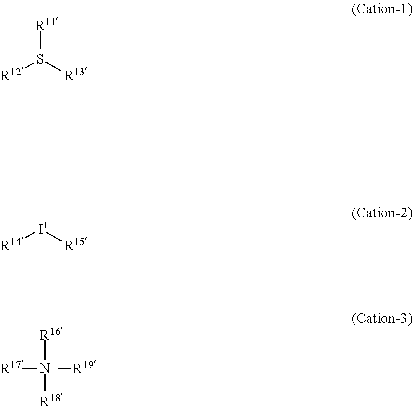

In the general formulae (Cation-1) to (Cation-3), R^{11′} to R^{19′} each independently represent a hydrocarbyl group having 1 to 30 carbon atoms and optionally having a heteroatom. The hydrocarbyl group may be a saturated or unsaturated group, and may be any of linear, branched, and cyclic groups. Specific examples thereof include: alkyl groups, such as a methyl group, an ethyl group, an n-propyl group, an isopropyl group, an n-butyl group, and a tert-butyl group; cyclic saturated hydrocarbyl groups, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups, such as a vinyl group, an allyl group, a propenyl group, a butenyl group, and a hexenyl group; cyclic unsaturated hydrocarbyl groups, such as a cyclohexenyl group; aryl groups, such as a phenyl group, a naphthyl group, and a thienyl group; aralkyl groups, such as a benzyl group, a 1-phenylethyl group, and a 2-phenylethyl group; and groups obtained by combining these groups. The hydrocarbyl group is preferably an aryl group. A part of hydrogen atoms in the hydrocarbyl group is optionally substituted with a group having a heteroatom such as an oxygen atom, a sulfur atom, a nitrogen atom, and a halogen atom. Between carbon atoms of these groups, a group having a heteroatom such as an oxygen atom, a sulfur atom, and a nitrogen atom is optionally interposed. As a result, optionally contained is a hydroxy group, a cyano group, a carbonyl group, an ether bond, an ester bond, a sulfonate ester bond, a carbonate bond, a lactone ring, a sultone ring, a carboxylic anhydride, or a haloalkyl group, etc.

R^{11′} and R^{12′} are optionally bonded to each other to form a ring together with the sulfur atom to which these groups are bonded. In this case, examples of the sulfonium cation represented by the formula (Cation-1) include the following cations.

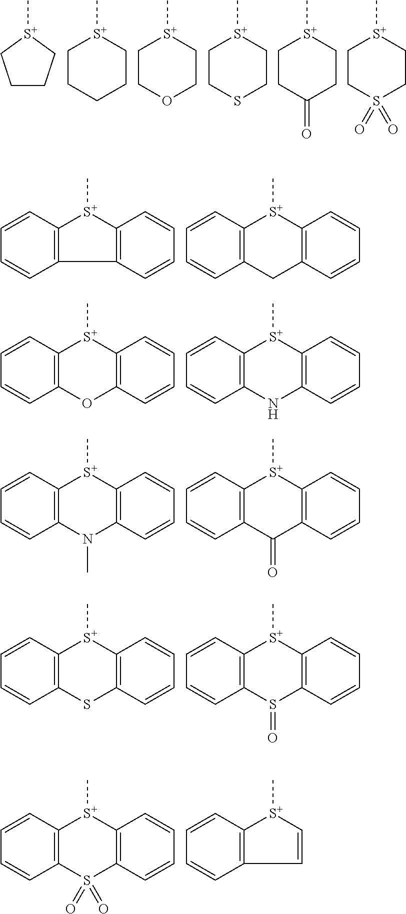

In the formulae, a broken line represents an attachment point to R¹³.

Examples of the cation of the sulfonium salt represented by the formula (Cation-1) include the following cations, but the cation is not limited thereto.

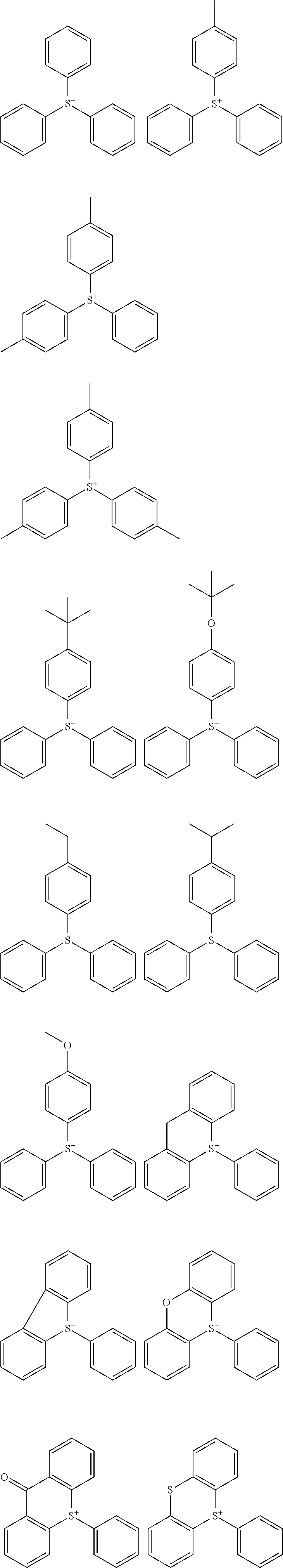

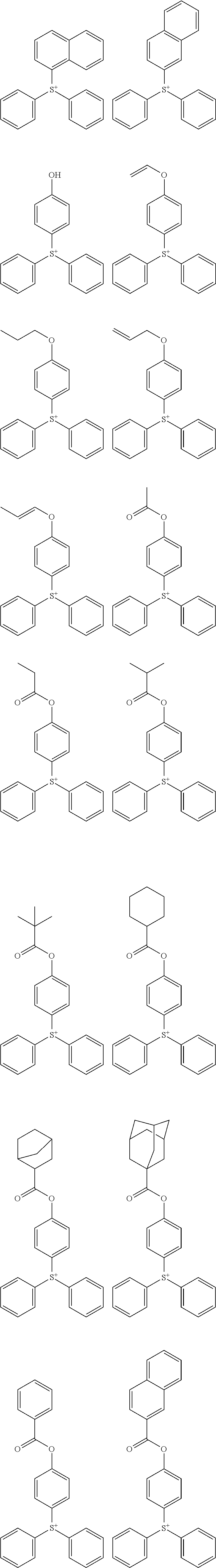

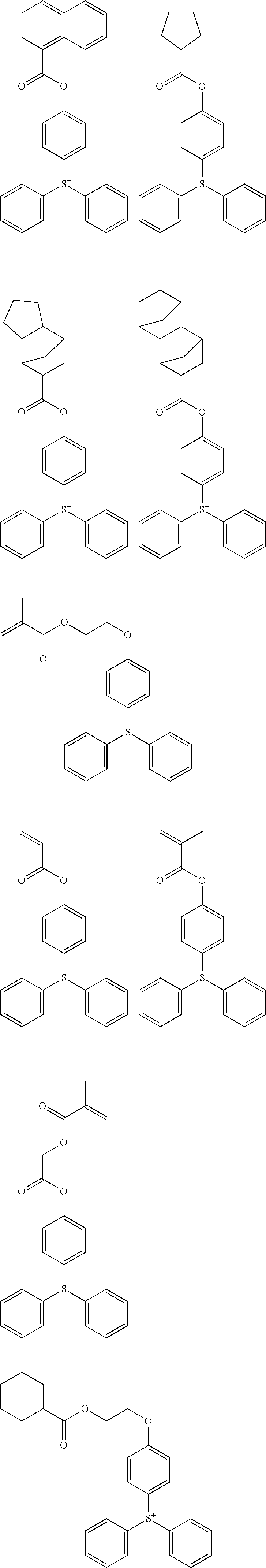

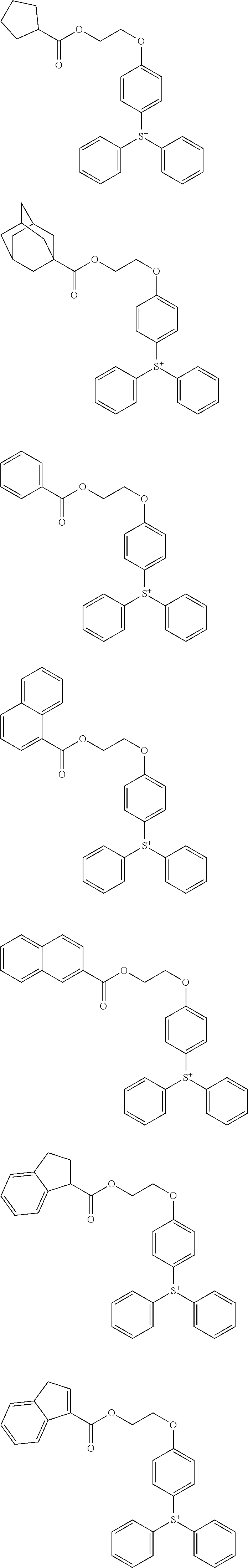

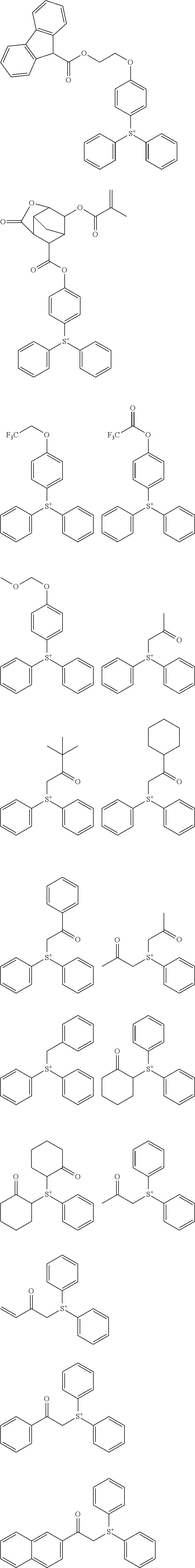

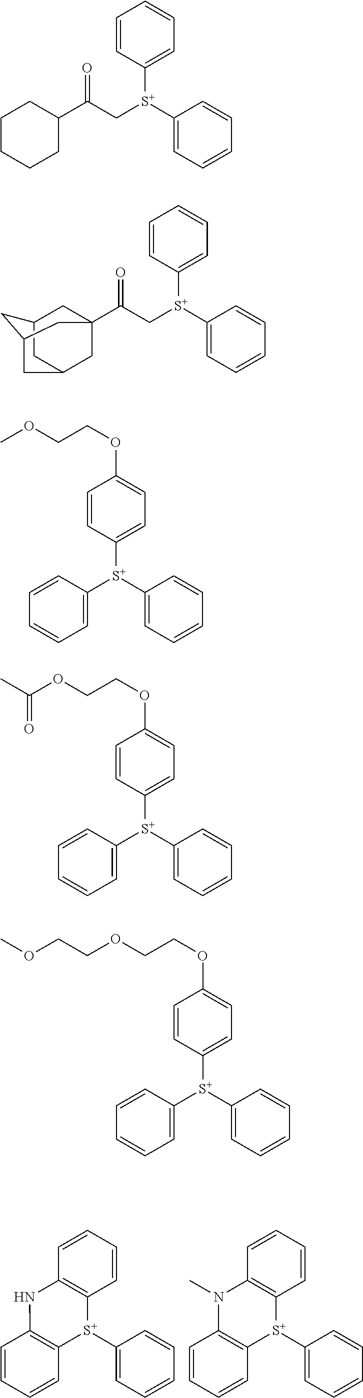

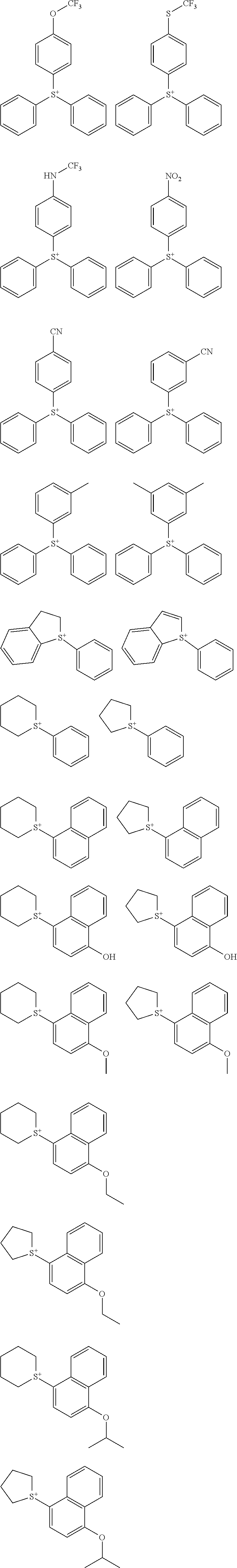

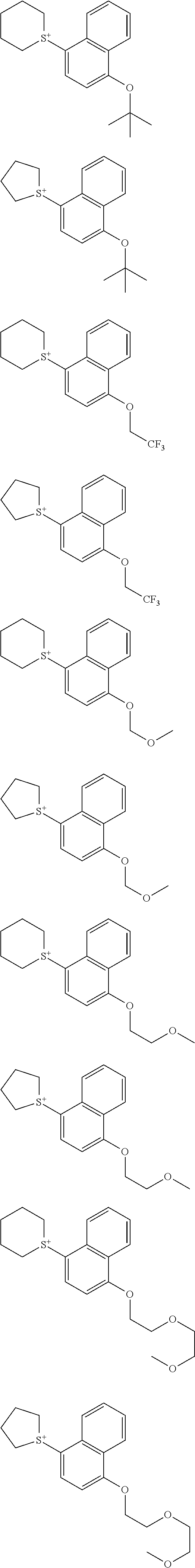

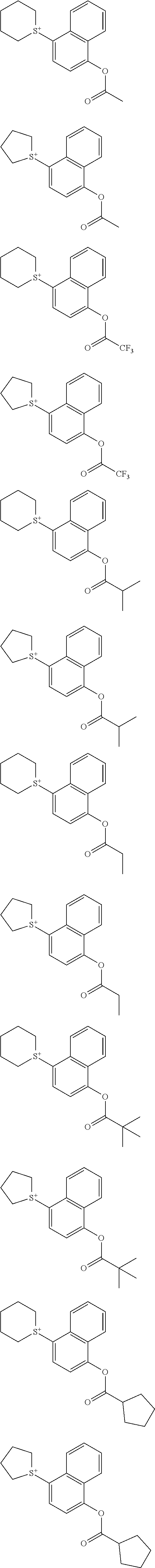

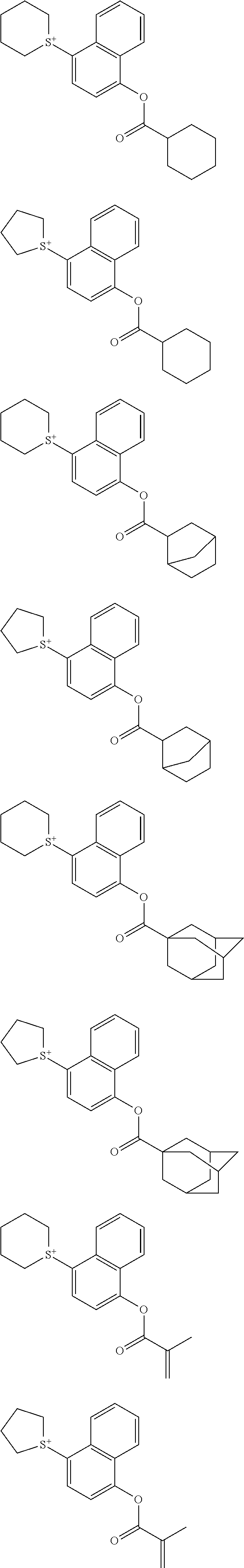

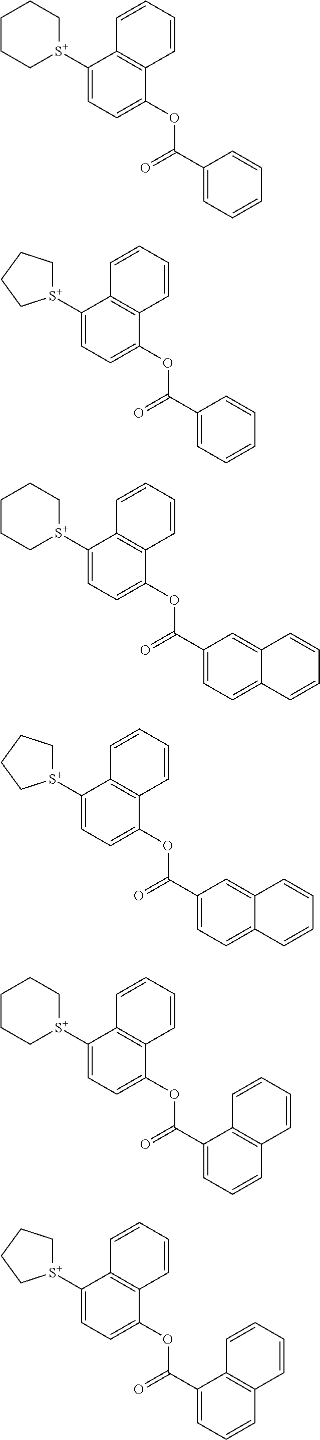 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

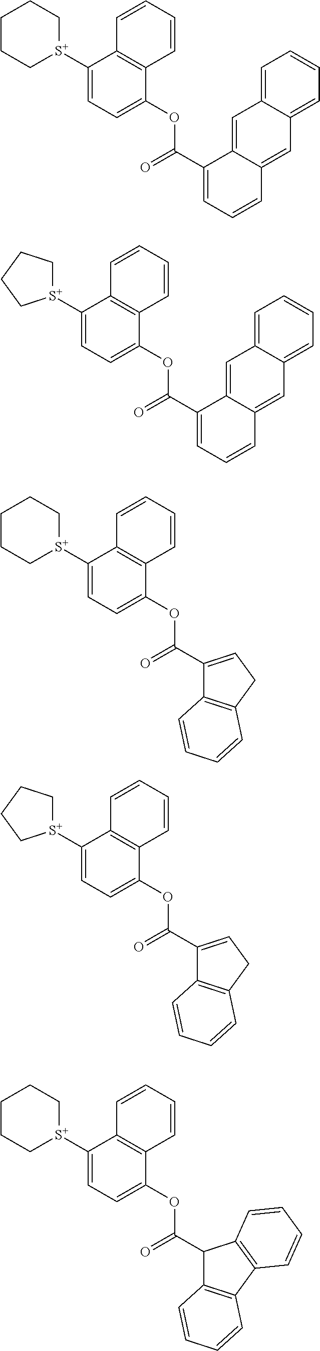 [SMILES: O=C(Oc1ccc([S+]2CCCCC2)c2ccccc12)c1cccc2cc3ccccc3cc12 ; O=C(Oc1ccc([S+]2CCCC2)c2ccccc12)c1cccc2cc3ccccc3cc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21]

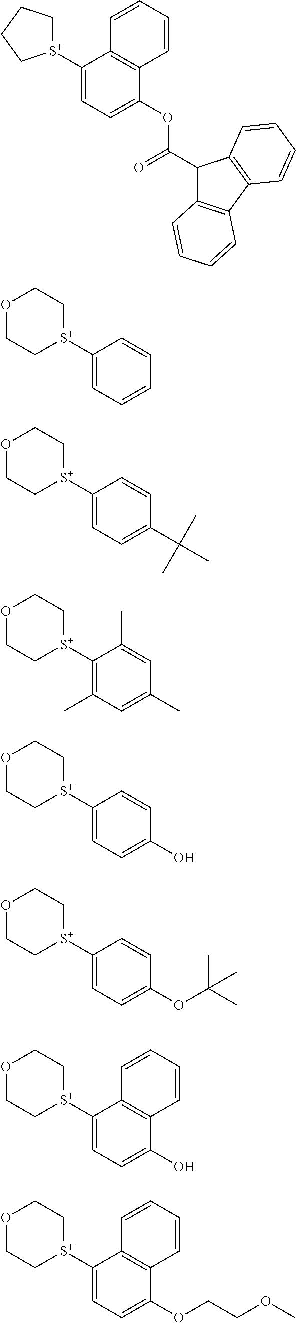

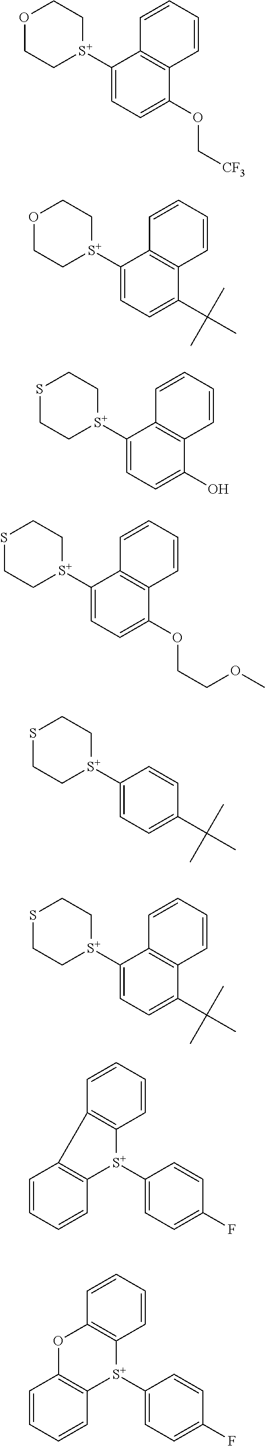

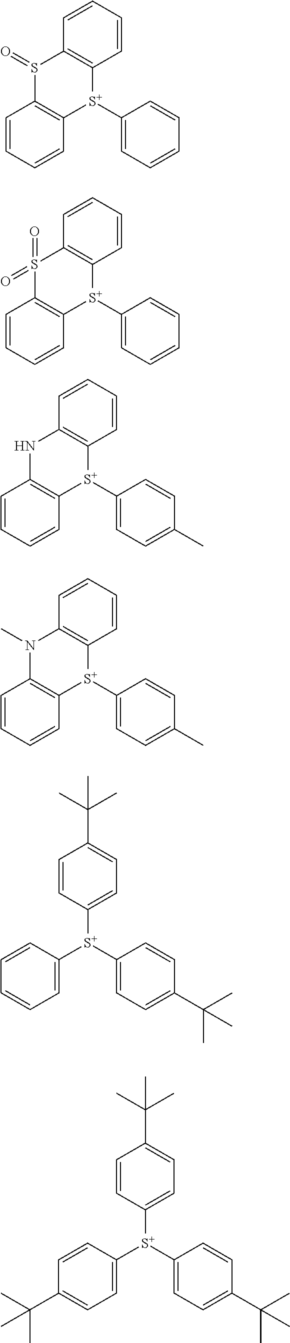

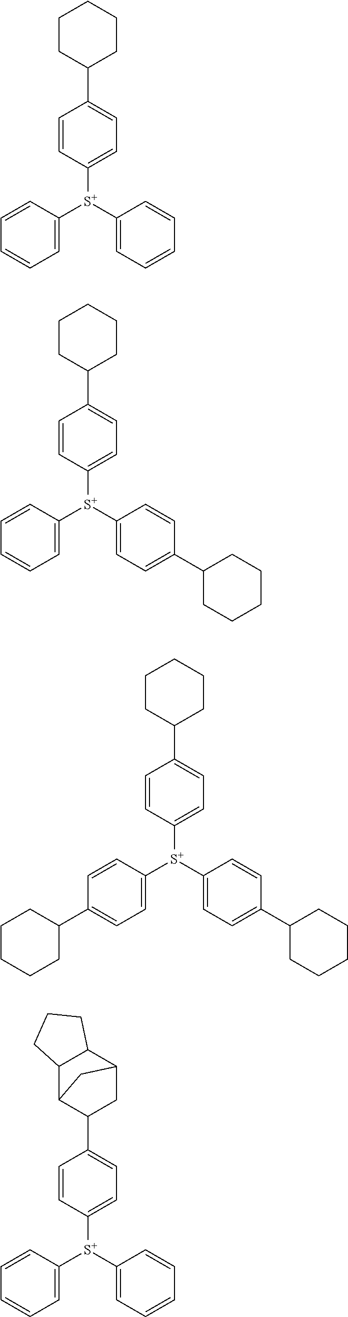 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1]

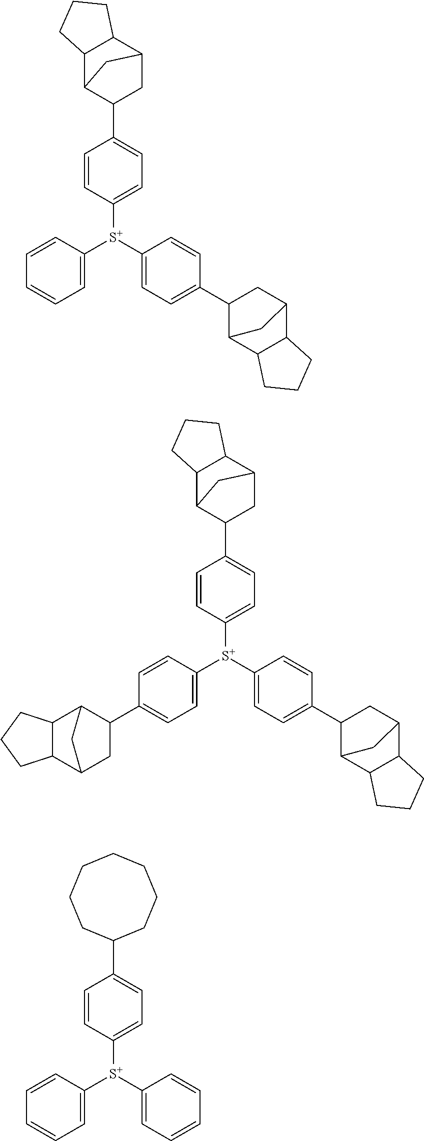 [SMILES: c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1]

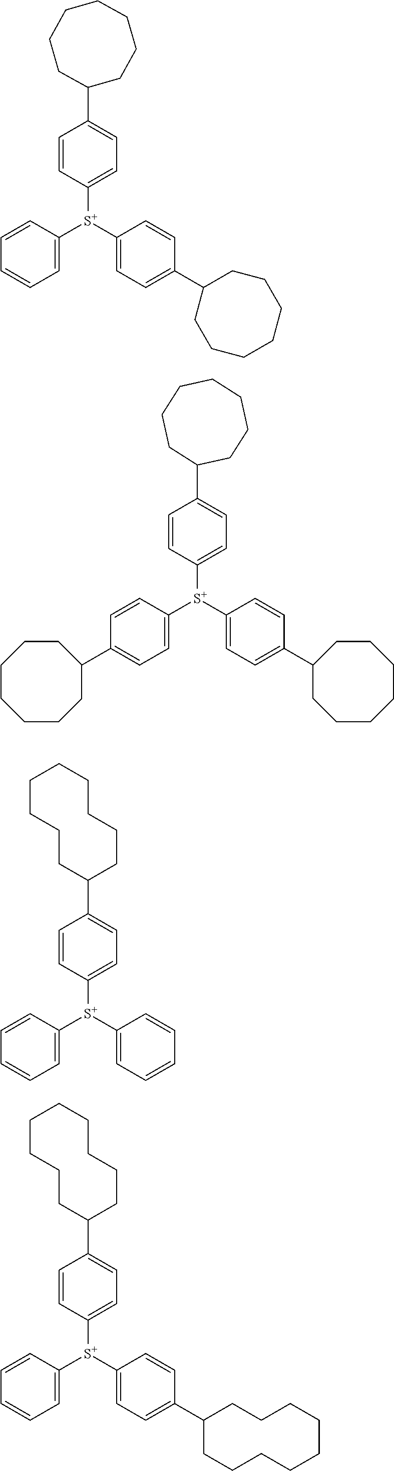 [SMILES: c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1]

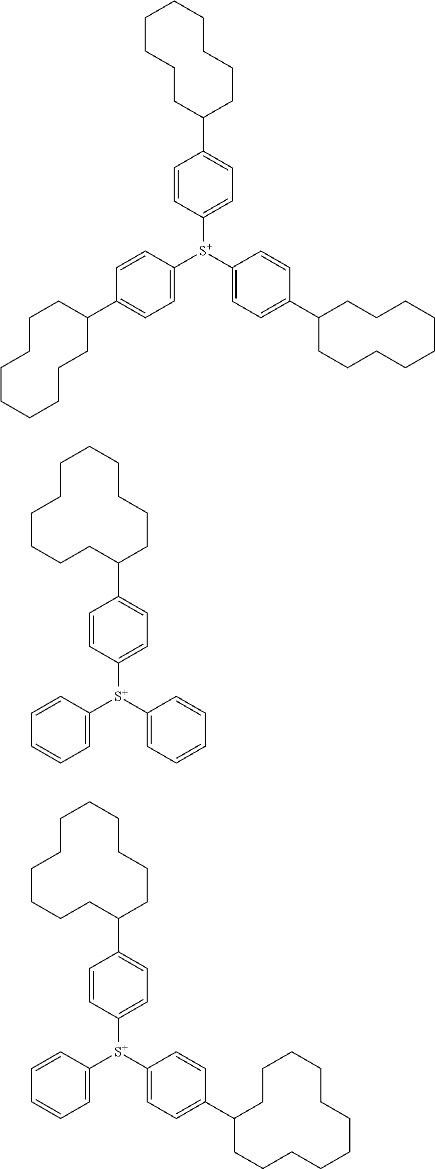 [SMILES: c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1]

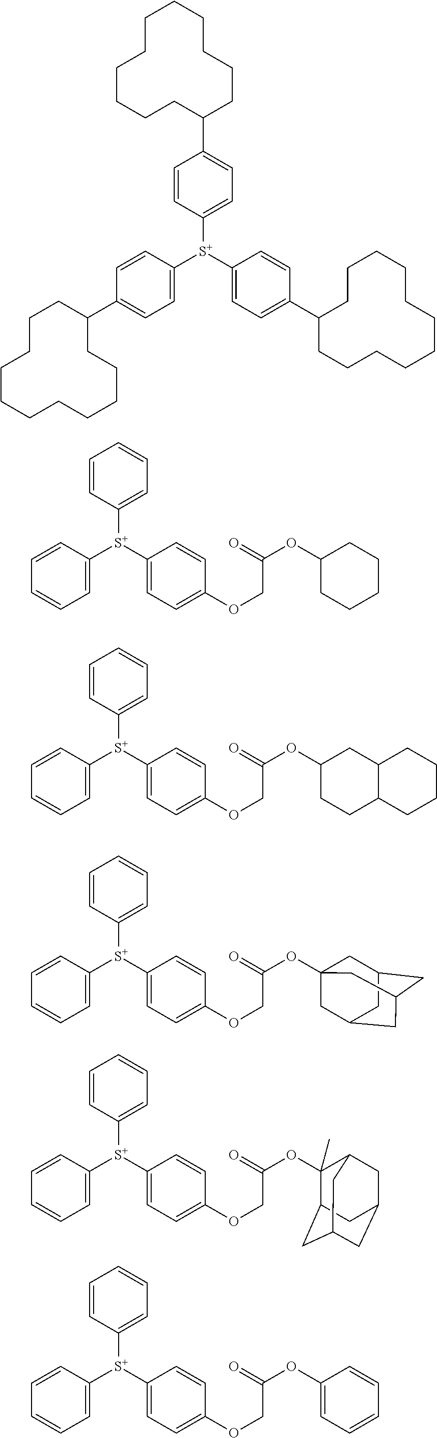 [SMILES: c1cc([S+](c2ccc(C3CCCCCCCCCCC3)cc2)c2ccc(C3CCCCCCCCCCC3)cc2)ccc1C1CCCCCCCCCCC1 ; O=C(COc1ccc([S+](c2ccccc2)c2ccccc2)cc1)OC1CCCCC1 ; O=C(COc1ccc([S+](c2ccccc2)c2ccccc2)cc1)OC1CCC2CCCCC2C1 ; O=C(COc1ccc([S+](c2ccccc2)c2ccccc2)cc1)OC12CC3CC(CC(C3)C1)C2 ; CC1(OC(=O)COc2ccc([S+](c3ccccc3)c3ccccc3)cc2)C2CC3CC(C2)CC1C3 ; O=C(COc1ccc([S+](c2ccccc2)c2ccccc2)cc1)Oc1ccccc1]

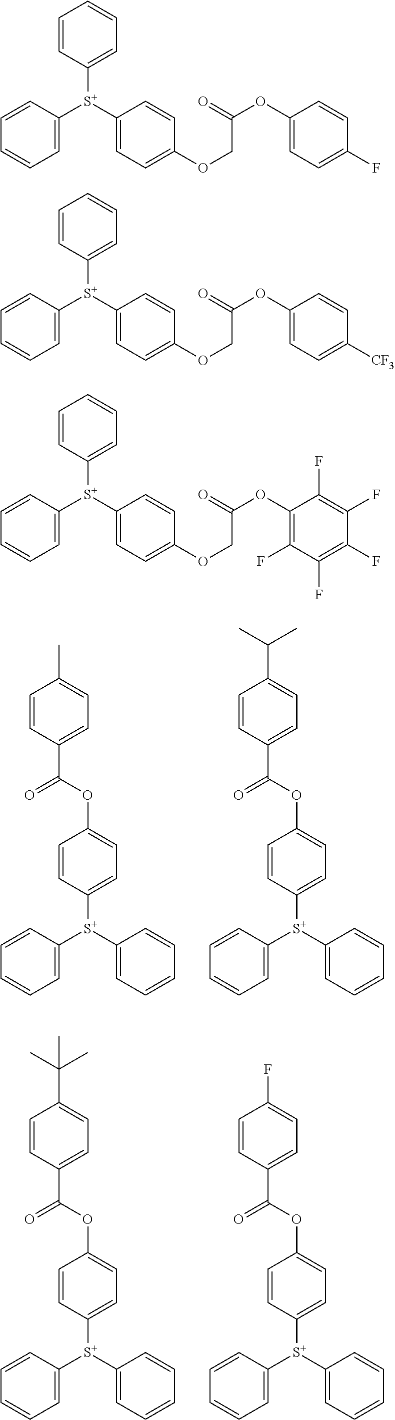

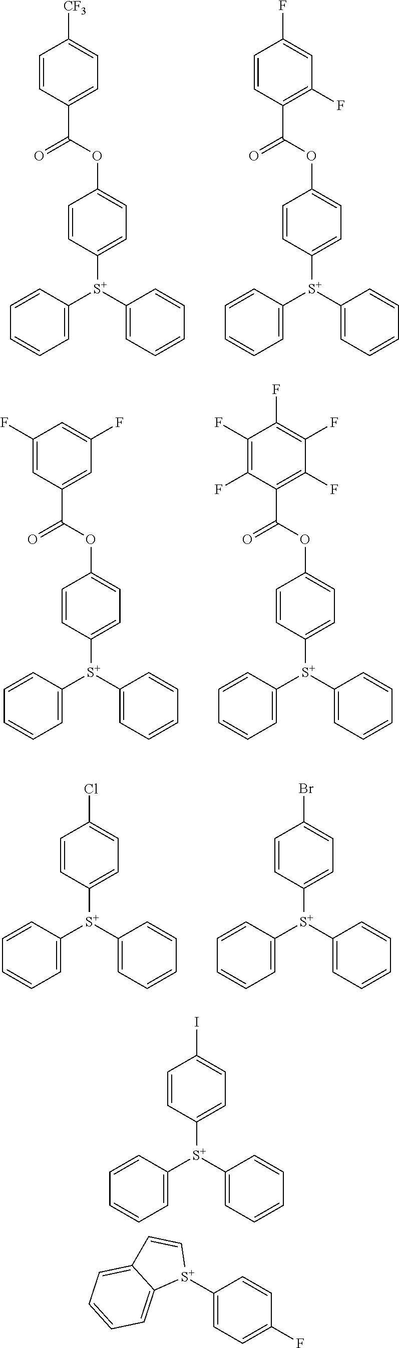

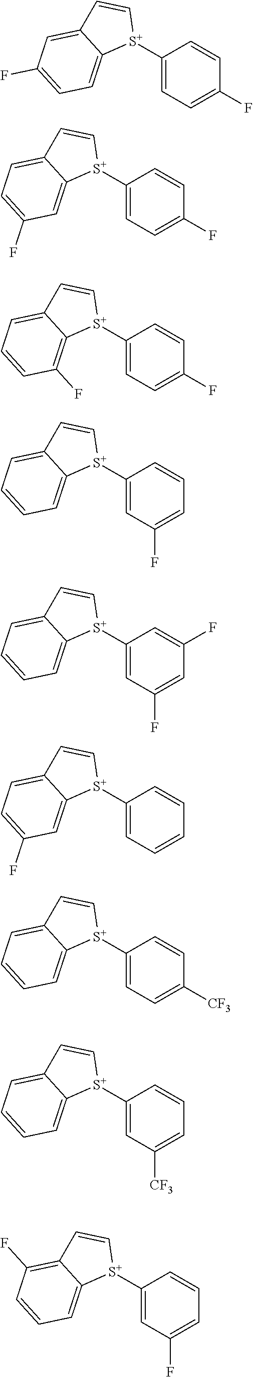

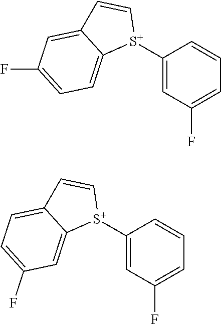 [SMILES: Fc1cccc(-[s+]2ccc3cc(F)ccc32)c1 ; Fc1cccc(-[s+]2ccc3ccc(F)cc32)c1]

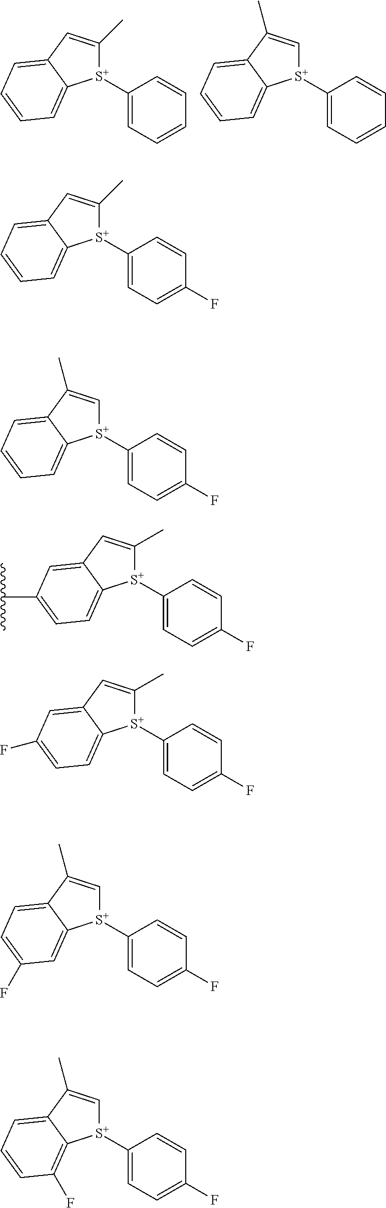

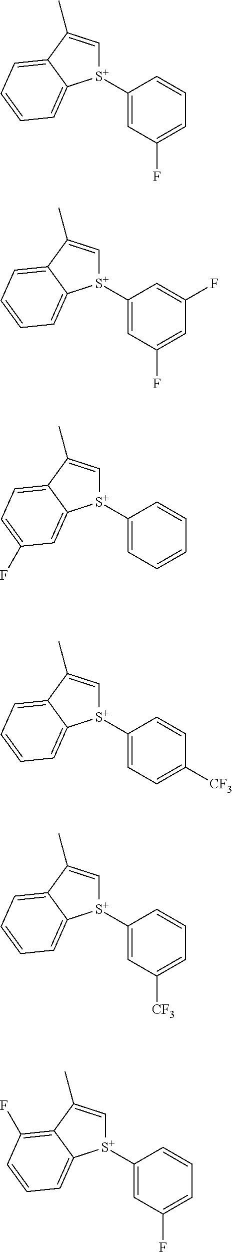 [SMILES: Cc1c[s+](-c2cccc(F)c2)c2ccccc12 ; Cc1c[s+](-c2cc(F)cc(F)c2)c2ccccc12 ; Cc1c[s+](-c2ccccc2)c2cc(F)ccc12 ; Cc1c[s+](-c2ccc(C(F)(F)F)cc2)c2ccccc12 ; Cc1c[s+](-c2cccc(C(F)(F)F)c2)c2ccccc12 ; Cc1c[s+](-c2cccc(F)c2)c2cccc(F)c12]

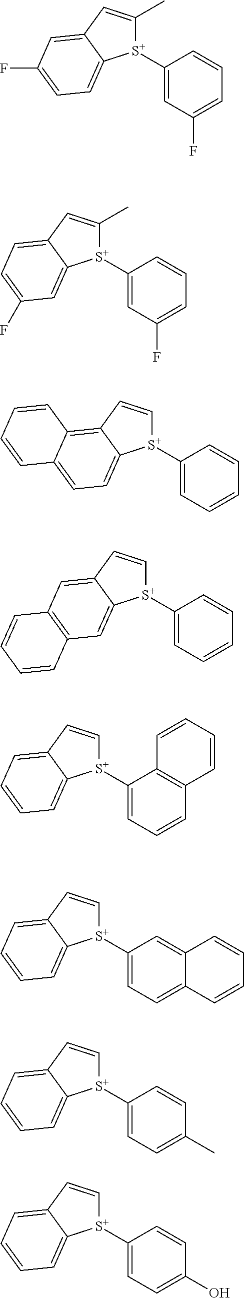

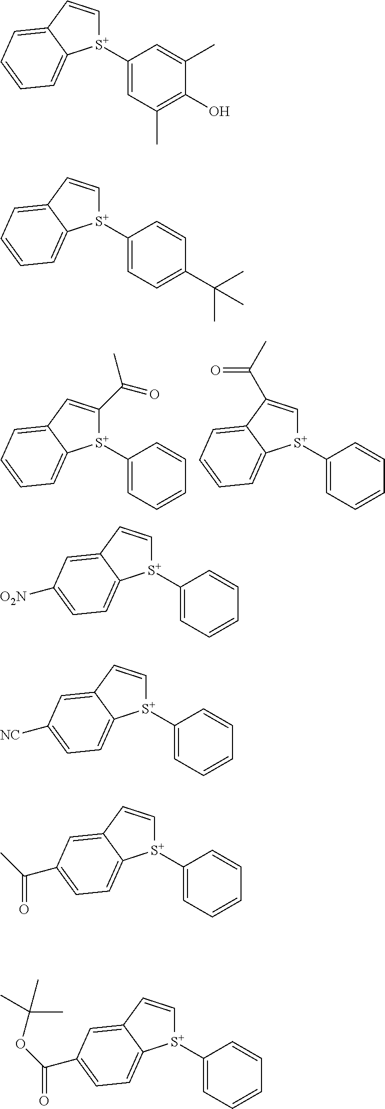

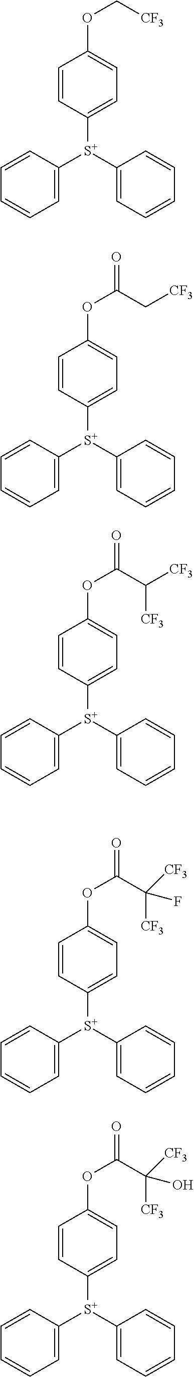 [SMILES: FC(F)(F)COc1ccc([S+](c2ccccc2)c2ccccc2)cc1 ; O=C(CC(F)(F)F)Oc1ccc([S+](c2ccccc2)c2ccccc2)cc1 ; O=C(Oc1ccc([S+](c2ccccc2)c2ccccc2)cc1)C(C(F)(F)F)C(F)(F)F ; O=C(Oc1ccc([S+](c2ccccc2)c2ccccc2)cc1)C(F)(C(F)(F)F)C(F)(F)F ; O=C(Oc1ccc([S+](c2ccccc2)c2ccccc2)cc1)C(O)(C(F)(F)F)C(F)(F)F]

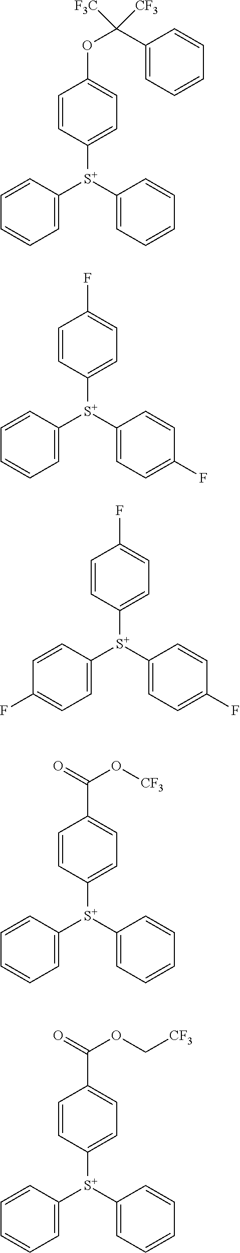 [SMILES: FC(F)(F)C(Oc1ccc([S+](c2ccccc2)c2ccccc2)cc1)(c1ccccc1)C(F)(F)F ; Fc1ccc([S+](c2ccccc2)c2ccc(F)cc2)cc1 ; Fc1ccc([S+](c2ccc(F)cc2)c2ccc(F)cc2)cc1 ; O=C(OC(F)(F)F)c1ccc([S+](c2ccccc2)c2ccccc2)cc1 ; O=C(OCC(F)(F)F)c1ccc([S+](c2ccccc2)c2ccccc2)cc1]

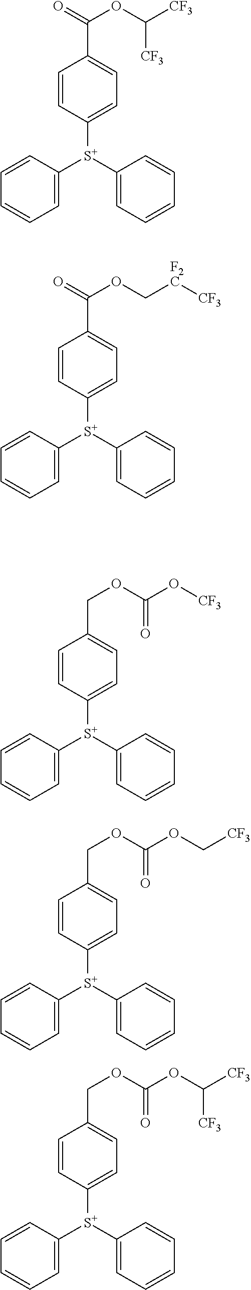 [SMILES: O=C(OC(C(F)(F)F)C(F)(F)F)c1ccc([S+](c2ccccc2)c2ccccc2)cc1 ; O=C(OCC(F)(F)C(F)(F)F)c1ccc([S+](c2ccccc2)c2ccccc2)cc1 ; O=C(OCc1ccc([S+](c2ccccc2)c2ccccc2)cc1)OC(F)(F)F ; O=C(OCc1ccc([S+](c2ccccc2)c2ccccc2)cc1)OCC(F)(F)F ; O=C(OCc1ccc([S+](c2ccccc2)c2ccccc2)cc1)OC(C(F)(F)F)C(F)(F)F]

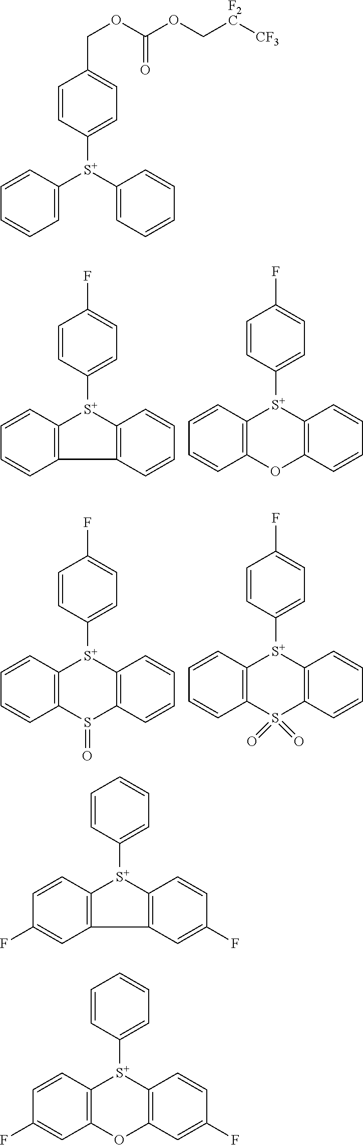

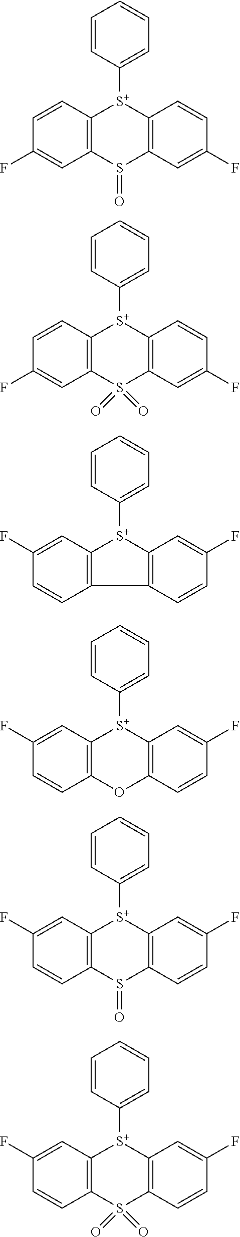 [SMILES: O=S1c2cc(F)ccc2[S+](c2ccccc2)c2ccc(F)cc21 ; O=S1(=O)c2cc(F)ccc2[S+](c2ccccc2)c2ccc(F)cc21 ; Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21]

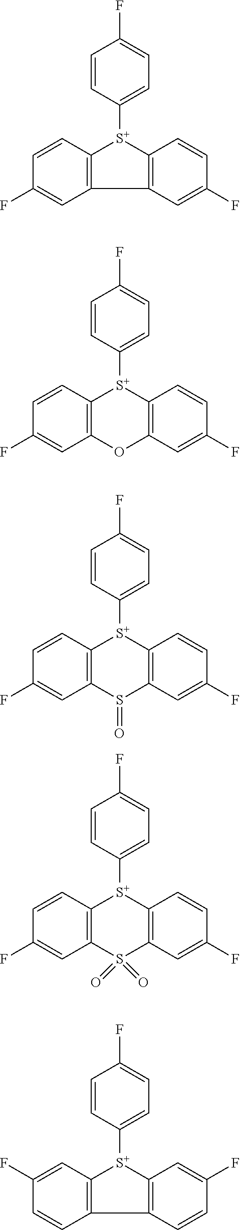 [SMILES: Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1]

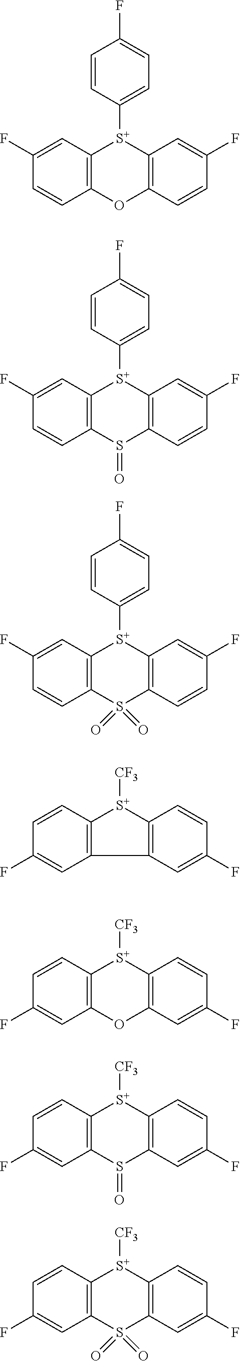

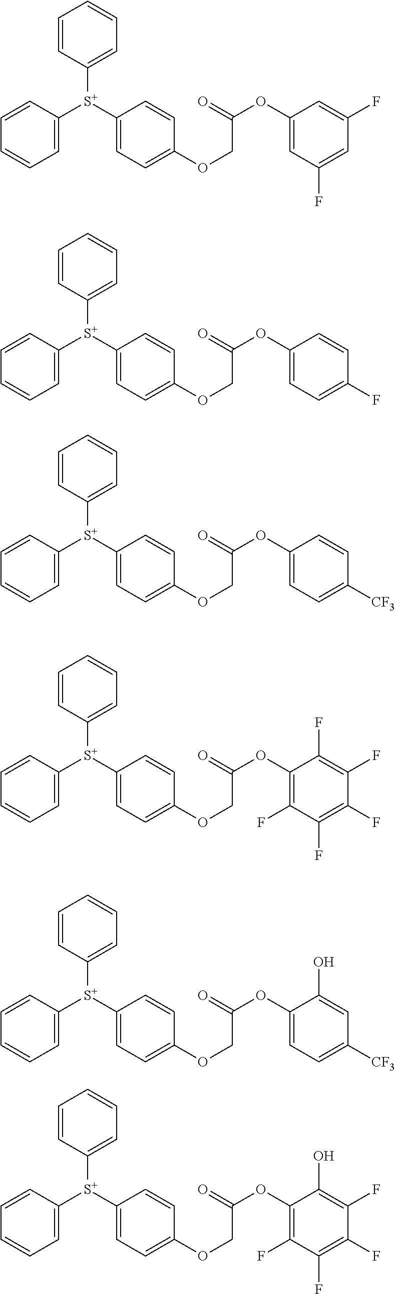 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

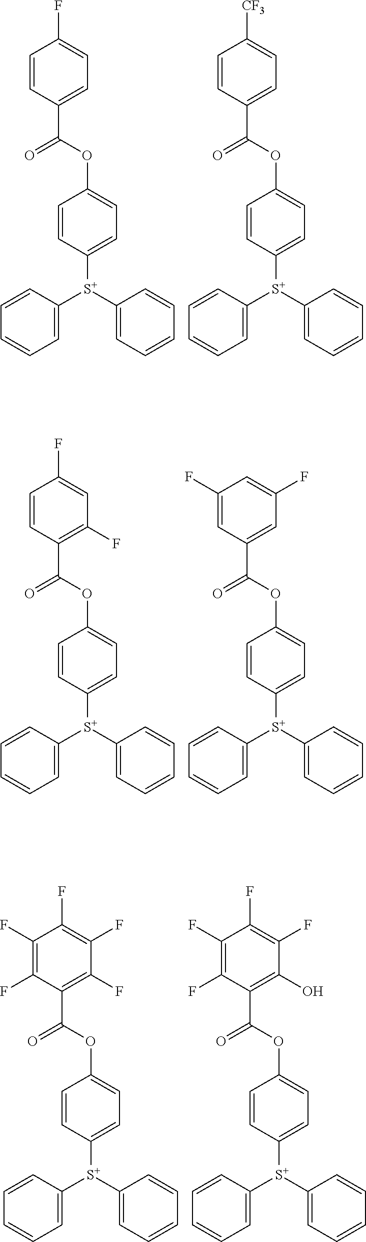 [SMILES: O=C(Oc1ccc([S+](c2ccccc2)c2ccccc2)cc1)c1ccc(F)cc1 ; O=C(Oc1ccc([S+](c2ccccc2)c2ccccc2)cc1)c1ccc(C(F)(F)F)cc1 ; O=C(Oc1ccc([S+](c2ccccc2)c2ccccc2)cc1)c1ccc(F)cc1F ; O=C(Oc1ccc([S+](c2ccccc2)c2ccccc2)cc1)c1cc(F)cc(F)c1 ; O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F]

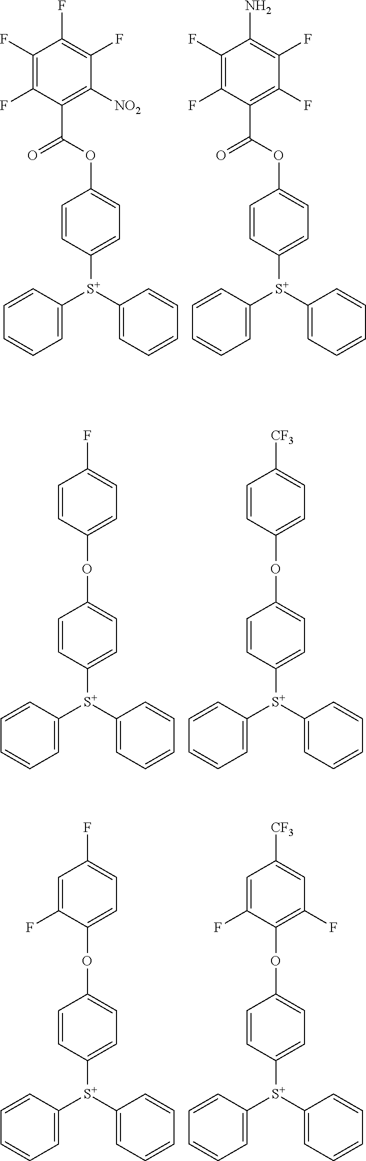 [SMILES: O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1 ; Fc1ccc(Oc2ccc([S+](c3ccccc3)c3ccccc3)cc2)c(F)c1 ; Fc1cc(C(F)(F)F)cc(F)c1Oc1ccc([S+](c2ccccc2)c2ccccc2)cc1]

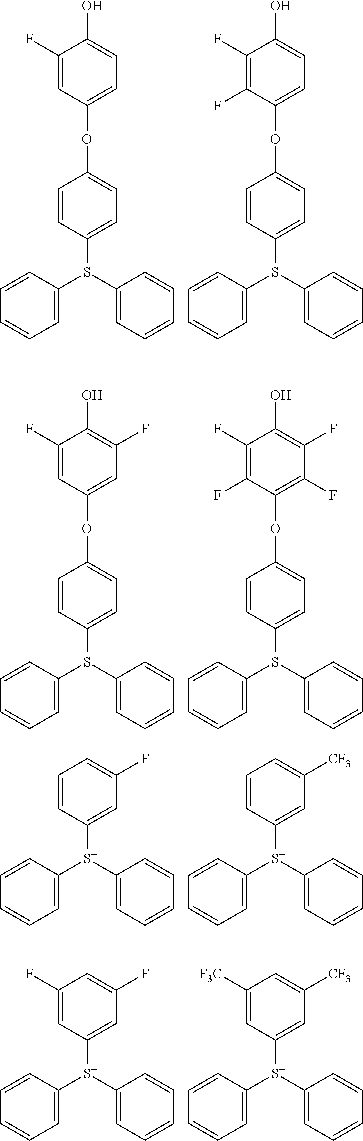

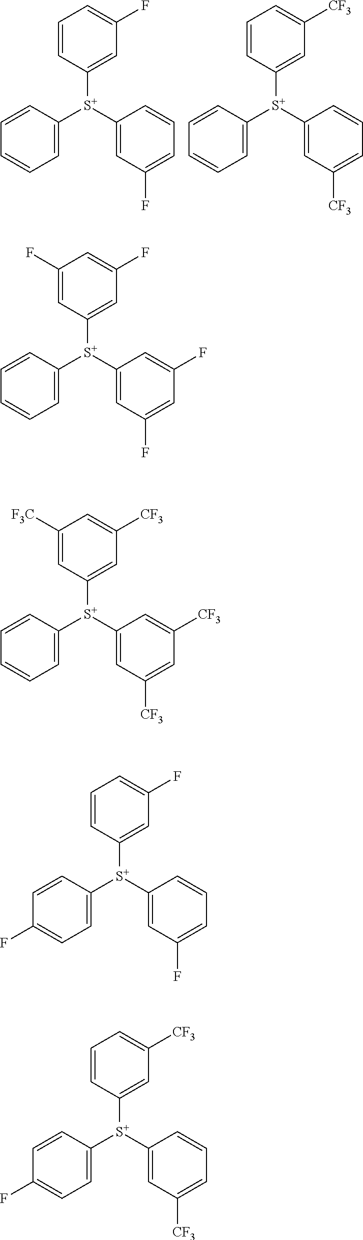 [SMILES: Fc1cccc([S+](c2ccccc2)c2cccc(F)c2)c1 ; FC(F)(F)c1cccc([S+](c2ccccc2)c2cccc(C(F)(F)F)c2)c1 ; Fc1cc(F)cc([S+](c2ccccc2)c2cc(F)cc(F)c2)c1 ; FC(F)(F)c1cc([S+](c2ccccc2)c2cc(C(F)(F)F)cc(C(F)(F)F)c2)cc(C(F)(F)F)c1 ; Fc1ccc([S+](c2cccc(F)c2)c2cccc(F)c2)cc1 ; Fc1ccc([S+](c2cccc(C(F)(F)F)c2)c2cccc(C(F)(F)F)c2)cc1]

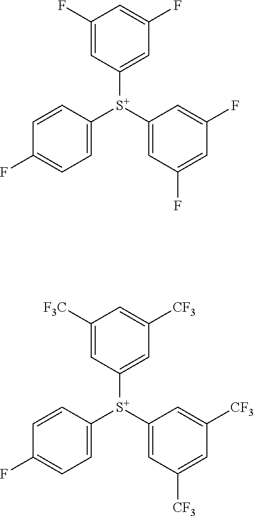 [SMILES: Fc1ccc([S+](c2cc(F)cc(F)c2)c2cc(F)cc(F)c2)cc1 ; Fc1ccc([S+](c2cc(C(F)(F)F)cc(C(F)(F)F)c2)c2cc(C(F)(F)F)cc(C(F)(F)F)c2)cc1]

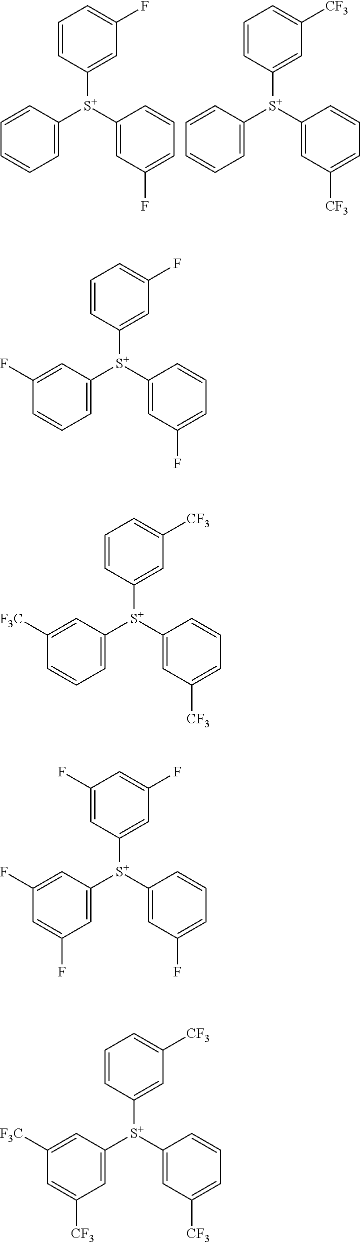 [SMILES: Fc1cccc([S+](c2ccccc2)c2cccc(F)c2)c1 ; FC(F)(F)c1cccc([S+](c2ccccc2)c2cccc(C(F)(F)F)c2)c1 ; Fc1cccc([S+](c2cccc(F)c2)c2cccc(F)c2)c1 ; FC(F)(F)c1cccc([S+](c2cccc(C(F)(F)F)c2)c2cccc(C(F)(F)F)c2)c1 ; Fc1cccc([S+](c2cc(F)cc(F)c2)c2cc(F)cc(F)c2)c1 ; FC(F)(F)c1cccc([S+](c2cccc(C(F)(F)F)c2)c2cc(C(F)(F)F)cc(C(F)(F)F)c2)c1]

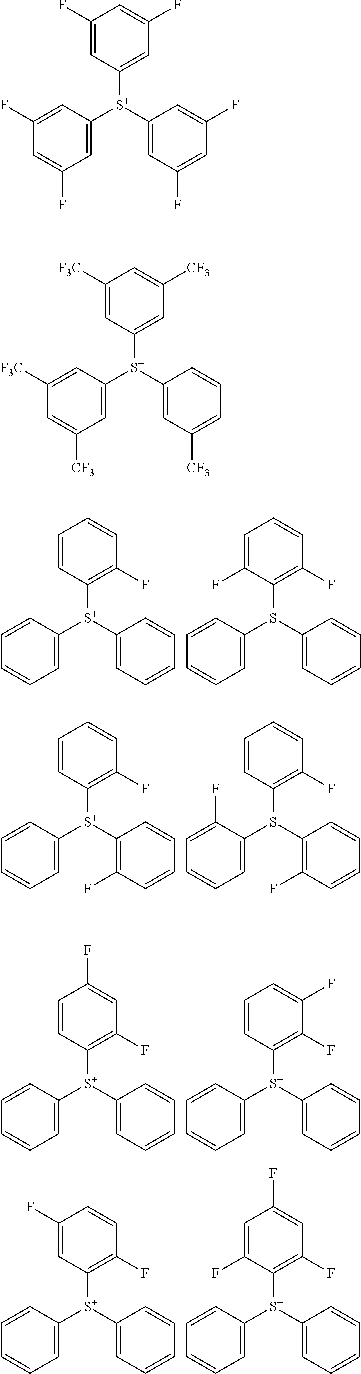

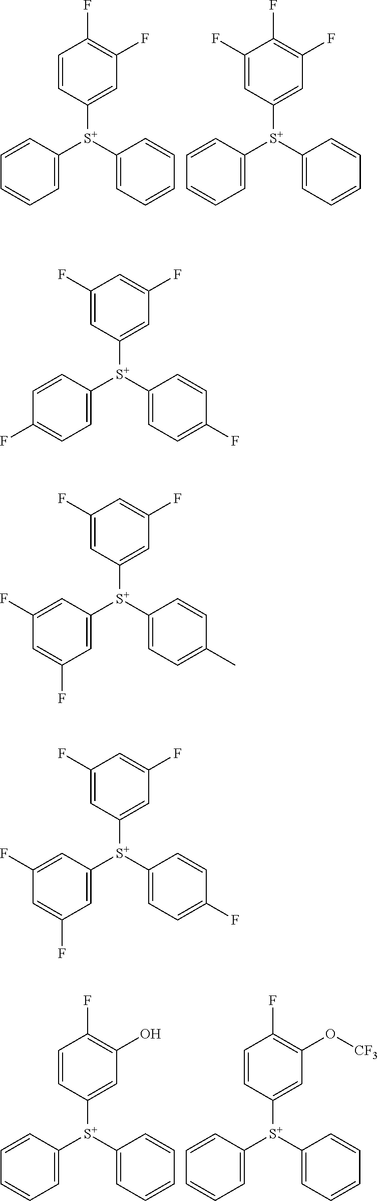

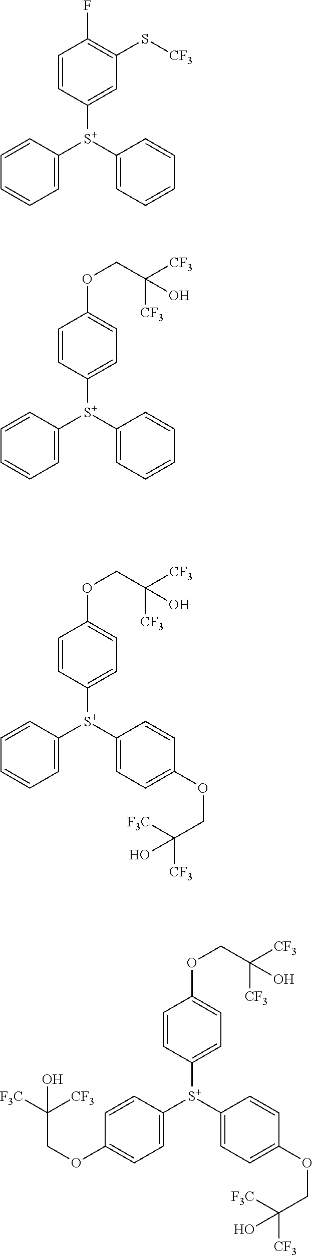 [SMILES: Fc1ccc([S+](c2ccccc2)c2ccccc2)cc1SC(F)(F)F ; OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F ; OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F]

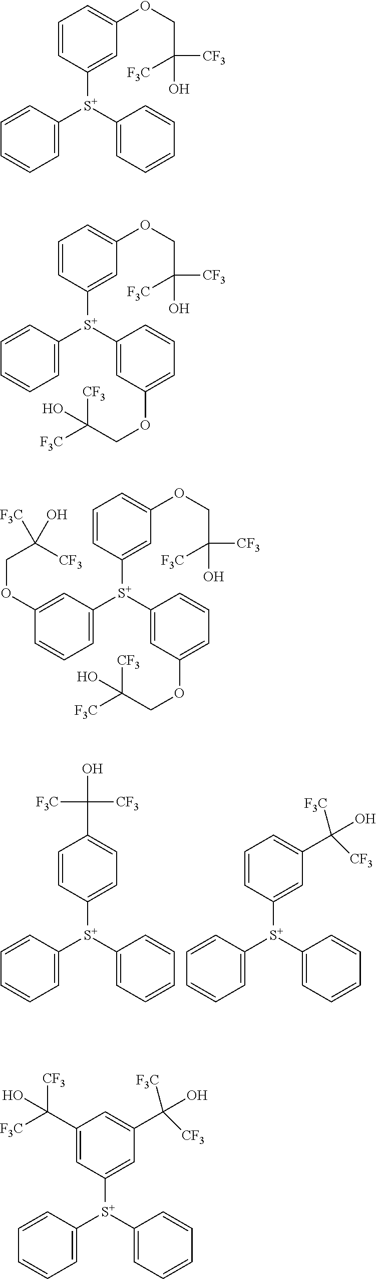 [SMILES: OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F ; OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F ; OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F]

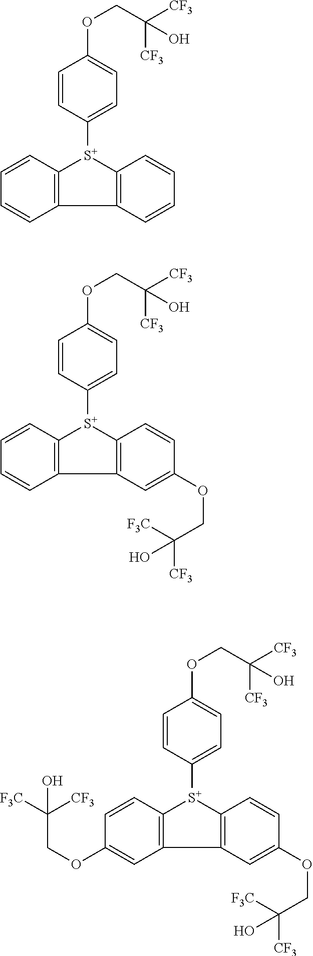 [SMILES: OC(COc1ccc(-[s+]2c3ccccc3c3ccccc32)cc1)(C(F)(F)F)C(F)(F)F ; OC(COc1ccc(-[s+]2c3ccccc3c3cc(OCC(O)(C(F)(F)F)C(F)(F)F)ccc32)cc1)(C(F)(F)F)C(F)(F)F ; OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F]

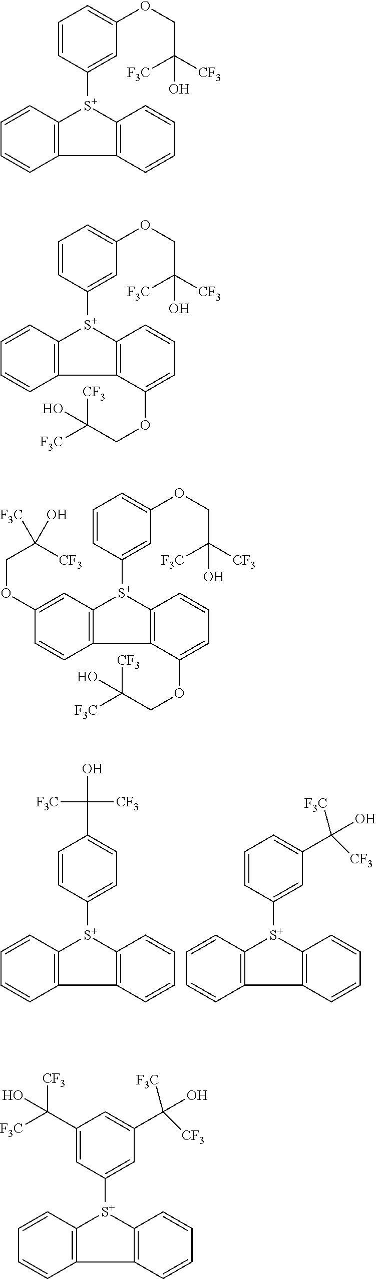 [SMILES: OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F ; OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F ; OC(c1ccc(-[s+]2c3ccccc3c3ccccc32)cc1)(C(F)(F)F)C(F)(F)F ; OC(c1cccc(-[s+]2c3ccccc3c3ccccc32)c1)(C(F)(F)F)C(F)(F)F ; OC(c1cc(-[s+]2c3ccccc3c3ccccc32)cc(C(O)(C(F)(F)F)C(F)(F)F)c1)(C(F)(F)F)C(F)(F)F]

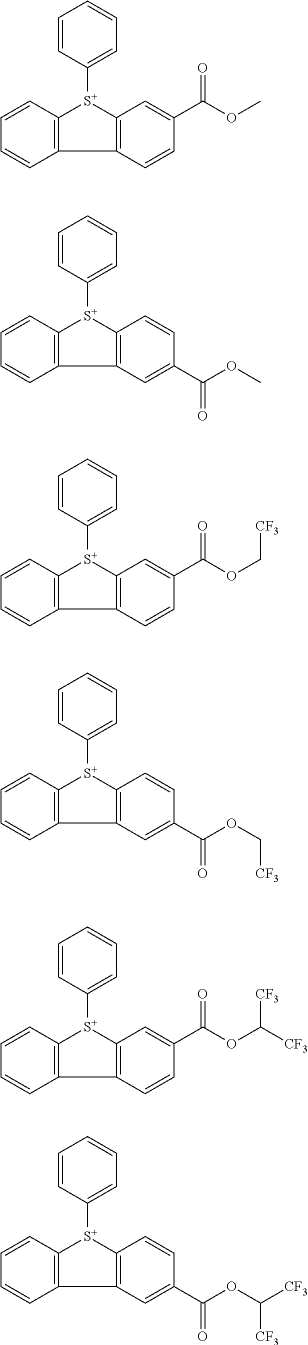 [SMILES: COC(=O)c1ccc2c3ccccc3[s+](-c3ccccc3)c2c1 ; COC(=O)c1ccc2c(c1)c1ccccc1[s+]2-c1ccccc1 ; O=C(OCC(F)(F)F)c1ccc2c3ccccc3[s+](-c3ccccc3)c2c1 ; O=C(OCC(F)(F)F)c1ccc2c(c1)c1ccccc1[s+]2-c1ccccc1 ; O=C(OC(C(F)(F)F)C(F)(F)F)c1ccc2c3ccccc3[s+](-c3ccccc3)c2c1 ; O=C(OC(C(F)(F)F)C(F)(F)F)c1ccc2c(c1)c1ccccc1[s+]2-c1ccccc1]

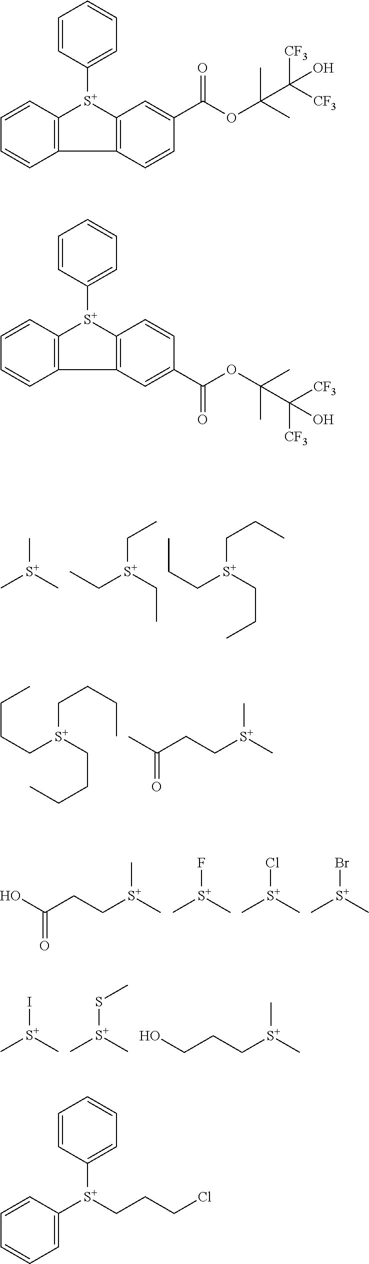

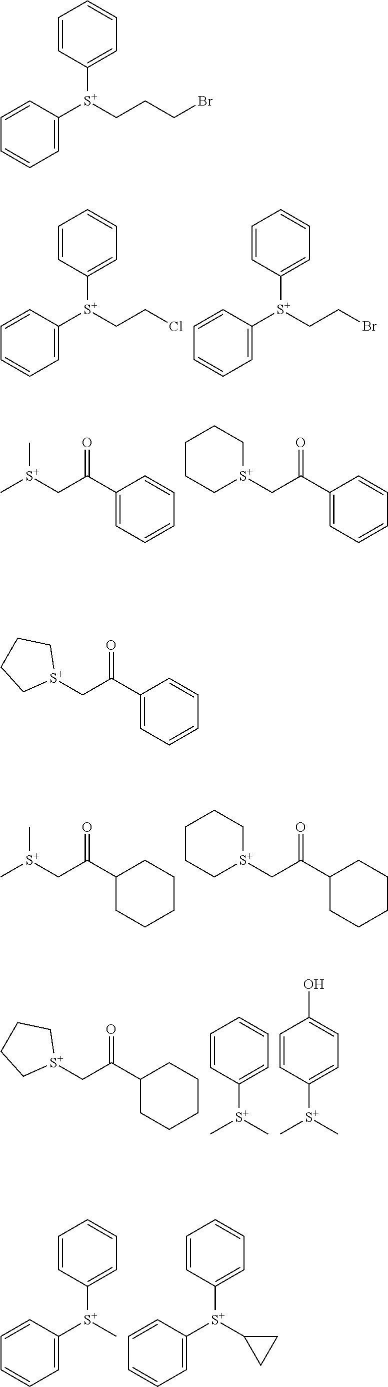

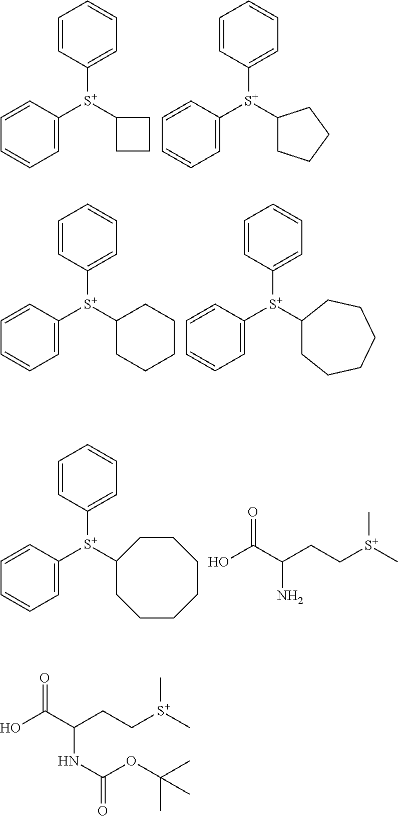

Examples of the iodonium cation represented by the general formula (Cation-2) include the following cations, but the cation is not limited thereto.

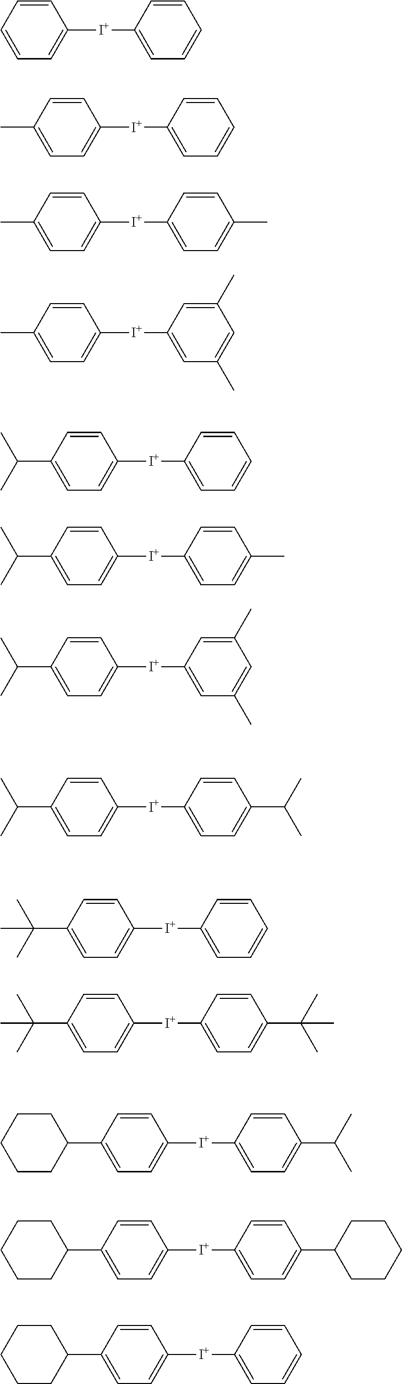

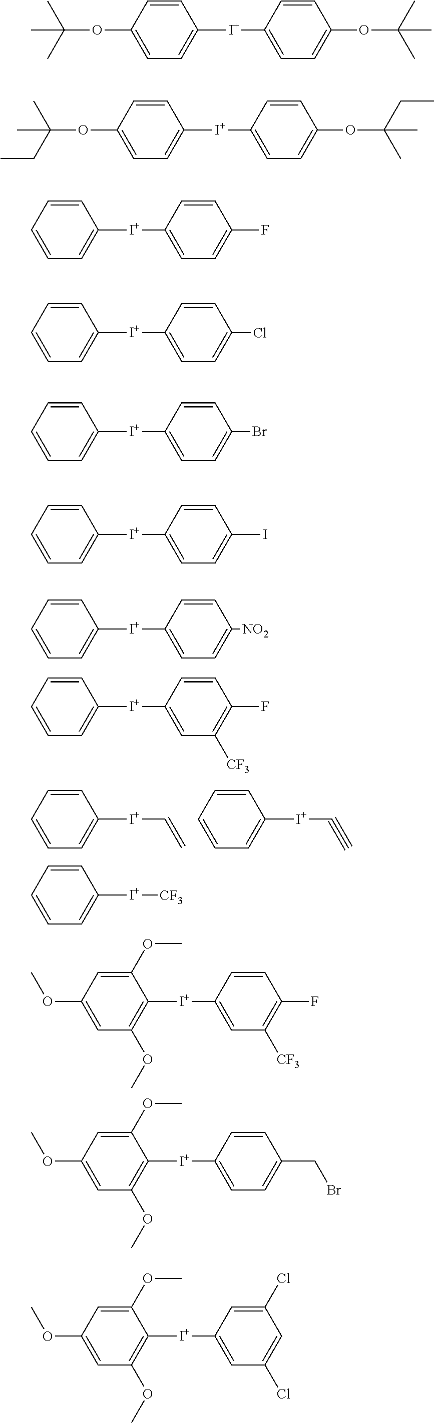

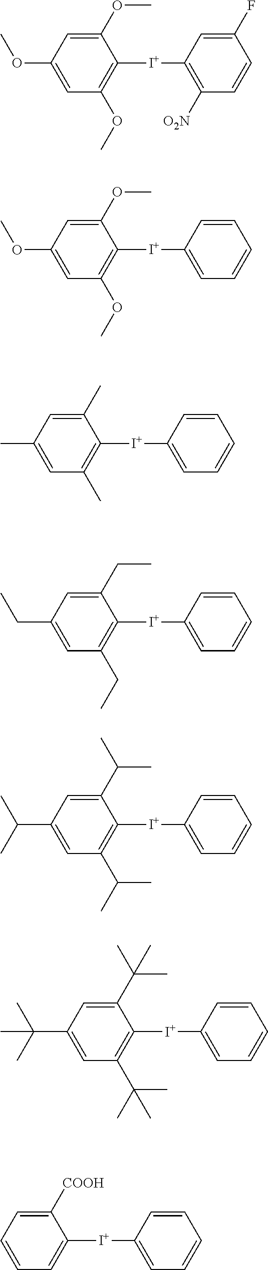

Examples of the ammonium cation represented by the general formula (Cation-3) include the following cations, but the cation is not limited thereto.

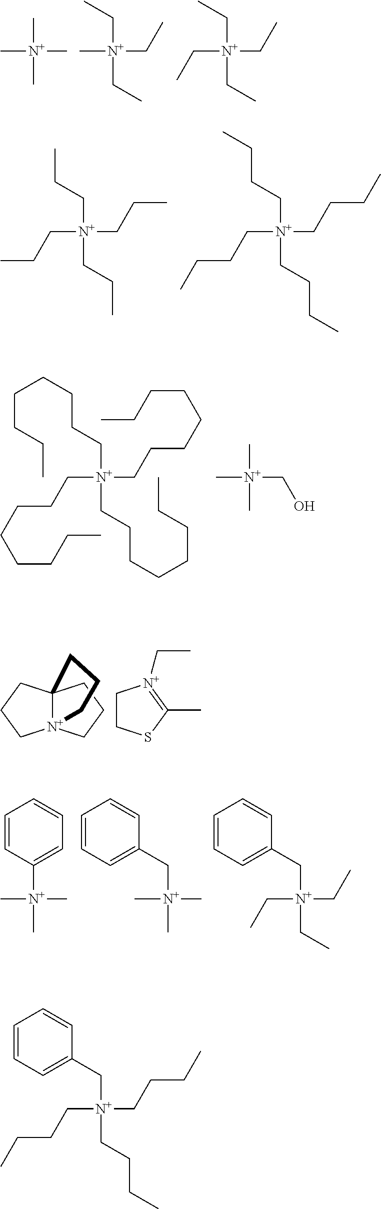

Examples of specific structures of the inventive onium salt include any combination of the aforementioned anions and cations.

The inventive onium salt can be synthesized by, for example, ion-exchanging a hydrochloride salt or carbonate salt having the onium cation with a corresponding benzoic acid derivative.

If both of the inventive onium salt and an onium salt to generate strong acid such as an α-fluorinated sulfonic acid, an imide acid, or a methide acid (hereinafter, these acids are collectively defined as “strong acid”) are present, the corresponding carboxylic acid and the strong acid are generated with light irradiation. Meanwhile, many undecomposed onium salts are present in a low exposure-dose portion. The strong acid functions as a catalyst to cause the deprotection reaction of the base resin, but the inventive onium salt hardly causes the deprotection reaction. The strong acid is ion-exchanged with the remained sulfonium carboxylate salt to form an onium salt of the strong acid, and the carboxylic acid is simultaneously released. In other words, the ion exchange neutralizes the strong acid with the onium carboxylate salt. That is, the inventive onium salt functions as a quencher (acid diffusion inhibitor). This onium salt quencher typically tends to reduce LWR of a resist pattern compared with a quencher using an amine compound.

The salt exchange between the strong acid and the onium carboxylate salt is unlimitedly repeated. A place where the strong acid is generated with exposure in a final stage differs from a place where the strong acid-generating onium salt is originally present. It is presumed that the cycle of the acid generation with light and the salt exchange is repeated many times to uniformize acid generating points, resulting in reduction of LWR of a resist pattern after the development.

As noted above, the inventive onium salt functions as an acid diffusion inhibitor, specifically as an acid diffusion inhibitor (quencher) in a resist composition. The acid diffusion inhibitor comprising the inventive onium salt is preferably contained in a resist composition. More specifically, a resist composition containing the inventive onium salt as the acid diffusion inhibitor, with both of the positive-type and negative-type resist compositions in lithography, has excellent sensitivity and resolution of a resist film, and reduced LWR of a line pattern and CDU of a hole pattern or dot pattern, inhibits swelling during development, and can inhibit collapse of a resist pattern, which is extremely effective for precise fine patterning.

In the inventive onium salt, the tertiary alkyl or acetal acid-labile group is introduced to improve fat-solubility. Thus, the inventive onium salt can exhibit sufficient solubility in an organic solvent despite having an iodine atom. Therefore, the inventive onium salt has no concern about precipitation in the resist solvent.

### [Acid Diffusion Inhibitor]

The inventive acid diffusion inhibitor comprises the inventive onium salt. The inventive acid diffusion inhibitor can be referred to as, for example, a benzoic acid-type PDQ (photodegradable quencher) having a tertiary ether structure protected with an iodophenol.

As noted above, the inventive onium salt can be used as an acid diffusion inhibitor (quencher) for a resist composition. Thus, the acid diffusion inhibitor comprising the inventive onium salt is an acid diffusion inhibitor for a resist composition which has excellent sensitivity and resolution of a resist film, and reduced LWR of a line pattern and CDU of a hole pattern and dot pattern, inhibits swelling during development, and can inhibit collapse of a resist pattern, which is extremely effective for precise fine patterning.

In addition, the inventive acid diffusion inhibitor can exhibit sufficient solubility in an organic solvent, and has no concern about precipitation in a resist solvent.

### [Resist Composition]

The inventive resist composition comprises the inventive acid diffusion inhibitor.

The inventive resist composition may be a positive-type (chemically amplified positive-type) resist composition or a negative-type (chemically amplified negative-type) resist composition.

Since comprising the acid diffusion inhibitor comprising the inventive onium salt, the inventive resist composition has excellent sensitivity and resolution of a resist film, reduced LWR of a line pattern and CDU of a hole pattern and dot pattern, inhibits swelling during development, and can inhibit collapse of a resist pattern, which is extremely effective for precise fine patterning.

Since the inventive acid diffusion inhibitor can exhibit sufficient solubility in an organic solvent, the acid diffusion inhibitor has no concern about precipitation in a resist solvent.

The content of the inventive onium salt in the resist composition is preferably 0.001 to 50 parts by mass, and more preferably 0.01 to 40 parts by mass, relative to 100 parts by mass of a base polymer, described later. As the inventive onium salt, one kind may be used, or combination of two or more kinds may be used.

The inventive resist composition can comprise, for example, the following components in addition to the inventive acid diffusion inhibitor.

### Base Polymer

The inventive resist composition (resist material) may further comprise a base polymer. The base polymer has a repeating unit having an acid-labile group in a case of the positive-type resist composition. The repeating unit having an acid-labile group is preferably a repeating unit represented by the following general formula (a1) (hereinafter, also referred to as the repeating unit a1) and/or a repeating unit represented by the following general formula (a2) (hereinafter, also referred to as the repeating unit a2).

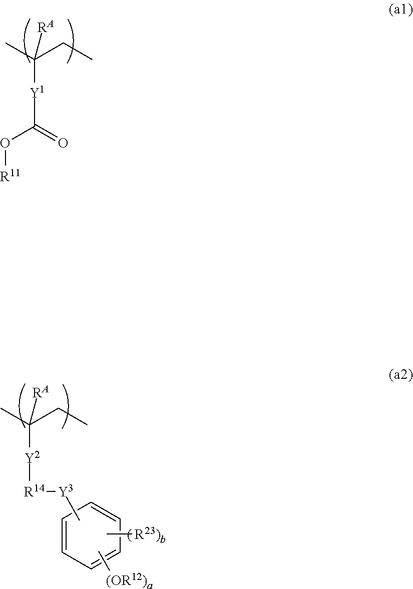

In the general formulae (a1) and (a2), R^{A} each independently represents a hydrogen atom or a methyl group. Y¹ represents a single bond, a phenylene group, a naphthylene group, or a linking group having 1 to 12 carbon atoms and having at least one selected from an ester bond and a lactone ring. Y² represents a single bond or an ester bond. Y³ represents a single bond, an ether bond, or an ester bond. R¹¹ and R¹² each independently represents an acid-labile group. When the base polymer comprises both of the repeating unit a1 and the repeating unit a2, R¹¹ and R¹² may be same as or different from each other. R¹³ represents a fluorine atom, a trifluoromethyl group, a cyano group, or a saturated hydrocarbyl group having 1 to 6 carbon atoms. R¹⁴ represents a single bond or an alkanediyl group having 1 to 6 carbon atoms, a part of carbon atoms therein being optionally substituted with an ether bond or an ester bond. “a” represents 1 or 2, “b” represents an integer of 0 to 4, and 1≤a+b≤5.

Examples of a monomer to yield the repeating unit a1 include the following monomers, but the monomer is not limited thereto. In the following formulae, R^{A} and R¹² represent the same as above.

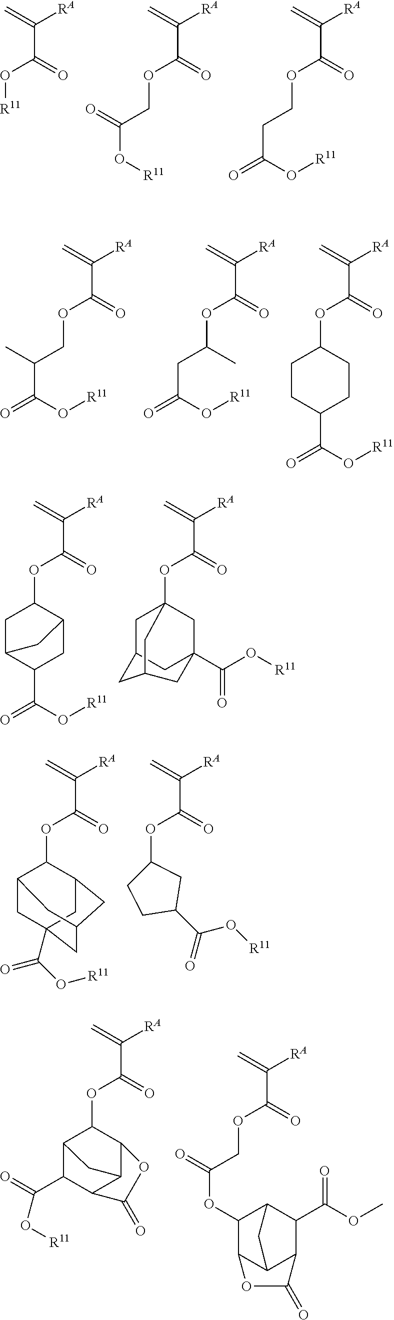

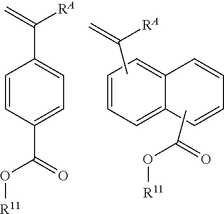

Examples of a monomer to yield the repeating unit a2 include the following monomers, but the monomer is not limited thereto. In the following formulae, R^{A} and R¹² represent the same as above.

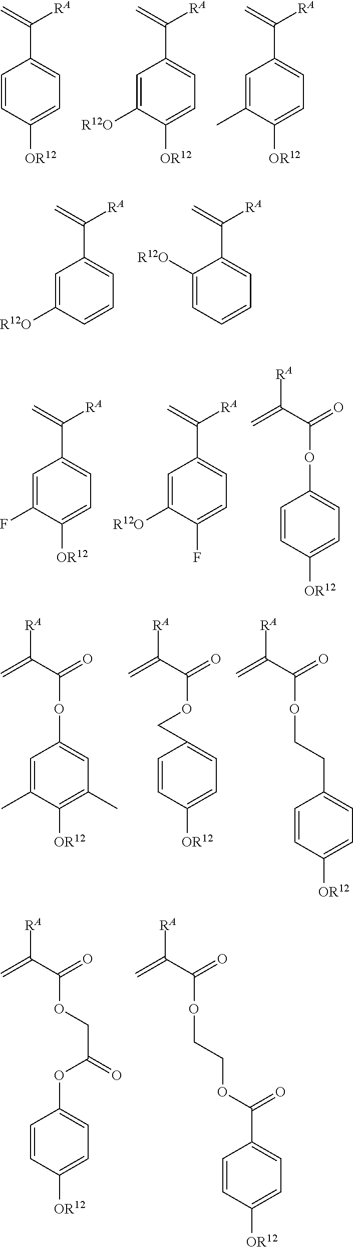

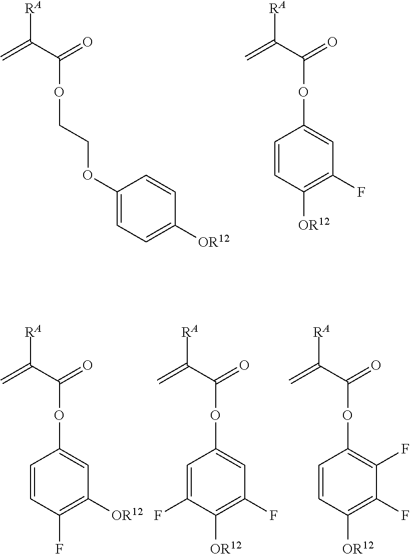

Examples of the acid-labile group represented by R^{ALU}, R¹¹, and R¹² in the general formulae (1), (a1), and (a2) include groups described in JP 2013-80033 A and JP 2013-83821 A.

Typical examples of the acid-labile group include groups represented by the following formulae (AL-1) to (AL-3).

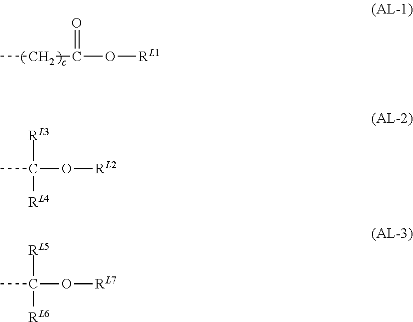

In the formulae, a broken line represents an attachment point.

In the general formulae (AL-1) and (AL-2), R^{L1} and R^{L2} each independently represent a hydrocarbyl group having 1 to 40 carbon atoms and optionally having a heteroatom such as an oxygen atom, a sulfur atom, a nitrogen atom, and a fluorine atom. The hydrocarbyl group may be a saturated or unsaturated group, and any of linear, branched, and cyclic groups. The hydrocarbyl group is preferably a saturated hydrocarbyl group having 1 to 40 carbon atoms, and more preferably a saturated hydrocarbyl group having 1 to 20 carbon atoms.

In the general formula (AL-1), “c” represents an integer of 0 to 10, and preferably an integer of 1 to 5.

In the general formula (AL-2), R^{L3} and R^{L4} each independently represent a hydrogen atom or a hydrocarbyl group having 1 to 20 carbon atoms and optionally having a heteroatom such as an oxygen atom, a sulfur atom, a nitrogen atom, and a fluorine atom. The hydrocarbyl group may be a saturated or unsaturated group, and any of linear, branched, and cyclic groups. The hydrocarbyl group is preferably a saturated hydrocarbyl group having 1 to 20 carbon atoms. Any two of R^{L2}, R^{L3}, and R^{L4} are optionally bonded to each other to form a ring having 3 to 20 carbon atoms together with the carbon atom or the carbon atom and oxygen atom to which these groups are bonded. The ring is preferably a ring having 4 to 16 carbon atoms, and particularly preferably an aliphatic ring.

In the formula (AL-3), R^{L5}, R^{L6}, and R^{L7} each independently represent a hydrocarbyl group having 1 to 20 carbon atoms and optionally having a heteroatom such as an oxygen atom, a sulfur atom, a nitrogen atom, and a fluorine atom. The hydrocarbyl group may be a saturated or unsaturated group, and any of linear, branched, and cyclic groups. The hydrocarbyl group is preferably a saturated hydrocarbyl group having 1 to 20 carbon atoms. Any two of R^{L5}, R^{L6}, and R^{L7} are optionally bonded to each other to form a ring having 3 to 20 carbon atoms together with the carbon atom to which these groups are bonded. The ring is preferably a ring having 4 to 16 carbon atoms, and particularly preferably an aliphatic ring.

When the base polymer has the repeating unit a1 and/or a2, the resist composition is a chemically amplified positive-type resist composition.

The base polymer in the resist composition also preferably has no acid-labile group. In this case, the resist composition is a chemically amplified negative-type resist composition.

The base polymer may have a repeating unit b having a phenolic hydroxy group as an adhesive group. Examples of a monomer to yield the repeating unit b include the following monomers, but the monomer is not limited thereto. In the following formulae, R^{A} represents the same as above.

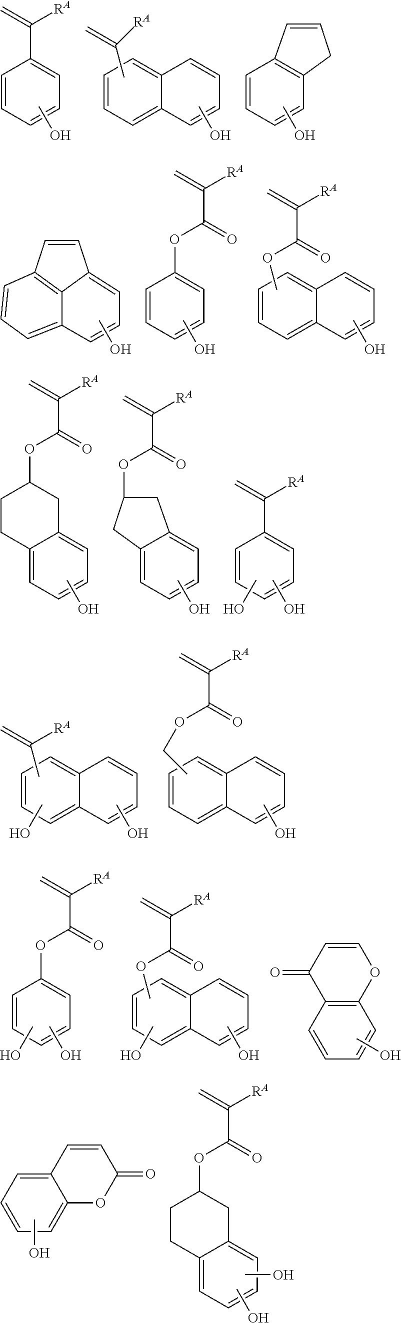

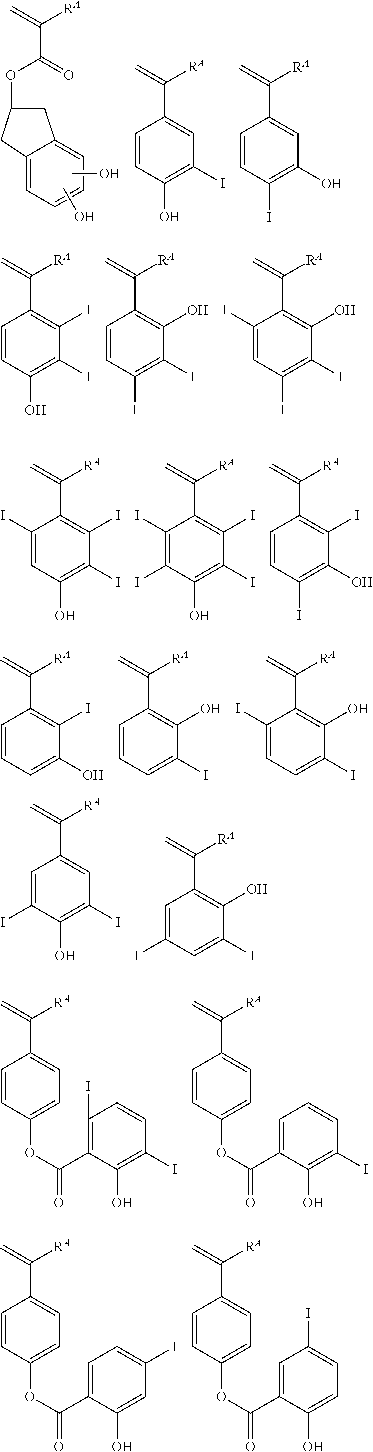

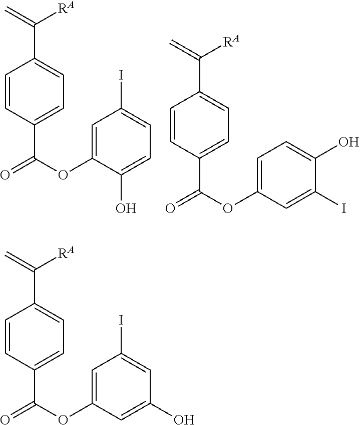

The base polymer may have a repeating unit c having, as another adhesive group, a hydroxy group other than a phenolic hydroxy group, a lactone ring, a sultone ring, an ether bond, an ester bond, a sulfonate ester bond, a carbonyl group, a sulfonyl group, a cyano group, and/or a carboxy group. Examples of a monomer to yield the repeating unit c include the following monomers, but the monomer is not limited thereto. In the following formulae, R^{A} represents the same as above.

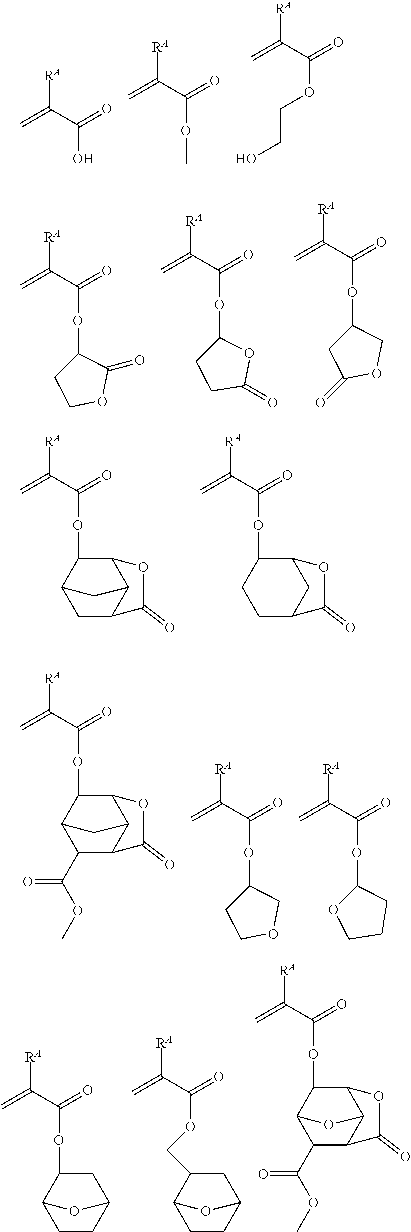

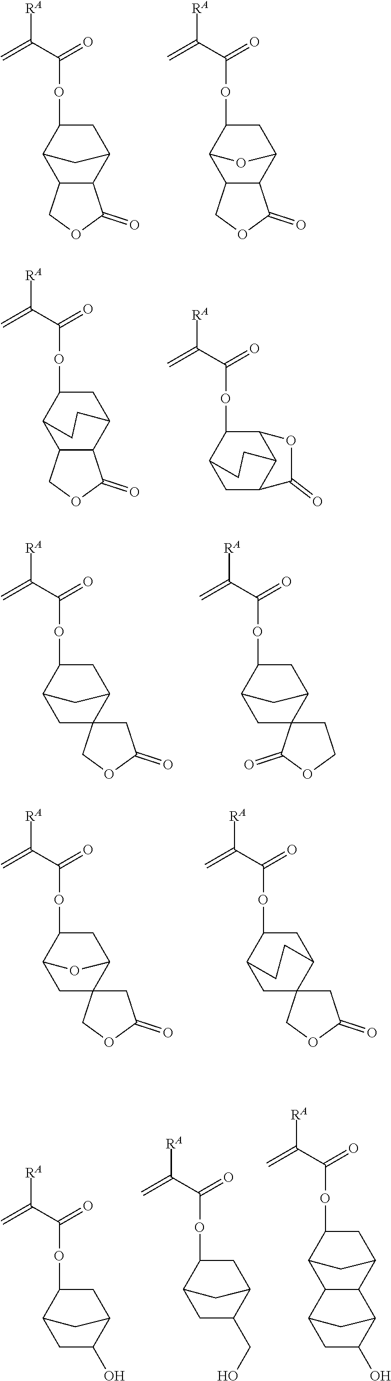

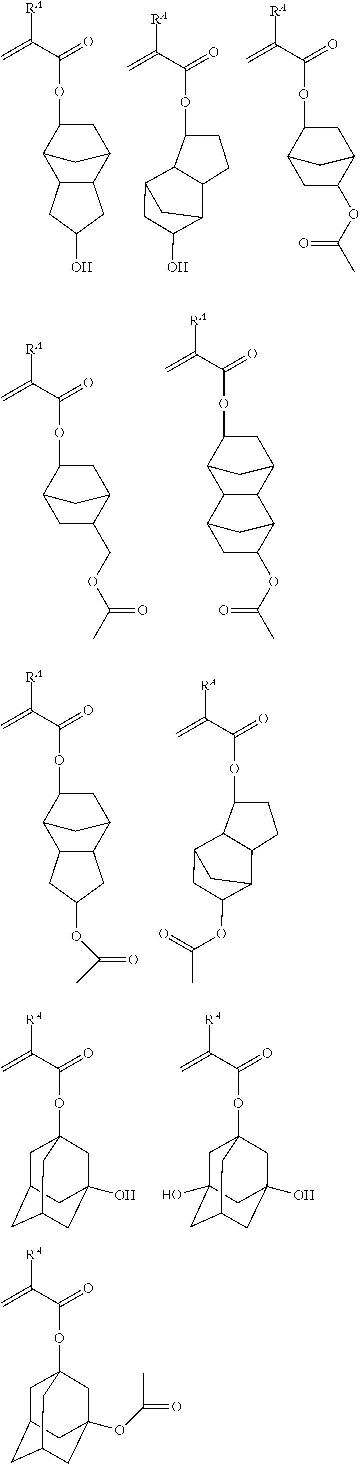

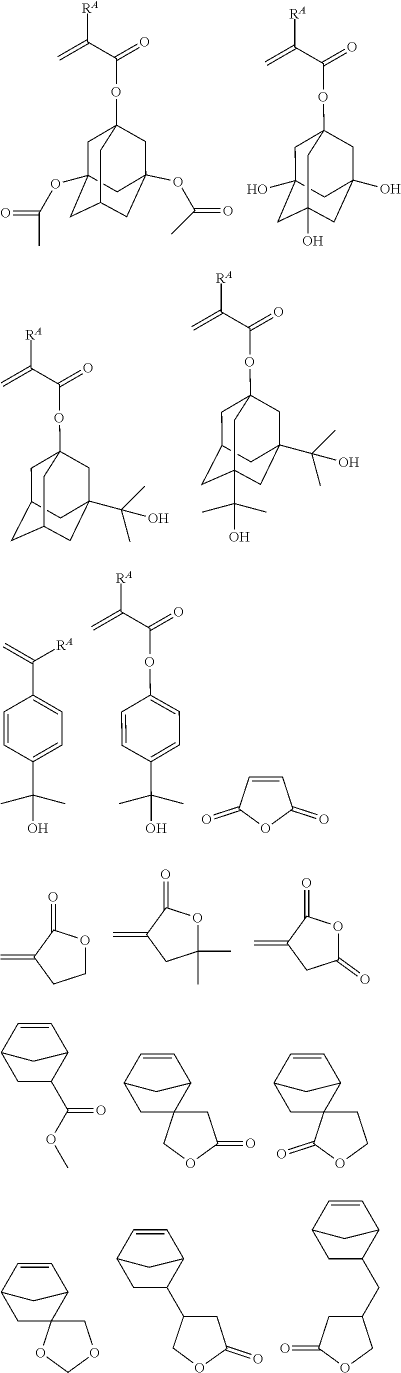

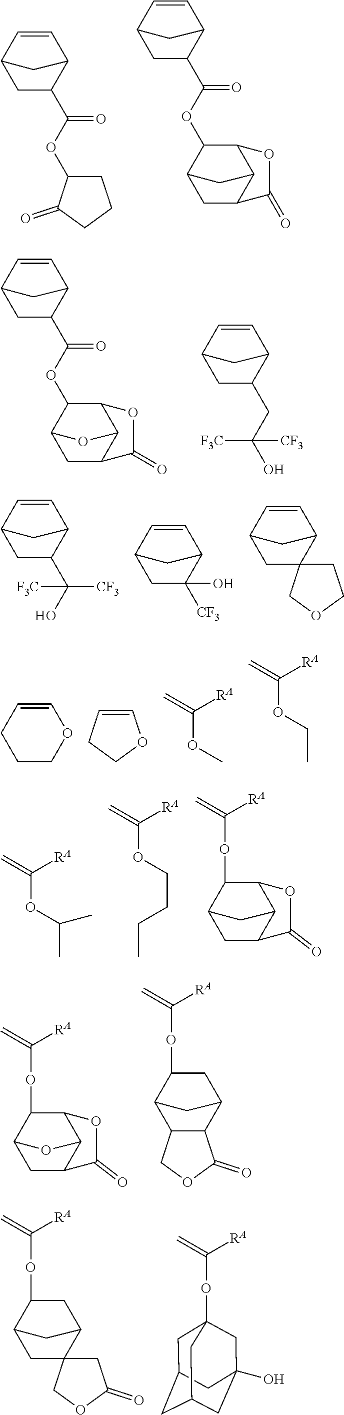

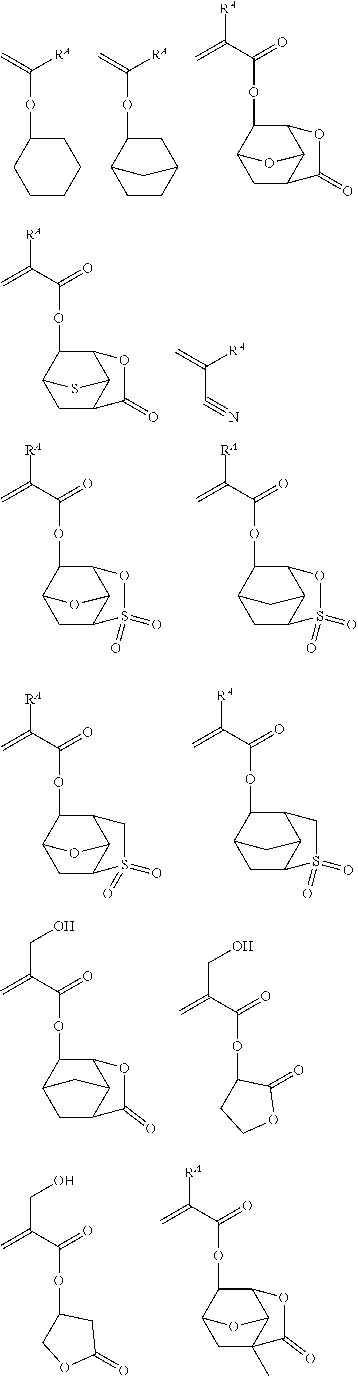

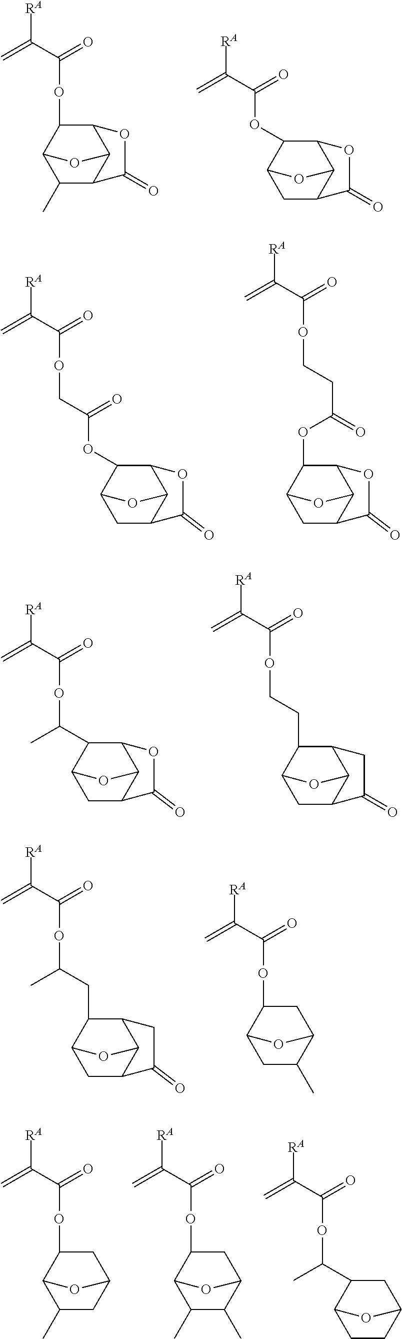

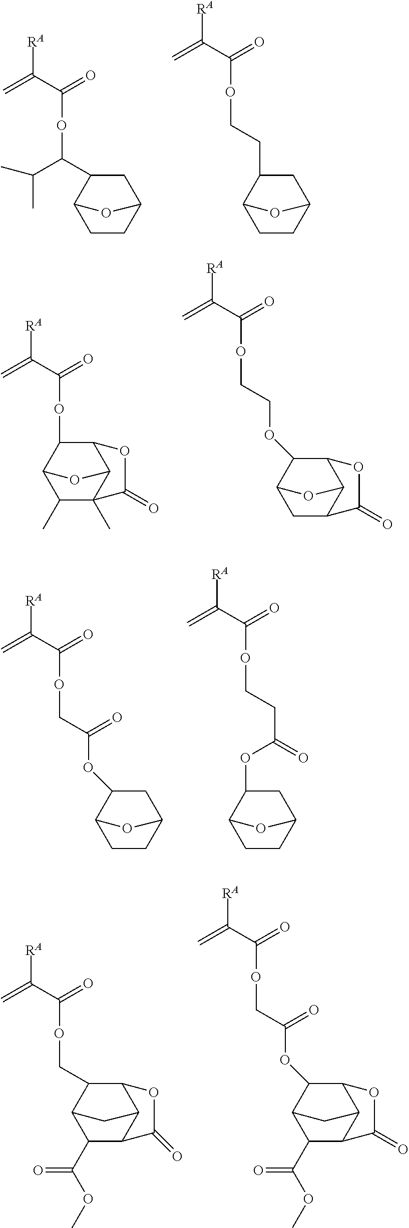

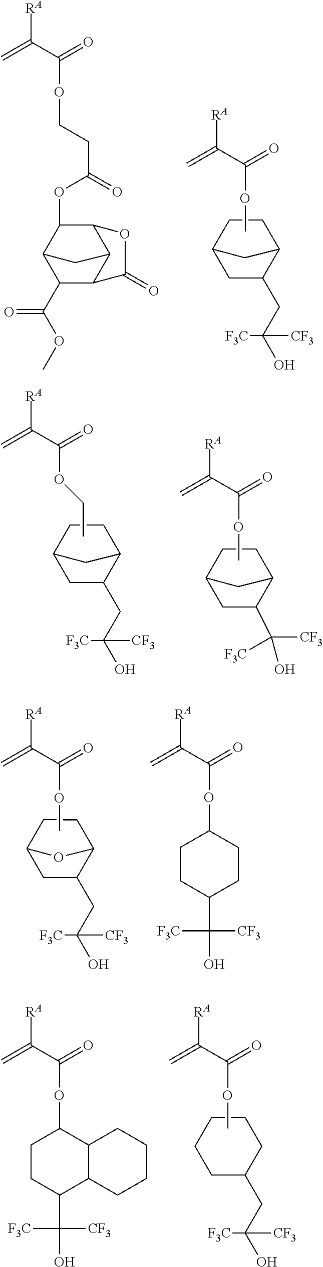

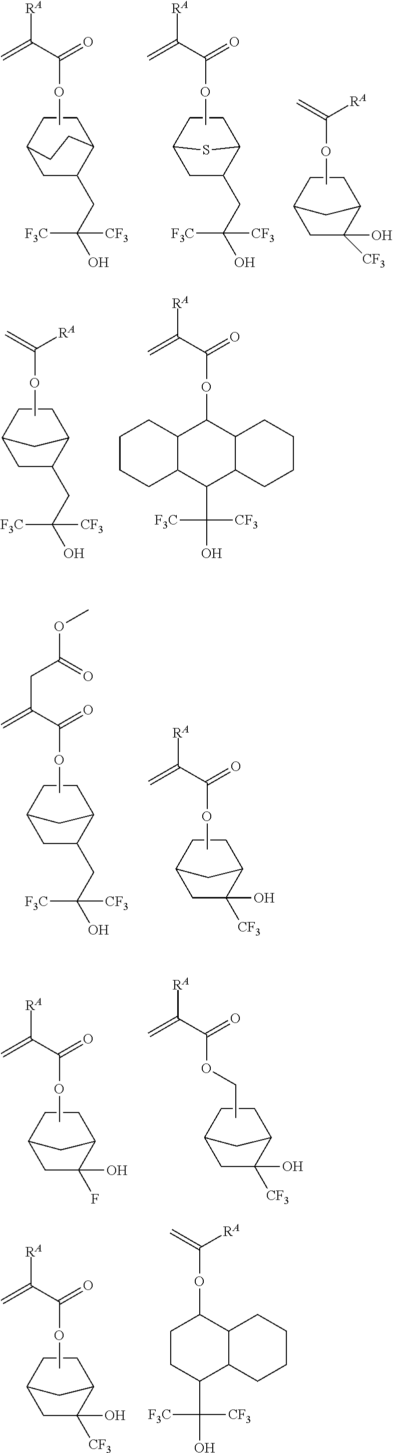

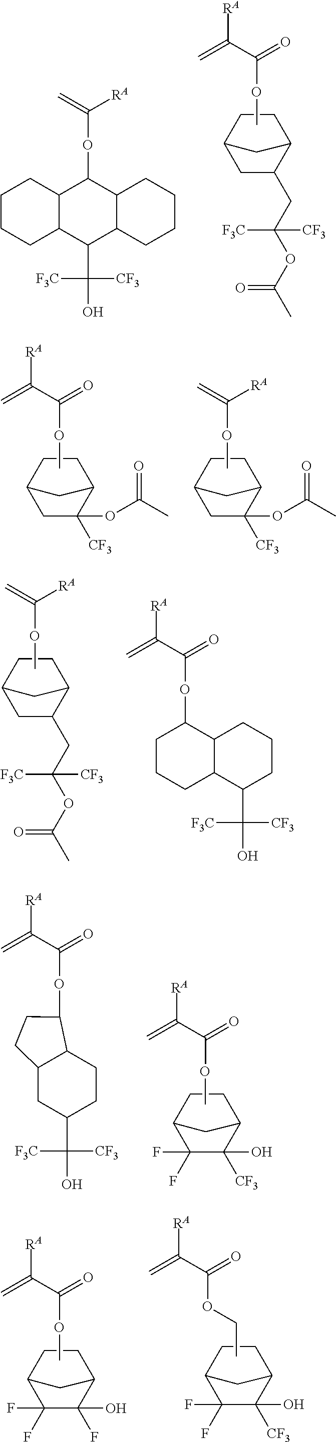

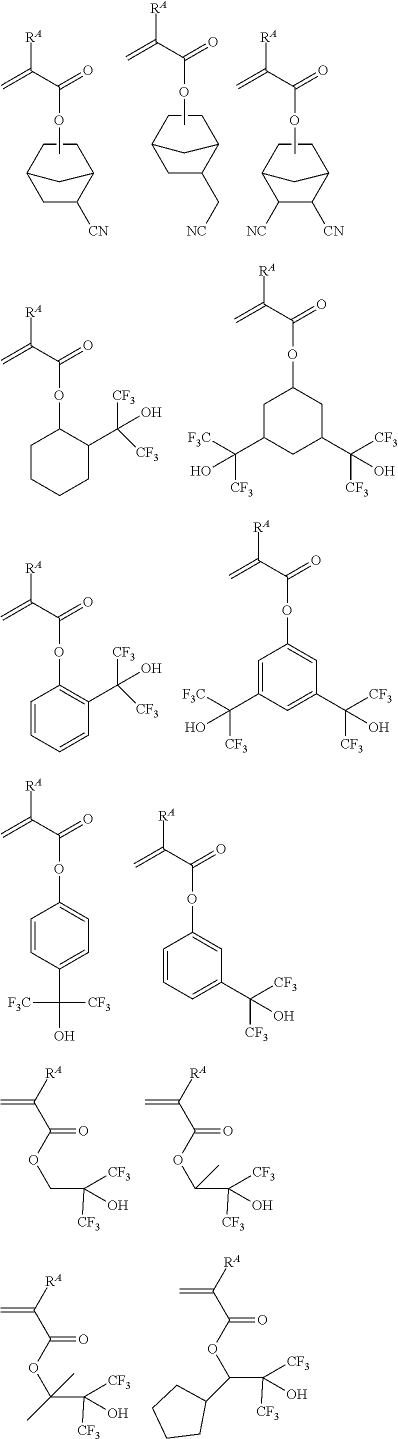

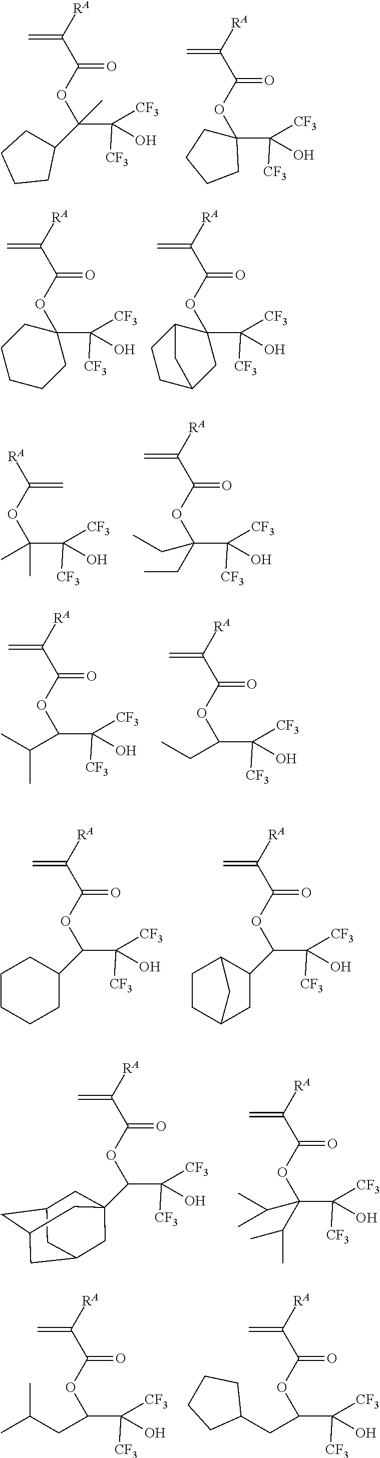

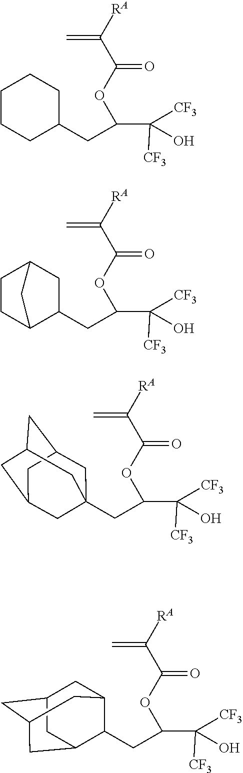

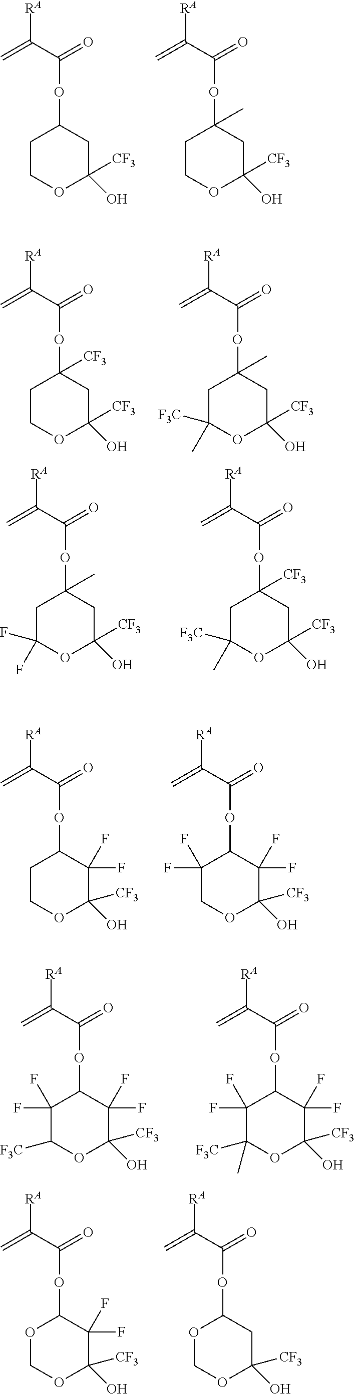

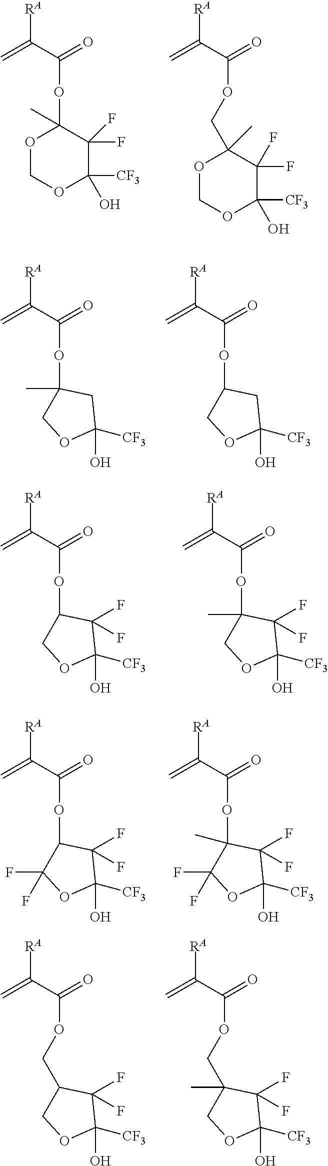

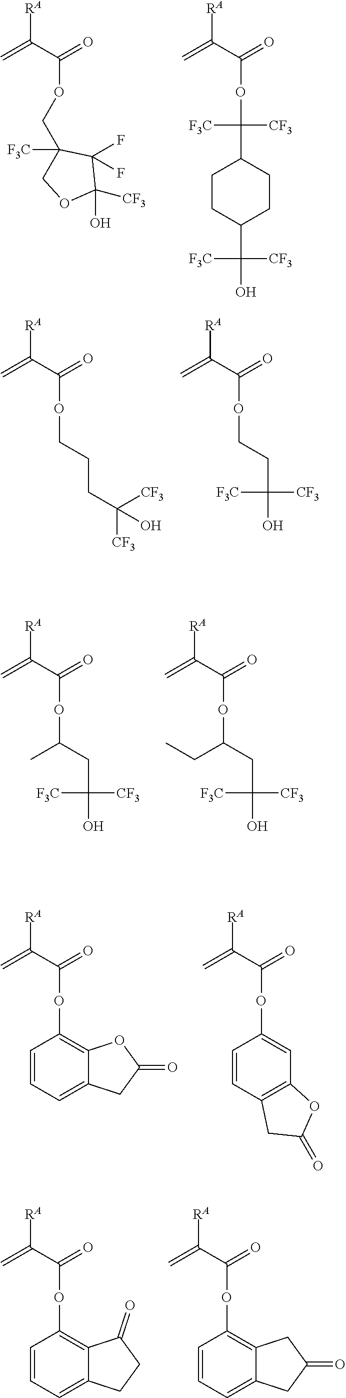

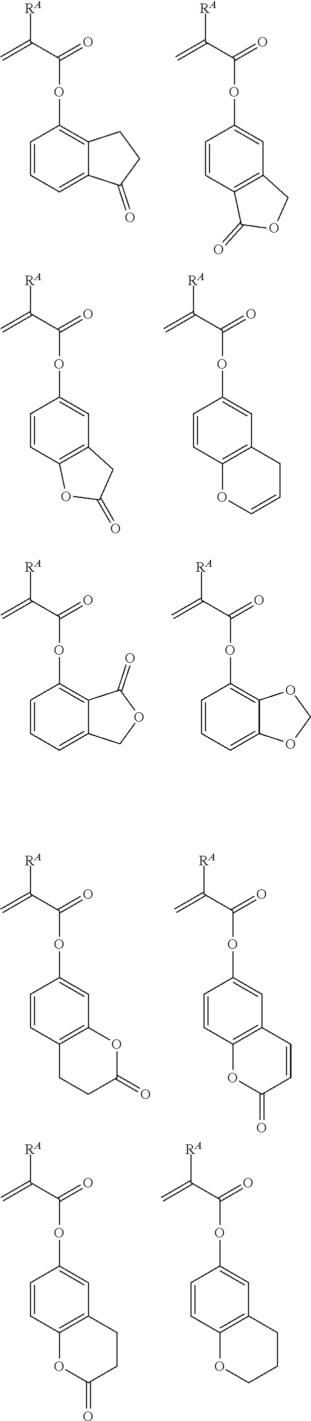

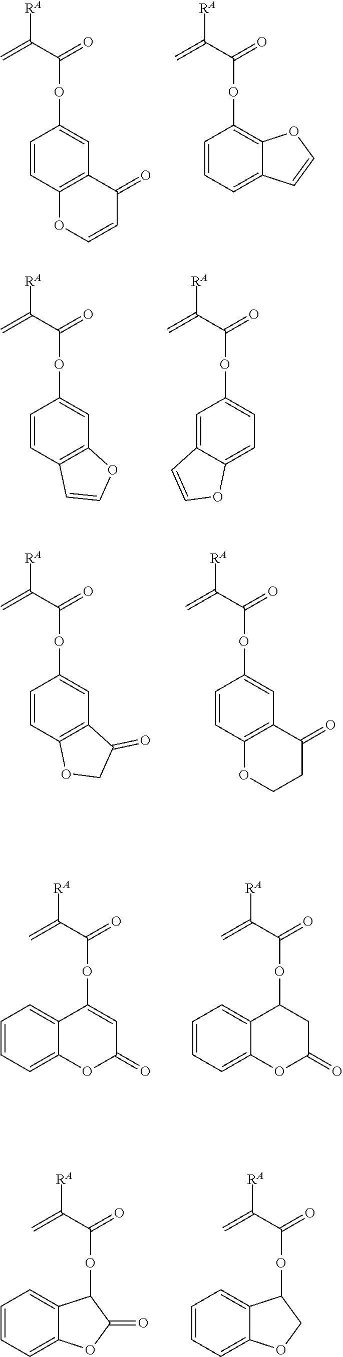

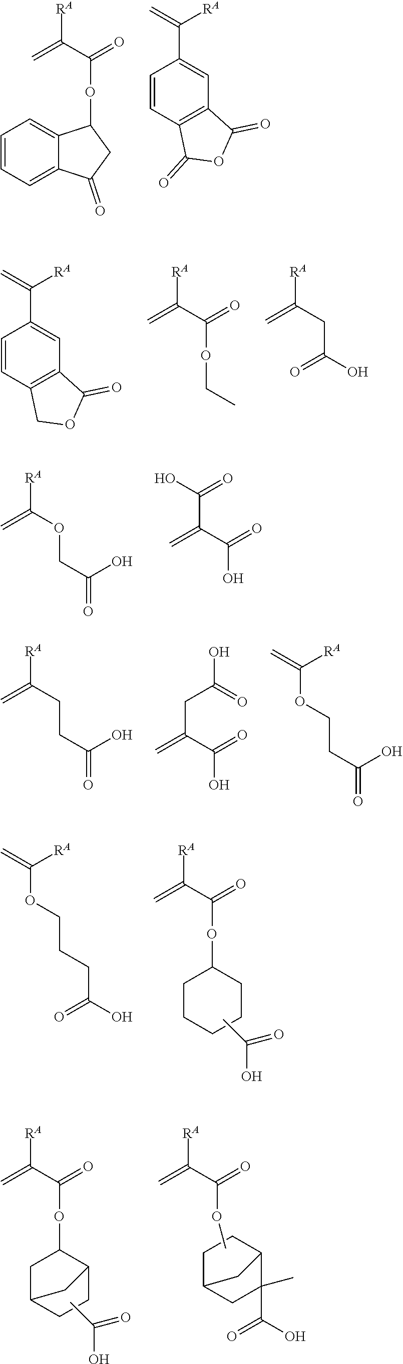

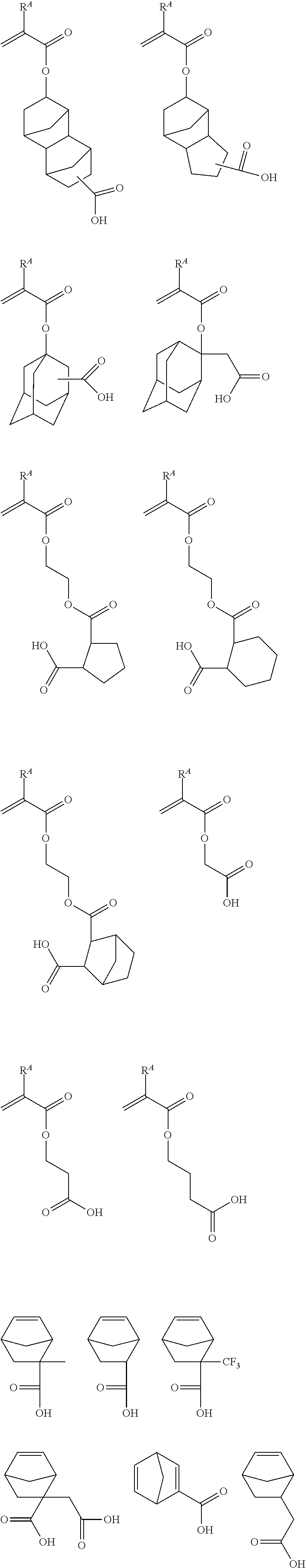

The base polymer may have a repeating unit d derived from indene, benzofuran, benzothiophene, acenaphthylene, chromone, coumarin, norbornadiene, or a derivative thereof. Examples of a monomer to yield the repeating unit d include the following monomers, but the monomer is not limited thereto.

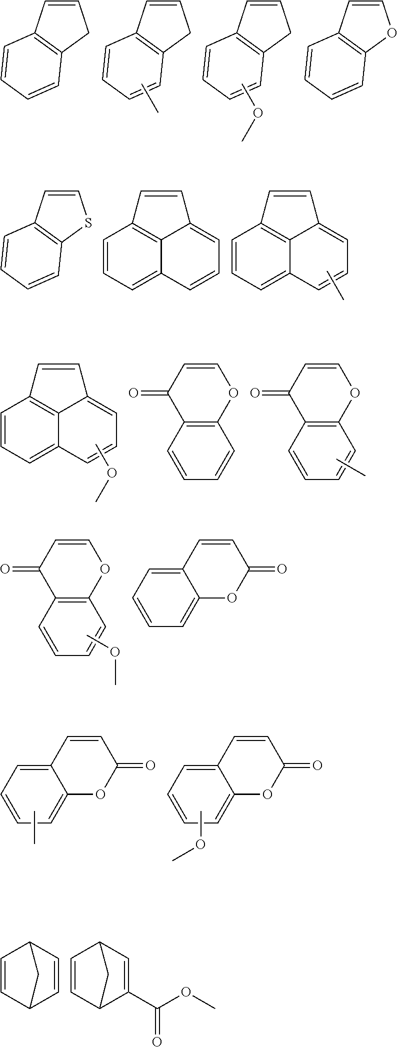

The base polymer may have a repeating unit e derived from styrene, vinylnaphthalene, vinylanthracene, vinylpyrene, methyleneindane, vinylpyridine, or vinylcarbazole.

The base polymer may have a repeating unit f derived from an onium salt having a polymerizable unsaturated bond. Examples of a preferable repeating unit f include a repeating unit represented by the following general formula (f1) (hereinafter, also referred to as the repeating unit f1), a repeating unit represented by the following general formula (f2) (hereinafter, also referred to as the repeating unit f2), and a repeating unit represented by the following general formula (f3) (hereinafter, also referred to as the repeating unit f3). As the repeating units f1 to f3, one kind may be used, or combination of two or more kinds may be used.

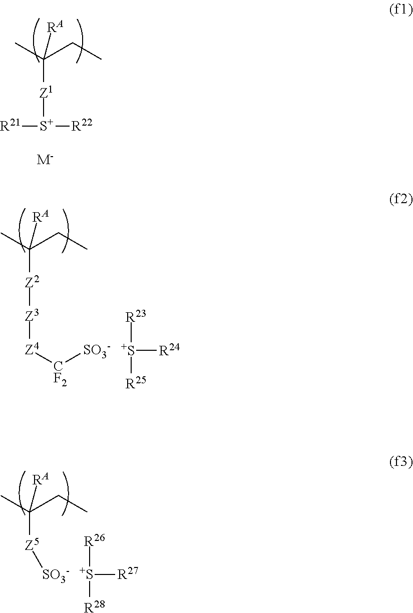

In the general formulae (f1) to (f3), R^{A} each independently represents a hydrogen atom or a methyl group. Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)—O—Z¹¹—, or —C(═O)—NH—Z¹—. Z¹¹ represents an aliphatic hydrocarbylene group having 1 to 6 carbon atoms, a phenylene group, a naphthylene group, a group having 7 to 18 carbon atoms obtained by combining these groups, Z¹¹ optionally having a carbonyl group, an ester bond, an ether bond, or a hydroxy group. Z² represents a single bond or an ester bond. Z³ represents a single bond, —Z³¹—C(═O)—O—, —Z³¹—O—, or —Z³¹—O—C(═O)—. Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom. Z⁴ represents a methylene group, a 2,2,2-trifluoro-1,1-ethanediyl group, or a carbonyl group. Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—. Z⁵¹ represents an aliphatic hydrocarbylene group having 1 to 6 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group.

In the general formulae (f1) to (f3), R²¹ to R²⁸ each independently represent a halogen atom or a hydrocarbyl group having 1 to 20 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R^{11′} to R^{19′} in the description of the general formulae (Cation-1) to (Cation-3). A part or all of hydrogen atoms in the hydrocarbyl group are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained is a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, or a haloalkyl group, etc. R²³ and R²⁴ or R²⁶ and R²⁷ are optionally bonded to each other to form a ring together with the sulfur atom to which these groups are bonded. In this case, examples of the ring include rings same as those exemplified as the rings that can be formed by bonding R^{11′} and R^{12′} together with the sulfur atom to which these groups are bonded, described in the general formula (Cation-1).

In the general formula (f1), M⁻ represents a non-nucleophilic counterion. Examples of the non-nucleophilic counterion include: halide ions, such as a chloride ion and a bromide ion; fluoroalkylsulfonate ions, such as a triflate ion, a 1,1,1-trifluoroethanesulfonate ion, and a nonafluorobutanesulfonate ion; arylsulfonate ions, such as a tosylate ion, a benzenesulfonate ion, a 4-fluorobenzenesulfonate ion, and a 1,2,3,4,5-pentafluorobenzenesulfonate ion; alkylsulfonate ions, such as a mesylate ion and a butanesulfonate ion; imide ions, such as a bis(trifluoromethylsulfonyl)imide ion, a bis(perfluoroethylsulfonyl)imide ion, and a bis(perfluorobutylsulfonyl)imide ion; and methide ions, such as a tris(trifluoromethylsulfonyl)methide ion and a tris(perfluoroethylsulfonyl)methide ion.

Other examples of the non-nucleophilic counterion include: a sulfonate ion in which the α-position is substituted with a fluorine atom, represented by the following general formula (f1-1); and a sulfonate ion in which the α-position is substituted with a fluorine atom and the β-position is substituted with a trifluoromethyl group, represented by the following general formula (f1-2).

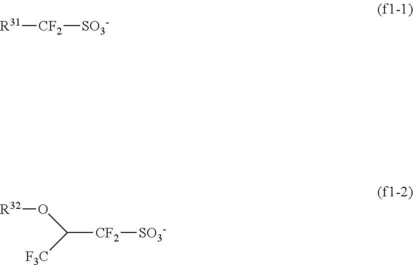

In the general formula (f1-1), R³¹ represents a hydrogen atom or a hydrocarbyl group having 1 to 20 carbon atoms. The hydrocarbyl group optionally has an ether bond, an ester bond, a carbonyl group, a lactone ring, or a fluorine atom. The hydrocarbyl group may be a saturated or unsaturated group, and may be any of linear, branched, and cyclic groups. Specific examples thereof include groups same as those exemplified as a hydrocarbyl group represented by R¹¹¹ in the formula (3A′), described later.

In the general formula (f1-2), R³² represents a hydrogen atom, a hydrocarbyl group having 1 to 30 carbon atoms, or a hydrocarbylcarbonyl group having 6 to 20 carbon atoms. The hydrocarbyl group and the hydrocarbylcarbonyl group optionally have an ether bond, an ester bond, a carbonyl group, or a lactone ring. Each of the hydrocarbyl group and a hydrocarbyl part of the hydrocarbylcarbonyl group may be a saturated or unsaturated group, and may be any of linear, branched, and cyclic groups. Specific examples thereof include groups same as those exemplified as a hydrocarbyl group represented by R¹¹¹ in the formula (3A′), described later.

Examples of a cation of a monomer to yield the repeating unit f1 include the following cations, but the cation is not limited thereto. In the following formulae, R^{A} represents the same as above.

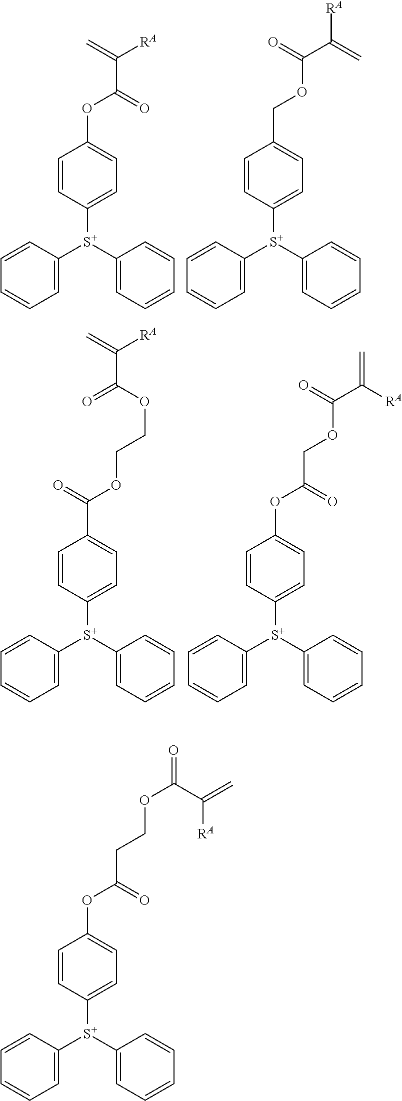

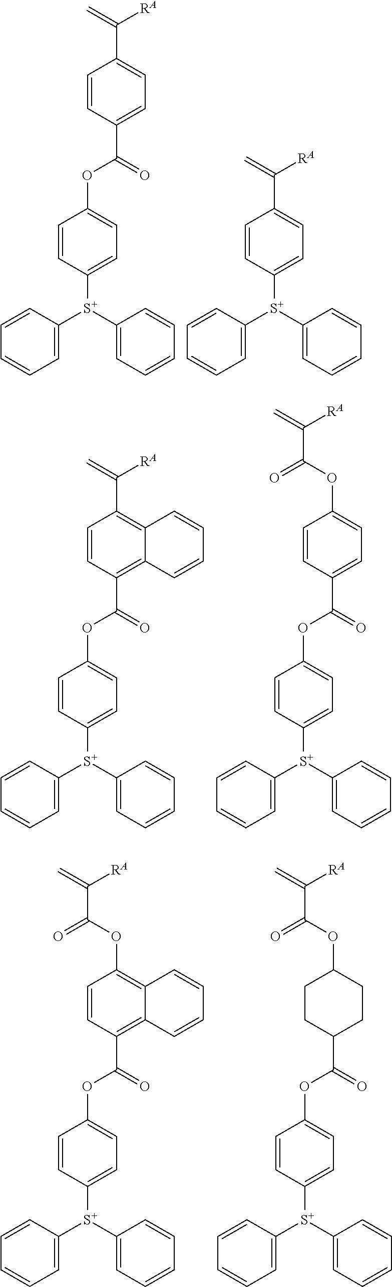

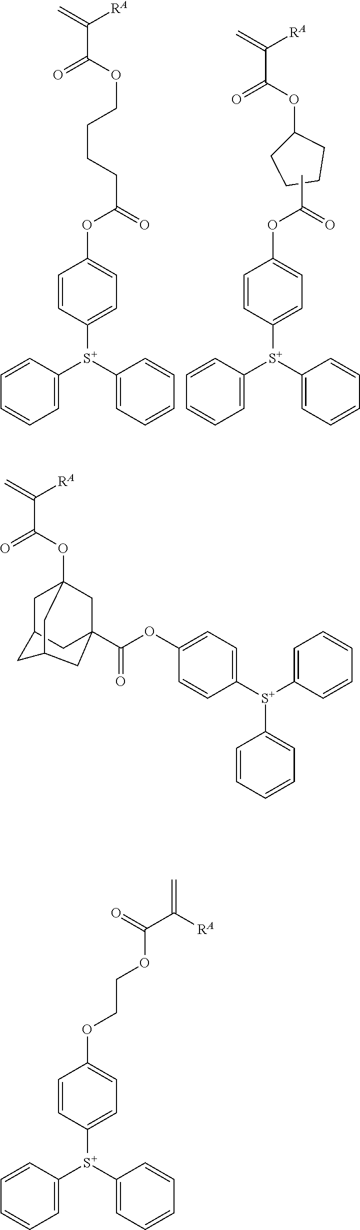

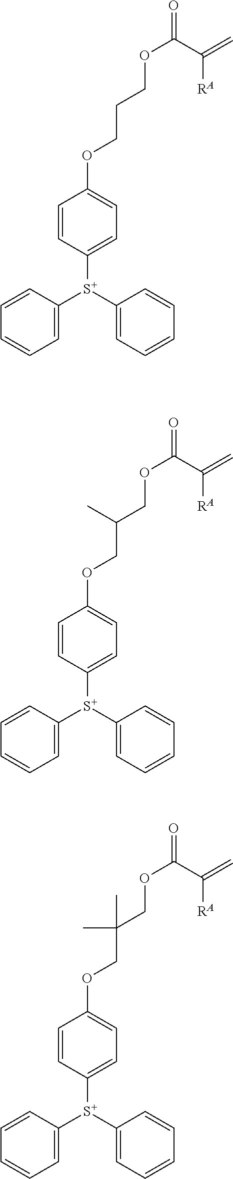

Specific examples of a cation of a monomer to yield the repeating unit f2 or f3 include cations same as those exemplified as the cation of the sulfonium salt represented by the formula (Cation-1).

Examples of an anion of a monomer to yield the repeating unit f2 include the following anions, but the anion is not limited thereto. In the following formulae, R^{A} represents the same as above.

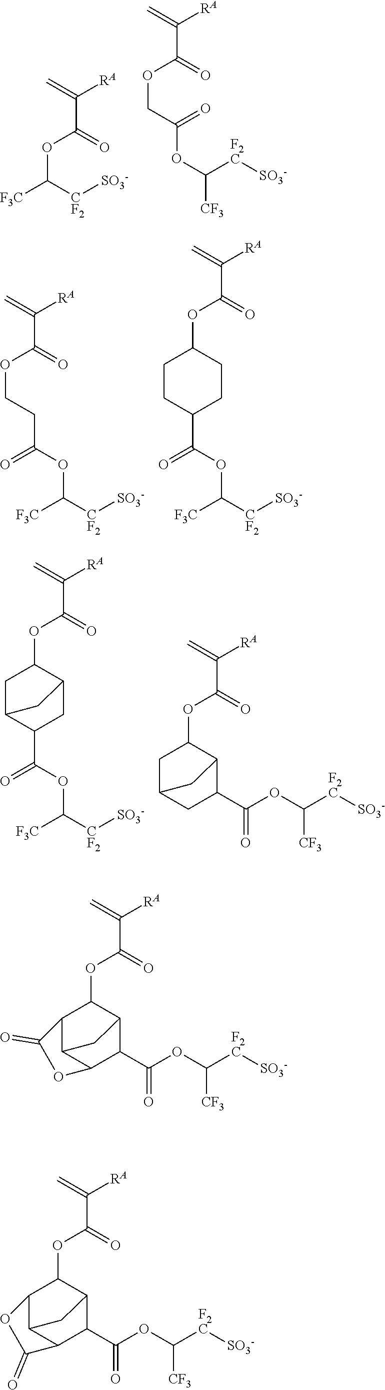

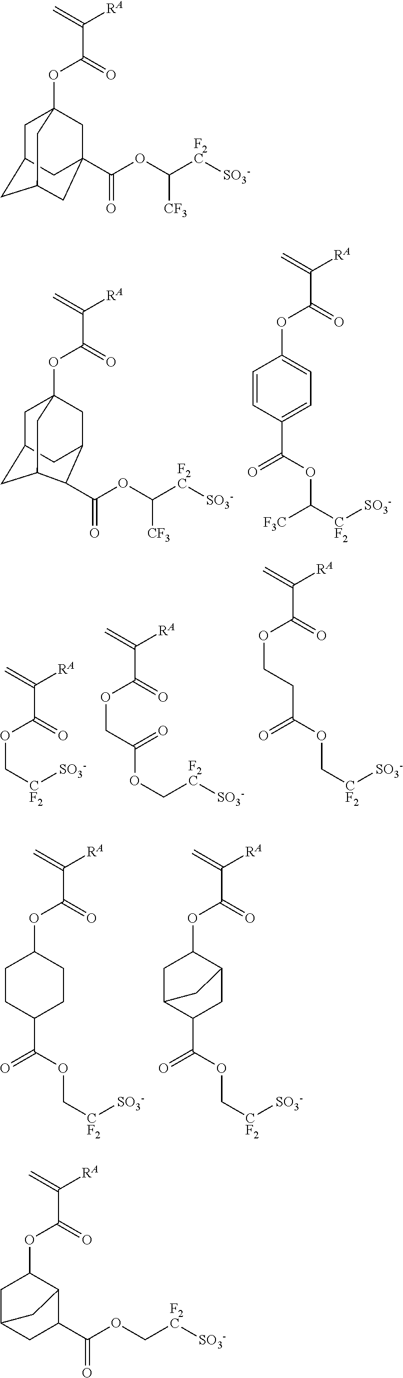

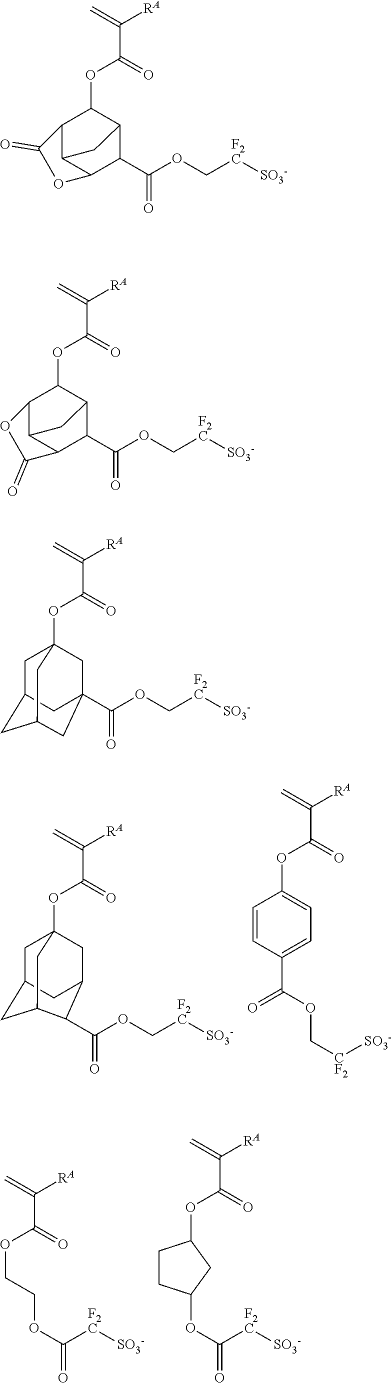

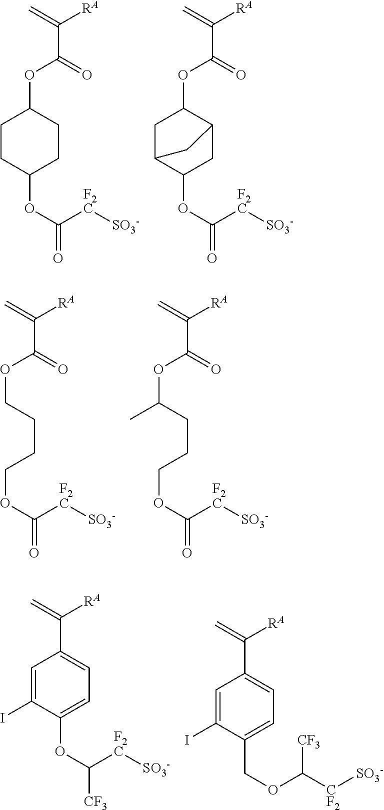

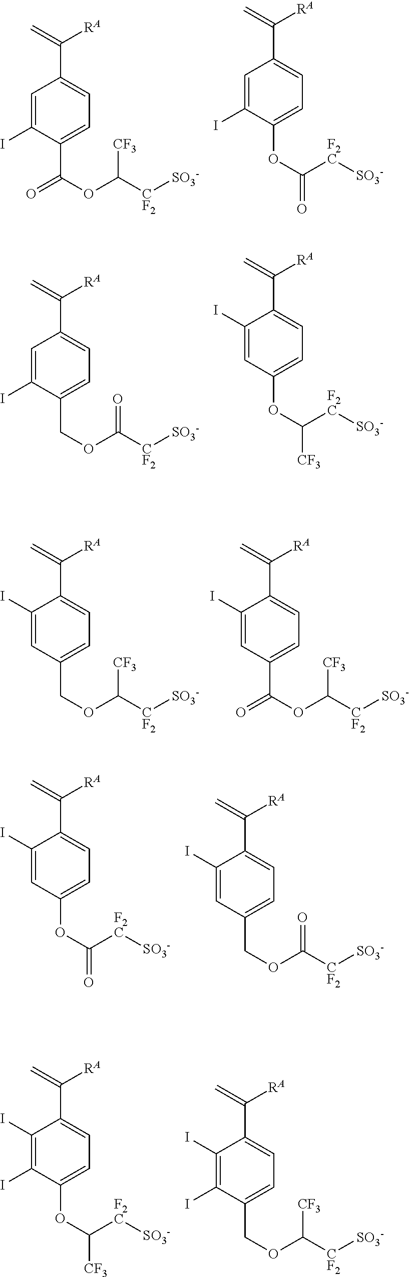

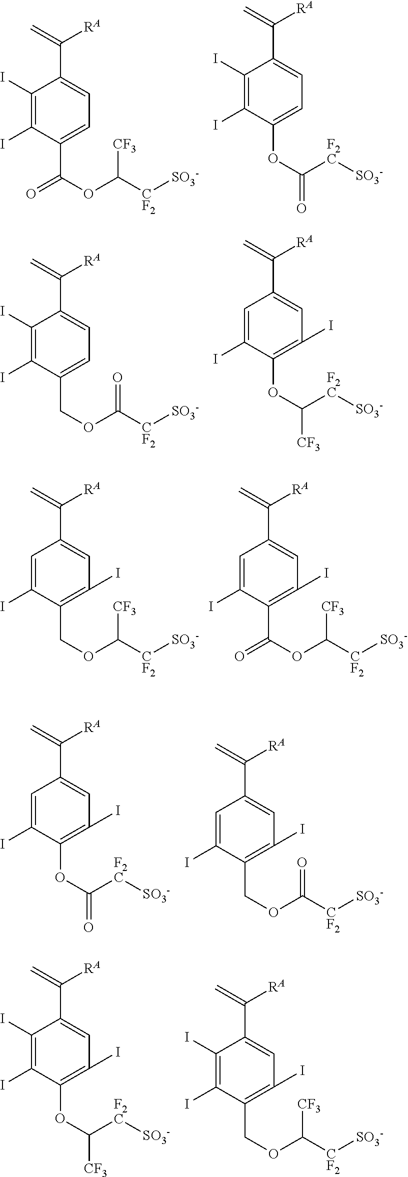

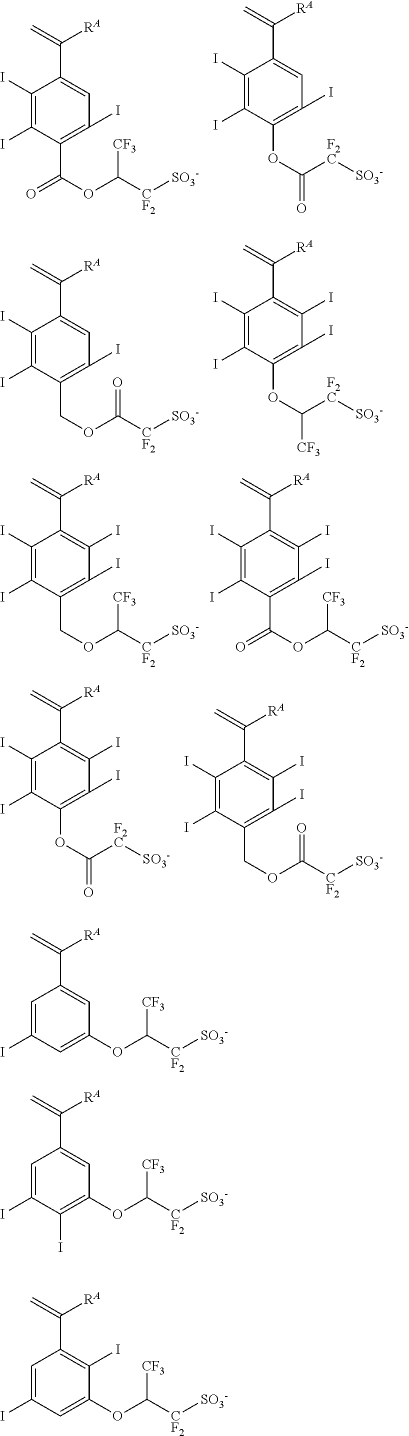

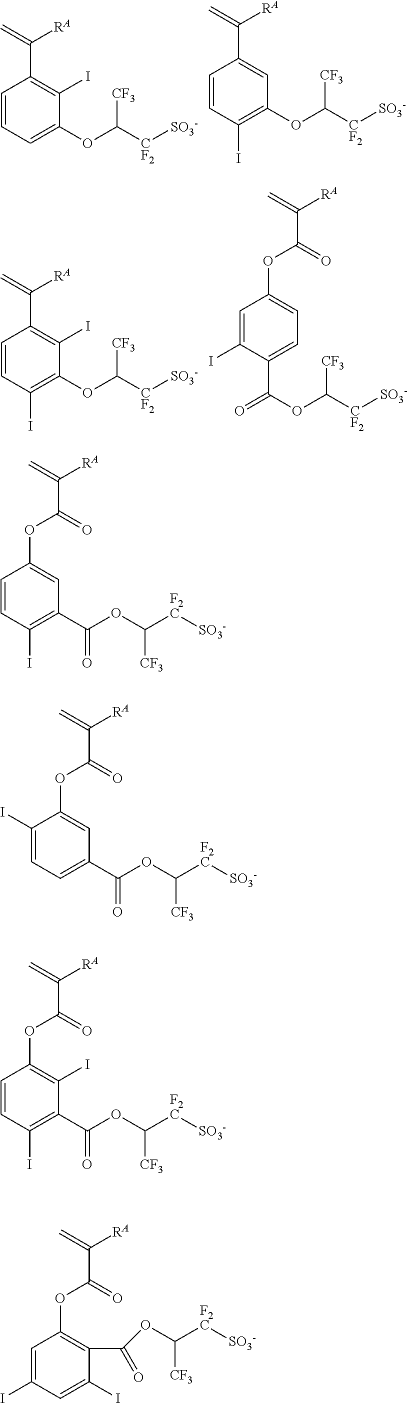

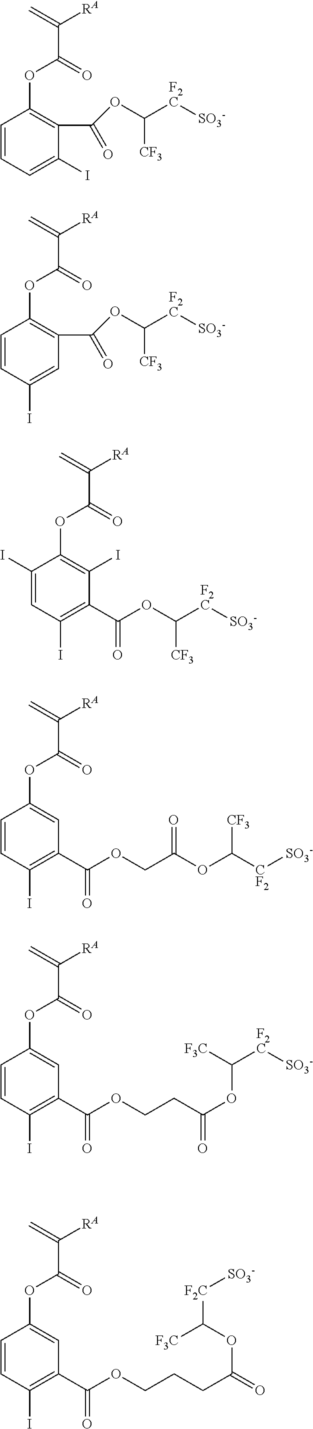

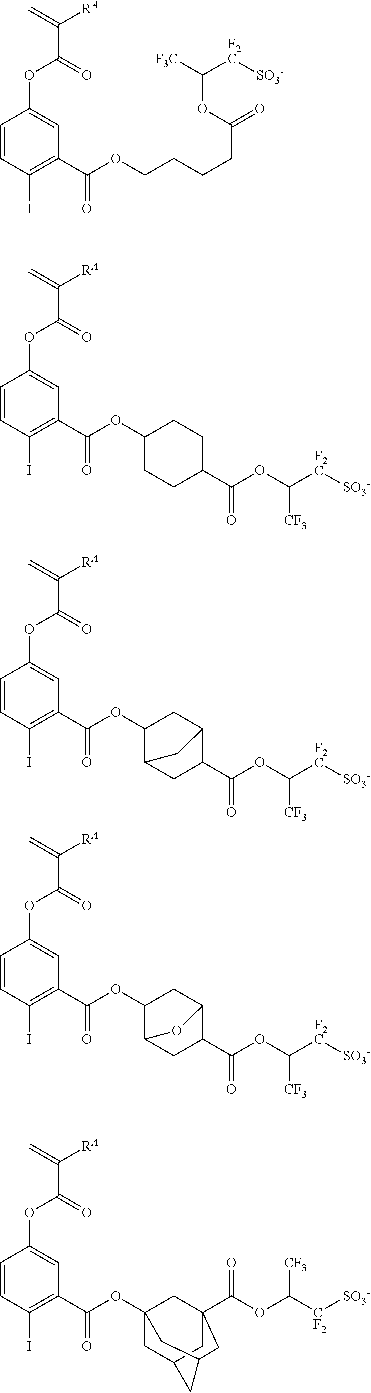

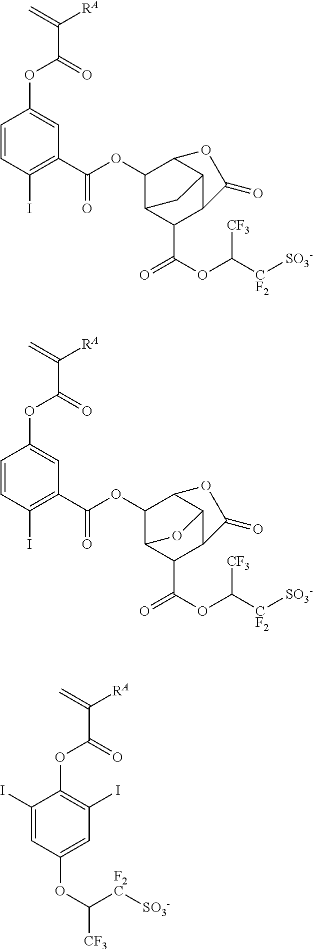

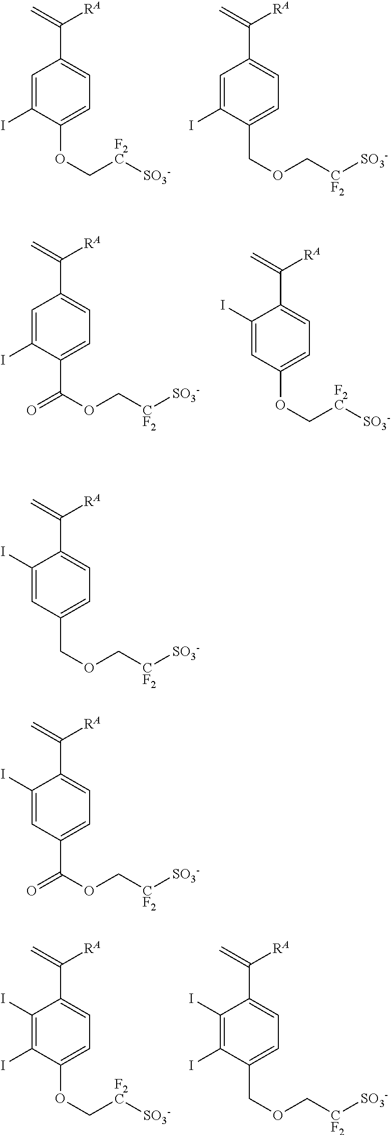

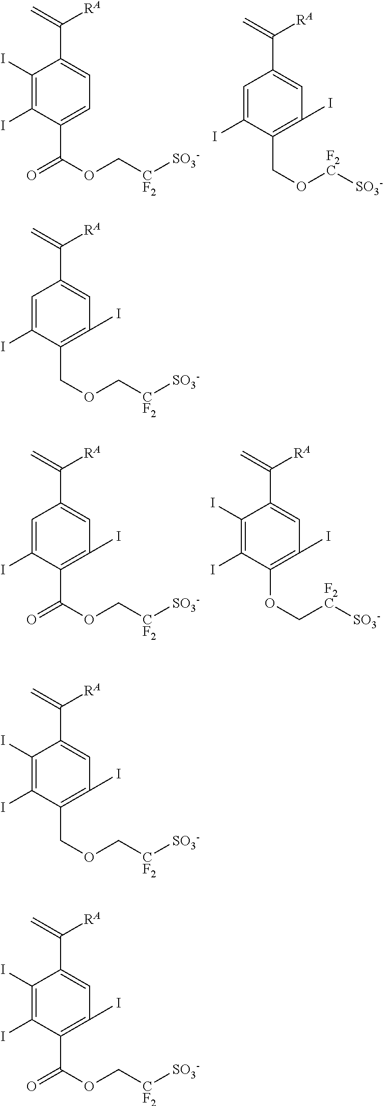

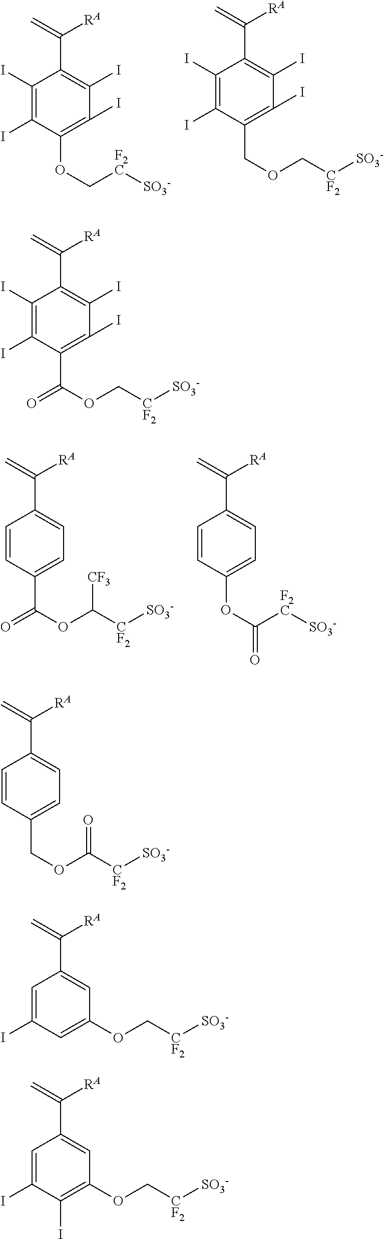

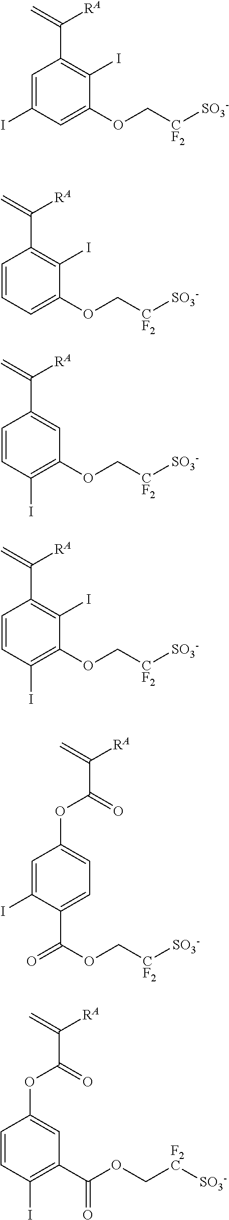

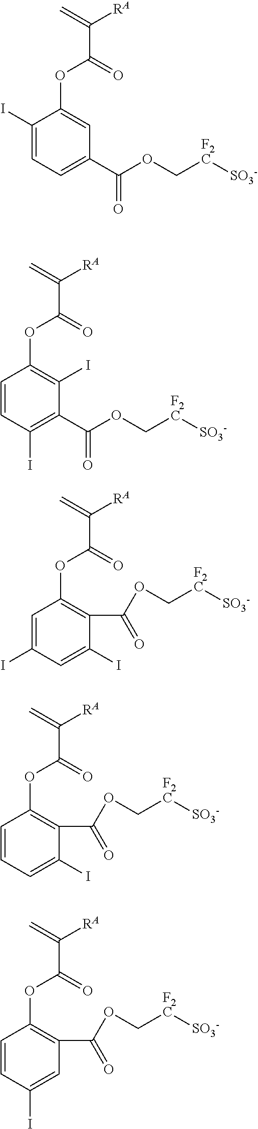

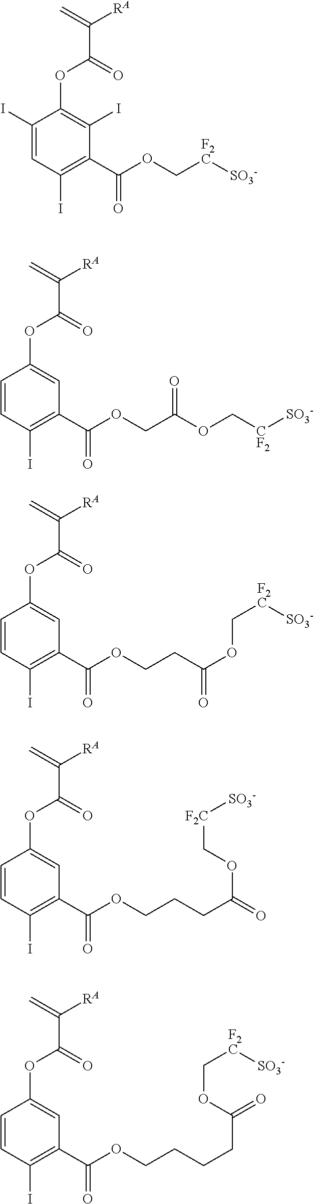

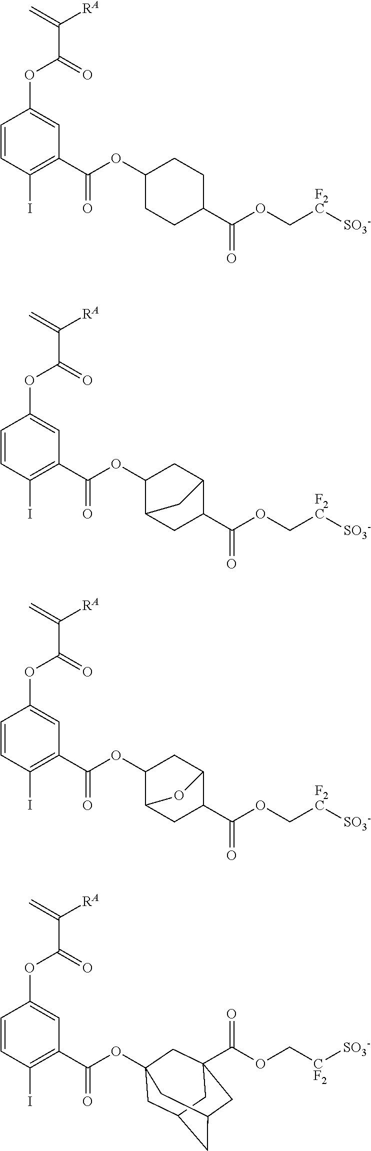

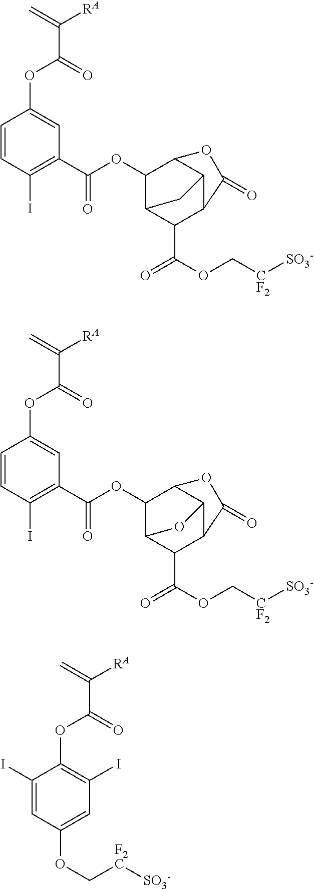

Examples of an anion of a monomer to yield the repeating unit f3 include the following anions, but the anion is not limited thereto. In the following formulae, R^{A} represents the same as above.

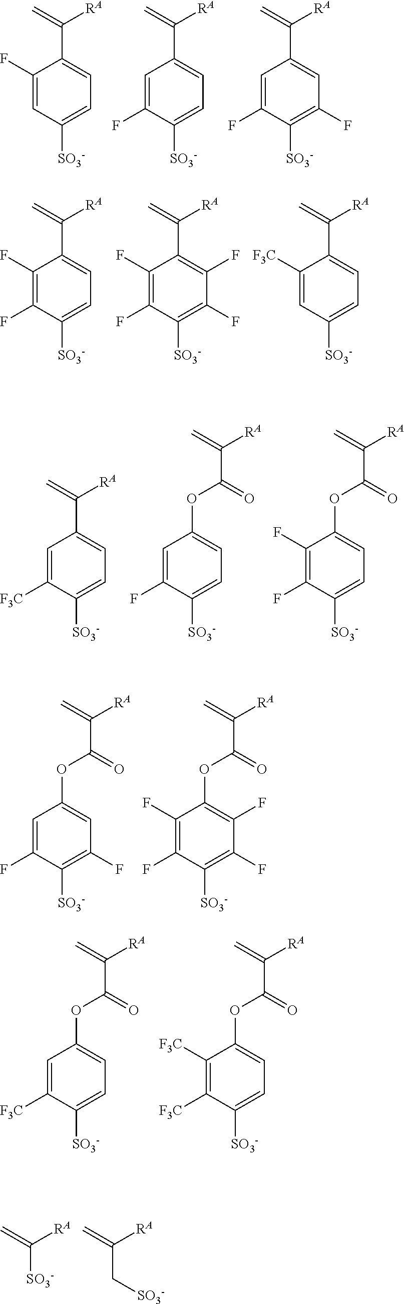

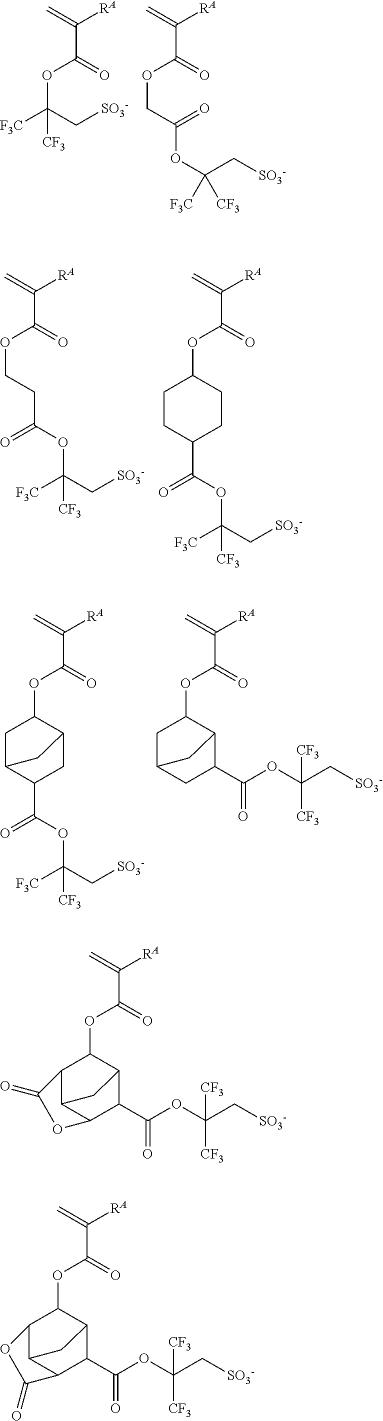

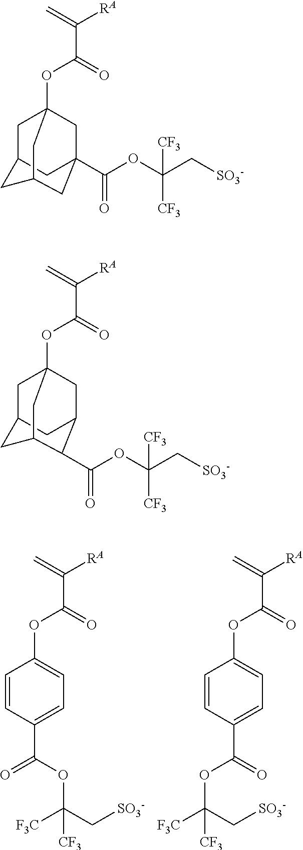

The repeating units f1 to f3 have a function of an acid generator. By binding the acid generator to the polymer main chain, the acid diffusion can be reduced, and deterioration of resolution due to blur with the acid diffusion can be prevented. In addition, by uniformly dispersing the acid generator, LWR and CDU can be improved. When the base polymer having the repeating unit f is used, blending an additive-type acid generator, described later, can be omitted.

In the base polymer, content rates of the repeating units a1, a2, b, c, d, e, f1, f2, and f3 are preferably 0≤a1≤0.9, 0≤a2≤0.9, 0≤a1+a2≤0.9, 0≤b≤0.9, 0≤c≤0.9, 0≤d≤0.5, 0≤e≤0.5, 0≤f1≤0.5, 0≤f2≤0.5, 0≤f3≤0.5, and 0≤f1+f2+f3≤0.5, more preferably 0≤a1≤0.8, 0≤a2≤0.8, 0≤a1+a2≤0.8, 0≤b≤0.8, 0≤c≤0.8, 0≤d 0.4, 0≤e≤0.4, 0≤f1≤0.4, 0≤f2≤0.4, 0≤f3≤0.4, and 0≤f1+f2+f3≤0.4, and further preferably 0≤a1≤0.7, 0≤a2≤0.7, 0≤a1+a2≤0.7, 0≤b 0.7, 0≤c≤0.7, 0≤d≤0.3, 0≤e≤0.3, 0≤f1≤0.3, 0≤f2≤0.3, 0≤f3≤0.3, and 0≤f1+f2+f3≤0.3. Note that, a1+a2+b+c+d+f1+f2+f3+e=1.0.

To synthesize the base polymer, the monomers to yield the aforementioned repeating units are heated in an organic solvent with adding a radical polymerization initiator to perform polymerization, for example.

Examples of the organic solvent used for the polymerization include toluene, benzene, tetrahydrofuran (THF), diethyl ether, and dioxane. Examples of the polymerization initiator include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. A temperature for the polymerization is preferably 50 to 80° C. The reaction time is preferably 2 to 100 hours, and more preferably 5 to 20 hours.

When a monomer having a hydroxy group is copolymerized, the hydroxy group may be substituted with an acetal group, such as an ethoxyethyl group, easily deprotected by an acid, for the polymerization, and the protected hydroxy group may be deprotected by a weak acid and water after the polymerization. Alternatively, the hydroxy group may be substituted with an acetyl group, a formyl group, a pivaloyl group, etc. and hydrolyzed with an alkali after the polymerization.

When hydroxystyrene or hydroxyvinylnaphthalene is copolymerized, acetoxystyrene or acetoxyvinylnaphthalene may be used instead of hydroxystyrene or hydroxyvinylnaphthalene, and after the polymerization, the acetoxy group may be deprotected by the above alkaline hydrolysis to form hydroxystyrene or hydroxyvinylnaphthalene.

As a base in the alkaline hydrolysis, aqueous ammonia, triethylamine, etc. can be used. The reaction temperature is preferably −20 to 100° C., and more preferably 0 to 60° C. The reaction time is preferably 0.2 to 100 hours, and more preferably 0.5 to 20 hours.

The base polymer preferably has a weight-average molecular weight (Mw) of 1,000 to 500,000, and more preferably 2,000 to 30,000. The Mw is in terms of polystyrene by gel permeation chromatography (GPC) using THF as a solvent. If Mw is within the above range, good heat resistance of the resist film and solubility in an alkaline developer can be provided.

When the base polymer has sufficiently narrow molecular weight distribution (Mw/Mn), a low molecular-weight and high molecular-weight polymers are absent, and thereby there is no risk of a foreign matter observed on a pattern and deterioration in a pattern shape after the exposure. Since Mw and Mw/Mn have a larger effect as the pattern rule becomes smaller, the Mw/Mn of the base polymer is preferably 1.0 to 2.0 and particularly preferably 1.0 to 1.5, which indicates narrow distribution, in order to obtain the resist composition suitably used for a fine pattern size.

The base polymer may comprise two or more kinds of polymers having different composition ratios, Mw, and Mw/Mn.

### [Acid Generator]

The inventive resist composition may comprise an acid generator to generate a strong acid (hereinafter, also referred to as the additive-type acid generator). The strong acid herein means: a compound having sufficient acidity for causing the deprotection reaction of the acid-labile group in the base polymer in a case of a chemically amplified positive-type resist composition; or a compound having sufficient acidity for causing a polarity-changing reaction or a crosslinking reaction with the acid in a case of a chemically amplified negative-type resist composition. By containing such an acid generator, the aforementioned onium salt can function as a quencher, and the inventive resist composition can function as a chemically amplified positive-type resist composition or a chemically amplified negative-type resist composition.

Examples of the acid generator include a compound to generate an acid by sensitizing with active ray or radiation (photoacid generator). The photoacid generator may be any compound that generates an acid by high-energy ray irradiation, but the photoacid generator preferably generates a sulfonic acid, an imide acid, or a methide acid. Examples of preferable photoacid generators include sulfonium salts, iodonium salts, sulfonyldiazomethanes, N-sulfonyloxyimides, and oxime-O-sulfonate-type acid generators. Specific examples of the photoacid generator include those described in paragraphs [0122] to [0142] of JP 2008-111103 A.

As the photoacid generator, a sulfonium salt represented by the following general formula (3-1) and an iodonium salt represented by the following general formula (3-2) can also be preferably used.

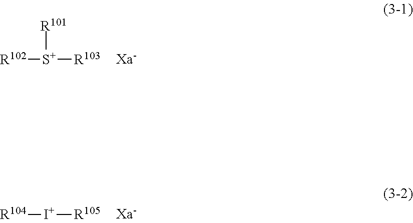

In the general formulae (3-1) and (3-2), R¹⁰¹ to R¹⁰⁵ each independently represent a halogen atom or a hydrocarbyl group having 1 to 20 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R^{11′} to R^{19′} in description of the formulae (Cation-1) to (Cation-3). R¹⁰¹ and R¹⁰² are optionally bonded to each other to form a ring together with the sulfur atom to which these groups are bonded. In this case, examples of the ring include rings same as those exemplified as the rings that can be formed by bonding R^{11′} and R^{12′} together with the sulfur atom to which these groups are bonded, described in the formula (Cation-1).

Examples of the cation of the sulfonium salt represented by the general formula (3-1) include cations same as those exemplified as the cation of the sulfonium salt represented by the formula (Cation-1), but the cation is not limited thereto.

Examples of the cation of the iodonium salt represented by the general formula (3-2) include cations same as those exemplified as the cation of the iodonium salt represented by the formula (Cation-2), but the cation is not limited thereto.

In the general formulae (3-1) and (3-2), Xa⁻ represents an anion selected from the following formulae (3A) to (3D).

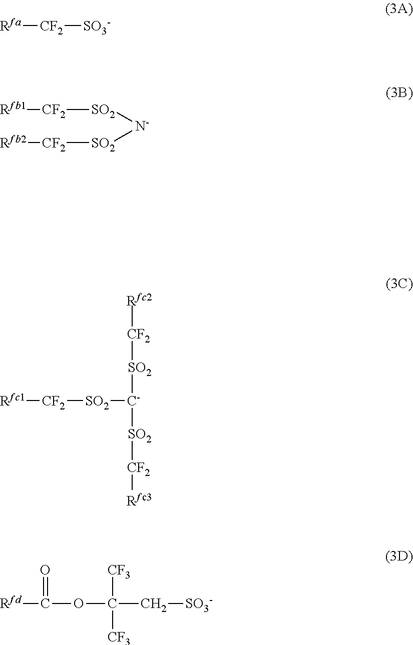

In the general formula (3A), R^{fa} represents a fluorine atom or a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as a hydrocarbyl group represented by R¹¹¹ in the formula (3A′), described later.

The anion represented by the formula (3A) is preferably represented by the following general formula (3A′).

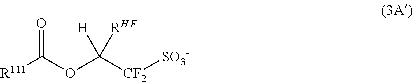

In the general formula (3A′), R^{HF} represents a hydrogen atom or a trifluoromethyl group, and preferably a trifluoromethyl group. R¹¹¹ represents a hydrocarbyl group having 1 to 38 carbon atoms and optionally having a heteroatom. The heteroatom is preferably an oxygen atom, a nitrogen atom, a sulfur atom, a halogen atom, etc., and more preferably an oxygen atom. The hydrocarbyl group particularly preferably has 6 to 30 carbon atoms in terms of obtaining high resolution in fine patterning.

The hydrocarbyl group represented by R¹¹¹ may be a saturated or unsaturated group, and may be any of linear, branched, and cyclic groups. Specific examples thereof include: alkyl groups having 1 to 38 carbon atoms, such as a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosanyl group; cyclic saturated hydrocarbyl groups having 3 to 38 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; unsaturated aliphatic hydrocarbyl groups having 2 to 38 carbon atoms, such as an allyl group and a 3-cyclohexenyl group; aryl groups having 6 to 38 carbon atoms, such as a phenyl group, a 1-naphthyl group, and a 2-naphthyl group; aralkyl groups having 7 to 38 carbon atoms, such as a benzyl group and a diphenylmethyl group; and groups obtained by combining these groups.

A part or all of hydrogen atoms in these groups are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, a haloalkyl group, etc. Examples of the hydrocarbyl group having a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

Synthesis of the sulfonium salt having the anion represented by the general formula (3A′) is described in detail in JP 2007-145797 A, JP 2008-106045 A, JP 2009-7327 A, and JP 2009-258695 A. Synthesis of sulfonium salts described in JP 2010-215608 A, JP 2012-41320 A, JP 2012-106986 A, JP 2012-153644 A, etc. are also preferably used.

Examples of the anion represented by the general formula (3A) include anions same as those exemplified as anions represented by the formula (1A) of JP 2018-197853 A.

In the general formula (3B), R^{fb1} and R^{fb2} each independently represent a fluorine atom or a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the general formula (3A′). R^{fb1} and R^{fb2} preferably represent a fluorine atom or a linear fluorinated alkyl group having 1 to 4 carbon atoms. R^{fb1} and R^{fb2} are optionally bonded to each other to form a ring together with the group to which these groups are bonded (—CF₂—SO₂—N⁻—SO₂—CF₂—), and the group obtained in this case by bonding R^{fb1} and R^{fb2} each other is preferably a fluorinated ethylene group or a fluorinated propylene group.

In the general formula (3C), R^{fc1}, R^{fc2}, and R^{fc3} each independently represent a fluorine atom or a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the general formula (3A′). R^{fc1}, R^{fc2}, and R^{fc3} preferably represent a fluorine atom or a linear fluorinated alkyl group having 1 to 4 carbon atoms. R^{fc1} and R^{fc2} are optionally bonded to each other to form a ring together with the group to which these groups are bonded (—CF₂—SO₂—C⁻—SO₂—CF₂—), and the group obtained in this case by bonding R^{fc1} and R^{fc2} each other is preferably a fluorinated ethylene group or a fluorinated propylene group.

In the general formula (3D), R^{fd} represents a hydrocarbyl group having 1 to 40 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the formula (3A′).

Synthesis of the sulfonium salt having the anion represented by the general formula (3D) is described in detail in JP 2010-215608 A and JP 2014-133723 A.

Examples of the anion represented by the general formula (3D) include anions same as those exemplified as the anions represented by the formula (1D) in JP 2018-197853 A.

Although having no fluorine atom at the α-position of the sulfo group, the photoacid generator having the anion represented by the general formula (3D) has sufficient acidity for cleaving the acid-labile group in the base polymer by virtue of the two trifluoromethyl groups at the β-position. Thus, this photoacid generator can be used as the photoacid generator.

As the photoacid generator, a compound represented by the following general formula (4) can also be preferably used.

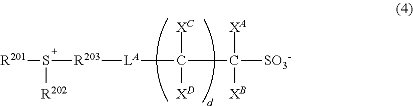

In the general formula (4), R²⁰¹ and R²⁰² each independently represent a halogen atom or a hydrocarbyl group having 1 to 30 carbon atoms and optionally having a heteroatom. R²⁰³ represents a hydrocarbylene group having 1 to 30 carbon atoms and optionally having a heteroatom. Any two of R²⁰¹, R²⁰² and R²⁰³ are optionally bonded to each other to form a ring together with the sulfur atom to which these groups are bonded. In this case, examples of the ring include rings same as those exemplified as the rings that can be formed by bonding R^{11′} and R^{12′} together with the sulfur atom to which these groups are bonded, described in the general formula (Cation-1).

The hydrocarbyl group represented by R²⁰¹ and R²⁰² may be a saturated or unsaturated group, and may be any of linear, branched, and cyclic groups. Specific examples thereof include: alkyl groups having 1 to 30 carbon atoms, such as a methyl group, an ethyl group, a propyl group, an isopropyl group, an n-butyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decanyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups obtained by combining these groups. A part or all of hydrogen atoms in these groups are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained are a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, or a haloalkyl group, etc.

The hydrocarbylene group represented by R²⁰³ may be a saturated or unsaturated group, and may be any of linear, branched, and cyclic groups. Specific examples thereof include: alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentanediyl group, a cyclohexanediyl group, a norbornanediyl group, and an adamantanediyl group; arylene groups having 6 to carbon atoms, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group; and groups obtained by combining these groups. A part or all of hydrogen atoms in these groups are optionally substituted with a group having a heteroatom such as an oxygen atom, a sulfur atom, a nitrogen atom, and a halogen atom. A part of carbon atoms in these groups is optionally substituted with a group having a heteroatom such as an oxygen atom, a sulfur atom, and a nitrogen atom. As a result, optionally contained is a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride, or a haloalkyl group, etc. The heteroatom is preferably an oxygen atom.

In the general formula (4), L^{A} represents a single bond, an ether bond, or a hydrocarbylene group having 1 to 20 carbon atoms and optionally having a heteroatom. The hydrocarbylene group may be a saturated or unsaturated group, and may be any of linear, branched, and cyclic groups. Specific examples thereof include groups same as those exemplified as the hydrocarbylene group represented by R²⁰³.

In the general formula (4), X^{A}, X^{B}, X^{C}, and X^{D} each independently represent a hydrogen atom, a fluorine atom, or a trifluoromethyl group. Note that, at least one of X^{A}, X^{B}, X^{C}, and X^{D} represents a fluorine atom or a trifluoromethyl group.

In the general formula (4), “d” represents an integer of 0 to 3.

The photoacid generator represented by the general formula (4) is preferably represented by the following general formula (4′).

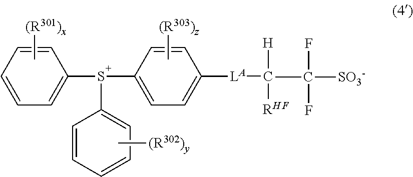

In the general formula (4′), L^{A} represents the same as above. R^{HF} represents a hydrogen atom or a trifluoromethyl group, and preferably a trifluoromethyl group. R³⁰¹, R³⁰², and R³⁰³ each independently represent a hydrogen atom or a hydrocarbyl group having 1 to 20 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R¹¹¹ in the formula (3A′). “x” and “y” each independently represent an integer of 0 to 5, and “z” represents an integer of 0 to 4.

Examples of the photoacid generator represented by the general formula (4) include photoacid generators same as those exemplified as photoacid generators represented by the formula (2) in JP 2017-026980 A.

Among the above photoacid generators, the photoacid generators having the anion represented by the general formula (3A′) or (3D) are particularly preferable since having small acid diffusion and excellent solubility in the solvent. The photoacid generators represented by the formula (4′) are particularly preferable since having extremely small acid diffusion.

As the photoacid generator, a sulfonium salt or iodonium salt having an anion having an aromatic ring substituted with an iodine atom or a bromine atom can also be used. Examples of such a salt include salts represented by the following general formula (5-1) or (5-2).

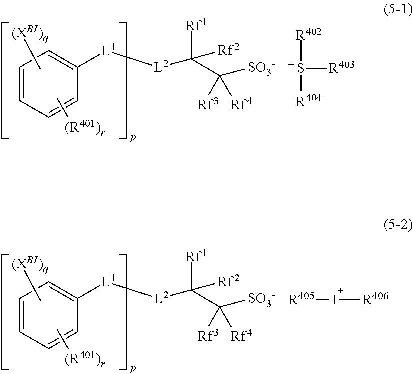

In the general formulae (5-1) and (5-2), “p” represents an integer satisfying 1≤p≤3. “q” and “r” represent an integer satisfying 1≤q≤5, 0≤r≤3, and 1≤q+r≤5. “q” preferably represents an integer satisfying 1≤q≤3, and more preferably 2 or 3. “r” preferably represents an integer satisfying 0≤r≤2.

In the general formulae (5-1) and (5-2), X^{BI} represents an iodine atom or a bromine atom. When “p” and/or “q” represent 2 or more, X^{BI} may be same as or different from each other.

In the general formulae (5-1) and (5-2), L¹ represents a single bond, an ether bond, an ester bond, or a saturated hydrocarbylene group having 1 to 6 carbon atoms and optionally having an ether bond or an ester bond. The saturated hydrocarbylene group may be any of linear, branched, and cyclic groups.

In the general formulae (5-1) and (5-2), L² represents a single bond or a divalent linking group having 1 to 20 carbon atoms when “p” represents 1, and L² represents a (p+1)-valent linking group having 1 to 20 carbon atoms when “p” represents 2 or 3. The linking group optionally has an oxygen atom, a sulfur atom, or a nitrogen atom.

In the general formulae (5-1) and (5-2), R⁴⁰¹ represents a hydroxy group, a carboxy group, a fluorine atom, a chlorine atom, a bromine atom, an amino group, a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, a hydrocarbylsulfonyloxy group having 1 to 20 carbon atoms, —N(R^{401A}) (R^{401B}), —N(R^{401C})—C(═O)—R^{401D}, or —N(R^{401C})—C(═O)—O—R^{401D}. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group optionally have a fluorine atom, a chlorine atom, a bromine atom, a hydroxy group, an amino group, an ether bond, an ester bond, or an amide bond. R^{401A} and R^{401B} each independently represent a hydrogen atom or a saturated hydrocarbyl group having 1 to 6 carbon atoms. R^{401C} represents a hydrogen atom or a saturated hydrocarbyl group having 1 to 6 carbon atoms, and optionally having a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} represents an aliphatic hydrocarbyl group having 1 to 16 carbon atoms, an aryl group having 6 to 14 carbon atoms, or an aralkyl group having 7 to 15 carbon atoms, and optionally having a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be a saturated or unsaturated group, and may be any of linear, branched, and cyclic groups. The saturated hydrocarbyl group, the saturated hydrocarbyloxy group, the saturated hydrocarbyloxycarbonyl group, the saturated hydrocarbylcarbonyl group, and the saturated hydrocarbylcarbonyloxy group may be any of linear, branched, and cyclic groups. When “p” and/or “r” represent 2 or more, each R⁴⁰¹ may be same as or different from each other.

Among these, R⁴⁰¹ preferably represents a hydroxy group, —N(R^{401C})—C(═O)—R^{401D}, —N(R^{401C})—C(═O)—O—R^{401D}, a fluorine atom, a chlorine atom, a bromine atom, a methyl group, a methoxy group, etc.

In the general formulae (5-1) and (5-2), Rf¹ to Rf⁴ each independently represent a hydrogen atom, a fluorine atom, or a trifluoromethyl group, and at least one of them represents a fluorine atom or a trifluoromethyl group. Rf¹ and Rf² are optionally integrated to form a carbonyl group. In particular, both of Rf³ and Rf⁴ preferably represent fluorine atoms.

In the general formulae (5-1) and (5-2), R⁴⁰² to R⁴⁰⁶ each independently represent a halogen atom or a hydrocarbyl group having 1 to 20 carbon atoms and optionally having a heteroatom. The hydrocarbyl group may be a saturated or unsaturated group, and may be any of linear, branched, and cyclic groups. Specific examples thereof include groups same as those exemplified as the hydrocarbyl group represented by R^{11′} to R^{19′} in the description of the general formula (Cation-1). A part or all of hydrogen atoms in these groups are optionally substituted with a hydroxy group, a carboxy group, a halogen atom, a cyano group, a nitro group, a mercapto group, a sultone ring, a sulfone group, or a sulfonium-salt-containing group. A part of carbon atoms in these groups is optionally substituted with an ether bond, an ester bond, a carbonyl group, an amide bond, a carbonate bond, or a sulfonate ester bond. Furthermore, R⁴⁰² and R⁴⁰³ are optionally bonded to each other to form a ring together with the sulfur atom to which these groups are bonded. In this case, examples of the ring include rings same as those exemplified as the ring that can be formed by bonding R^{11′} and R^{12′} each other together with the sulfur atom to which these groups are bonded, described in the general formula (Cation-1).

Examples of the cation of the sulfonium salt represented by the general formula (5-1) include cations same as those exemplified as the cation of the sulfonium salt represented by the general formula (Cation-1). Examples of the cation of the iodonium salt represented by the general formula (5-2) include cations same as those exemplified as the cation of the iodonium salt represented by the general formula (Cation-2).

Specific examples of the anion of the onium salt represented by the general formula (5-1) or (5-2) include the following anions, but the anion is not limited thereto. In the following formulae, X^{BI} represents the same as above.

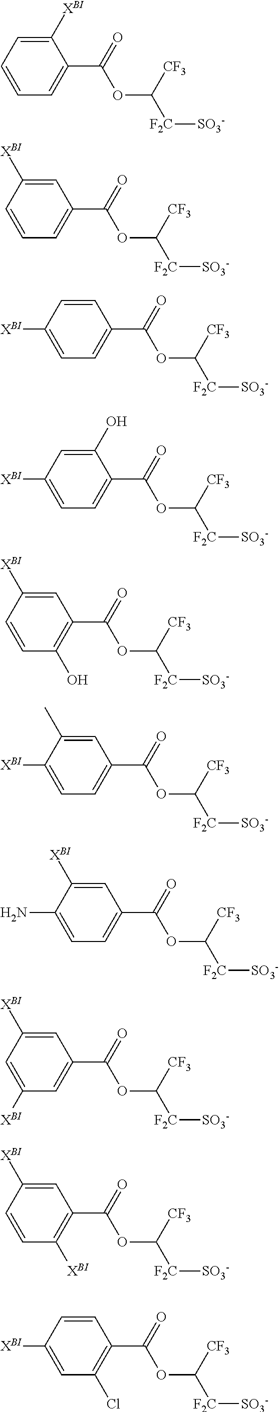

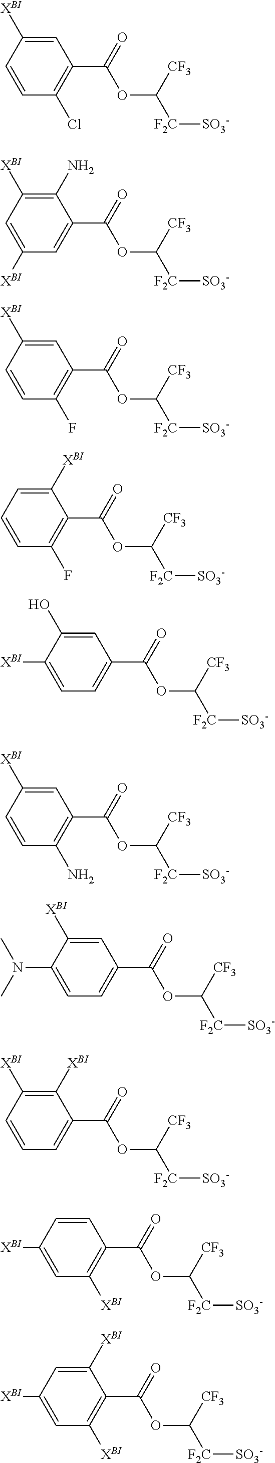

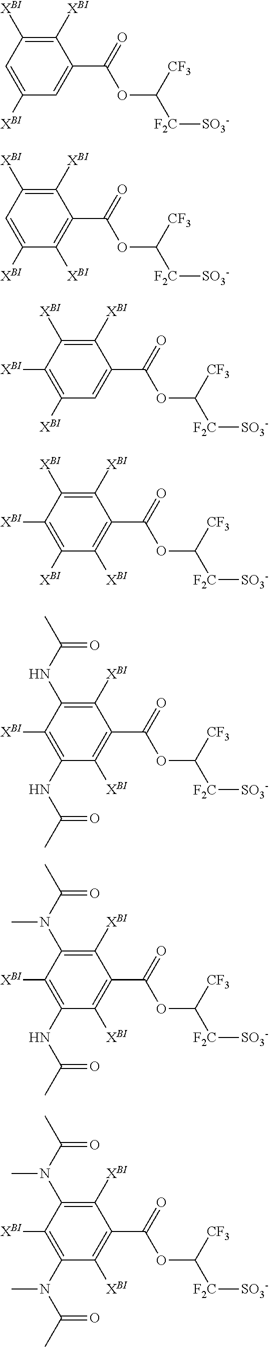

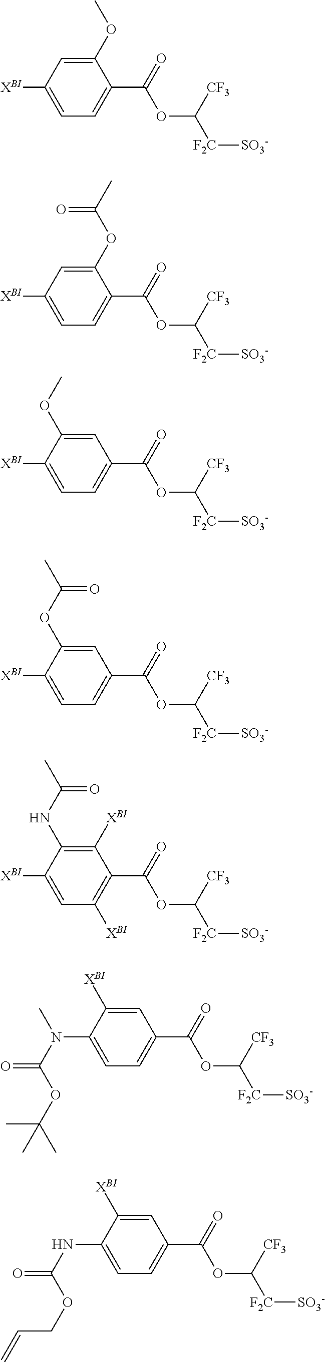

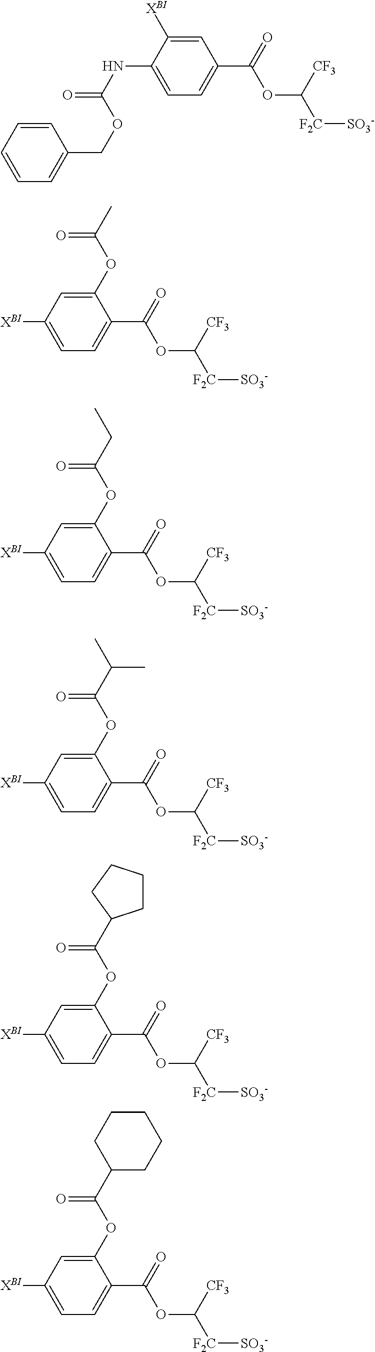

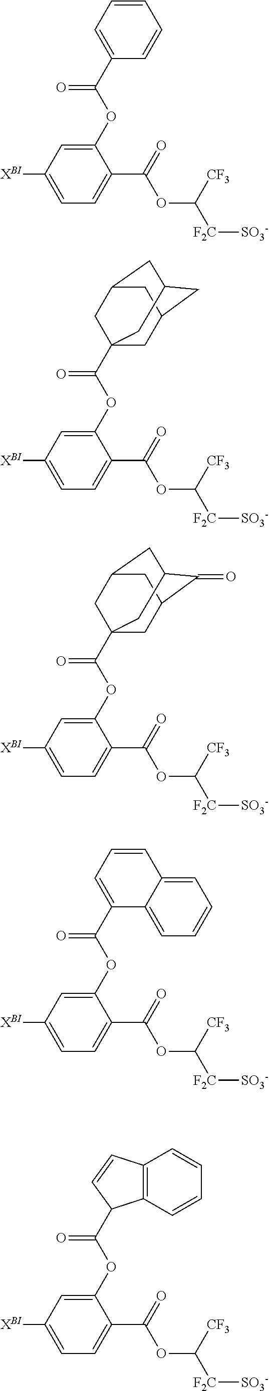

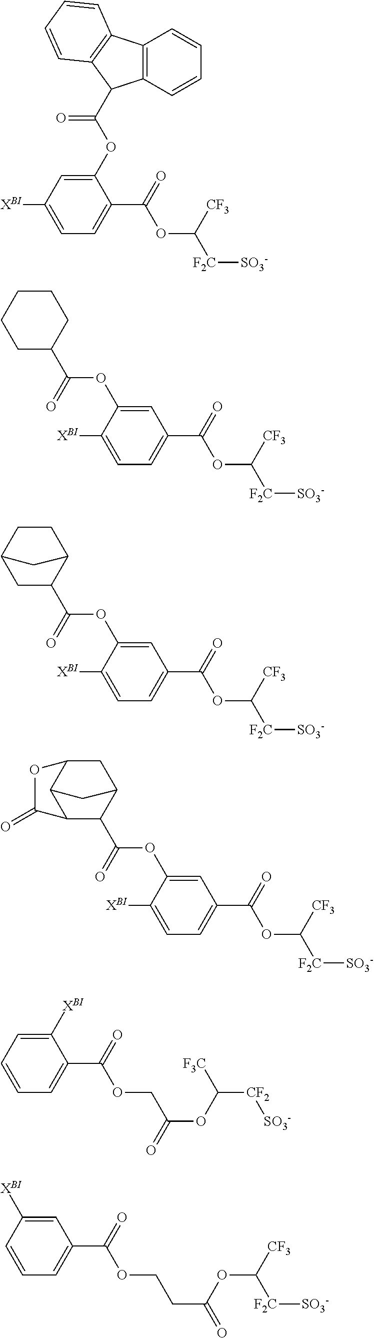

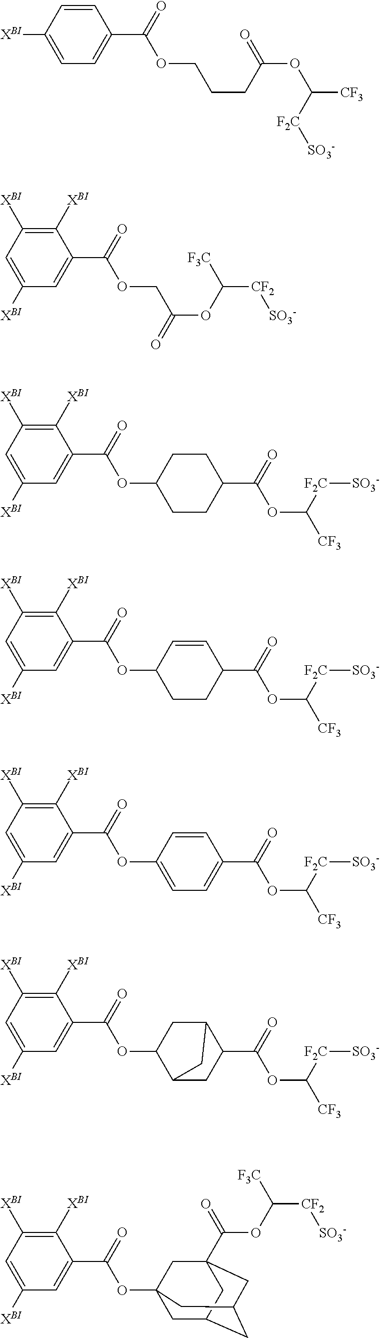

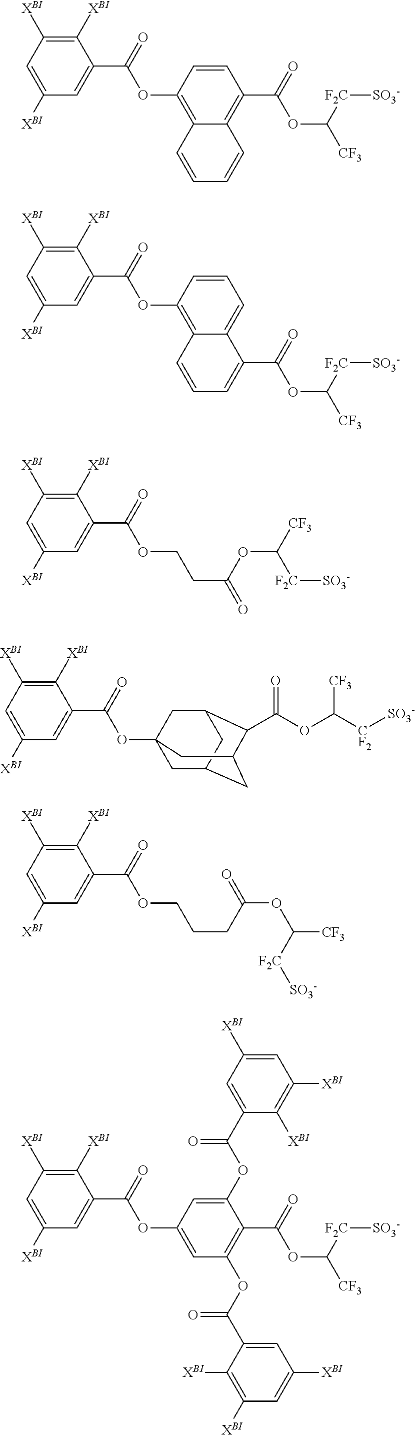

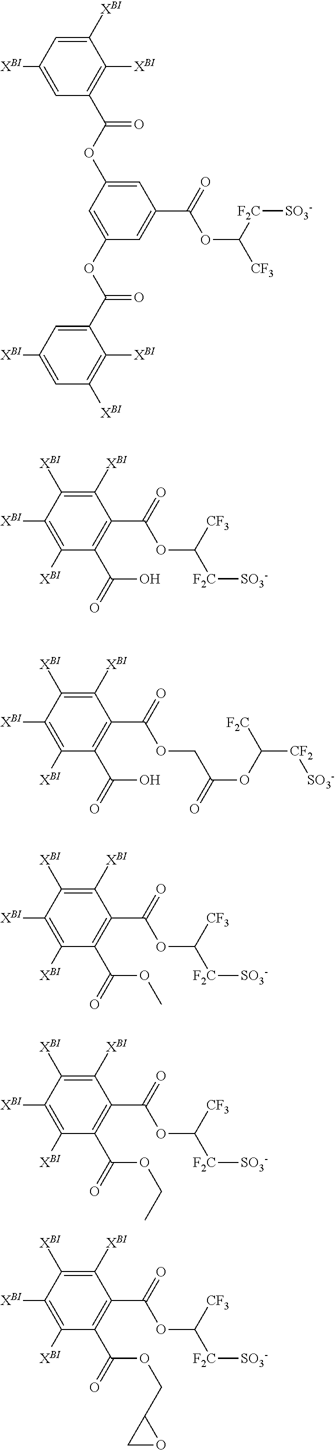

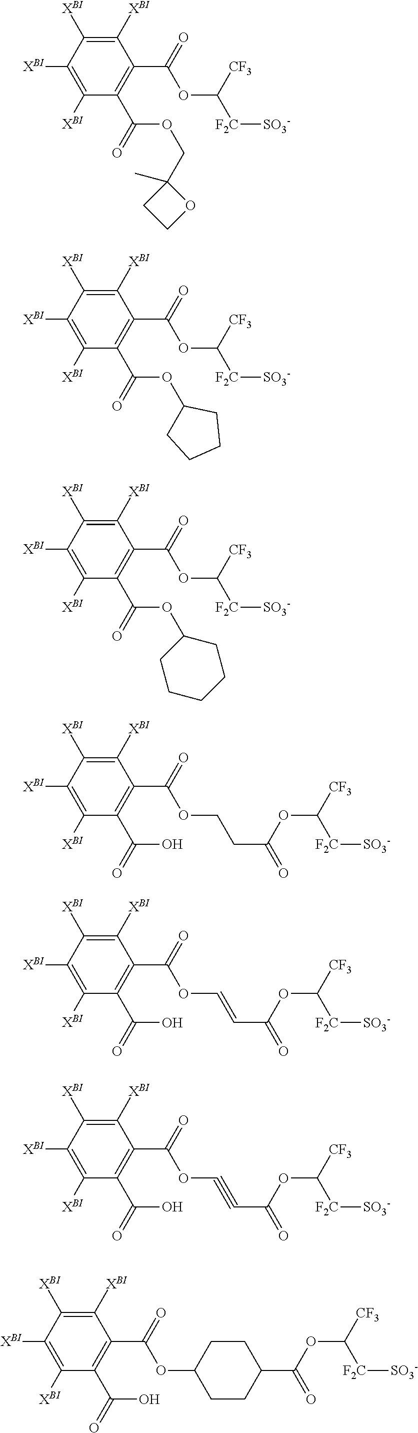

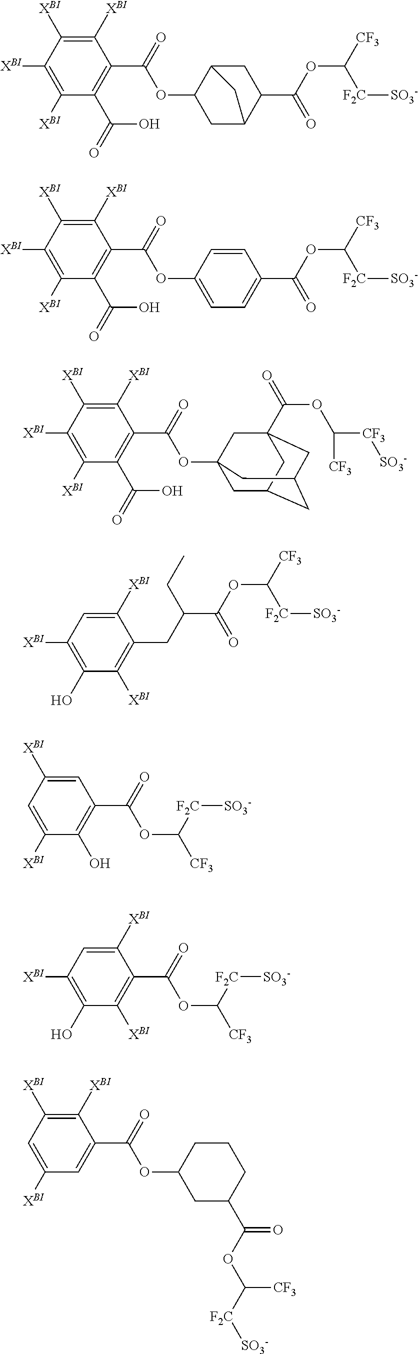

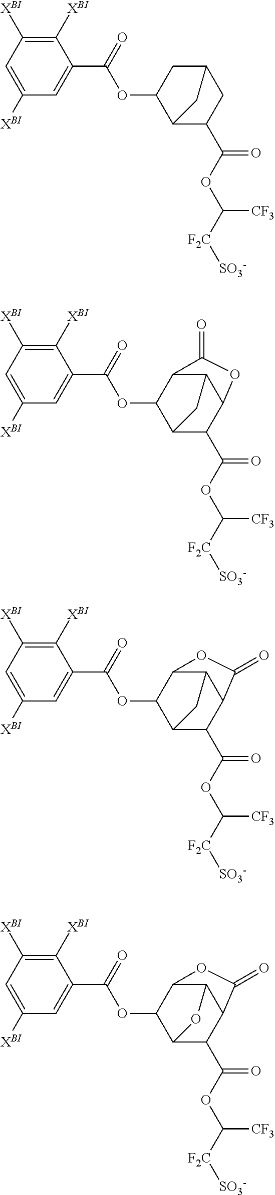

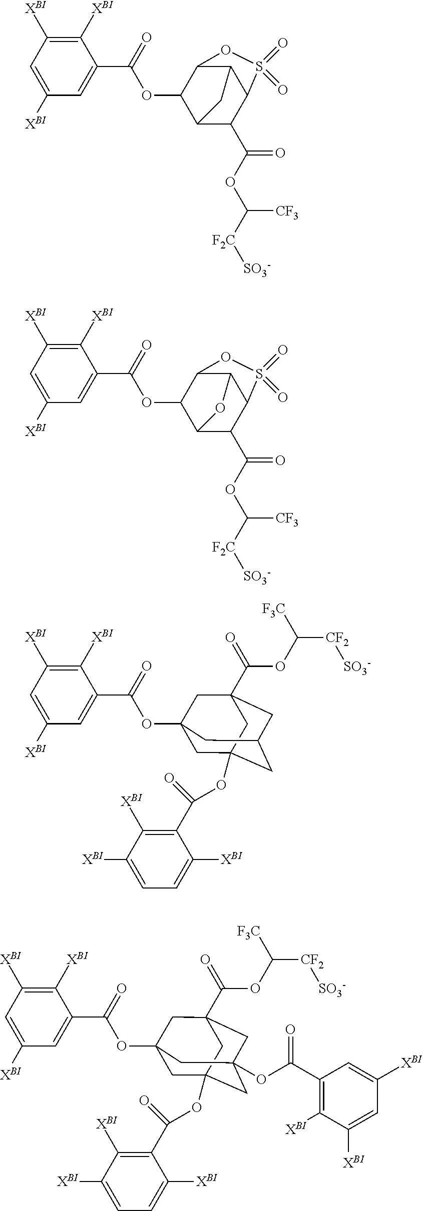

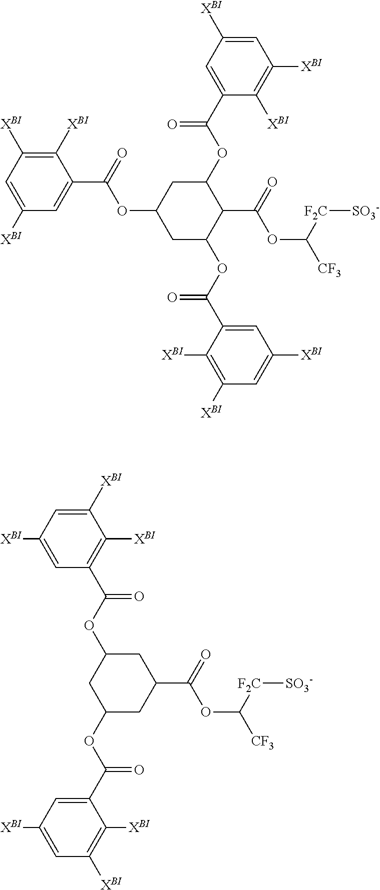

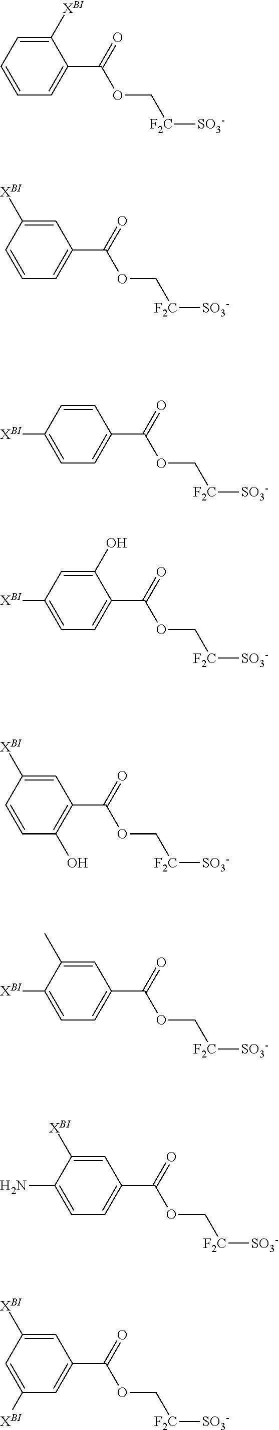

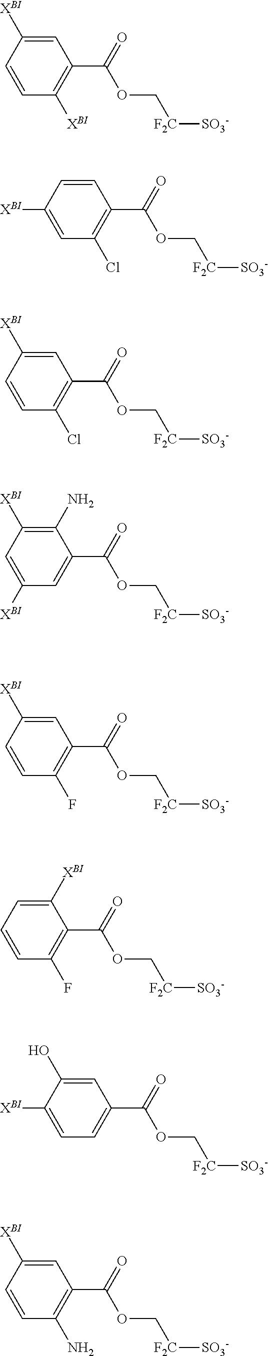

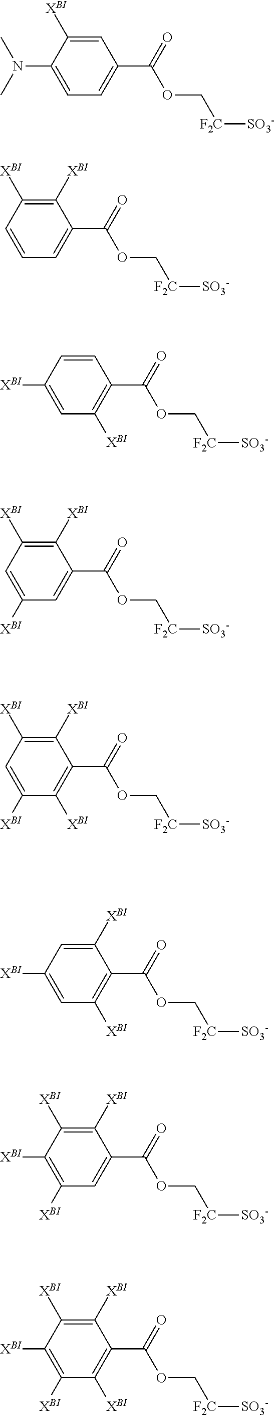

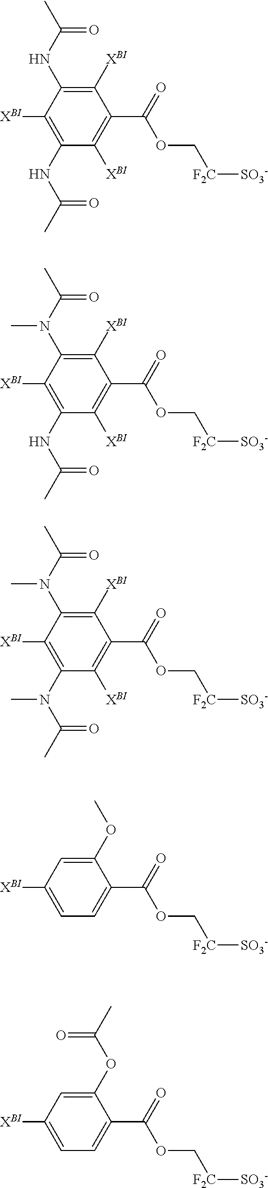

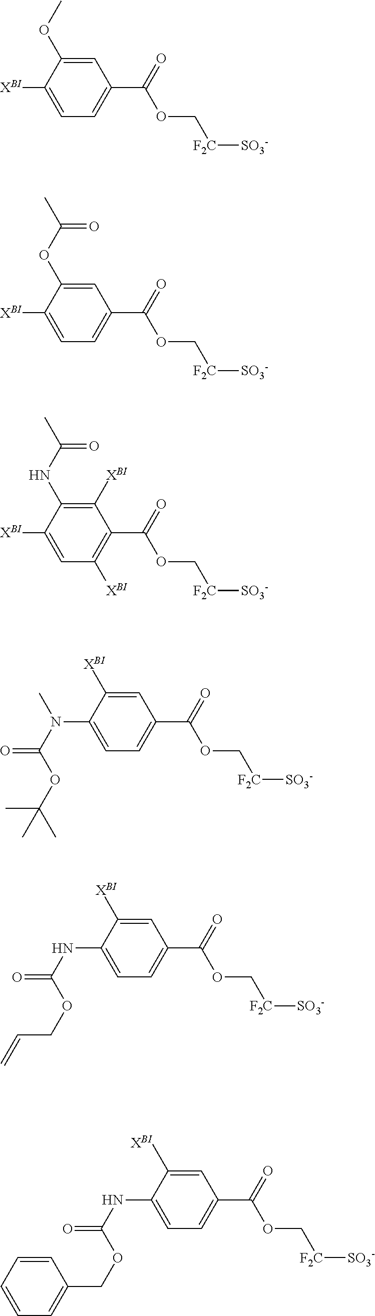

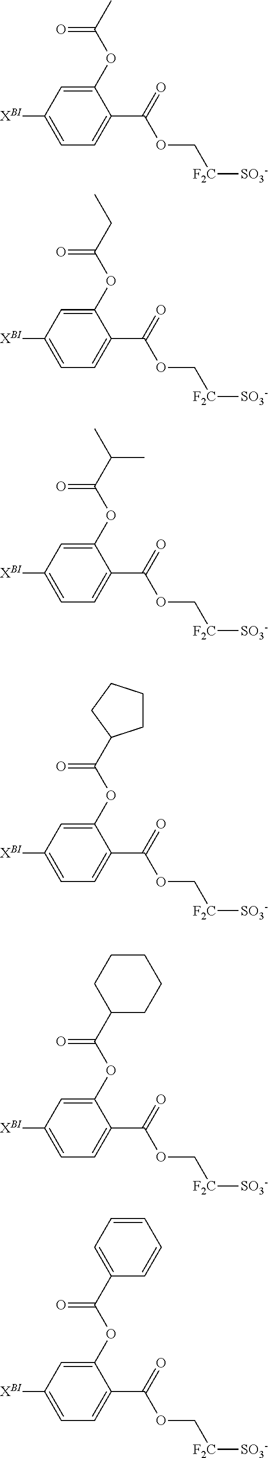

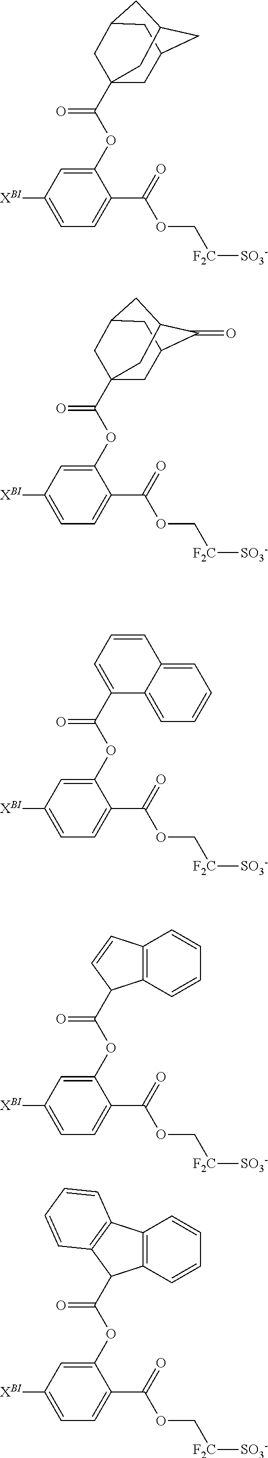

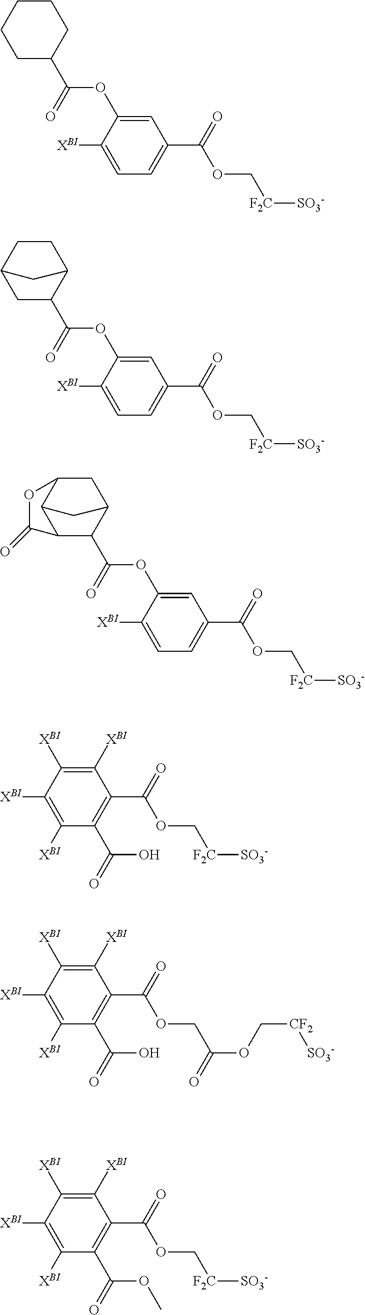

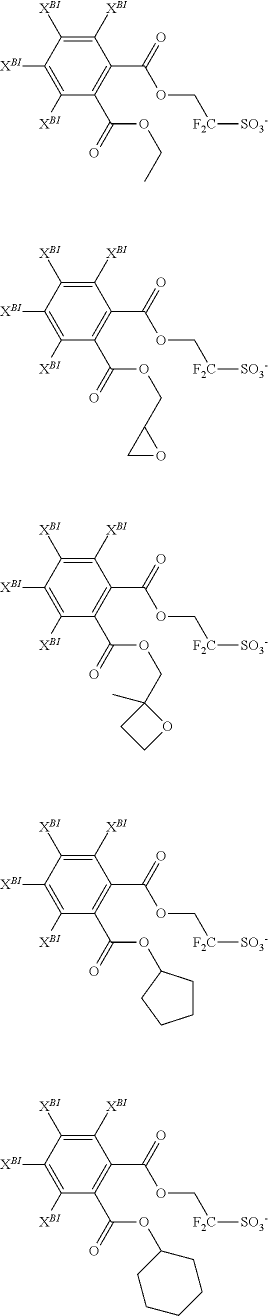

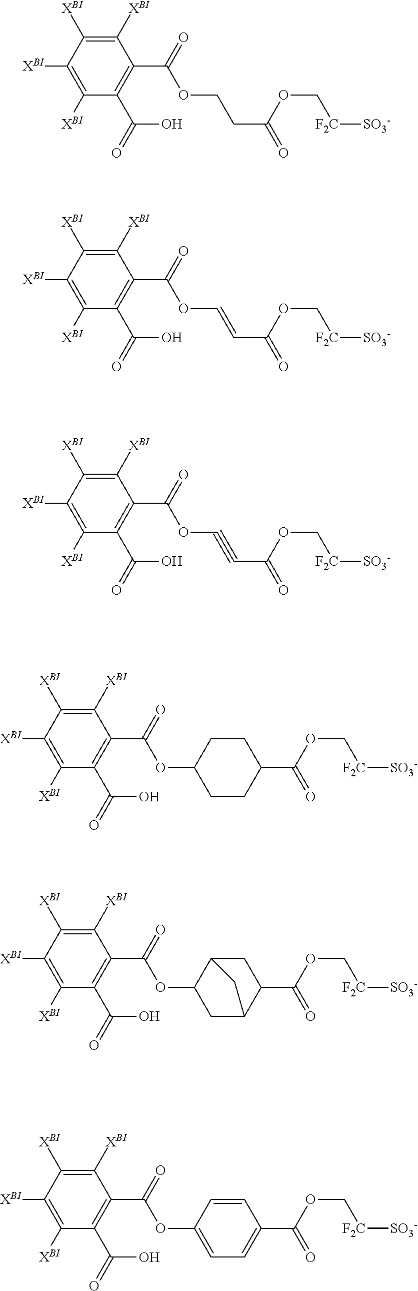

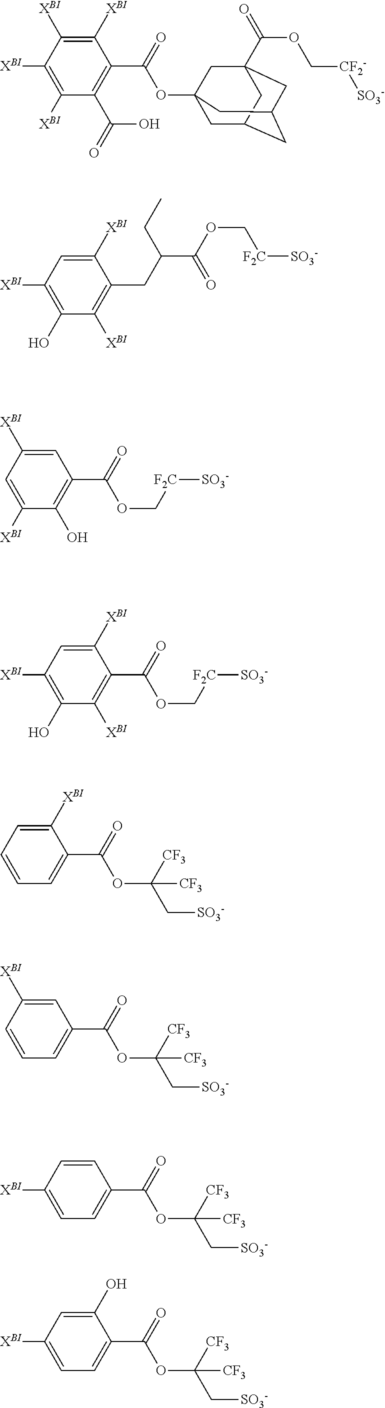

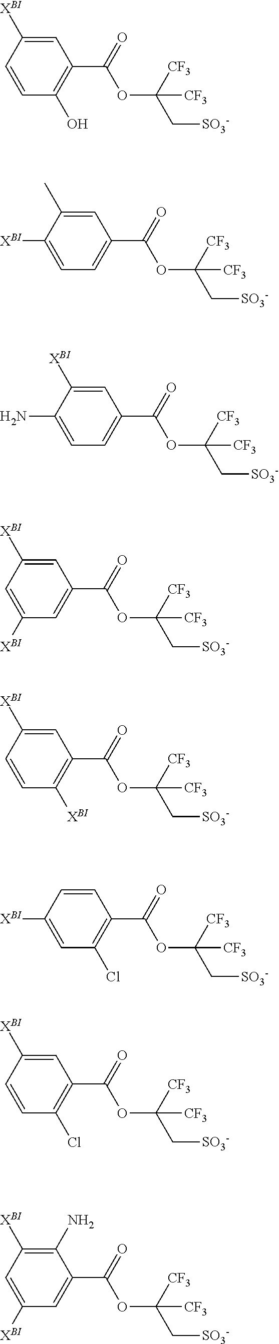

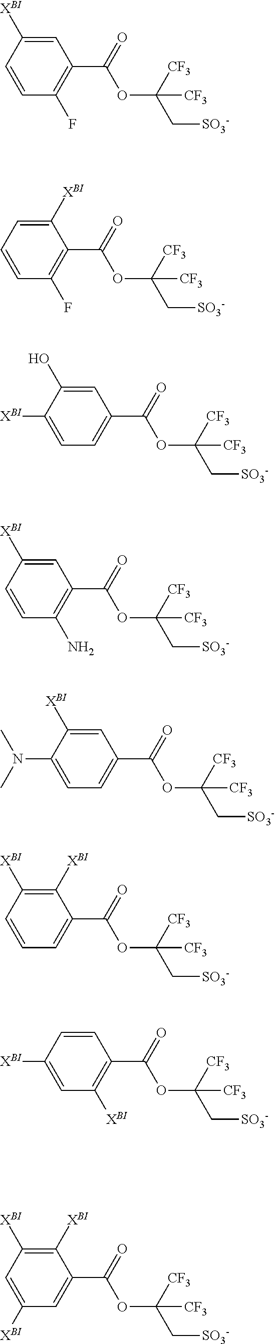

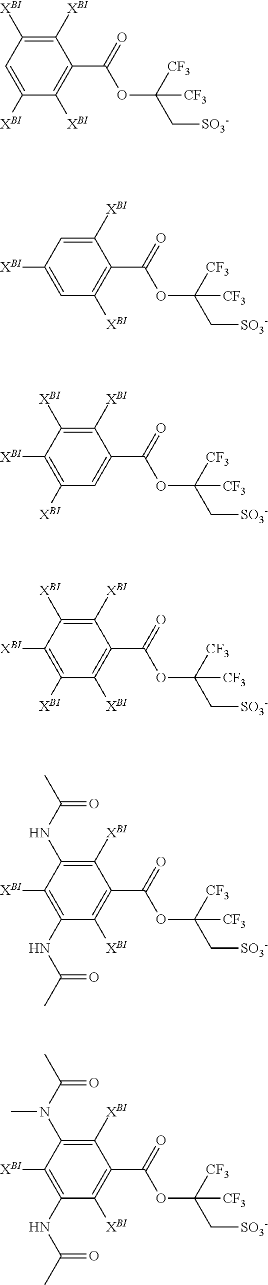

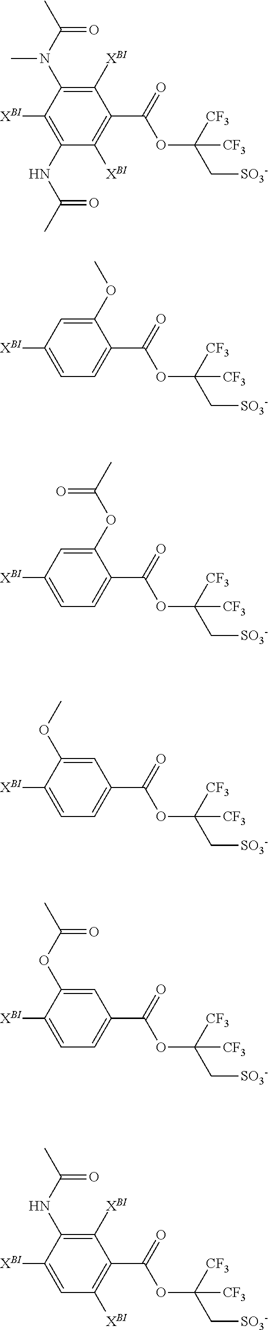

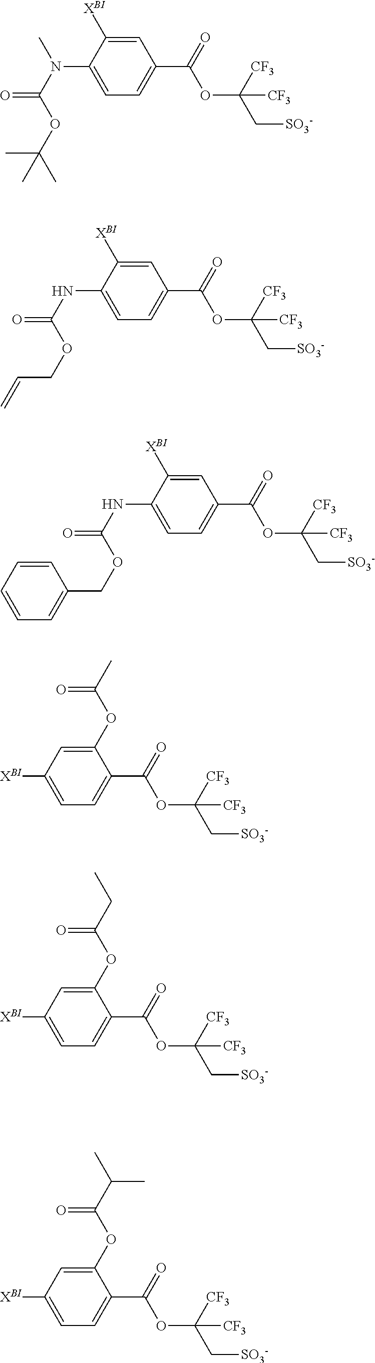

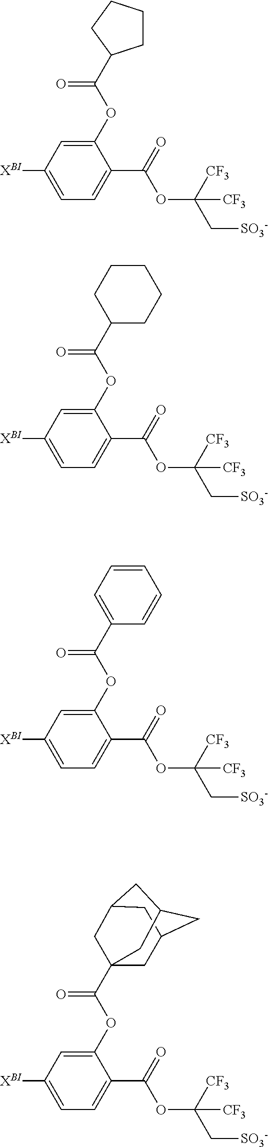

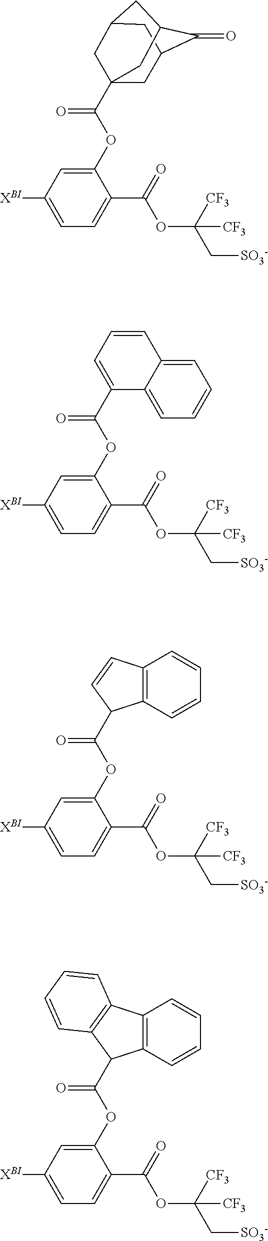

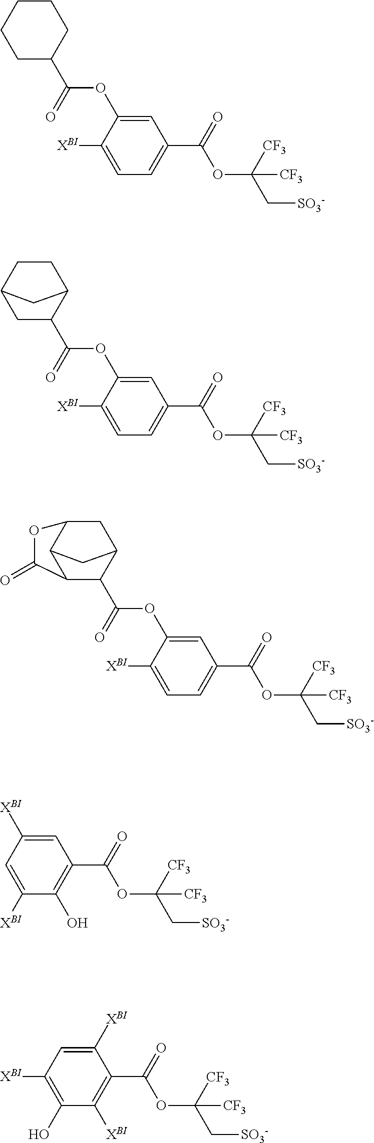

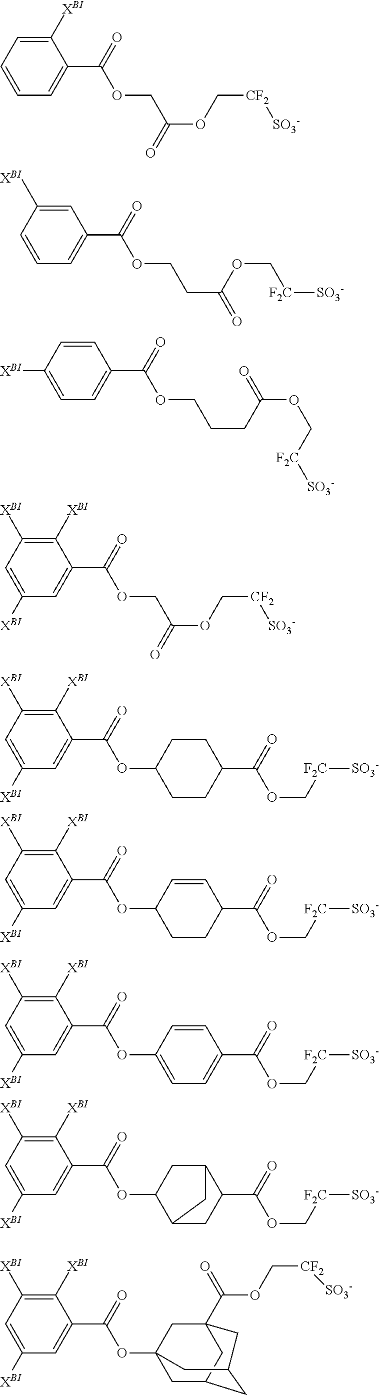

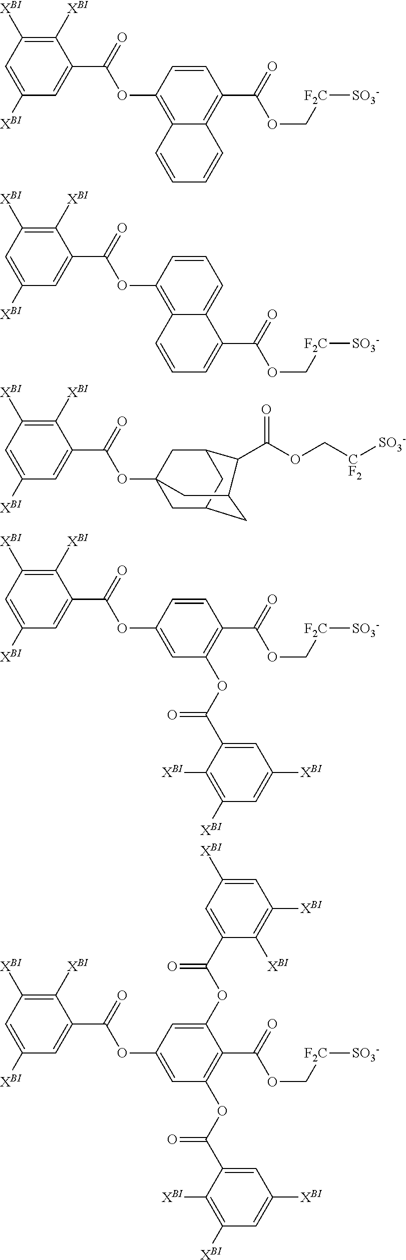

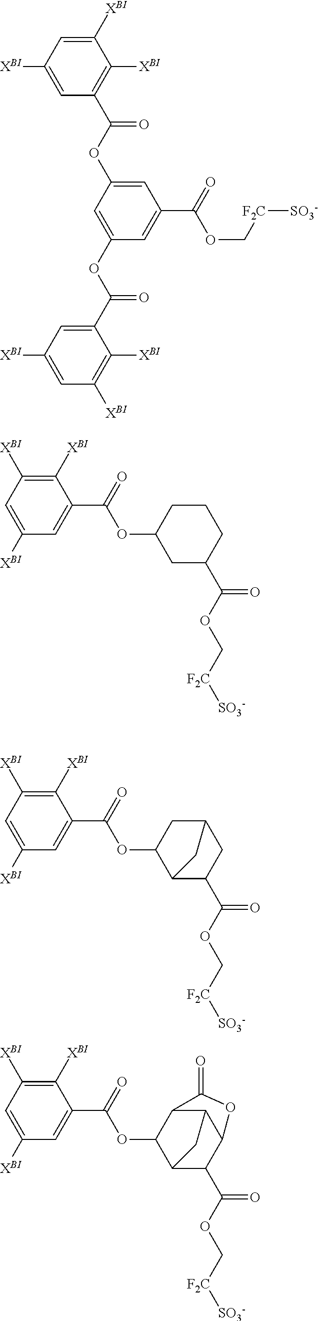

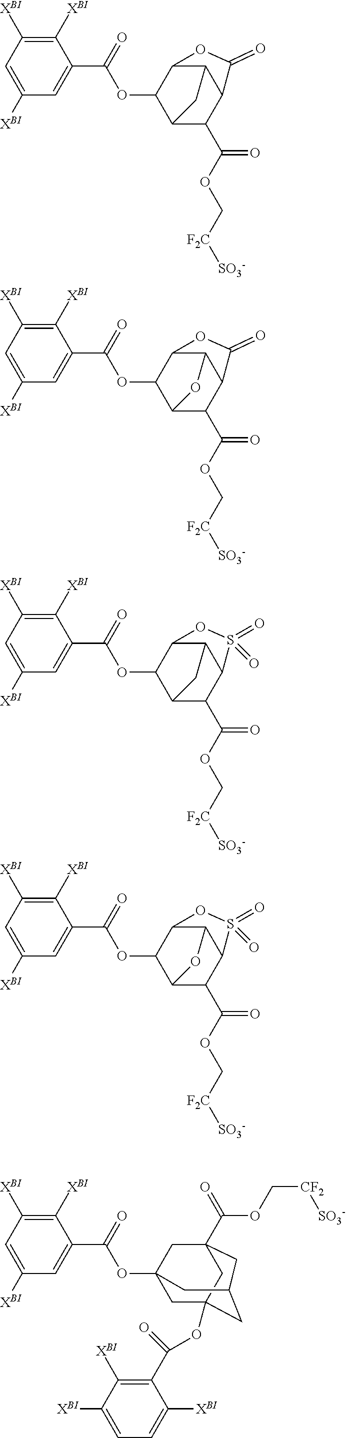

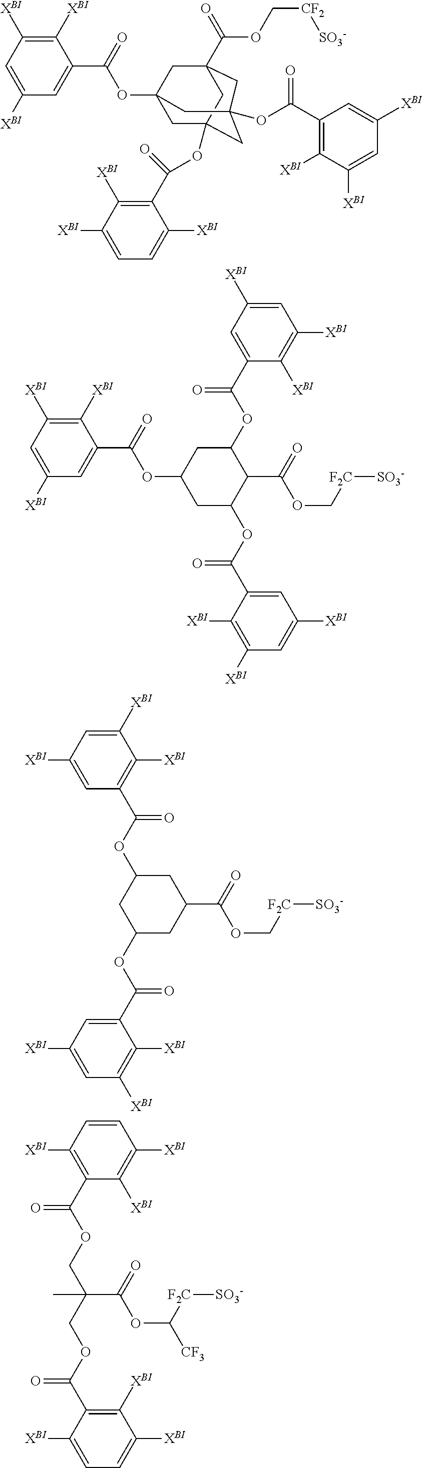

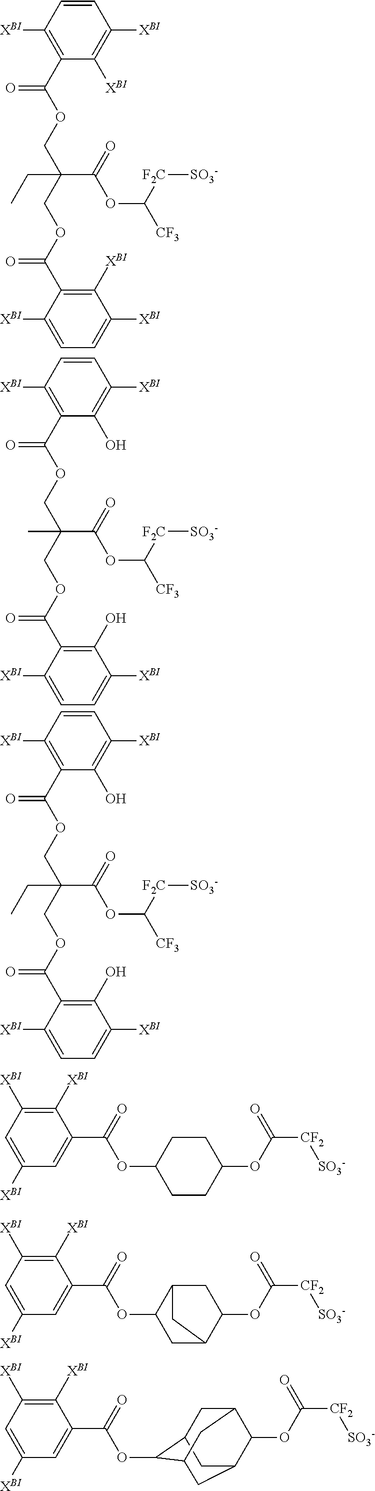

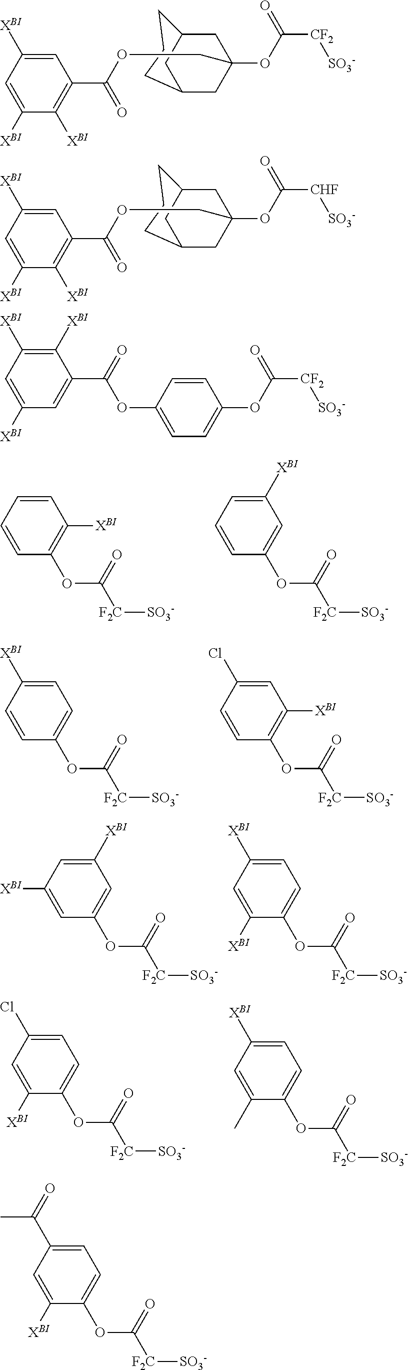

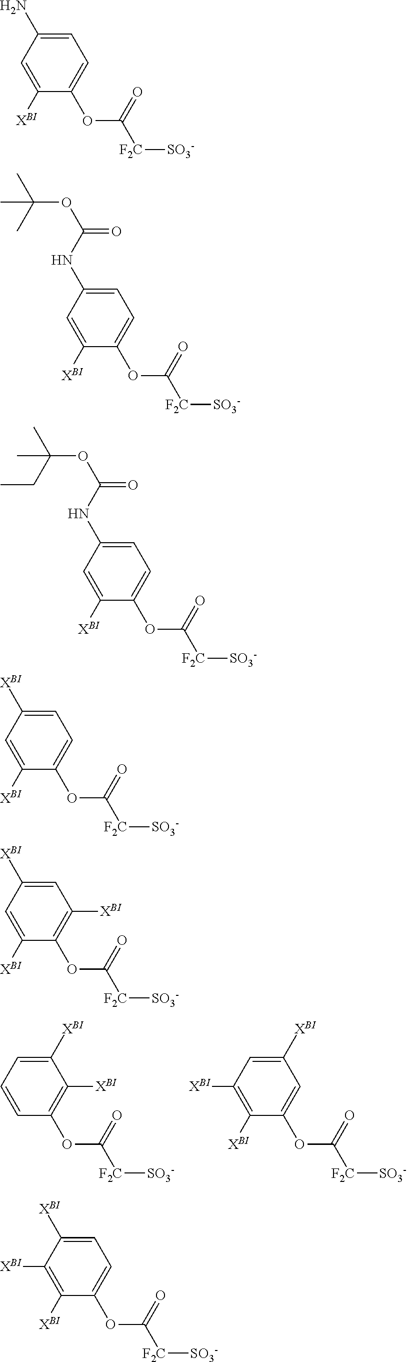

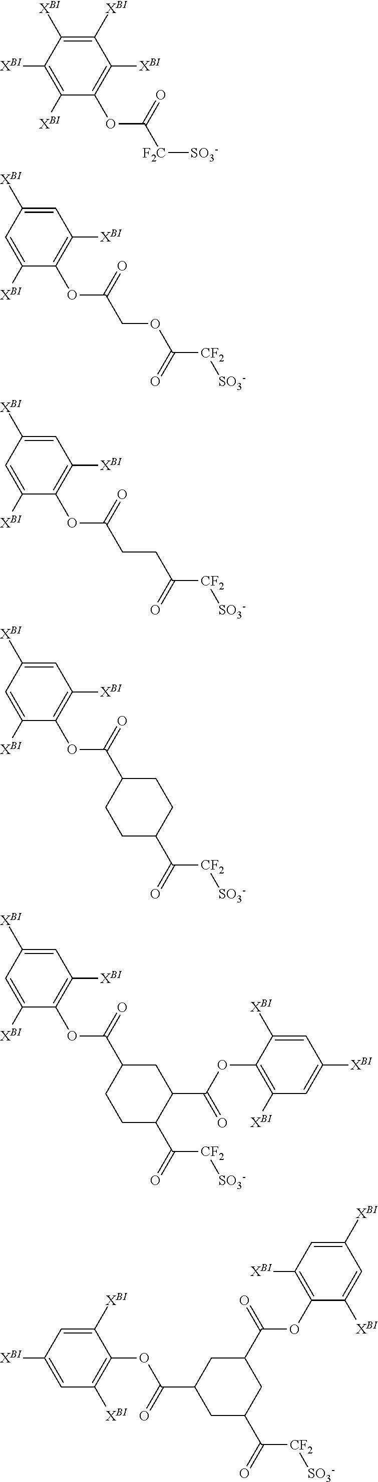

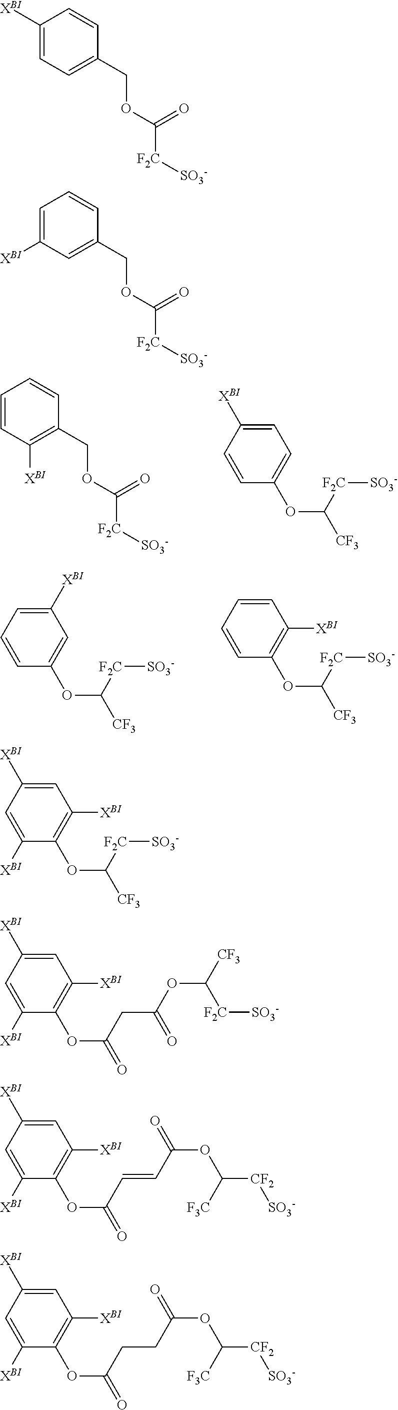

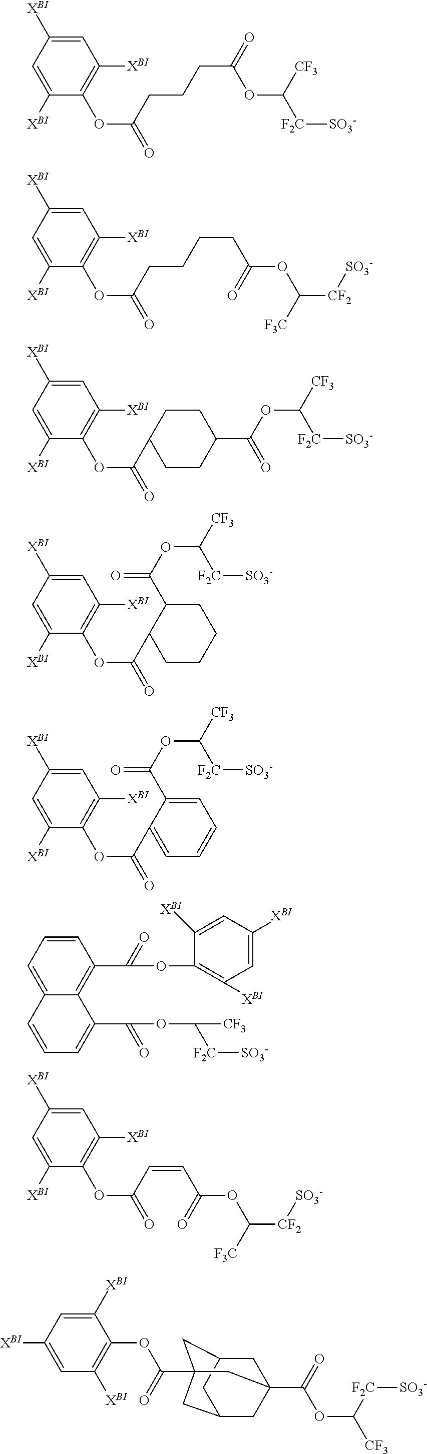

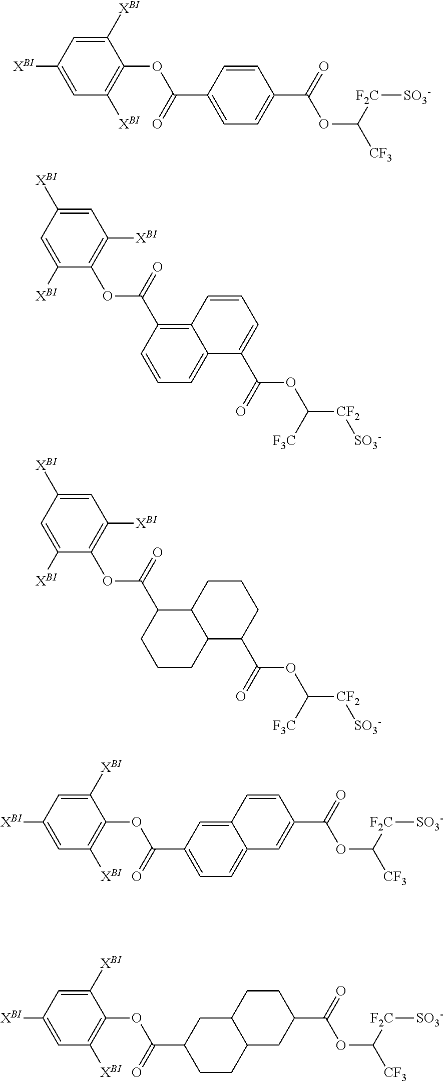

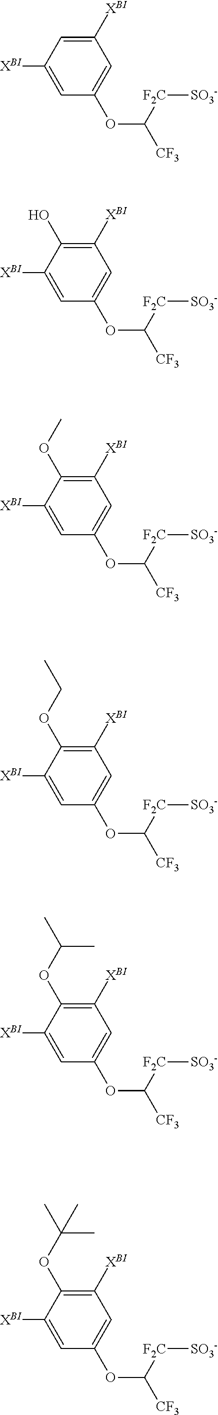

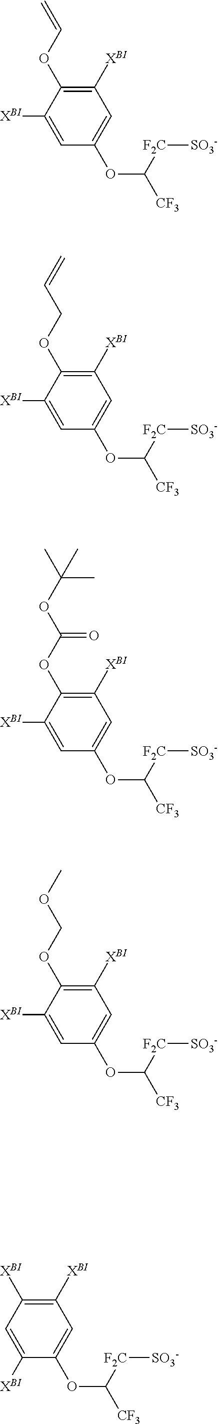

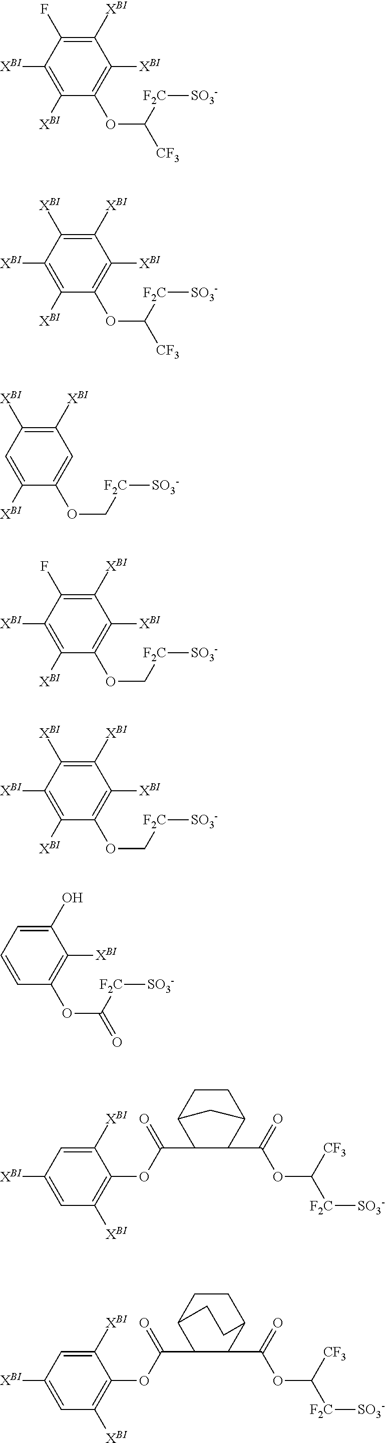

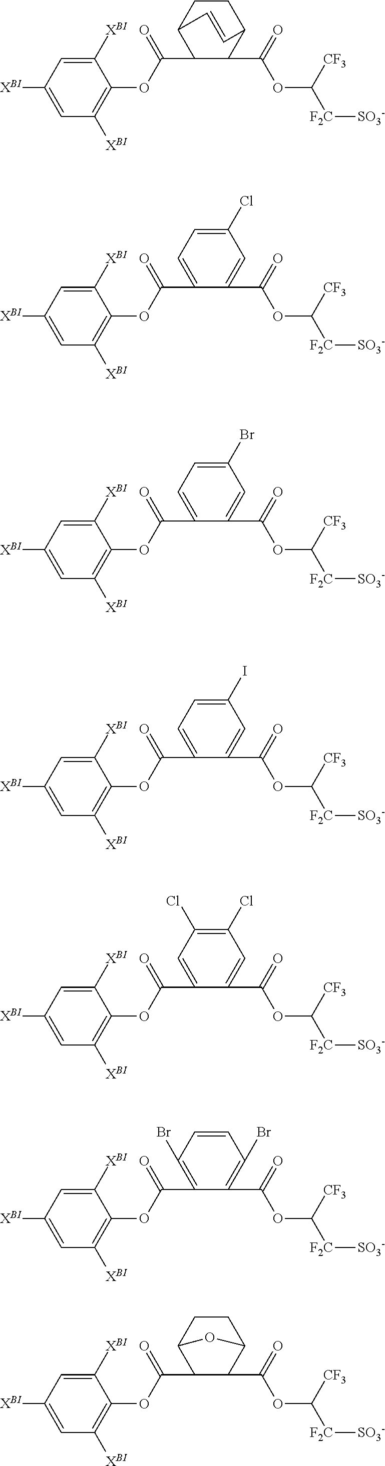

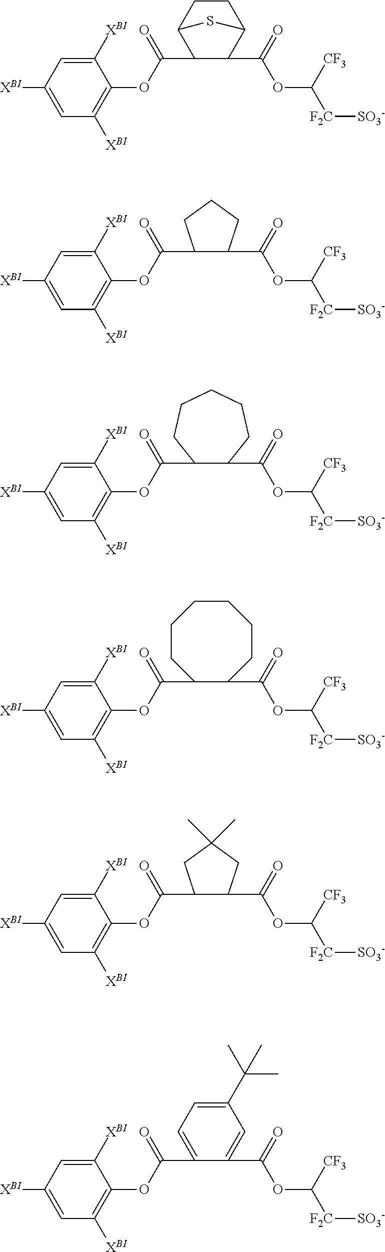

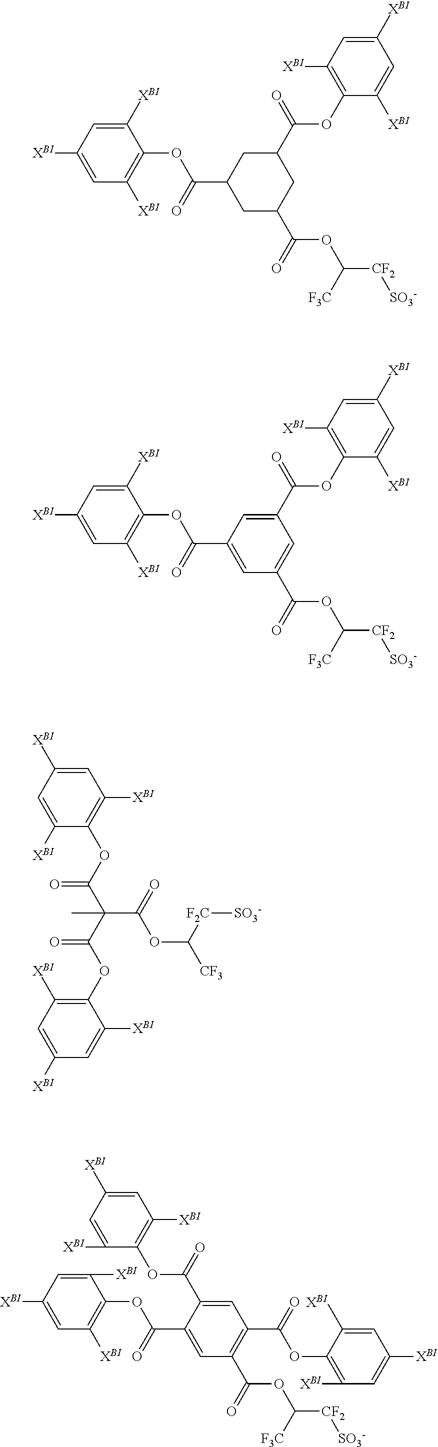

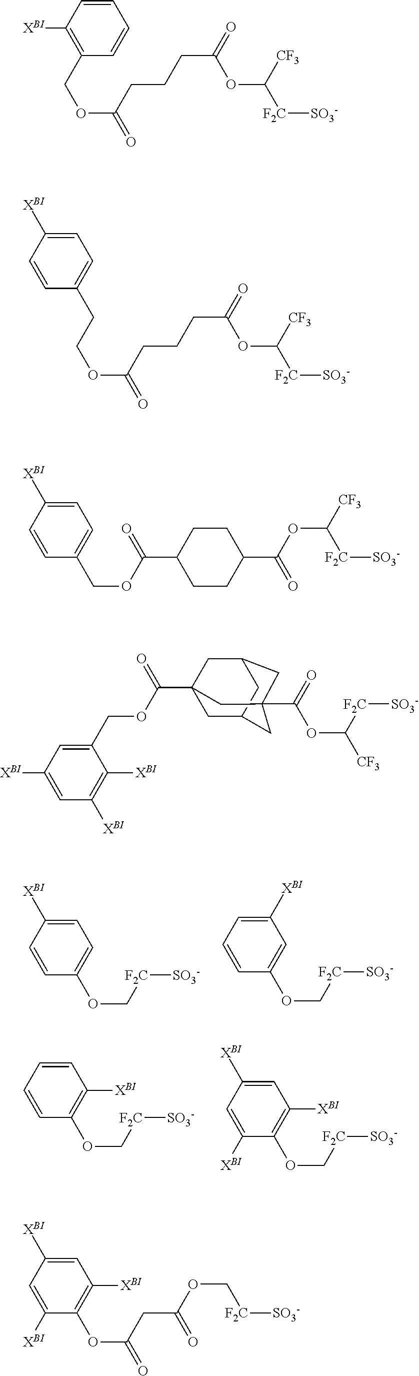

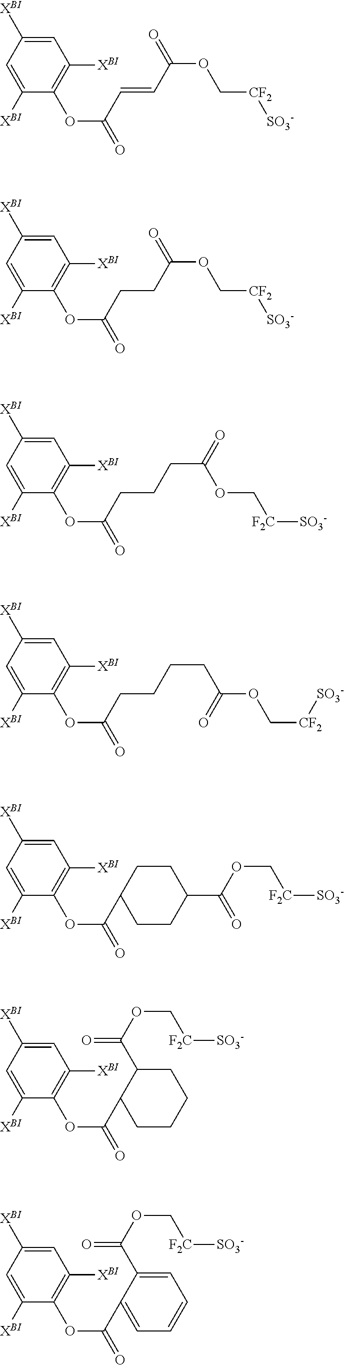

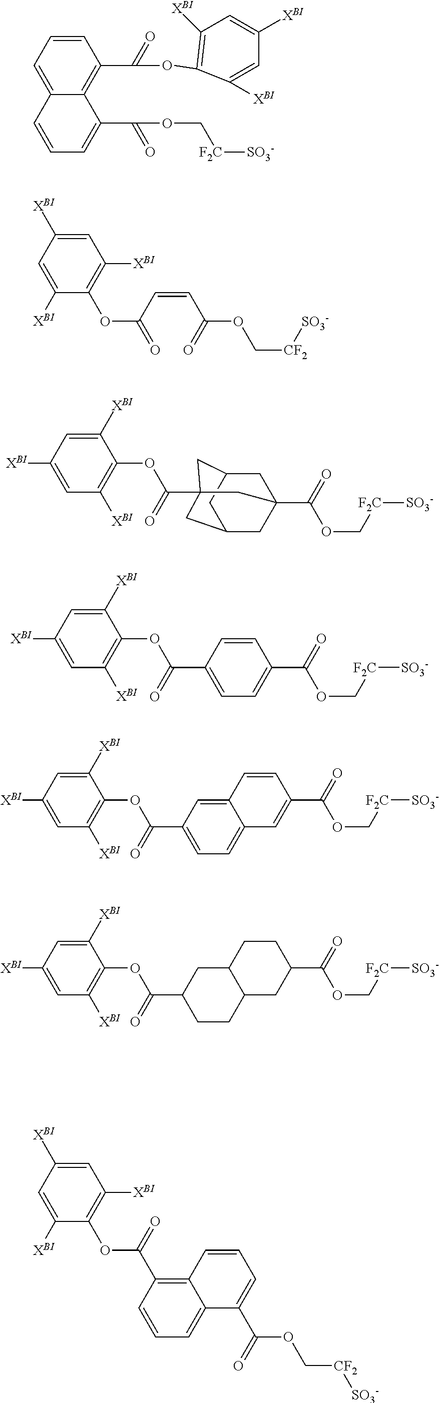

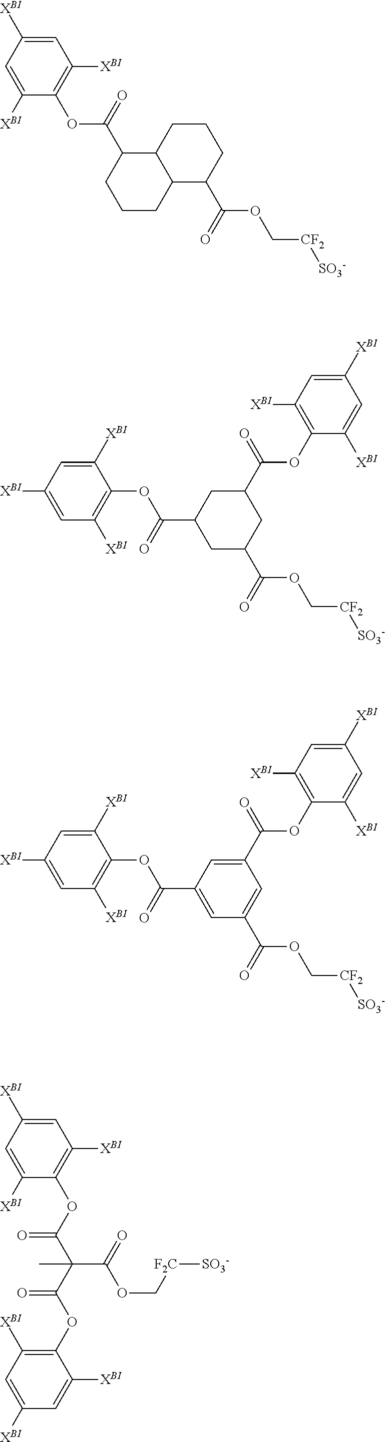

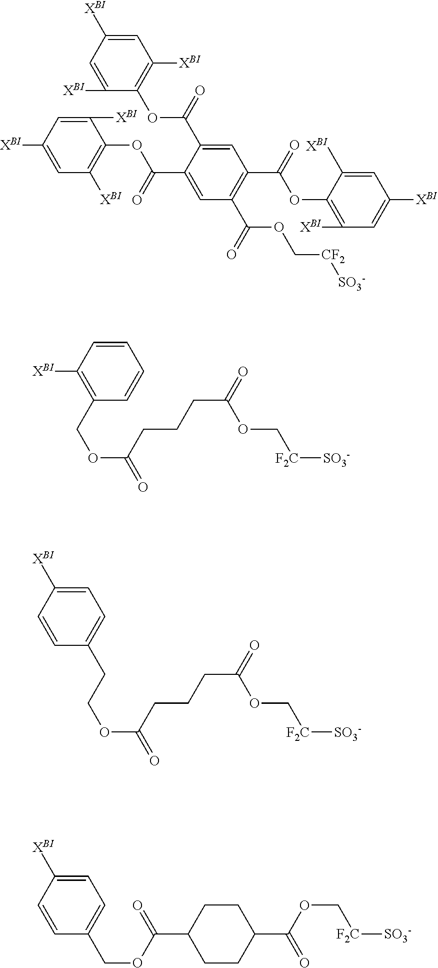

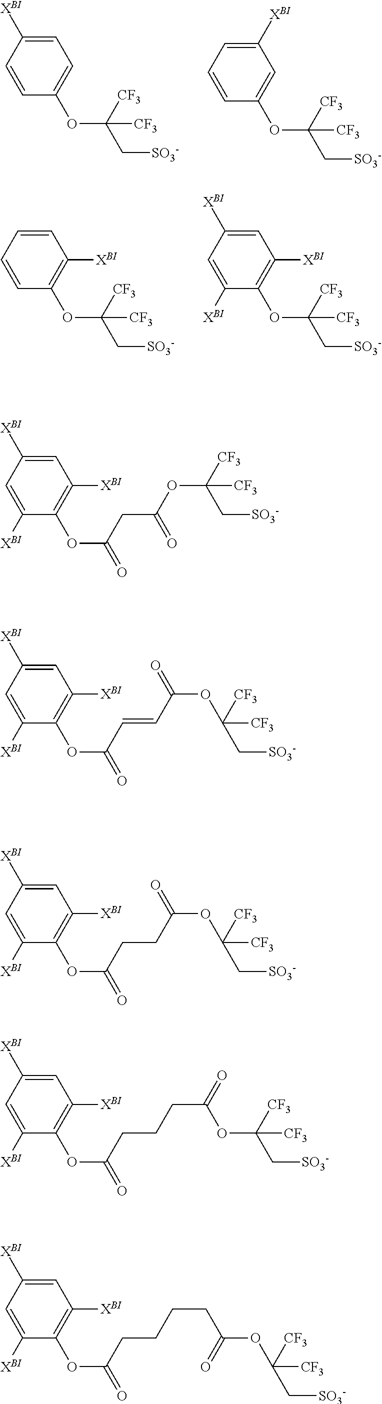

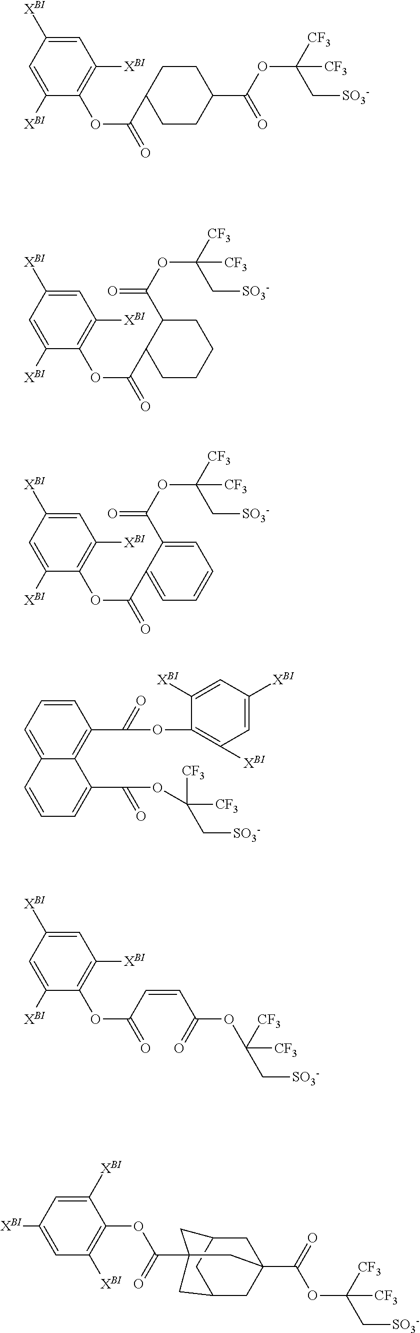

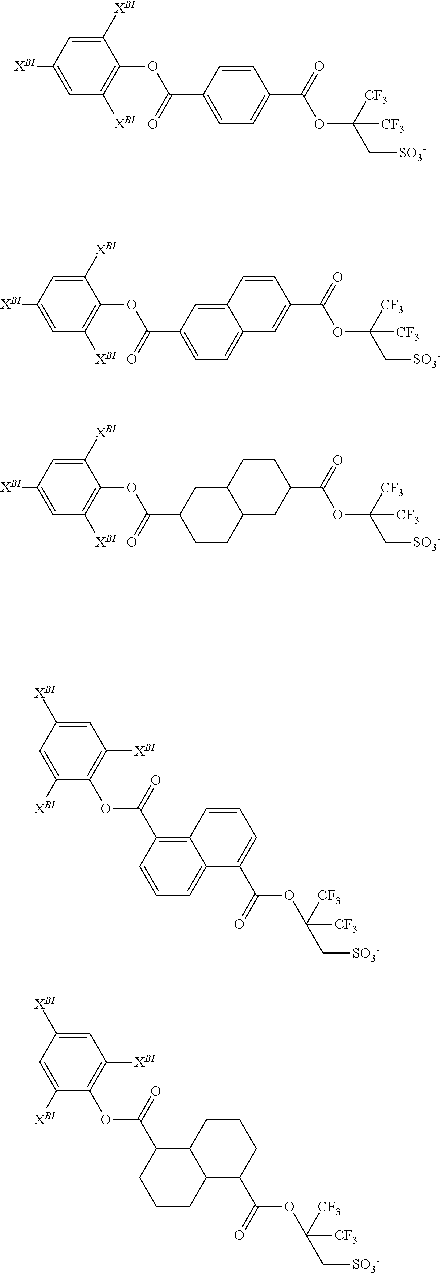

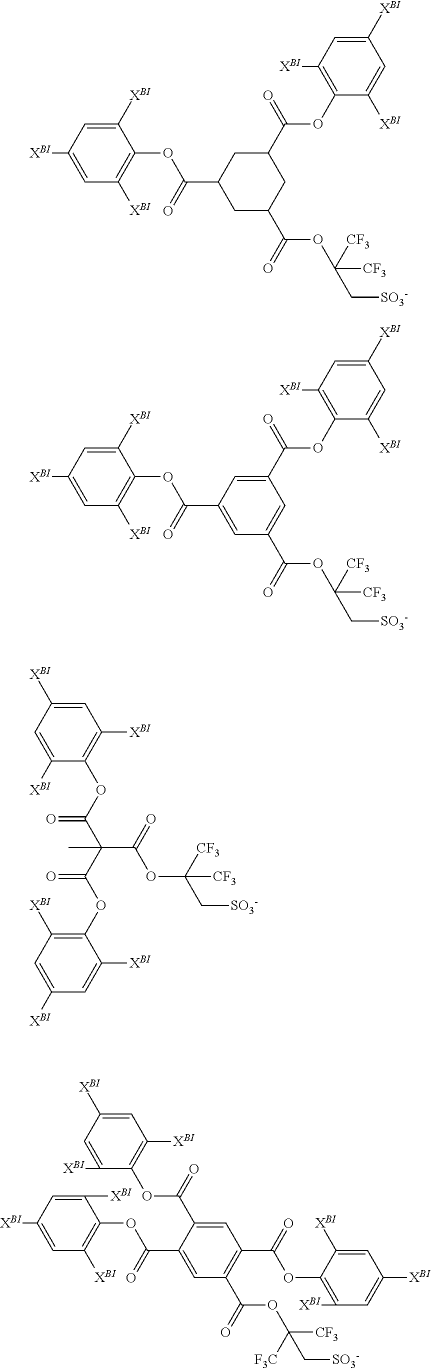

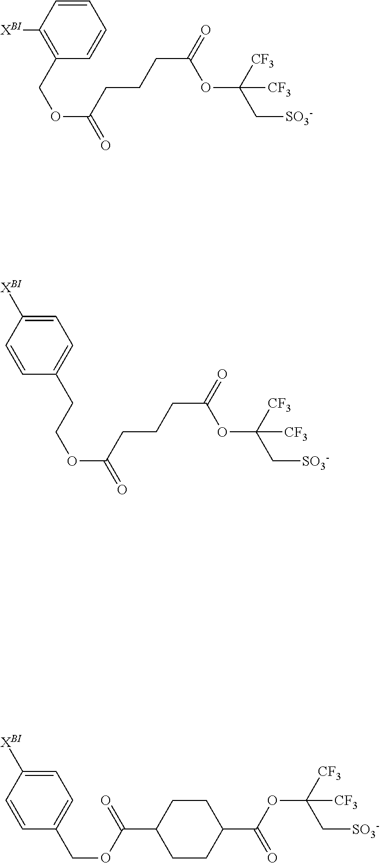

When the inventive resist composition contains the additive-type acid generator, the content thereof is preferably 0.1 to 50 parts by mass, and more preferably 1 to 40 parts by mass, relative to 100 parts by mass of the base polymer. If the inventive resist composition contains the base polymer having any one of the repeating units f1 to f3 and/or the additive-type acid generator, the inventive resist composition can function as the chemically amplified resist composition.

### [Organic Solvent]

The inventive resist composition may further comprise an organic solvent. The organic solvent is not particularly limited as long as the solvent can dissolve each of the aforementioned components and each component described later. Examples of the organic solvent include, described in paragraphs [0144] to [0145] of JP 2008-111103 A: ketones, such as cyclohexanone, cyclopentanone, methyl-2-n-pentyl ketone, and 2-heptanone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones, such as γ-butyrolactone.

The content of the organic solvent in the inventive resist composition is preferably 100 to 10,000 parts by mass, and more preferably 200 to 8,000 parts by mass, relative to 100 parts by mass of the base polymer. As the organic solvent, one kind may be used, or the mixture of two or more kinds may be used.

As described above, the inventive onium salt can exhibit sufficient solubility in the organic solvent. Therefore, the inventive resist composition has no concern about precipitation of the acid diffusion inhibitor.

### [Other Components]

The inventive resist composition may comprise, in addition to the aforementioned components, a surfactant, a dissolution inhibitor, a crosslinker, a quencher other than the inventive onium salt (hereinafter, referred to as the other quencher), a water repellency enhancer, and acetylene alcohols.

Examples of the surfactant include surfactants described in paragraphs [0165] to [0166] of JP 2008-111103 A. Adding the surfactant can further improve or regulate the coatability of the resist composition. When the inventive resist composition contains the surfactant, the content thereof is preferably 0.0001 to 10 parts by mass relative to 100 parts by mass of the base polymer. As the surfactant, one kind may be used, or combination of two or more kinds may be used.

When the inventive resist composition is the positive type resist composition, blending a dissolution inhibitor can further increase the difference in the dissolution rate between the exposed portion and the unexposed portion to further improve the resolution. Examples of the dissolution inhibitor include: a compound having a molecular weight of preferably 100 to 1,000, more preferably 150 to 800, and having two or more phenolic hydroxy groups in the molecule in which 0 to 100 mol % as an entirety of hydrogen atoms thereof are substituted with an acid-labile group; or a compound having a carboxy group in the molecule in which average 50 to 100 mol % as an entirety of hydrogen atoms of the carboxy groups are substituted with an acid-labile group. Specific examples thereof include compounds in which hydrogen atoms of hydroxy groups or carboxy groups in bisphenol A, trisphenol, phenolphthalein, cresol novolac, naphthacenecarboxylic acid, adamantanecarboxylic acid, and cholic acid are substituted with an acid-labile group. These dissolution inhibitors are described in paragraphs [0155] to [0178] of JP 2008-122932 A, for example.

When the inventive resist composition is the positive type resist composition containing the dissolution inhibitor, the content thereof is preferably 0 to 50 parts by mass, and more preferably 5 to 40 parts by mass, relative to 100 parts by mass of the base polymer. As the dissolution inhibitor, one kind may be used, or combination of two or more kind may be used.

Meanwhile, when the inventive resist composition is the negative-type resist composition, adding a crosslinker can reduce the dissolution rate in the exposed portion to obtain a negative pattern. Examples of the crosslinker include epoxy compounds, melamine compounds, guanamine compounds, glycoluryl compounds, urea compounds, isocyanate compounds, azide compounds, or compounds having a double bond such as an alkenyloxy group, these compounds being substituted with at least one group selected from a methylol group, an alkoxymethyl group, and an acyloxymethyl group. These compounds may be used as an additive, or may be introduced into the polymer side chain as a pendant group. Compounds having a hydroxy group can also be used as the crosslinker.

Examples of the epoxy compound include tris(2,3-epoxypropyl)isocyanurate, trimethylolethane triglycidyl ether, trimethylolpropane triglycidyl ether, and trimethylolethane triglycidyl ether.

Examples of the melamine compound include hexamethylolmelamine, hexamethoxymethylmelamine, a compound in which 1 to 6 methylol groups in hexamethylolmelamine are methoxymethylated or a mixture thereof, hexamethoxymethylmelamine, hexaacyloxymethylmelamine, and a compound in which 1 to 6 methylol groups in hexamethylolmelamine are acyloxymethylated or a mixture thereof.

Examples of the guanamine compound include tetramethylolguanamine, tetramethoxymethylguanamine, a compound in which 1 to 4 methylol groups in tetramethylolguanamine are methoxymethylated or a mixture thereof, tetramethoxyethylguanamine, tetraacyloxyguanamine, and a compound in which 1 to 4 methylol groups in tetramethylolguanamine are acyloxymethylated or a mixture thereof.

Examples of the glycoluryl compound include tetramethylolglycoluril, tetramethoxyglycoluril, tetramethoxymethylglycoluril, a compound in which 1 to 4 methylol groups in tetramethylolglycoluril are methoxymethylated or a mixture thereof, and a compound in which 1 to 4 methylol groups in tetramethylolglycoluril are acyloxymethylated or a mixture thereof.

Examples of the urea compound include tetramethylurea, tetramethoxymethylurea, a compound in which 1 to 4 methylol groups in tetramethylurea are methoxymethylated or a mixture thereof, and tetramethoxyethylurea.

Examples of the isocyanate compound include tolylene diisocyanate, diphenylmethane diisocyanate, hexamethylene diisocyanate, and cyclohexane diisocyanate.

Examples of the azide compound include 1,1′-biphenyl-4,4′-bisazide, 4,4′-methylidenebisazide, and 4,4′-oxybisazide.

Examples of the compound having an alkenyloxy group include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylolpropane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylolpropane trivinyl ether.

When the inventive resist composition is the negative-type resist composition containing the crosslinker, the content thereof is preferably 0.1 to 50 parts by mass, and more preferably 1 to 40 parts by mass, relative to 100 parts by mass of the base polymer. As the crosslinker, one kind may be used, or combination of two or more kinds may be used.

Examples of the other quencher include conventional basic compounds. Examples of the conventional basic compound include primary, secondary, or tertiary aliphatic amines, mixed amines, aromatic amines, heterocyclic amines, nitrogen-containing compounds having a carboxy group, nitrogen-containing compounds having a sulfonyl group, nitrogen-containing compounds having a hydroxy group, nitrogen-containing compounds having a hydroxyphenyl group, alcoholic nitrogen-containing compounds, amides, imides, and carbamates. Specifically, preferable are: primary, secondary, or tertiary amine compounds described in paragraphs [0146] to [0164] in JP 2008-111103 A; particularly, amine compounds having a hydroxy group, an ether bond, an ester bond, a lactone ring, a cyano group, or a sulfonate ester bond; or compounds having a carbamate group described in JP 3790649 B. Adding such a basic compound can further reduce the diffusion rate of the acid in the resist film and modify the shape, for example.

Examples of the other quencher also include onium salts of sulfonium salts, iodonium salts, or ammonium salts of a sulfonic acid having no-fluorinated α-position or a carboxylic acid, described in JP 2008-158339 A. Although the α-fluorinated sulfonic acid, the imide acid, or the methide acid is required for deprotecting the acid-labile group of the carboxylate ester, the acids is to be subjected to salt-exchange with the onium salt having no-fluorinated α-position releases a sulfonic acid having no-fluorinated α-position or a carboxylic acid. Since the sulfonic acid having no-fluorinated α-position and the carboxylic acid cause no deprotection reaction, such onium salts function as a quencher.

Examples of the other quencher further include a polymer quencher described in JP 2008-239918 A. This polymer quencher are orientated on a resist film surface to improve rectangularity of the resist pattern. The polymer quencher also has an effect of preventing film reduction of a pattern and rounding of a pattern top when applied for a protective film of immersion exposure.

When the inventive resist composition contains the other quencher, the content thereof is preferably 0 to 5 parts by mass, and more preferably 0 to 4 parts by mass, relative to 100 parts by mass of the base polymer. As the other quencher, one kind may be used, or combination of two or more kinds may be used.

The water repellency enhancer improves water repellency on the resist film surface, and can be used for immersion lithography without a top coating. As the water repellency enhancer, a polymer having a fluorinated alkyl group, a polymer having a 1,1,1,3,3,3-hexafluoro-2-propanol residue with a specific structure, etc. are preferable, and water repellency enhancers exemplified in JP 2007-297590 A, JP 2008-111103 A, etc. are more preferable. The water repellency enhancer is necessarily dissolved in an alkaline developer or an organic solvent developer. The aforementioned water repellency enhancer having the specific 1,1,1,3,3,3-hexafluoro-2-propanol residue has good solubility in the developer. A water repellency enhancer being a polymer having a repeating unit having an amino group or an amine salt prevents evaporation of the acid during post exposure bake (PEB) to highly effectively prevent opening failure of a hole pattern after the development. When the inventive resist composition (chemically amplified resist material) contains the above water repellency enhancer, the content thereof is preferably 0 to 20 parts by mass, and more preferably 0.5 to 10 parts by mass, relative to 100 parts by mass of the base polymer. As the water repellency enhancer, one kind may be used, or combination of two or more kinds may be used.

Examples of the acetylene alcohols include those described in paragraphs [0179] to [0182] in JP 2008-122932 A. When the inventive chemically amplified resist composition contains the acetylene alcohols, the content thereof is preferably 0 to 5 parts by mass relative to 100 parts by mass of the base polymer. As the acetylene alcohol, one kind may be used, or combination of two or more kinds may be used.

### [Patterning Process]

When the inventive resist composition is used for various integrated circuit manufacturing, known lithographic technology can be applied. Examples of the inventive patterning process include a method comprising steps of: forming a resist film on a substrate by using the aforementioned inventive resist composition (chemically amplified resist material); exposing the resist film to high-energy ray; and developing the exposed resist film by using a developer.

Hereinafter, an example of the inventive patterning process will be described.

First, the inventive resist composition is applied on a substrate for integrated circuit manufacturing (for example, Si, SiO₂, SiN, SiON, TiN, WSi, BPSG, SOG, an organic anti-reflective film, etc.) or a substrate for mask circuit manufacturing (for example, Cr, CrO, CrON, MoSi₂, SiO₂, etc.) by an appropriate coating method such as spin-coating, roll-coating, flow-coating, dip-coating, spray-coating, and doctor-coating so that the coating film thickness is 0.01 to 2 μm. This film is pre-baked on a hot plate at preferably 60 to 150° C. for 10 seconds to 30 minutes, more preferably at 80 to 120° C. for 30 seconds to 20 minutes to form a resist film.

Then, the resist film is exposed by using high-energy ray. Examples of the high-energy ray include ultraviolet ray, far ultraviolet ray, electron beam (EB), extreme ultraviolet ray (EUV) having a wave length of 3 to 15 nm, X-ray, soft X-ray, excimer laser light, γ-ray, and synchrotron radiation. When ultraviolet ray, far ultraviolet ray, EUV, X-ray, soft X-ray, excimer laser light, γ-ray, synchrotron radiation, etc. is used as the high-energy ray, irradiation is performed directly or by using a mask for forming a target pattern so that an exposure dose is preferably approximately 1 to 200 mJ/cm², more preferably approximately 10 to 100 mJ/cm². When EB is used as the high-energy ray, writing is performed directly or by using a mask for forming a target pattern at an exposure dose of preferably approximately 0.1 to 300 μC/cm², more preferably approximately 0.5 to 200 μC/cm². The inventive resist composition is particularly suitable for fine pattering with, among the high-energy ray, KrF excimer laser light, ArF excimer laser light, EB, EUV, X-ray, soft X-ray, γ-ray, and synchrotron radiation. Among them, KrF excimer laser light, ArF excimer laser light, EB, or EUV having a wave length of 3 to 15 nm is preferably used, and the inventive resist composition is particularly suitable for fine patterning with EB or EUV.

After the exposure, PEB may be performed on a hot plate or in an oven at preferably 30 to 150° C. for 10 seconds to 30 minutes, more preferably 50 to 120° C. for 30 seconds to 20 minutes. The PEB may not be performed.

After the exposure or the PEB, the exposed resist film is developed by a common method such as a dip method, a puddle method, and a spray method for 3 seconds to 3 minutes, preferably 5 seconds to 2 minutes to form a target pattern. In the common method, used are a developer of a 0.1 to 10 mass %, preferably 2 to 5 mass %, alkaline aqueous solution such as tetramethylammonium hydroxide (TMAH), tetraethylammonium hydroxide, tetrapropylammonium hydroxide, and tetrabutylammonium hydroxide, for example. In a case of the positive-type resist composition, the light-irradiated portion is dissolved in the developer and the unexposed portion is not dissolved to form a target positive-type pattern on the substrate. In a case of the negative-type resist composition, in contrast to the case of the positive-type resist composition, the light-irradiated portion is insoluble in the developer and the unexposed portion is dissolved.

The negative pattern can also be obtained by using the positive-type resist composition containing the base polymer having an acid-labile group with the organic solvent development. Examples of the developer used in this case include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. As the organic solvent, one kind can be used singly, or the mixture of two or more kinds used.

After the development, rinsing is preferably performed. The rinsing liquid is preferably a solvent that can mix with the developer and that does not dissolve the resist film. Preferably used as such a solvent are alcohols having 3 to 10 carbon atoms, ether compounds having 8 to 12 carbon atoms, alkanes, alkenes, or alkynes having 6 to 12 carbon atoms, and aromatic solvents, for example.

Examples of the alcohols having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Examples of the ether compounds having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Examples of the alkanes having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Examples of the alkenes having 6 to 12 carbon atoms include hexene, heptene, octene, cyclohexene, methylcyclohexene, dimethylcyclohexene, cycloheptene, and cyclooctene. Examples of the alkynes having 6 to 12 carbon atoms include hexyne, heptyne, and octyne.

Examples of the aromatic solvents include toluene, xylene, ethylbenzene, isopropylbenzene, tert-butylbenzene, and mesitylene.

The rinsing can reduce collapse of the resist pattern and occurrence of defects. Rinsing is not essential, and no rinsing can reduce a use amount of the solvent.

A hole pattern or trench pattern after the development can be shrunk by thermal flow, RELACS technology, or DSA technology. A shrinking agent is applied on the hole pattern, and during baking, an acid catalyst is diffused from the resist film to cause the crosslinking of the shrinking agent on the resist film surface, and the shrinking agent adheres to the side wall of the hole pattern. The baking temperature is preferably 70 to 180° C., and more preferably 80 to 170° C. A baking time is preferably 10 to 300 seconds. The extra shrinking agent is removed to shrink the hole pattern.

Since using the inventive resist composition, the inventive patterning process can form a resist film that has excellent sensitivity and resolution of the resist film, that can reduce LWR of a line pattern and CDU of a hole pattern and dot pattern, that can inhibit swelling during the development, that can inhibit collapse of the resist pattern, and that can precisely perform fine processing.

### EXAMPLE

Hereinafter, the present invention will be specifically described by showing Synthesis Examples, Examples, and Comparative Examples, but the present invention is not limited to the following Examples. Used apparatus was as follows.
MALDI TOF-MS: S3000, manufactured by JEOL Ltd.

### [1] Synthesis of Onium Salt

### [Example 1-1] Synthesis of SQ-1

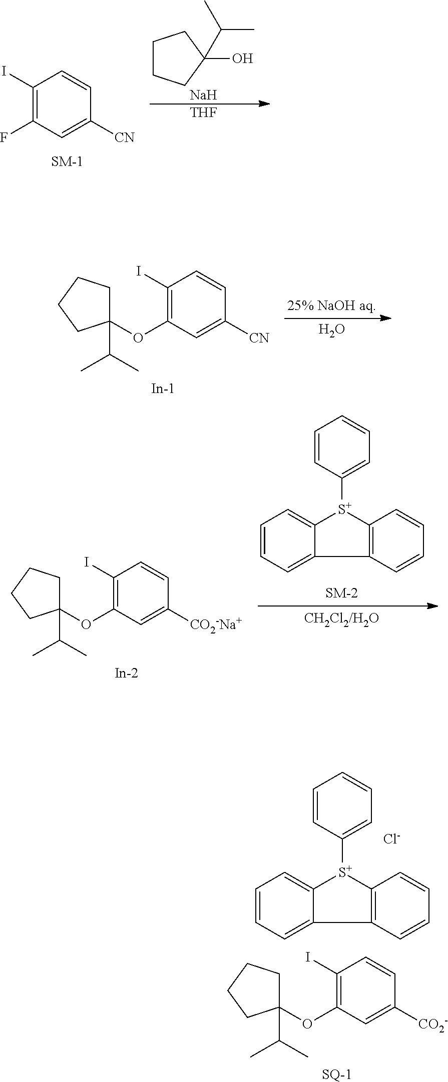

### (1) Synthesis of Intermediate In-1

Under a nitrogen atmosphere, sodium hydride (purity: 55 mass %, 4.8 g) was dispersed in THF (60 ml), and a solution composed of 1-isopropylcyclopentanol (14.1 g) and THF (15 ml) was added dropwise thereinto. After the dropwise addition, the mixture was heated with reflux for 4 hours to prepare a metal alkoxide. Then, a raw material SM-1 (24.7 g) was added dropwise, and the mixture was heated with reflux and aged for 18 hours. The reaction liquid was cooled with an ice bath, and the reaction was terminated with water (100 ml). The target product was extracted with s solvent composed of toluene (100 ml) and hexane (100 ml), common aqueous work-up was performed, the solvent was evaporated, and then purification was performed with silica gel chromatography to obtain 28.8 g of an intermediate In-1 as a colorless oil (81% yield).

### (2) Synthesis of Intermediate In-2

Under a nitrogen atmosphere, the intermediate In-1 (28.8 g) was aged at an internal temperature of 100° C. for 24 hours in a solution composed of a 25 mass % aqueous solution of sodium hydroxide (38.9 g) and water (100 ml). After the aging, the reaction liquid was cooled, and azeotropic dehydration was performed five times with adding toluene (500 ml). The residue after the azeotropic dehydration was recrystallized with hexane to obtain 18.0 g of an intermediate In-2 as a white crystal (56% yield).

### (3) Synthesis of Onium Salt SQ-1

Under a nitrogen atmosphere, the intermediate In-2 (7.9 g) and a raw material SM-2 (6.5 g) were dissolved in methylene chloride (50 g) and water (40 g), and the mixture was stirred for 20 minutes. The reaction liquid was separated, the organic layer was collected, then common aqueous work-up was performed, and the solvent was evaporated to obtain 11.0 g of an onium salt SQ-1 as a colorless oil (87% yield).

The results of TOF-MS on the onium salt SQ-1 are shown below.
MALDI TOF-MS: POSITIVE M⁺261 (corresponding to C₁₈H₁₃S⁺)
   NEGATIVE M⁻373 (corresponding to C₁₅H₁₈IO3⁻)

### [Examples 1-2 to 1-9] Syntheses of SQ-2 to SQ-9

Each onium salt was synthesized with each organic synthesis reaction. The structures of onium salts used for chemically amplified resist compositions are shown below.

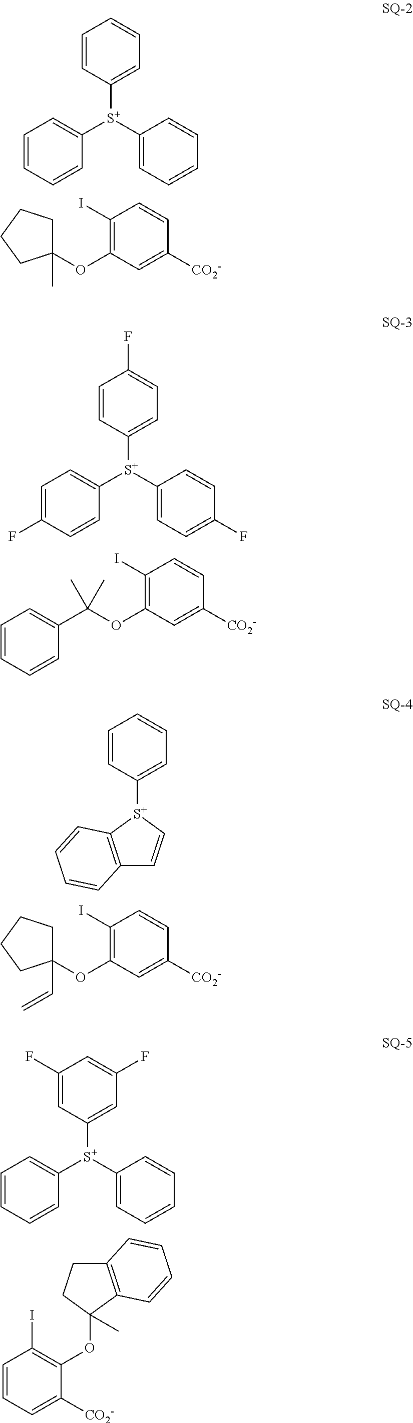

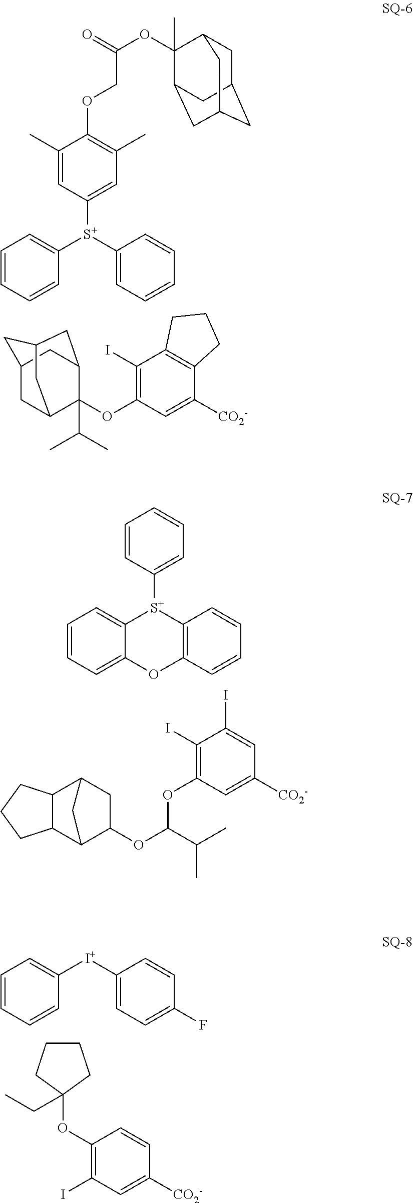

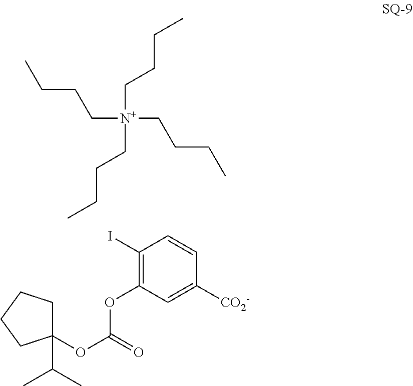

### [2] [Synthesis Example] Syntheses of Base Polymers (P-1 to 5)

Monomers were combined to perform a copolymerization reaction in a THF solvent, the product was precipitated with methanol, further washed repeatedly with hexane, then isolated, and dried to obtain each of base polymers (P-1 to 5) having the following composition. The composition of the obtained base polymer was determined by ¹H-NMR, and Mw and Mw/Mn thereof were determined by GPC (solvent: THF, standard: polystyrene).

| | | | | |
|---|---|---|---|---|
| P-1 Mw = 4,900 Mw/Mn = 1.51 | 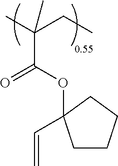 | 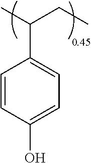 | | |
| P-2 Mw = 8,900 Mw/Mn = 1.89 | 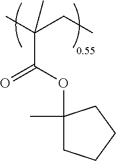 | 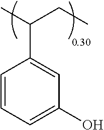 | 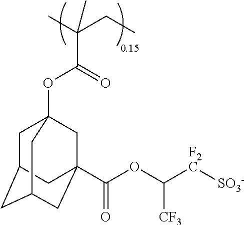 | |
| | | | 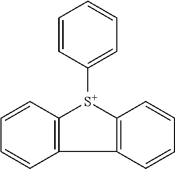 [SMILES: c1ccc(-[s+]2c3ccccc3c3ccccc32)cc1] | |
| P-3 Mw = 7,800 Mw/Mn = 1.75 | 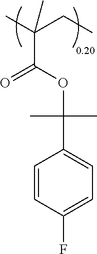 | 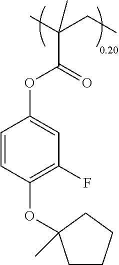 | 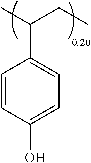 | 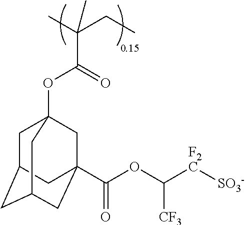 |
| | | | | 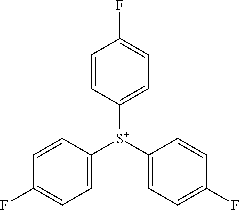 [SMILES: Fc1ccc([S+](c2ccc(F)cc2)c2ccc(F)cc2)cc1] |
| P-4 Mw = 8,300 Mw/Mn = 1.81 | 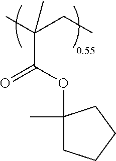 | 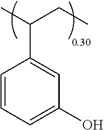 | 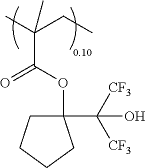 | 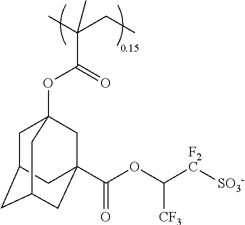 |
| | | | | 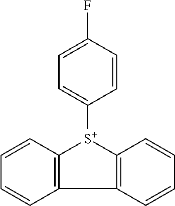 [SMILES: Fc1ccc(-[s+]2c3ccccc3c3ccccc32)cc1] |
| P-5 Mw = 6,900 Mw/Mn = 1.62 | 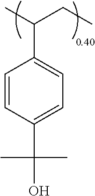 | 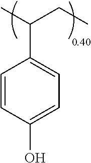 | 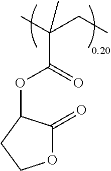 | |

### [3] [Examples 2-1 to 2-20 and Comparative Examples 1-1 to 1-12] Preparation of Resist Composition

### (1) Preparation of Resist Composition

A solution dissolving each component at a composition shown in Table 1 and Table 2 was filtered with a filter with 0.2 μm in size to prepare a resist composition. Resist compositions of Examples 2-1 to 2-18 and Comparative Examples 1-1 to 1-10 were positive-type resist compositions, and resist compositions of Examples 2-19 and 2-20 and Comparative Examples 1-11 and 1-12 were negative-type resist compositions.

Each component in Table 1 was as follows.

### Organic Solvent:

PGMEA (propylene glycol monomethyl ether acetate)
DAA (diacetone alcohol)

### Photoacid Generator: PAG-1 to PAG-5

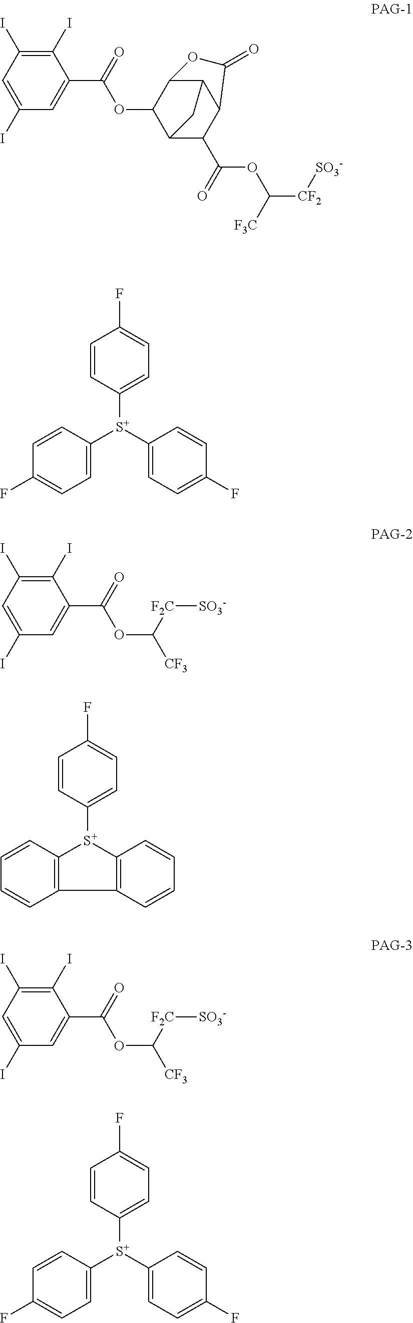

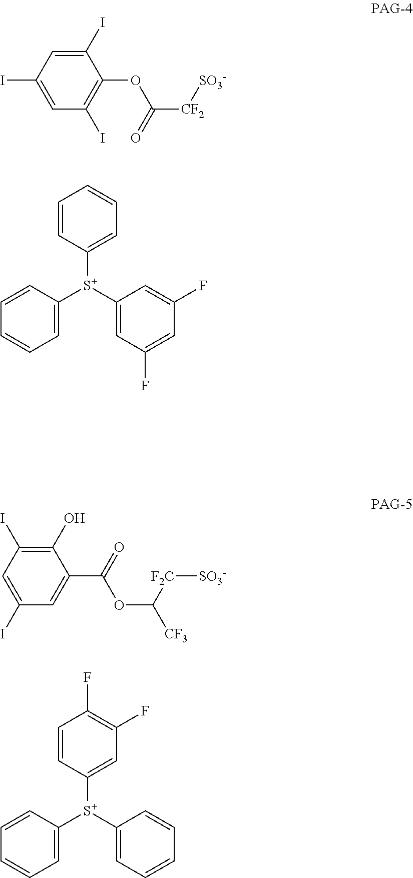

Blended Quencher: bQ-1 and bQ-2

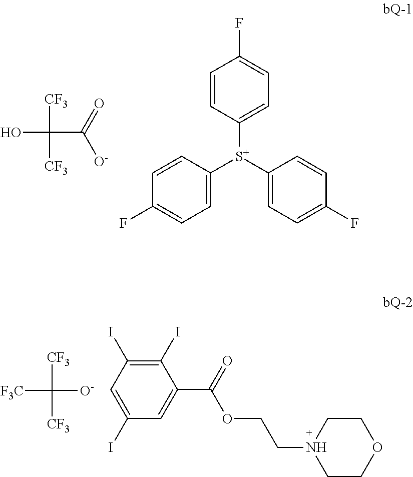 [SMILES: O=C([O-])C(O)(C(F)(F)F)C(F)(F)F ; Fc1ccc([S+](c2ccc(F)cc2)c2ccc(F)cc2)cc1 ; [O-]C(C(F)(F)F)(C(F)(F)F)C(F)(F)F ; O=C(OCCN1CCOCC1)c1cc(I)cc(I)c1I]

Comparative Quencher: cSQ-1 to cSQ-4

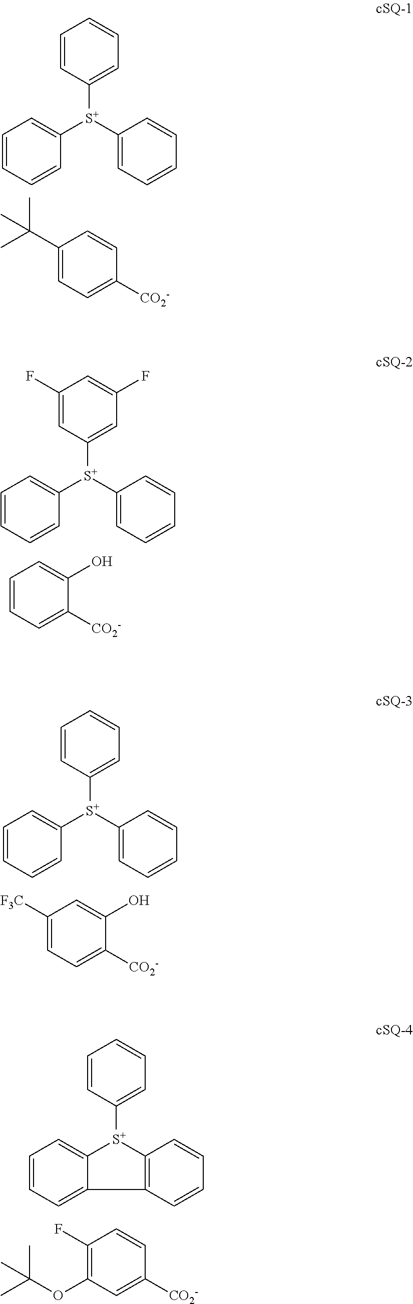

TABLE 1

| | | Base | Photoacid | | Organic | Organic |
|---|---|---|---|---|---|---|
| | | polymer | generator | Quencher | solvent 1 | solvent 2 |
| | Resist | (parts by | (parts by | (parts by | (parts by | (parts by |
| | composition | mass) | mass) | mass) | mass) | mass) |
| Example | R-1 | P-1 | PAG-1 | SQ-1 | PGMEA | DAA |
| 2-1 | | (100) | (30.2) | (4.8) | (3,000) | (500) |
| Example | R-2 | P-1 | PAG-2 | SQ-2 | PGMEA | DAA |
| 2-2 | | (100) | (24.8) | (4.7) | (3,000) | (500) |
| Example | R-3 | P-1 | PAG-2 | SQ-3 | PGMEA | DAA |
| 2-3 | | (100) | (24.8) | (4.8) | (3,000) | (500) |
| Example | R-4 | P-1 | PAG-2 | SQ-4 | PGMEA | DAA |
| 2-4 | | (100) | (24.8) | (4.7) | (3,000) | (500) |
| Example | R-5 | P-1 | PAG-2 | SQ-5 | PGMEA | DAA |
| 2-5 | | (100) | (24.8) | (4.8) | (3,000) | (500) |
| Example | R-6 | P-1 | PAG-3 | SQ-6 | PGMEA | DAA |
| 2-6 | | (100) | (25.2) | (4.9) | (3,000) | (500) |
| Example | R-7 | P-1 | PAG-4 | SQ-7 | PGMEA | DAA |
| 2-7 | | (100) | (25.2) | (4.8) | (3,000) | (500) |
| Example | R-8 | P-1 | PAG-3 | SQ-8 | PGMEA | DAA |
| 2-8 | | (100) | (25.2) | (4.8) | (3,000) | (500) |
| Example | R-9 | P-1 | PAG-1 | SQ-9 | PGMEA | DAA |
| 2-9 | | (100) | (30.2) | (2.4) | (3,000) | (500) |
| | | | | bQ-1 | | |
| | | | | (2.4) | | |
| Example | R-10 | P-2 | — | SQ-1 | PGMEA | DAA |
| 2-10 | | (100) | | (4.6) | (3,000) | (500) |
| Example | R-11 | P-2 | — | SQ-2 | PGMEA | DAA |
| 2-11 | | (100) | | (4.9) | (3,000) | (500) |
| Example | R-12 | P-2 | — | SQ-3 | PGMEA | DAA |
| 2-12 | | (100) | | (4.8) | (3,000) | (500) |
| Example | R-13 | P-3 | PAG-3 | SQ-4 | PGMEA | DAA |
| 2-13 | | (100) | (10.5) | (4.8) | (3,000) | (500) |
| Example | R-14 | P-3 | — | SQ-6 | PGMEA | DAA |
| 2-14 | | (100) | | (4.7) | (2,200) | (900) |
| Example | R-15 | P-3 | — | SQ-8 | PGMEA | DAA |
| 2-15 | | (100) | | (4.6) | (2,200) | (900) |
| Example | R-16 | P-4 | — | SQ-1 | PGMEA | DAA |
| 2-16 | | (100) | | (2.3) | (2,200) | (900) |
| | | | | bQ-2 | | |
| | | | | (2.3) | | |
| Example | R-17 | P-4 | — | SQ-5 | PGMEA | DAA |
| 2-17 | | (100) | | (4.7) | (2,200) | (900) |
| Example | R-18 | P-4 | — | SQ-2 | PGMEA | DAA |
| 2-18 | | (100) | | (4.7) | (2,200) | (900) |
| Example | R-19 | P-5 | PAG-1 | SQ-1 | PGMEA | DAA |
| 2-19 | | (100) | (30.2) | (4.5) | (2,200) | (900) |
| Example | R-20 | P-5 | PAG-5 | SQ-3 | PGMEA | DAA |
| 2-20 | | (100) | (25.2) | (4.6) | (2,200) | (900) |

TABLE 2

| | | Base | Photoacid | | Organic | Organic |
|---|---|---|---|---|---|---|
| | | polymer | generator | Quencher | solvent 1 | solvent 2 |
| | Resist | (parts by | (parts by | (parts by | (parts by | (parts by |
| | composition | mass) | mass) | mass) | mass) | mass) |
| Comparative | CR-1 | P-1 | PAG-1 | cSQ-1 | PGMEA | DAA |
| Example 1-1 | | (100) | (30.2) | (4.8) | (3,000) | (500) |
| Comparative | CR-2 | P-1 | PAG-2 | cSQ-2 | PGMEA | DAA |
| Example 1-2 | | (100) | (24.9) | (4.6) | (3,000) | (500) |
| Comparative | CR-3 | P-1 | PAG-3 | cSQ-3 | PGMEA | DAA |
| Example 1-3 | | (100) | (25.3) | (4.7) | (3,000) | (500) |
| Comparative | CR-4 | P-1 | PAG-4 | cSQ-4 | PGMEA | DAA |
| Example 1-4 | | (100) | (25.1) | (4.8) | (3,000) | (500) |
| Comparative | CR-5 | P-2 | — | cSQ-4 | PGMEA | DAA |
| Example 1-5 | | (100) | | (4.8) | (3,000) | (500) |
| Comparative | CR-6 | P-2 | — | cSQ-2 | PGMEA | DAA |
| Example 1-6 | | (100) | | (4.6) | (3,000) | (500) |
| Comparative | CR-7 | P-3 | — | cSQ-3 | PGMEA | DAA |
| Example 1-7 | | (100) | | (4.7) | (3,000) | (500) |
| Comparative | CR-8 | P-3 | PAG-3 | cSQ-2 | PGMEA | DAA |
| Example 1-8 | | (100) | (10.5) | (4.6) | (3,000) | (500) |
| Comparative | CR-9 | P-4 | — | cSQ-4 | PGMEA | DAA |
| Example 1-9 | | (100) | | (4.6) | (3,000) | (500) |
| Comparative | CR-10 | P-4 | — | cSQ-3 | PGMEA | DAA |
| Example 1-10 | | (100) | | (4.7) | (3,000) | (500) |
| Comparative | CR-11 | P-5 | PAG-1 | cSQ-2 | PGMEA | DAA |
| Example 1-11 | | (100) | (30.2) | (4.6) | (3,000) | (500) |
| Comparative | CR-12 | P-5 | PAG-5 | cSQ-4 | PGMEA | DAA |
| Example 1-12 | | (100) | (24.6) | (4.5) | (3,000) | (500) |

### [4] EUV Lithography Evaluation (1)

### Examples 3-1 to 3-20 and Comparative Examples 2-1 to 2-12

Each of the chemically amplified resist compositions (R-1 to R-20 and CR-1 to CR-12) shown in Table 1 and Table 2 was applied by spin-coating on a Si substrate on which a silicon-containing spin-on hard mask SHB-A940 (silicon content of 43 mass %), manufactured by Shin-Etsu Chemical Co., Ltd., was formed with 20 nm in film thickness. Then, the applied composition was prebaked at 100° C. for 60 seconds by using a hot plate to produce a resist film with 50 nm in film thickness. This resist film was exposed by using an EUV scanner NXE3300 (NA 0.33, σ 0.9/0.6, dipole illumination), manufactured by ASML Holding N.V. The exposure was performed with a LS pattern with 18 nm on wafer size and 36 nm in pitch, and with changing an exposure dose and focus (exposure dose pitch: 1 mJ/cm², focus pitch: 0.020 μm). After the exposure, PEB was performed at a temperature shown in Table 3 and Table 4 for 60 seconds. Thereafter, puddle development with a 2.38 mass % aqueous TMAH solution for 30 seconds, rinsing with a surfactant-containing rinsing material, and spin-drying were performed to obtain a positive-type pattern in Examples 3-1 to 3-18 and Comparative Examples 2-1 to 2-10. In Examples 3-19 and 3-20 and Comparative Examples 2-11 and 2-12, a negative-type pattern was obtained.

The obtained LS pattern was observed with a length-measurement SEM (CG6300), manufactured by Hitachi High-Technologies Corporation, to evaluate sensitivity, exposure latitude (EL), LWR, depth of focus (DOF), and collapse limit in accordance with the following methods. Table 3 and Table 4 show the results.

### [Sensitivity Evaluation]

An optimum exposure dose Eₒₚ(mJ/cm²) to yield the LS pattern with 18 nm in line width and 36 nm in pitch was determined to specify this value as a sensitivity. A smaller sensitivity value indicates higher sensitivity.

### [EL Evaluation]

From exposure doses that formed LS patterns within a range of the 18 nm space width±10% (16.2 to 19.8 nm), EL (unit: %) was determined by the following equation. The larger the EL value, the better the performance.

EL (%)=(|E₁−E₂|/Eₒₚ)×100
E₁: An optimum exposure dose to yield a LS pattern with 16.2 nm in line width and 36 nm in pitch.
E₂: An optimum exposure dose to yield a LS pattern with 19.8 nm in line width and 36 nm in pitch.
Eₒₚ: An optimum exposure dose to yield the LS pattern with 18 nm in line width and 36 nm in pitch.

### [LWR Evaluation]

In the LS pattern obtained by irradiation at Eₒₚ, sizes at 10 positions in the longitudinal direction of the line were measured. From the results, a tripled value (3σ) of a standard variation (σ) was determined as LWR. A smaller LWR value can yield a pattern with smaller roughness and uniform line width.

### [DOF Evaluation]

As evaluation of depth of focus, determined was a focus range that formed LS patterns within a range of the 18 nm size±10% (16.2 to 19.8 nm). A larger DOF value indicates wider depth of focus.

### [Evaluation of Collapse Limit of Line Pattern]

Line sizes of the LS patterns at each exposure dose with the optimum focus were measured at 10 positions in the longitudinal direction. A narrowest line size obtained without collapse was specified as a collapse limit size. A smaller limit size value indicates excellent collapse limit.

TABLE 3

| | | | Optimal | | | | |
|---|---|---|---|---|---|---|---|
| | | PEB | exposure | | | | Collapse |
| | Resist | temperature | dose | EL | LWR | DOF | limit |
| | composition | (° C.) | (mJ/cm²) | (%) | (nm) | (nm) | (nm) |
| Example | R-1 | 105 | 32 | 17 | 2.8 | 110 | 10.1 |
| 3-1 | | | | | | | |
| Example | R-2 | 100 | 33 | 18 | 2.9 | 120 | 10.4 |
| 3-2 | | | | | | | |
| Example | R-3 | 105 | 33 | 17 | 3 | 110 | 10.3 |
| 3-3 | | | | | | | |
| Example | R-4 | 100 | 32 | 17 | 2.8 | 110 | 10.2 |
| 3-4 | | | | | | | |
| Example | R-5 | 100 | 34 | 18 | 2.7 | 100 | 10.7 |
| 3-5 | | | | | | | |
| Example | R-6 | 100 | 33 | 17 | 2.8 | 120 | 10.6 |
| 3-6 | | | | | | | |
| Example | R-7 | 100 | 32 | 18 | 2.9 | 120 | 10.7 |
| 3-7 | | | | | | | |
| Example | R-8 | 105 | 32 | 16 | 3 | 110 | 11.1 |
| 3-8 | | | | | | | |
| Example | R-9 | 100 | 33 | 18 | 2.9 | 120 | 11.2 |
| 3-9 | | | | | | | |
| Example | R-10 | 100 | 28 | 17 | 2.9 | 120 | 10.7 |
| 3-10 | | | | | | | |
| Example | R-11 | 105 | 29 | 17 | 2.7 | 120 | 10.5 |
| 3-11 | | | | | | | |
| Example | R-12 | 100 | 28 | 18 | 2.8 | 110 | 10.4 |
| 3-12 | | | | | | | |
| Example | R-13 | 95 | 26 | 19 | 2.8 | 100 | 10.5 |
| 3-13 | | | | | | | |
| Example | R-14 | 100 | 28 | 18 | 3 | 120 | 10.8 |
| 3-14 | | | | | | | |
| Example | R-15 | 100 | 29 | 17 | 2.7 | 120 | 10.4 |
| 3-15 | | | | | | | |
| Example | R-16 | 100 | 29 | 18 | 2.8 | 110 | 10.8 |
| 3-16 | | | | | | | |
| Example | R-17 | 105 | 28 | 19 | 2.9 | 100 | 10.8 |
| 3-17 | | | | | | | |
| Example | R-18 | 105 | 28 | 18 | 2.8 | 120 | 10.8 |
| 3-18 | | | | | | | |
| Example | R-19 | 110 | 37 | 16 | 3.1 | 120 | 11.1 |
| 3-19 | | | | | | | |
| Example | R-20 | 110 | 36 | 17 | 3 | 120 | 11.2 |
| 3-20 | | | | | | | |

TABLE 4

| | | | Optimal | | | | |
|---|---|---|---|---|---|---|---|
| | | PEB | exposure | | | | Collapse |
| | Resist | temperature | dose | EL | LWR | DOF | limit |
| | composition | (° C.) | (mJ/cm²) | (%) | (nm) | (nm) | (nm) |
| Comparative | CR-1 | 100 | 41 | 16 | 3.4 | 80 | 14.3 |
| Example 2-1 | | | | | | | |
| Comparative | CR-2 | 105 | 42 | 15 | 3.4 | 90 | 14.5 |
| Example 2-2 | | | | | | | |
| Comparative | CR-3 | 100 | 40 | 15 | 3.3 | 80 | 14.2 |
| Example 2-3 | | | | | | | |
| Comparative | CR-4 | 100 | 42 | 15 | 3.2 | 90 | 13.7 |
| Example 2-4 | | | | | | | |
| Comparative | CR-5 | 105 | 35 | 14 | 3.3 | 70 | 14.2 |
| Example 2-5 | | | | | | | |
| Comparative | CR-6 | 105 | 34 | 16 | 3.4 | 90 | 13.4 |
| Example 2-6 | | | | | | | |
| Comparative | CR-7 | 100 | 33 | 15 | 3.5 | 80 | 12.9 |
| Example 2-7 | | | | | | | |
| Comparative | CR-8 | 95 | 34 | 16 | 3.2 | 80 | 13.5 |
| Example 2-8 | | | | | | | |
| Comparative | CR-9 | 100 | 32 | 16 | 3.6 | 90 | 13.4 |
| Example 2-9 | | | | | | | |
| Comparative | CR-10 | 105 | 33 | 15 | 3.5 | 70 | 13.6 |
| Example 2-10 | | | | | | | |
| Comparative | CR-11 | 110 | 41 | 16 | 3.6 | 100 | 12.9 |
| Example 2-11 | | | | | | | |
| Comparative | CR-12 | 105 | 42 | 15 | 3.6 | 90 | 14.1 |
| Example 2-12 | | | | | | | |

From the results shown in Table 3 and Table 4, Examples 3-1 to 3-18, which formed the positive-type pattern, had a mostly smaller optimal exposure dose than Comparative Examples 2-1 to 2-10, which formed a similar positive-type pattern, and confirmed to have good sensitivity. Similarly, Examples 3-19 and 3-20, which formed the negative-type pattern, had a smaller optimal exposure dose than Comparative Examples 2-11 and 2-12, which formed a similar negative-type pattern, and confirmed to have good sensitivity.

From the results shown in Table 3 and Table 4, Examples 3-1 to 3-18, which formed the positive-type pattern, were confirmed to have a mostly larger EL than Comparative Examples 2-1 to 2-10, which formed a similar positive-type pattern. Similarly, Examples 3-19 and 3-20, which formed the negative-type pattern, were confirmed to have a mostly larger EL than Comparative Examples 2-11 and 2-12, which formed a similar negative-type pattern.

From the results shown in Table 3 and Table 4, Examples 3-1 to 3-18, which formed the positive-type pattern, were confirmed to have a lower LWR, larger DOF, and smaller collapse limit size than Comparative Examples 2-1 to 2-10, which formed a similar positive-type pattern. Similarly, from the results shown in Table 3 and Table 4, Examples 3-19 and 3-20, which formed the negative-type pattern, were confirmed to have a lower LWR, larger DOF, and smaller collapse limit size than Comparative Examples 2-11 and 2-12, which formed a similar negative-type pattern.

That is, from the results shown in Table 3 and Table 4, the chemically amplified resist composition containing the inventive quencher (acid diffusion inhibitor) was found to have good sensitivity and excellent EL, LWR, and DOF with both of the positive-type and negative-type resist patterns. In addition, it was confirmed that the above chemically amplified resist composition has a small collapse limit value, and hardly causes pattern collapse even in fine patterning.

Meanwhile, the resist compositions CR-1 to CR-12, which contained no inventive onium salt as a quencher and, instead thereof, which contained any one of the onium salts cSQ-1 to cSQ-3 with the anion structure not having the —OR^{ALU} group nor iodine atom in the general formula (1) or contained the onium salt cSQ-4 with the anion structure not having an iodine atom, were found to have poor LWR and DOF compared with the examples of the inventive resist compositions and found to be likely to cause pattern collapse.

### [5] EUV Lithography Evaluation (2)

### Examples 4-1 to 4-20 and Comparative Examples 3-1 to 3-12

Each of the resist compositions shown in Table 1 and Table 2 was applied by spin-coating on a Si substrate on which a silicon-containing spin-on hard mask SHB-A940 (silicon content of 43 mass %), manufactured by Shin-Etsu Chemical Co., Ltd., was formed with 20 nm in film thickness. The applied composition was prebaked at 100° C. for 60 seconds using a hot plate to produce a resist film with 60 nm in film thickness. Then, the resist film was exposed by using an EUV scanner NXE3400 (NA 0.33, σ 0.9/0.6, quadrupole illumination, hole pattern mask with 44 nm in pitch on wafer size and +20% bias), manufactured by ASML Holding N.V. Then, PEB was performed at a temperature shown in Table 5 and Table 6 for 60 seconds on a hot plate, and development was performed with a 2.38 mass % aqueous TMAH solution for 30 seconds to obtain a hole pattern with 22 nm in size in Examples 4-1 to 4-18 and Comparative Examples 3-1 to 3-10, and a dot pattern with 22 nm in size in Examples 4-19 and 4-20 and Comparative Examples 3-11 and 3-12.

Using a length-measurement SEM (CG6300), manufactured by Hitachi High-Technologies Corporation, an exposure dose when the hole or dot pattern was formed with 22 nm in size was measured to specify this exposure dose as a sensitivity. Sizes of 50 holes or dots in this time were measured, and a tripled value (3σ) of a standard variation (σ) calculated from the results was determined as CDU. Table 5 and Table 6 show the results.

TABLE 5

| | | | Optimal | |
|---|---|---|---|---|
| | | PEB | exposure | |
| | Resist | temperature | dose | CDU |
| | composition | (° C.) | (mJ/cm²) | (nm) |
| Example 4-1 | R-1 | 85 | 32 | 3.2 |
| Example 4-2 | R-2 | 80 | 33 | 3.1 |
| Example 4-3 | R-3 | 85 | 32 | 3.2 |
| Example 4-4 | R-4 | 80 | 31 | 3.2 |
| Example 4-5 | R-5 | 85 | 32 | 3.2 |
| Example 4-6 | R-6 | 80 | 32 | 3.1 |
| Example 4-7 | R-7 | 80 | 31 | 3 |
| Example 4-8 | R-8 | 80 | 33 | 3.1 |
| Example 4-9 | R-9 | 85 | 32 | 3.4 |
| Example 4-10 | R-10 | 80 | 29 | 3.3 |
| Example 4-11 | R-11 | 85 | 28 | 3.1 |
| Example 4-12 | R-12 | 80 | 27 | 3.2 |
| Example 4-13 | R-13 | 80 | 27 | 3.3 |
| Example 4-14 | R-14 | 85 | 28 | 3.2 |
| Example 4-15 | R-15 | 85 | 26 | 3.1 |
| Example 4-16 | R-16 | 80 | 27 | 3.2 |
| Example 4-17 | R-17 | 80 | 28 | 3.1 |
| Example 4-18 | R-18 | 85 | 27 | 3 |
| Example 4-19 | R-19 | 105 | 33 | 3.3 |
| Example 4-20 | R-20 | 110 | 34 | 3.4 |

TABLE 6

| | | | Optimal | |
|---|---|---|---|---|
| | | PEB | exposure | |
| | Resist | temperature | dose | CDU |
| | composition | (° C.) | (mJ/cm²) | (nm) |
| Comparative | CR-1 | 80 | 35 | 3.6 |
| Example 3-1 | | | | |
| Comparative | CR-2 | 85 | 36 | 3.5 |
| Example 3-2 | | | | |
| Comparative | CR-3 | 85 | 36 | 3.6 |
| Example 3-3 | | | | |
| Comparative | CR-4 | 80 | 35 | 3.4 |
| Example 3-4 | | | | |
| Comparative | CR-5 | 80 | 36 | 3.8 |
| Example 3-5 | | | | |
| Comparative | CR-6 | 80 | 37 | 3.6 |
| Example 3-6 | | | | |
| Comparative | CR-7 | 85 | 35 | 3.7 |
| Example 3-7 | | | | |
| Comparative | CR-8 | 80 | 36 | 3.6 |
| Example 3-8 | | | | |
| Comparative | CR-9 | 85 | 35 | 3.7 |
| Example 3-9 | | | | |
| Comparative | CR-10 | 80 | 37 | 3.8 |
| Example 3-10 | | | | |
| Comparative | CR-11 | 105 | 41 | 4.5 |
| Example 3-11 | | | | |
| Comparative | CR-12 | 110 | 39 | 4.9 |
| Example 3-12 | | | | |

From the results shown in Table 5 and Table 6, it was confirmed that Examples 4-1 to 4-18, which formed the hole pattern, exhibited smaller CDU than Comparative Examples 3-1 to 3-10, which formed a similar hole pattern, and had excellent CDU. In addition, it was confirmed that Examples 4-19 and 4-20, which formed the dot pattern, exhibited smaller CDU than Comparative Examples 3-11 and 3-12, which formed a similar dot pattern, and had excellent CDU. That is, from the results shown in Table 5 and Table 6, the chemically amplified resist composition containing the inventive quencher (acid diffusion inhibitor) was confirmed to have good sensitivity and excellent CDU with both of the positive-type and negative-type resist pattern.

Meanwhile, the resist compositions CR-1 to CR-12, which contained no inventive onium salt as a quencher and which contained any one of the onium salts cSQ-1 to cSQ-3 having the onium anion not having the —OR^{ALU} group nor iodine atom in the general formula (1) or which contained the onium salt cSQ-4 not having an iodine atom, were found to have poor CDU compared with the examples of the inventive resist compositions.

With each resist composition, precipitation of the quencher was not observed from preparation of the resist compositions R-1 to R-20 containing the components shown in Table 1 to use in each of the above Examples.

The present specification includes the following aspects.

[1] An onium salt represented by the following general formula (1),

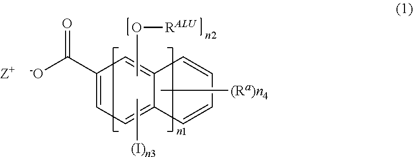

wherein R^{ALU} represents any one of a tertiary ether, tertiary carbonate or acetal formed together with the adjacent oxygen atom, and optionally having a heteroatom; I represents an iodine atom; R^{a} represents a hydrocarbyl group having 1 to 20 carbon atoms and optionally having a heteroatom; n1 represents an integer of 0 or 1; n2 and n3 represent an integer of 1 or 2, when n2 and n3 represent 1, I and —O—R^{ALU} are bonded to carbon atoms adjacent to each other, and when any one or both of n2 and n3 represent 2, one of I and one of —O—R^{ALU} are bonded to carbon atoms adjacent to each other; n4 represents an integer of 0 to 3; when n4≥2, a plurality of R^{a} are optionally bonded to each other to form a cyclic structure together with the carbon atom to which these are bonded; and Z⁺ represents an onium cation.

[2] The onium salt according to [1], wherein the structure of R^{ALU} in the general formula (1) is represented by the following general formula (ALU-1) or (ALU-2),

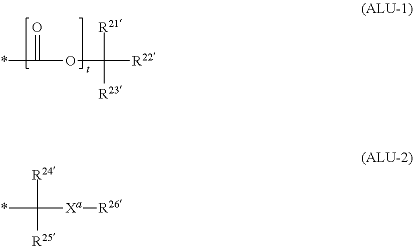

wherein in the formula (ALU-1), R^{21′}, R^{22′}, and R^{23′} each independently represent a hydrocarbyl group having 1 to 12 carbon atoms and optionally having a heteroatom, any two of R^{21′}, R^{22′}, and R^{23′} being optionally bonded to each other to form a ring; “t” represents an integer of 0 or 1; in the formula (ALU-2), R^{24′} and R^{25′} each independently represent a hydrogen atom or a hydrocarbyl group having 1 to 10 carbon atoms; R^{26′} represents a hydrocarbyl group having 1 to 20 carbon atoms, or is optionally bonded to R^{24′} or R^{25′} each other to form a heterocyclic group having 3 to 20 carbon atoms together with X^{a} and the carbon atom to which these are bonded; each of —CH₂— contained in the hydrocarbyl group and the heterocyclic group is optionally substituted with —O— or —S—; X^{a} represents an oxygen atom or a sulfur atom; and “*” represents a bond to the adjacent oxygen atom.

[3] The onium salt according to [1] or [2], wherein Z⁺ in the general formula (1) represents an onium cation represented by any one of the following general formulae (Cation-1) to (Cation-3),

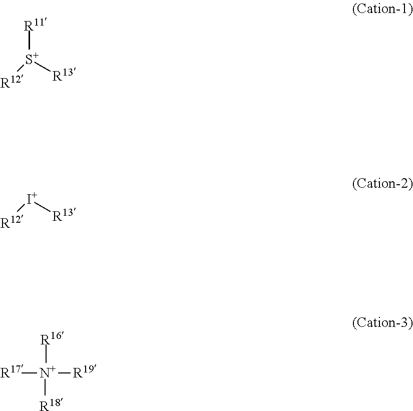

wherein in the formulae (Cation-1) to (Cation-3), R^{11′} to R^{19′} each independently represent a saturated or unsaturated, linear, branched, or cyclic hydrocarbyl group having 1 to 30 carbon atoms and optionally having a heteroatom.

[4] An acid diffusion inhibitor, comprising the onium salt according to any one of [1] to [3].

[5] A resist composition, comprising the acid diffusion inhibitor according to [4].

[6] The resist composition according to [5], further comprising an acid generator to generate an acid.

[7] The resist composition according to [6], wherein the acid generator generates a sulfonic acid, an imide acid, or a methide acid.

[8] The resist composition according to any one of [5] to [7], further comprising an organic solvent.

[9] The resist composition according to any one of [5] to [8], further comprising a base polymer.

[10] The resist composition according to [9], wherein the base polymer comprises a repeating unit represented by the following general formula (a1) and/or a repeating unit represented by the following general formula (a2),

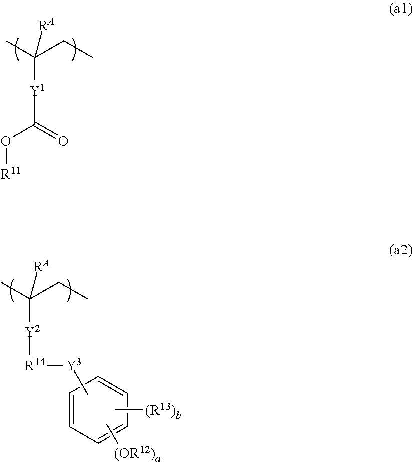

wherein R^{A} each independently represents a hydrogen atom or a methyl group; Y¹ represents a single bond, a phenylene group, a naphthylene group, or a linking group having 1 to 12 carbon atoms and having at least one selected from an ester bond and a lactone ring; Y² represents a single bond or an ester bond; Y³ represents a single bond, an ether bond, or an ester bond; R¹¹ and R¹² each independently represents an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, a part of carbon atoms therein being optionally substituted with an ether bond or an ester bond; “a” represents 1 or 2; “b” represents an integer of 0 to 4, and 1≤a+b≤5.

[11] The resist composition according to any one of [5] to [10], wherein the resist composition is a chemically amplified positive-type resist composition.

[12] The resist composition according to any one of [5] to [9], wherein the resist composition is a chemically amplified negative-type resist composition.

[13] The resist composition according to [9], wherein the base polymer has no acid-labile group.

[14] The resist composition according to [9], [10], or [13], wherein the base polymer further comprises at least one selected from repeating units represented by the following general formulae (f1) to (f3),

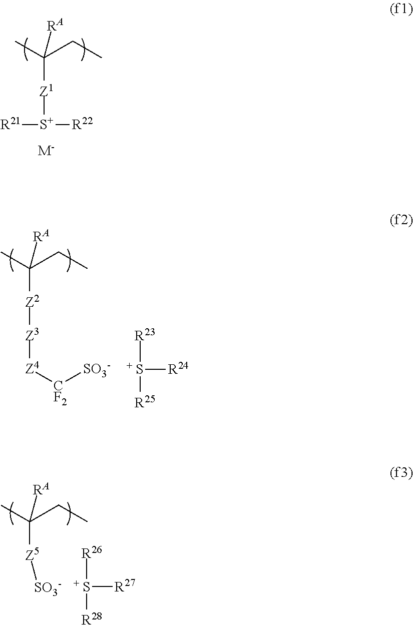

wherein R^{A} each independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)—O—Z¹¹—, or —C(═O)—NH—Z¹¹—; Z¹¹ represents an aliphatic hydrocarbylene group having 1 to 6 carbon atoms, a phenylene group, a naphthylene group, or a group having 7 to 18 carbon atoms obtained by combining these groups, Z¹¹ optionally having a carbonyl group, an ester bond, an ether bond, or a hydroxy group; Z² represents a single bond or an ester bond; Z³ represents a single bond, —Z³¹—C(═O)—O—, —Z³¹—O—, or —Z³¹—O—C(═O)—; Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—; Z⁵¹ represents an aliphatic hydrocarbylene group having 1 to 6 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group; R²¹ to R²⁸ each independently represent a halogen atom or a hydrocarbyl group having 1 to 20 carbon atoms and optionally having a heteroatom; R²³ and R²⁴ or R²⁶ and R²⁷ are optionally bonded to each other to form a ring together with the sulfur atom to which these groups are bonded; and M⁻ represents a non-nucleophilic counterion.

[15] The resist composition according to any one of [5] to [14], further comprising a surfactant.

[16] A patterning process, comprising steps of: forming a resist film on a substrate by using the resist composition according to any one of [5] to [15]; exposing the resist film to high-energy ray; and developing the exposed resist film by using a developer.

[17] The patterning process according to [16], wherein KrF excimer laser light, ArF excimer laser light, electron beam, or extreme ultraviolet ray having a wavelength of 3 to 15 nm is used as the high-energy ray.

It should be noted that the present invention is not limited to the above-described embodiments. The embodiments are just examples, and any examples that substantially have the same feature and demonstrate the same functions and effects as those in the technical concept disclosed in claims of the present invention are included in the technical scope of the present invention.

1. An onium salt represented by the following general formula (1), 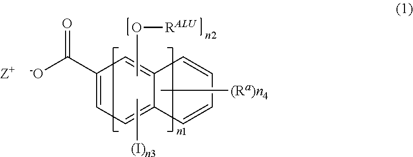
wherein R^{ALU} represents any one of a tertiary ether, tertiary carbonate or acetal formed together with the adjacent oxygen atom, and optionally having a heteroatom; I represents an iodine atom; R^{a} represents a hydrocarbyl group having 1 to 20 carbon atoms and optionally having a heteroatom; n1 represents an integer of 0 or 1; n2 and n3 represent an integer of 1 or 2, when n2 and n3 represent 1, I and —O—R^{ALU} are bonded to carbon atoms adjacent to each other, and when any one or both of n2 and n3 represent 2, one of I and one of —O—R^{ALU} are bonded to carbon atoms adjacent to each other; n4 represents an integer of 0 to 3; when n4≥2, a plurality of R^{a} are optionally bonded to each other to form a cyclic structure together with the carbon atom to which these are bonded; and Z⁺ represents an onium cation.

2. The onium salt according to claim 1, wherein the structure of R^{ALU} in the general formula (1) is represented by the following general formula (ALU-1) or (ALU-2), 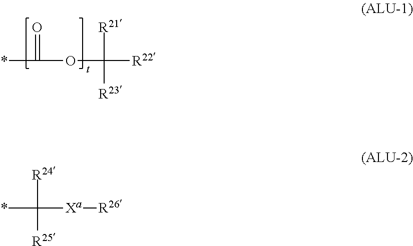
wherein in the formula (ALU-1), R^{21′}, R^{22′}, and R^{23′} each independently represent a hydrocarbyl group having 1 to 12 carbon atoms and optionally having a heteroatom, any two of R^{21′}, R^{22′}, and R^{23′} being optionally bonded to each other to form a ring; “t” represents an integer of 0 or 1; in the formula (ALU-2), R^{24′} and R^{25′} each independently represent a hydrogen atom or a hydrocarbyl group having 1 to 10 carbon atoms; R^{26′} represents a hydrocarbyl group having 1 to 20 carbon atoms, or is optionally bonded to R^{24′} or R^{25′} each other to form a heterocyclic group having 3 to 20 carbon atoms together with X^{a} and the carbon atom to which these are bonded; each of —CH₂— contained in the hydrocarbyl group and the heterocyclic group is optionally substituted with —O— or —S—; X^{a} represents an oxygen atom or a sulfur atom; and “*” represents a bond to the adjacent oxygen atom.

3. The onium salt according to claim 1, wherein Z⁺ in the general formula (1) represents an onium cation represented by any one of the following general formulae (Cation-1) to (Cation-3), 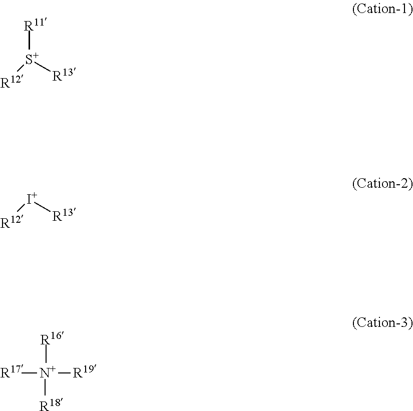
wherein in the formulae (Cation-1) to (Cation-3), R^{11′} to R^{19′} each independently represent a saturated or unsaturated, linear, branched, or cyclic hydrocarbyl group having 1 to 30 carbon atoms and optionally having a heteroatom.

4. An acid diffusion inhibitor, comprising the onium salt according to claim 1.

5. A resist composition, comprising the acid diffusion inhibitor according to claim 4.

6. The resist composition according to claim 5, further comprising an acid generator to generate an acid.

7. The resist composition according to claim 6, wherein the acid generator generates a sulfonic acid, an imide acid, or a methide acid.

8. The resist composition according to claim 5, further comprising an organic solvent.

9. The resist composition according to claim 5, further comprising a base polymer.

10. The resist composition according to claim 9, wherein the base polymer comprises a repeating unit represented by the following general formula (a1) and/or a repeating unit represented by the following general formula (a2), 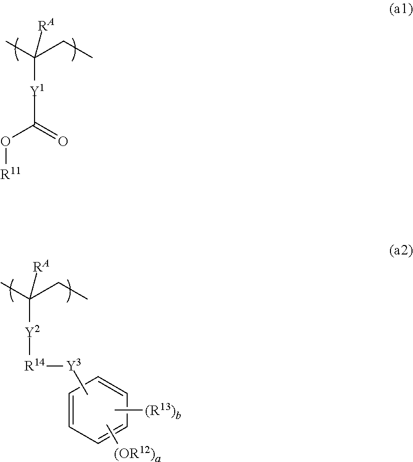
wherein R^{A} each independently represents a hydrogen atom or a methyl group; Y¹ represents a single bond, a phenylene group, a naphthylene group, or a linking group having 1 to 12 carbon atoms and having at least one selected from an ester bond and a lactone ring; Y² represents a single bond or an ester bond; Y³ represents a single bond, an ether bond, or an ester bond; R¹¹ and R¹² each independently represents an acid-labile group; R¹³ represents a fluorine atom, a trifluoromethyl group, a cyano group, or a saturated hydrocarbyl group having 1 to 6 carbon atoms; R¹⁴ represents a single bond or an alkanediyl group having 1 to 6 carbon atoms, a part of carbon atoms therein being optionally substituted with an ether bond or an ester bond; “a” represents 1 or 2; “b” represents an integer of 0 to 4, and 1≤a+b≤5.

11. The resist composition according to claim 5, wherein the resist composition is a chemically amplified positive-type resist composition.

12. The resist composition according to claim 5, wherein the resist composition is a chemically amplified negative-type resist composition.

13. The resist composition according to claim 9, wherein the base polymer has no acid-labile group.

14. The resist composition according to claim 9, wherein the base polymer further comprises at least one selected from repeating units represented by the following general formulae (f1) to (f3), 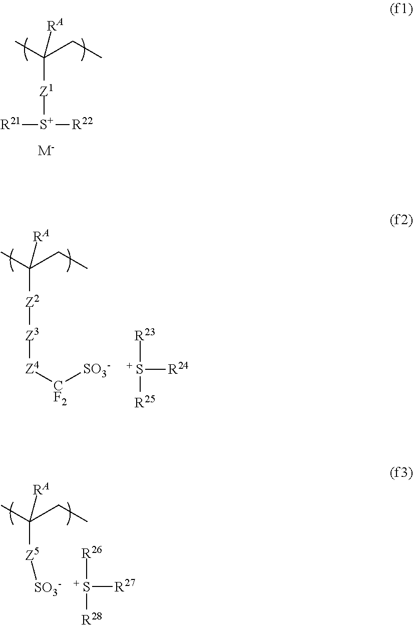
wherein R^{A} each independently represents a hydrogen atom or a methyl group; Z¹ represents a single bond, an aliphatic hydrocarbylene group having 1 to 6 carbon atoms, a phenylene group, a naphthylene group, an ester bond, a group having 7 to 18 carbon atoms obtained by combining these groups, —O—Z¹¹—, —C(═O)—O—Z¹¹—, or —C(═O)—NH—Z¹¹—; Z¹¹ represents an aliphatic hydrocarbylene group having 1 to 6 carbon atoms, a phenylene group, a naphthylene group, or a group having 7 to 18 carbon atoms obtained by combining these groups, Z¹¹ optionally having a carbonyl group, an ester bond, an ether bond, or a hydroxy group; Z² represents a single bond or an ester bond; Z³ represents a single bond, —Z³¹—C(═O)—O—, —Z³¹—O—, or —Z³¹—O—C(═O)—; Z³¹ represents a hydrocarbylene group having 1 to 12 carbon atoms, a phenylene group, or a group having 7 to 18 carbon atoms obtained by combining these groups, Z³¹ optionally having a carbonyl group, an ester bond, an ether bond, an iodine atom, or a bromine atom; Z⁴ represents a methylene group, a 2,2,2-trifluoro-1,1-ethanediyl group, or a carbonyl group; Z⁵ represents a single bond, a methylene group, an ethylene group, a phenylene group, a fluorinated phenylene group, a phenylene group substituted with a trifluoromethyl group, —O—Z⁵¹—, —C(═O)—O—Z⁵¹—, or —C(═O)—NH—Z⁵¹—; Z⁵¹ represents an aliphatic hydrocarbylene group having 1 to 6 carbon atoms, a phenylene group, a fluorinated phenylene group, a phenylene group substituted with a trifluoromethyl group, or a combination thereof, Z⁵¹ optionally having a carbonyl group, an ester bond, an ether bond, a halogen atom, and/or a hydroxy group; R²¹ to R²⁸ each independently represent a halogen atom or a hydrocarbyl group having 1 to 20 carbon atoms and optionally having a heteroatom; R²³ and R²⁴ or R²⁶ and R²⁷ are optionally bonded to each other to form a ring together with the sulfur atom to which these groups are bonded; and M⁻ represents a non-nucleophilic counterion.

15. The resist composition according to claim 5, further comprising a surfactant.

16. A patterning process, comprising steps of:
forming a resist film on a substrate by using the resist composition according to claim 5;
exposing the resist film to high-energy ray; and
developing the exposed resist film by using a developer.

17. The patterning process according to claim 16, wherein KrF excimer laser light, ArF excimer laser light, electron beam, or extreme ultraviolet ray having a wavelength of 3 to 15 nm is used as the high-energy ray.
